(19) United States
(12) Patent Application Publication
(10) Pub. No.: US 2011/0301228 A1
(43) Pub. Date: Dec. 8, 2011

(54) GENE THERAPY VECTOR FOR TREATMENT OF STEROID GLAUCOMA

(75) Inventors: Teresa Borras, Chapel Hill, NC (US); Maria-Grazia Spiga, Cary, NC (US)

(73) Assignee: THE UNIVERSITY OF NORTH CAROLINA AT CHAPEL HILL, Chapel Hill, NC (US)

(21) Appl. No.: 13/147,013

(22) PCT Filed: Feb. 11, 2010
(86) PCT No.: PCT/US2010/023933

§ 371 Date: Aug. 25, 2011

Related U.S. Application Data
(60) Provisional application No. 61/151,654, filed on Feb. 11, 2009.

(51) Int. Cl. A61K 48/00; A61P 29/00; A61P 27/02; A61P 27/06; C12N 15/85; C12N 15/861

(52) U.S. Cl. 514/44 R; 435/320.1

(57) ABSTRACT

The presently disclosed subject matter provides an inducible vector comprising a therapeutic gene. In some embodiments a method is provided for treating steroid glaucoma. In some embodiments a method is provided for preventing elevated intraocular pressure in a subject receiving steroid treatment. In some embodiments a method is provided for reversing elevated intraocular pressure in a subject receiving steroid treatment. In some embodiments a steroid treatment method is provided. Also provided are pharmaceutical compositions comprising an inducible vector.

### CROSS-REFERENCE TO RELATED APPLICATIONS

This application claims benefit of U.S. Provisional Patent Application Ser. No. 61/151,654, entitled “Gene Therapy Vector for Treatment of Steroid Glaucoma”, filed Feb. 11, 2009, which is herein incorporated by reference in its entirety.

### GOVERNMENT INTEREST

This invention was made with United States government support under Grant Nos. EY11906 and EY13126 awarded by National Institutes of Health of the United States. The United States government has certain rights in the invention.

### TECHNICAL FIELD

This presently disclosed subject matter generally relates to gene therapy, to gene therapy vectors and constructs, to inducible gene therapy vectors and constructs, and to compositions and pharmaceutical compositions comprising the same. The presently disclosed subject matter relates to uses for the gene therapy vectors, constructs and compositions. The presently disclosed subject matter also relates to gene therapy methods, to methods for treating conditions associated with steroid therapy. The presently disclosed subject matter also relates to gene therapy methods for treating steroid glaucoma and associated conditions, and to methods for preventing steroid glaucoma. Further embodiments are described below.

### BACKGROUND

Glucocorticosteroids, also known as glucocorticoids, exhibit therapeutic versatility given their common usage as anti-inflammatory, immunosuppressive, and anti-angiogenic agents (Clark A. F., 1997 Expert Opin. Investig. Drugs 6:1867-1877; Clark A. F., 2007 Surv. Ophthalmol. 52 (Suppl 1):S26-34). However, glucocorticosteroids and treatments and therapies designed to use glucocorticoids also elicit adverse ocular effects. Steroid treatments, and in some cases ocular steroid treatment, can cause cataracts and lead to increased extracellular matrix (ECM) deposition and elevated intraocular pressure (IOP) in a substantial number of eye patients. The resulting condition is referred to as steroid glaucoma. Studies report glucocorticoid treatment leads to increased ECM deposition in the trabecular meshwork (TM) and to elevated IOP in 40% of patients (Gillies et al., 2005 Ophthalmology 112:139-143). Individuals susceptible to steroid-induced elevated IOP can require treatment for glaucoma.

The phenomenon of glucocorticosteroid-induced ocular hypertension has been recognized for decades (McLean J., 1950 Trans Am Ophthalmol Soc 48:293-296), and a number of predisposing risk factors have been identified among patients receiving various corticosteroid treatments (Jones, R. and Rhee, D. J., 2006 Curr. Opin. Ophthalmol. 17:163-167; Kersey, J. P, and D.C. Broadway, 2006 Eye 20:407-416). Yet the mechanisms by which glucocorticosteroids induce the IOP elevation have not been determined.

As such, there remains a need for an improved understanding of the cellular processes eliciting corticosteroid-induced ocular hypertension and steroid glaucoma. There also remains a need for therapeutic compositions and methods for treating and/or preventing the untoward effects of glucocorticoid therapy.

### SUMMARY

In some embodiments the presently disclosed subject matter provides a steroid-inducible vector comprising a coding sequence for a polypeptide of interest, a minimal promoter and a steroid response element (SRE), wherein the coding sequence is under the transcriptional control of the SRE, wherein the coding sequence corresponds to a gene susceptible to altered expression in the presence of a steroid in vivo. In some embodiments, the vector is an adenovirus vector. In some embodiments, the SRE is a glucocorticoid response element (GRE). In some embodiments, the GRE increases transcription of the coding sequence in the presence of a steroid selected from the group consisting of dexamethasone, triamcinalone acetonide, prednisolone acetate, and combinations thereof. In some embodiments, the polypeptide of interest is MMP1. In some embodiments, the coding sequence for MMP1 comprises a nucleotide sequence of SEQ ID NO: 3, or a nucleotide sequence 95% identical to SEQ ID NO: 3. In some embodiments, the MMP1 polypeptide comprises an amino acid sequence of SEQ ID NO: 4, or an amino acid sequence 95% identical to SEQ ID NO: 4.

In some embodiments the presently disclosed subject matter provides a method of treating steroid glaucoma in a subject in need thereof, the method comprising: providing a subject suffering from steroid glaucoma, providing a vector comprising a coding sequence for a polypeptide of interest, a minimal promoter and a SRE, wherein the coding sequence is under the transcriptional control of the SRE, wherein the coding sequence corresponds to a gene susceptible to altered expression in the presence of a steroid in vivo, and administering the vector to the subject, wherein the steroid glaucoma is treated. In some embodiments, the steroid glaucoma comprises elevated intraocular pressure (IOP). In some embodiments, the elevated IOP is decreased. In some embodiments, the steroid glaucoma comprises increased extracellular matrix (ECM) deposition. In some embodiments, the ECM deposition is decreased. In some embodiments, the vector is an adenovirus vector. In some embodiments, the SRE is a glucocorticoid response element (GRE). In some embodiments, the polypeptide of interest is MMP1. In some embodiments, administering the vector comprises administering the vector to an ocular tissue of the subject. In some embodiments, the subject is a mammal. In some embodiments, the subject is receiving a steroid treatment, wherein the steroid is a glucocorticoid selected from the group consisting of dexamethasone, triamcinalone acetonide, prednisolone acetate, and combinations thereof.

In some embodiments the presently disclosed subject matter provides a method of preventing elevated intraocular pressure (IOP) in a subject receiving steroid treatment, the method comprising: providing a subject receiving steroid treatment, providing a vector comprising a coding sequence for a polypeptide of interest, a minimal promoter and a SRE, wherein coding sequence is under the transcriptional control of the SRE, wherein the coding sequence corresponds to a gene susceptible to altered expression in the presence of a steroid in vivo, and administering the vector to the subject, wherein elevated IOP in the subject is prevented. In some embodiments, the vector is an adenovirus vector. In some embodiments, the SRE is a glucocorticoid response element (GRE). In some embodiments, the polypeptide of interest is MMP1. In some embodiments, the subject is a mammal. In some embodiments, the steroid treatment comprises the administration of a glucocorticoid to the subject, wherein the glucocorticoid is selected from the group consisting of dexamethasone, triamcinalone acetonide, prednisolone acetate, and combinations thereof. In some embodiments, administering the vector comprises administering the vector to an ocular tissue of the subject.

In some embodiments the presently disclosed subject matter provides a method of reversing elevated intraocular pressure (IOP) in a subject receiving steroid treatment, the method comprising: providing a subject receiving steroid treatment, wherein the subject has elevated IOP, providing a vector comprising a coding sequence for a polypeptide of interest, a minimal promoter and a SRE, wherein the coding sequence is under the transcriptional control of the SRE, wherein the coding sequence corresponds to a gene susceptible to altered expression in the presence of a steroid in vivo, and administering the vector to the subject, wherein the elevated IOP in the subject is reversed. In some embodiments, the vector is an adenovirus vector. In some embodiments, the SRE is a glucocorticoid response element (GRE). In some embodiments, the polypeptide of interest is MMP1. In some embodiments, the subject is a mammal. In some embodiments, the steroid treatment comprises the administration of a glucocorticoid to the subject, wherein the glucocorticoid is selected from the group consisting of dexamethasone, triamcinalone acetonide, prednisolone acetate, and combinations thereof. In some embodiments, administering the vector comprises administering the vector to an ocular tissue of the subject.

In some embodiments the presently disclosed subject matter provides a steroid treatment method comprising: providing a subject in need of steroid treatment, administering a vector comprising a coding sequence for a polypeptide of interest, a minimal promoter and a SRE, wherein the coding sequence is under the transcriptional control of the SRE, wherein the coding sequence corresponds to a gene susceptible to altered expression in the presence of a steroid in vivo, and administering a steroid to the subject. In some embodiments, the subject in need of steroid treatment comprises a subject suffering from inflammation, ocular inflammation, macular edema, choroidal neovascularization, or any other eye or systemic condition requiring administration of a steroid. In some embodiments, the vector is administered prior to, simultaneously, or after steroid administration. In some embodiments, the vector is an adenovirus vector. In some embodiments, the SRE is a glucocorticoid response element (GRE). In some embodiments, the polypeptide of interest is MMP1. In some embodiments, the subject is a mammal. In some embodiments, the steroid is selected from the group consisting of dexamethasone, triamcinalone acetonide, prednisolone acetate, and combinations thereof.

In some embodiments the presently disclosed subject matter provides a method of treating or preventing a condition associated with steroid treatment in a subject, the method comprising: providing a subject receiving steroid treatment, providing a vector comprising a coding sequence for a polypeptide of interest, a minimal promoter and a SRE, wherein the coding sequence is under the transcriptional control of the SRE, wherein the coding sequence corresponds to a gene susceptible to altered expression in the presence of a steroid in vivo, and administering the vector to the subject. In some embodiments, the vector is an adenovirus vector. In some embodiments, the SRE is a glucocorticoid response element (GRE). In some embodiments, the polypeptide of interest is MMP1. In some embodiments, the subject is a mammal. In some embodiments, the steroid treatment comprises the administration of a glucocorticoid to the subject, wherein the glucocorticoid is selected from the group consisting of dexamethasone, triamcinalone acetonide, prednisolone acetate, and combinations thereof. In some embodiments, administering the vector comprises administering the vector to an ocular tissue of the subject. In some embodiments, the vector is administered prior to, simultaneously, or after steroid administration.

In some embodiments the presently disclosed subject matter provides a composition comprising the steroid-inducible vector of claim 1 and a pharmaceutically acceptable carrier.

It is an object of the presently disclosed subject matter to provide a genetic construct that can in some examples be employed in the treatment of steroid glaucoma. This and others objects are achieved in whole or in part by the presently disclosed subject matter.

An object of the presently disclosed subject matter having been stated above, other objects and advantages of the presently disclosed subject matter will become apparent to those skilled in the art after a study of the following description and Examples.

### BRIEF DESCRIPTION OF THE DRAWINGS

FIGS. 1A-1D illustrate the effect of glucocorticoids on endogenous MMP1 expression in human trabecular meshwork cells. Primary HTM cells were treated with the indicated glucocorticoids in serum-containing medium. Control cells were treated with drug vehicle (untreated, UNT). After the treatment, cells were processed for RNA extraction, RT reaction and real-time TaqMan PCR using MMP1 and 18S TaqMan probes. Changes in gene expression were measured in relative quantitative units to the vehicle treated cells (n=number of measurements in a representative experiment). FIG. 1A is a bar graph showing relative quantities of MMP1 cDNA in confluent cells exposed to 0.1 μM DEX for up to 6 days (n=3 each). In one well, DEX was removed at 3 days and harvested 3 days later (3d DEX+3d No DEX; n=6). FIG. 1B is an autoradiograph of a Western blot of MMP1 and myocilin. Media from DEX-treated cells was assayed in 4-15% SDS-PAGE gels and probed with anti-human MMP1 antibody; after stripping, the membrane was re-probed with anti-human myocilin antibody as internal control. FIG. 1C is a bar graph of the relative quantity of MMP1 cDNA in subconfluent cells treated with triamcinolone acetonide (TA, 0.1 mg/ml) for 12 hours and untreated (UNT) cells (n=3 each). FIG. 1D is a bar graph of the relative quantity of MMP1 cDNA in subconfluent cells treated with prednisolone 21-acetate (PRED, 80 μg/ml) for 24 hours and untreated (UNT) cells (n=3 each). *p≦0.013. FIGS. 1A-1D illustrate that HTM cells treated with any one of three different glucocorticoids greatly down-regulated expression of endogenous MMP1.

FIGS. 2A-2C are schematic representations of the construction of glucocorticoid inducible virus vectors expressing MMP1. FIG. 2A is a schematic of a glucocorticoid inducible shuttle vector containing the full-coding MMP1 (pMG17) which was generated by first inserting the MMP1 amplified RT from AdhTIG3-infected cells downstream of the TrBlk.GRE.pTAL element of plasmid pGRE-Luc vector (pMG12); this was followed by subcloning the full GRE.MMP1 cassette into pShuttle vector using NotI/SalI enzymes. FIG. 2B is a schematic representation of AdhGRE.MMP1 recombinant virus DNA generated from a plasmid obtained by overlapping recombination of electroporated linear pMG17 DNA into BJ5183-Ad1 cells, which contain the adenovirus backbone vector. FIG. 2C is a schematic representation of AdhGRE.mutMMP1 recombinant virus DNA generated in a similar matter, except that the full coding MMP1 cDNA contained two single-point mutations; one of the mutations is at the catalytic site.

FIGS. 3A-3C show DEX-induced overproduction of recombinant MMP1 in HTM cells. Subconfluent primary HTM cells were infected with adenovirus vectors AdhGRE.MMP1 (wild-type) or AdhGRE.mutMMP1 (mutant). Cells were treated with 0.1 μM DEX at infection time and every 2-3 days thereafter. Control cells were treated with vehicle (untreated, UNT). Five days post-infection and treatment, cells were harvested for RNA or protein and processed for TaqMan assays or western blot analysis; (n=number of measurements in a representative experiment). FIG. 3A is a bar graph showing fold changes of wild-type and mutant MMP1 cDNA expression in infected/DEX treated cells over infected/untreated controls normalized to 18S (n=3 each). FIGS. 3B and 3C are autoradiographs of Western blot analyses of cell associated (FIG. 3B) and secreted (FIG. 3C) MMP1 in infected/DEX treated and infected/untreated samples probed with anti-human MMP1 (n=3 each). *p≦5×10⁻⁶. Expression of MMP1 in infected cells (mRNA and protein) is highly upregulated in the presence of DEX but it is barely expressed in the absence of glucocorticoid treatment. High levels of mutant mRNA and protein obtained were expected since the mutation only blocks MMP1 activity.

FIG. 4 is a bar graph showing the reversal of DEX induction of MMP1 in wild-type virus-infected cells upon removing the glucocorticoid. Subconfluent primary HTM cells in 6-well plates were infected with AdhGRE-MMP1 and treated with 0.1 μM DEX at t=0. One well was left untreated (UNT). DEX was removed from two infected wells at t=3 days and added again to one of the two at 6 days. Cells were harvested at the indicated time points and processed for RNA, RT and TaqMan real-time with MMP1 and 18S probes. (n=number of measurements in a representative experiment). MMP1 was upregulated at 3 and 6 days post-DEX treatment. This transgene MMP1 upregulation disappeared upon removal of DEX and returned upon the re-application of the corticosteroid (n=3 each). *p≦0.0002. Cells continuously carrying the MMP1 gene transfer vector can turn on and off the transgene in the presence or absence of the glucocorticoid.

FIGS. 5A-5D show the enzymatic activity of secreted recombinant MMP1 in HTM cells. Subconfluent primary HTM cells were infected with adenovirus vectors comprising wild-type or mutant MMP1 (AdhGRE.MMP1 and AdhGRE.mutMMP1, respectively) and treated with 0.1 μM DEX at t=0. Media was concentrated 40×. FIG. 5A is an autoradiograph of a Western blot analyses. In FIG. 5A ten μl of media, collected 5 days post-wild-type infection, were incubated with 1 mM APMA for 3 hours to cleave pro-MMP1 enzyme-inhibitor complex (51 kDa) and release the active MMP1 (41 kDa). Commercial purified pro-MMP1 protein was used as positive control. Western blots were probed with an anti-MMP1 antibody which detects both latent and active form. In FIG. 5B five μl of serum-free media, collected 7 days post-infection, untreated and treated with DEX, were activated and incubated with rat tail native collagen type I (10 μg) for 2 hours. Samples were run on a 4-15% PAGE gel, stained with Coomassie blue and photographed. FIG. 5C is a schematic representation of the FRET assay: a MMP substrate peptide labeled with a 5-FAM (fluorophore) and QXL520 (quencher) releases the fluorophore after cleavage of the peptide by MMP1. Fluorescence is read using 490/520 nm EX/EM filters. FIG. 5D is a bar graph showing MMP1 activity. Ten μl of serum-containing media, collected 5 days post-infection, untreated or treated with DEX, were activated and incubated with the FRET peptide for 40 minutes at 37° C. (n=3 independent experiments). MMP1 activity was expressed in relative fluorescence units of the sample with higher activity. While the MMP1 produced by the mutant was inactive, the MMP1 produced by the wild-type adenovirus was activated by APMA, degraded native collagen type I, and cleaved the FRET peptide with high efficiency. *p=1×10⁻⁹.

FIGS. 6A and 6B show the characterization of adenovirus-delivered MMP1 in perfused human anterior segments of whole eye globes. Eye pairs from non glaucomatous donors were perfused to stable baseline with DMEM and followed by media exchange containing 0.1 μM DEX in both eyes. At this time, eyes were injected through an HPLC loop and perfusion continued with DMEM/DEX media. One eye received virus vehicle (OD) while the contralateral eye received 6.2×10⁹ vg of AdhGRE.MMP1 (OS). The trabecular meshwork tissue was dissected at the end of the experiment and harvested for RNA and TaqMan assays. Effluents were collected at 3 and 5 days post-infection and processed for the analysis of secreted MMP1. FIG. 6A is a bar graph showing the fold changes of MMP1 transgene expression (OS) over vehicle-injected (OD), using MMP1 and 18S TaqMan probes (eye pair #1, 5 days post-injection, n=3, p=1×10⁻⁸). FIG. 6B includes autoradiographs of Western blot analyses. Equivalent aliquots of concentrated effluents from the same eye pair analyzed by western blot with a human anti-MMP1 antibody. Delivery of the MMP1 transgene to the OS eye by the adenovirus vector is highly efficient. Perfusion with DEX results in the secretion of pro-MMP1 and MMP1 forms (latent and active).

FIGS. 7A and 7B show the functional activity of transgene MMP1 delivered to perfused human anterior segments of whole eye globes. Eye pairs from non glaucomatous donors were perfused to stable baseline with DMEM and followed by media exchange containing 0.1 μM DEX in both eyes (t=0). Eyes were injected through an HPLC loop and perfusion continued in DMEM/DEX media. One eye received virus vehicle (OD) while the contralateral eye received 6.2×10⁹ vg per dose of AdhGRE.MMP1 (OS). Effluents were collected at pre- and post-injection times, concentrated 40×, activated with APMA and assayed for MMP1 activity. FIG. 7A is a bar graph of MMP1 activity using ten μl effluents from OD (vehicle) and OS (wild-type MMP1 adenovirus) incubated with the FRET peptide for 40 minutes at 37° C. MMP1 activity was measured by quantifying the released fluorescence from the substrate peptide, and it was expressed in ratio of relative fluorescence units of the viral-treated over vehicle (OS/OD) (eye pair #2, injected twice at t=0 and t=24 h). FIG. 7B shows Western blot analyses of equivalent aliquots of effluents from eye pair #1 (injected once at t=0) with human anti-MMP1 and anti-collagen type I antibodies. The MMP1 protein produced by the transgene had high enzymatic activity.

FIGS. 8A-8F are graphical plots of IOP measurements in 6 sheep treated with adenoviral vectors of the presently disclosed subject matter. Each graph represents data from one animal. All animals were treated with 2 drops of 0.5% prednisolone acetate in both eyes starting on Day 0. Prednisolone instillations continued thrice daily until the day indicated. Arrows indicate the day the animals received a unilateral, intracameral injection of an adenoviral (Ad) vector (solid diamond); the contralateral eyes were not treated with viral vectors (uninjected; open square). Two animals received a null Ad (without transgene; FIGS. 8A and 8B), two received an Ad that carried a mutated MMP1 transgene without catalytic activity (FIGS. 8C and 8D), and two received an active human MMP1 transgene (FIGS. 8E and 8F). One animal (FIG. 8E) was sacrificed on Day 20. Note that in the eyes that received the active MMP1 transgene (FIGS. 8E and 8F), IOP returned to normal levels for at least 15 days.

FIG. 9 is a graphical plot of the IOP from 4 sheep treated with triamcinolone acetonide and adenoviral vectors according to the following regimen. Both eyes of each sheep received a single sub-Tenon injection of triamcinolone on Day 0. On Day 4, one eye of each sheep received a single intracameral injection of an adenoviral vector (Ad) that carried a mutated MMP1 transgene without catalytic activity (solid diamonds), while the contralateral eye received an Ad carrying an active human MMP1 transgene (open squares). Points are means±SEMs of the 4 eyes receiving the mutated transgene, and of the 4 fellow eyes receiving the active transgene. In this set of experiments, treatment with the active transgene lowered IOP to normal levels for 3 days.

FIGS. 10A and 10B are graphical plots of the IOPs from two sheep treated with triamcinolone acetonide and adenoviral vectors according to the following regimen. All eyes received vectors carrying the active transgene on Day 0 and were administered a single sub-Tenon injection of triamcinolone the next day. FIG. 10A shows the individual IOP values from each eye. FIG. 10B shows the means±SEMs for the 4 eyes. The active transgene offered protection against triamcinolone administration for at least 3 days.

FIG. 11 is a graphical plot of the IOP from one sheep treated with triamcinolone acetonide, prednisolone acetate and adenoviral vectors according to the following regimen. The right eye received a single sub-Tenon injection of triamcinolone on Day-14 (not shown). IOP measurements began on Day 0, at which point thrice-daily prednisolone instillations were begun on the left eye. Both eyes received intracameral injections of adenoviral vectors carrying active transgene on Day 3. See text for additional details.

FIG. 12 is a graphical plot of the IOP from one sheep treated with triamcinolone acetonide, prednisolone acetate and adenoviral vectors according to the following regimen. The right eye received a single sub-Tenon injection of triamcinolone on Day-14 (not shown). Adenoviral (Ad) vectors carrying the active transgene were injected intracamerally into the left eye on Day 0 and into the right eye on Day 1. Prednisolone was administered to the left eye during the days indicated. See text for additional details.

### BRIEF SUMMARY OF THE SEQUENCE LISTING

SEQ ID NO: 1 is a 2,069 base pair polynucleotide sequence of an expression cassette of a gene therapy construct of the presently disclosed subject matter. The expression cassette of SEQ ID NO: 1 comprises, among other things, a glucocorticoid response element (GRE) and matrix metalloproteinase (MMP1) coding sequence.

SEQ ID NO: 2 is a polynucleotide sequence encoding a GRE.

SEQ ID NO: 3 is a polynucleotide sequence encoding MMP1.

SEQ ID NO: 4 is a polypeptide sequence of MMP1, which can be encoded by SEQ ID NO:3.

SEQ ID NO: 5 is a polynucleotide sequence encoding MMP3.

SEQ ID NO: 6 is a polypeptide sequence of MMP3, which can be encoded by SEQ ID NO:5.

SEQ ID NO: 7 is a polynucleotide sequence encoding MMP10.

SEQ ID NO: 8 is a polypeptide sequence of MMP10, which can be encoded by SEQ ID NO:7.

SEQ ID NO: 9 is a polynucleotide sequence encoding MMP12.

SEQ ID NO: 10 is a polypeptide sequence of MMP12, which can be encoded by SEQ ID NO:9.

SEQ ID NO: 11 is a polynucleotide sequence encoding ADAM10.

SEQ ID NO: 12 is a polypeptide sequence of ADAM10, which can be encoded by SEQ ID NO:11.

SEQ ID NO: 13 is a polynucleotide sequence encoding ADAM19.

SEQ ID NO: 14 is a polypeptide sequence of ADAM19, which can be encoded by SEQ ID NO:13.

SEQ ID NO: 15 is a polynucleotide sequence encoding a ADAMTS28.

SEQ ID NO: 16 is a polypeptide sequence of a ADAMTS28, which can be encoded by SEQ ID NO:15.

SEQ ID NO: 17 is a polynucleotide sequence encoding ADAMTS1.

SEQ ID NO: 18 is a polypeptide sequence of ADAMTS1, which can be encoded by SEQ ID NO:17.

SEQ ID NO: 19 is a polynucleotide sequence encoding ADAMTS3.

SEQ ID NO: 20 a polypeptide sequence of ADAMTS3, which can be encoded by SEQ ID NO:19

SEQ ID NO: 21 is a polynucleotide sequence encoding ADAMTS5.

SEQ ID NO: 22 is a polypeptide sequence of ADAMTS5, which can be encoded by SEQ ID NO:21.

SEQ ID NO: 23 is a polynucleotide sequence encoding Angiopoietin-like factor7/CDT6.

SEQ ID NO: 24 is a polypeptide sequence of Angiopoietin-like factor7/CDT6, which can be encoded by SEQ ID NO:23.

SEQ ID NO: 25 is a polynucleotide sequence encoding Angiopoietin-like factor2.

SEQ ID NO: 26 is a polypeptide sequence of Angiopoietin-like factor2, which can be encoded by SEQ ID NO:25.

SEQ ID NO: 27 is a polynucleotide sequence encoding Angiopoietin2.

SEQ ID NO: 28 is a polypeptide sequence of Angiopoietin2, which can be encoded by SEQ ID NO:27.

SEQ ID NO: 29 is a polynucleotide sequence encoding protein disulfide isomerase 2.

SEQ ID NO: 30 is a polypeptide sequence of protein disulfide isomerase 2, which can be encoded by SEQ ID NO:29.

SEQ ID NO: 31 is a polynucleotide sequence encoding protein disulfide isomerase 5.

SEQ ID NO: 32 is a polypeptide sequence of protein disulfide isomerase 5, which can be encoded by SEQ ID NO:31.

SEQ ID NO: 33 is a polynucleotide sequence encoding superoxide dismutase 2.

SEQ ID NO: 34 a polypeptide sequence of superoxide dismutase 3, which can be encoded by SEQ ID NO:33.

SEQ ID NO: 35 is a polynucleotide sequence encoding superoxide dismutase 3.

SEQ ID NO: 36 a polypeptide sequence of superoxide dismutase 2, which can be encoded by SEQ ID NO:35.

SEQ ID NO: 37 is a polynucleotide sequence encoding tropomyosin.

SEQ ID NO: 38 is a polypeptide sequence of tropomyosin, which can be encoded by SEQ ID NO:37.

SEQ ID NO: 39 is a polynucleotide sequence encoding Aldo-keto reductases 1C1.

SEQ ID NO: 40 is a polypeptide sequence of Aldo-keto reductases 1C1, which can be encoded by SEQ ID NO:39.

SEQ ID NO: 41 is a polynucleotide sequence encoding Aldo-keto reductases 1C3.

SEQ ID NO: 42 is a polypeptide sequence of Aldo-keto reductases 1C3, which can be encoded by SEQ ID NO:41.

SEQ ID NO: 43 is a polynucleotide sequence encoding Aldo-keto reductases 1B10.

SEQ ID NO: 44 is a polypeptide sequence of Aldo-keto reductases 1B10, which can be encoded by SEQ ID NO:43.

SEQ ID NO: 45 is a polynucleotide sequence encoding S100 calcium binding protein.

SEQ ID NO: 46 is a polypeptide sequence of S100 calcium binding protein, which can be encoded by SEQ ID NO:45.

SEQ ID NO: 47 is a polynucleotide sequence encoding Calreticulin.

SEQ ID NO: 48 is a polypeptide sequence of Calreticulin, which can be encoded by SEQ ID NO:47.

SEQ ID NO: 49 is a polynucleotide sequence encoding Chaperonin containing TCP1.

SEQ ID NO: 50 is a polypeptide sequence of Chaperonin containing TCP1, which can be encoded by SEQ ID NO:49.

SEQ ID NO: 51 is a polynucleotide sequence encoding Chitinase 3.

SEQ ID NO: 52 is a polypeptide sequence of Chitinase 3, which can be encoded by SEQ ID NO:51.

SEQ ID NO: 53 is a polynucleotide sequence encoding Connective tissue growth factor.

SEQ ID NO: 54 is a polypeptide sequence of Connective tissue growth factor, which can be encoded by SEQ ID NO:53.

SEQ ID NO: 55 is a polynucleotide sequence encoding Cytochrome P450.

SEQ ID NO: 56 is a polypeptide sequence of Cytochrome P450, which can be encoded by SEQ ID NO:55.

SEQ ID NO: 57 is a polynucleotide sequence encoding Cytochrome P451.

SEQ ID NO: 58 is a polypeptide sequence of Cytochrome P451, which can be encoded by SEQ ID NO:57.

SEQ ID NO: 59 is a polynucleotide sequence encoding HSPB1.

SEQ ID NO: 60 is a polypeptide sequence of HSPB1, which can be encoded by SEQ ID NO:59.

SEQ ID NO: 61 is a polynucleotide sequence encoding HSPA5.

SEQ ID NO: 62 is a polypeptide sequence of HSPA5, which can be encoded by SEQ ID NO:61.

SEQ ID NO: 63 is a polynucleotide sequence encoding IGF1.

SEQ ID NO: 64 is a polypeptide sequence of IGF1, which can be encoded by SEQ ID NO:63.

SEQ ID NO: 65 is a polynucleotide sequence encoding IGF2.

SEQ ID NO: 66 is a polypeptide sequence of IGF2, which can be encoded by SEQ ID NO:65.

SEQ ID NO: 67 is a polynucleotide sequence encoding IGFBP2.

SEQ ID NO: 68 is a polypeptide sequence of IGFBP2, which can be encoded by SEQ ID NO:67.

SEQ ID NO: 69 is a polynucleotide sequence encoding Myocilin.

SEQ ID NO: 70 is a polypeptide sequence of Myocilin, which can be encoded by SEQ ID NO:69.

SEQ ID NO: 71 is a polynucleotide sequence encoding Transgelin.

SEQ ID NO: 72 is a polypeptide sequence of Transgelin, which can be encoded by SEQ ID NO:71.

SEQ ID NO: 73 is a polynucleotide sequence encoding Thrombomodulin.

SEQ ID NO: 74 is a polypeptide sequence of Thrombomodulin, which can be encoded by SEQ ID NO:73.

SEQ ID NO: 75 is a polynucleotide sequence encoding Thrombospondin.

SEQ ID NO: 76 is a polypeptide sequence of Thrombospondin, which can be encoded by SEQ ID NO:75.

SEQ ID NO: 77 is a polynucleotide sequence encoding Apolipoprotein D.

SEQ ID NO: 78 is a polypeptide sequence of Apolipoprotein D, which can be encoded by SEQ ID NO:77.

SEQ ID NO: 79 is a polynucleotide sequence encoding α-1-antichymotrypsin (serpin).

SEQ ID NO: 80 is a polypeptide sequence of α-1-antichymotrypsin (serpin), which can be encoded by SEQ ID NO:79.

SEQ ID NO: 81 is a polynucleotide sequence encoding Cadherin (CDH2).

SEQ ID NO: 82 is a polypeptide sequence of Cadherin (CDH2), which can be encoded by SEQ ID NO:81.

SEQ ID NO: 83 is a polynucleotide sequence encoding Cadherin (CDH4).

SEQ ID NO: 84 is a polypeptide sequence of Cadherin (CDH4), which can be encoded by SEQ ID NO:83.

SEQ ID NO: 85 is a polynucleotide sequence encoding Cadherin (CDH15).

SEQ ID NO: 86 is a polypeptide sequence of Cadherin (CDH15), which can be encoded by SEQ ID NO:85.

SEQ ID NO: 87 is a polynucleotide sequence encoding Fibulin 1.

SEQ ID NO: 88 is a polypeptide sequence of Fibulin 1, which can be encoded by SEQ ID NO:87.

SEQ ID NO: 89 is a polynucleotide sequence encoding Pigment epithelium-derived factor.

SEQ ID NO: 90 is a polypeptide sequence of Pigment epithelium-derived factor, which can be encoded by SEQ ID NO:89.

SEQ ID NO: 91 is a polynucleotide sequence encoding Secretogranin II.

SEQ ID NO: 92 is a polypeptide sequence of Secretogranin II, which can be encoded by SEQ ID NO:91.

SEQ ID NO: 93 is a polynucleotide sequence encoding Serum amyloid A1.

SEQ ID NO: 94 is a polypeptide sequence of Serum amyloid A1, which can be encoded by SEQ ID NO:93.

SEQ ID NO: 95 is a polynucleotide sequence encoding Procollagen C-proteinase enhancer.

SEQ ID NO: 96 is a polypeptide sequence of Procollagen C-proteinase enhancer, which can be encoded by SEQ ID NO:95.

SEQ ID NO: 97 is a forward primer used in the presently disclosed subject matter.

SEQ ID NO: 98 is a reverse primer used in the presently disclosed subject matter.

### DETAILED DESCRIPTION

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a virus (e.g., titer), dose (e.g. an amount of a gene therapy construct), sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “cell” refers not only to the particular subject cell (e.g., a living biological cell), but also to the progeny or potential progeny of such a cell. Because certain modifications can occur in succeeding generations due to either mutation or environmental influences, such progeny might not, in fact, be identical to the parent cell, but are still included within the scope of the term as used herein.

The terms “nucleic acid molecule” or “nucleic acid” each refer to deoxyribonucleotides or ribonucleotides and polymers thereof in single-stranded, double-stranded, or triplexed form. Unless specifically limited, the term encompasses nucleic acids containing known analogues of natural nucleotides that have similar properties as the reference natural nucleic acid. The terms “nucleic acid molecule” or “nucleic acid” can also be used in place of “gene”, “cDNA”, or “mRNA”. Nucleic acids can be synthesized, or can be derived from any biological source, including any organism.

The term “heterologous nucleic acids” refers to a sequence that originates from a source foreign to an intended host cell or, if from the same source, is modified from its original form. Thus, a heterologous nucleic acid in a host cell includes a gene that is endogenous to the particular host cell, but which has been modified, for example by mutagenesis or by isolation from native cis-regulatory sequences. The term “heterologous nucleic acid” also includes non-naturally occurring multiple copies of a native nucleotide sequence. The term “heterologous nucleic acid” also encompasses a nucleic acid that is incorporated into a host cell's nucleic acids, however at a position wherein such nucleic acids are not ordinarily found.

The term “recombinant” generally refers to an isolated nucleic acid that is replicable in a non-native environment. Thus, a recombinant nucleic acid can comprise a non-replicable nucleic acid in combination with additional nucleic acids, for example vector nucleic acids, which enable its replication in a host cell. The term “recombinant” is also used to describe a vector (e.g., an adenovirus or an adeno-associated virus) comprising recombinant nucleic acids.

The term “gene” refers broadly to any segment of DNA associated with a biological function. A gene can comprise sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

As is understood in the art, a gene comprises a coding strand and a non-coding strand. As used herein, the terms “coding strand”, “coding sequence” and “sense strand” are used interchangeably, and refer to a nucleic acid sequence that has the same sequence of nucleotides as an mRNA from which the gene product is translated. As is also understood in the art, when the coding strand and/or sense strand is used to refer to a DNA molecule, the coding/sense strand includes thymidine residues instead of the uridine residues found in the corresponding mRNA. Additionally, when used to refer to a DNA molecule, the coding/sense strand can also include additional elements not found in the mRNA including, but not limited to promoters, enhancers, and introns. Similarly, the terms “template strand” and “antisense strand” are used interchangeably and refer to a nucleic acid sequence that is complementary to the coding/sense strand.

The term “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA syntheses, processing, and transport, as well as polypeptide synthesis, transport, and post-translational modification of polypeptides. Additionally, processes that affect protein-protein interactions within the cell can also affect gene expression as defined herein.

The terms “modulate” or “alter” are used interchangeably and refer to a change in the expression level of a gene, or a level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the terms “modulate” and/or “alter” can mean “inhibit” or “suppress”, but the use of the words “modulate” and/or “alter” are not limited to this definition.

As used herein, the terms “inhibit”, “suppress”, “downregulate”, “loss of function”, “block of function”, and grammatical variants thereof are used interchangeably and refer to an activity whereby gene expression (e.g., a level of an RNA encoding one or more gene products) is reduced below that observed in the absence of a composition of the presently disclosed subject matter. In some embodiments, inhibition results in a decrease in the steady state level of a target RNA. By way of example and not limitation, ocular glucocorticoid administration can downregulate expression of a number of genes below that observed in the absence of glucocorticoids.

The term “RNA” refers to a molecule comprising at least one ribonucleotide residue. By “ribonucleotide” is meant a nucleotide with a hydroxyl group at the 2′ position of a D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

The term “operatively linked”, as used herein, refers to a functional combination between a promoter region and a nucleic acid molecule such that the transcription of the nucleic acid molecule is controlled and regulated by the promoter region. Techniques for operatively linking a promoter region to a nucleic acid molecule are known in the art.

The terms “vector”, “expression vector”, and “construct” are used interchangeably and refer to a nucleic acid molecule having nucleotide sequences that enable its replication in a host cell. A vector can also include nucleic acids to permit ligation of nucleotide sequences within the vector, wherein such nucleic acids are also replicated in a host cell. Representative vectors include plasmids and viral vectors. The term “vector” is also used to describe an expression construct, wherein the expression construct comprises a vector and a nucleic acid operatively inserted with the vector, such that the nucleic acid is expressed in the host cell.

Vectors can also comprise nucleic acids including expression control elements, such as transcription/translation control signals, origins of replication, polyadenylation signals, internal ribosome entry sites, promoters, enhancers, etc., wherein the control elements are operatively associated with a nucleic acid encoding a gene product. Many such sequences can be derived from commercially available vectors. See e.g., Sambrook & Russell, 2001, and references cited therein.

The terms “cis-acting regulatory sequence” or “cis-regulatory motif” or “response element”, as used herein, each refer to a nucleotide sequence within a promoter region that enables responsiveness to a regulatory transcription factor. Responsiveness can encompass a decrease or an increase in transcriptional output and is mediated by binding of the transcription factor to the DNA molecule comprising the response element.

The term “transcription factor” generally refers to a protein that modulates gene expression by interaction with the cis-regulatory element and cellular components for transcription, including RNA Polymerase, Transcription Associated Factors (TAFs), chromatin-remodeling proteins, reverse tet-responsive transcriptional activator, and any other relevant protein that impacts gene transcription.

The term “promoter” defines a region within a gene that is positioned 5′ to a coding region of a same gene and functions to direct transcription of the coding region. The promoter region includes a transcriptional start site and at least one cis-regulatory element. The term “promoter” also includes functional portions of a promoter region, wherein the functional portion is sufficient for gene transcription. To determine nucleotide sequences that are functional, the expression of a reporter gene is assayed when variably placed under the direction of a promoter region fragment.

The terms “steroid glaucoma”, “primary open-angle glaucoma”, “open-angle glaucoma”, ‘glaucoma”, “steroid-induced glaucoma” and variants thereof are used interchangeably throughout the instant disclosure and refer to a glaucoma condition associated with or caused by ocular glucocorticoid administration. In some embodiments steroid glaucoma can be characterized by increased intraocular pressure (IOP), increased extracellular matrix (ECM) deposition, impaired aqueous humor outflow in trabecular meshwork tissue, decreased trabecular meshwork phagocytosis, induced glaucoma-linked gene myocilin, decreased expression and activity of matrix metalloproteinases MMPs, or any combination of any of the foregoing.

The terms “glucocorticosteroid”, “glucocorticoid”, “corticosteroid” and “steroid” are used interchangeably throughout the instant disclosure and refer to glucocorticosteroid compounds such as but not limited to dexamethasone, triamcinalone acetonide, and prednisolone acetate.

### II. Steroid-Induced Glaucoma

II.A. Glucocorticoids and Glaucoma

Glucocorticoids are potent immunosuppressants commonly used for the treatment of many inflammatory disorders including, for example, ocular inflammation. Glucocorticoid response can occur by the binding of the steroid hormone to the intracellular glucocorticoid receptor alpha (GRα). The ligand-receptor complex dimerizes, translocates to the nucleus and binds to a DNA cis-acting glucocorticoid response element (GRE) to modulate the expression of target genes (Aranda A. and A. Pascual, 2001 Physiol. Rev. 81:1269-1304).

However, the administration of steroids to subjects can have untoward effects on a number of biological processes. A number of conditions can be associated with the administration of steroids, including conditions related to ocular health. For example, administration of glucocorticoids can cause elevated intraocular pressure (IOP) and lead to open-angle glaucoma in steroid-responsive subjects. Topical ocular treatment with corticosteroids produces a dose-dependent IOP increase in 30% to 40% of the general population (Armaly M. F., 1963 Arch. Ophthalmol. 70:482-491; Armaly M. F., 1965 Fed Proc. 24:1274-1278) and 90% of patients with primary open-angle glaucoma (POAG; Tripathi et al., 1999 Drugs Aging 15:439-450). The ocular hypertension effect of the glucocorticoids is significantly greater in older age groups and is completely reversed after cessation of the treatment (Armaly M. F., 1963 Arch. Ophthalmol. 70:482-491; Becker B. and D. W. Mills, 1963 Arch Ophthalmol. 70:500-507). In some instances, steroid responsive individuals, i.e. steroid-responders, are more likely to develop POAG than their non-responder counterparts (Kitazawa Y. and T. Horie, 1981 Arch Ophthalmol. 99:819-823).

Glaucoma is a multifactorial ocular disease characterized by the death of the retinal ganglion cells and degeneration of the optic nerve. Glaucoma affects 70 million people worldwide and is the second leading cause of irreversible blindness (Quigley H. A., 1996 Br J Ophthalmol 80:389-393). It is well established that the major risk factor for the development of glaucoma is elevated IOP (Kass et al., 2002 Arch Ophthalmol. 120:701-713) and that, this elevated IOP is caused by an increased resistance to the aqueous humor outflow exerted by the trabecular meshwork tissue (Grant W. M., 1951 AMA Arch Ophthalmol. 46:113-131).

II.B. Extracellular Matrix and Matrix Metalloproteinases

The ECM has been shown to be relevant in the regulation of outflow facility (Keller et al., 2009 Exp Eye Res. 88:676-682). Under normal conditions matrix metalloproteinase enzymes (MMPs) control ECM deposition. However, the administration of glucocorticoids can lead to increased ECM deposition (Steely et al., 1992 Invest Ophthalmol Vis Sci. 33:2242-2250). It is applicants' belief that steroid administration down-regulates the expression and/or activity MMPs, thereby contributing to increased ECM deposition which can subsequently result in elevated IOP. As such, in some embodiments the presently disclosed subject matter provides methods and compositions directed to the regulation of the availability and/or activity MMPs in a cell, tissue or subject. In some embodiments, the presently disclosed subject matter provides for the regulation of MMPs to counteract the adverse effects of glucocorticoid administration.

MMP enzymes comprise a family of zinc-containing proteases, which are secreted as inactive pro-enzymes and are frequently regulated at the level of transcription. MMPs play a role in the turnover and maintenance of the trabecular meshwork's ECM. In some embodiments of the presently disclosed subject matter the MMP family includes MMP1, MMP3, MMP10 and MMP12. The member MMP1 is an interstitial collagenase that breaks down ECM collagens types I, II, and III. Collagen type I is an integral component of the trabecular meshwork extracellular scaffold and it forms part of the central core of the trabecular meshwork beams.

### III. Gene Therapy Compositions

The administration of steroids to subjects can have untoward effects on a number of biological processes. A number of conditions can be associated with the administration of steroids, including conditions related to ocular health. While not being limited to one particular theory, these untoward effects of steroid administration can be attributed to the alteration of the expression of a number of genes, including those in ocular tissues.

In accordance with the presently disclosed subject matter, genes susceptible to altered expression in the presence of a steroid in vivo can be used as a tool to develop gene therapy compositions to be used in therapeutic applications to treat, prevent or minimize effects on ocular health associated with glucocorticoid administration to subjects. In some embodiments, a gene therapy composition and method can be used in conjunction (i.e. before, during, after, or a combination thereof) with ocular glucocorticoid treatments to counteract the side effects of the glucocorticoid treatment on ocular health. The instant disclosure represents the first development of compositions and methods for the delivery and expression (in some embodiments overexpression) of transgenes to the trabecular meshwork by the use of viral vectors for the treatment of conditions associated with steroid administration.

The general strategy of gene therapy is the insertion of an introduced non-native sequence of DNA, e.g. a coding sequence for a polypeptide of interest, into a cell, tissue or organ of a subject, and in some instances incorporation into the subject's native DNA, in order to facilitate a biological change. By way of example and not limitation, the nucleic acid sequence SEQ ID NO: 3 coding for the MMP1 polypeptide having an amino acid sequence of SEQ ID NO: 4 can be introduced and expressed, constitutively or by induction, in a cell or tissue, e.g. trabecular meshwork, of a subject to thereby affect a change in the expression and/or activity of MMP1 in the cell or tissue. This approach can be used with cells capable of being grown in culture in order to study the function of the nucleic acid sequence, as well as in vivo as a therapeutic strategy. General representative gene therapy methods are described in U.S. Pat. Nos. 5,279,833; 5,286,634; 5,399,346; 5,646,008; 5,651,964; 5,641,484; and 5,643,567, the contents of each of which are herein incorporated by reference.

Gene therapy methods and compositions of the presently disclosed subject matter are directed toward modulation of the expression and/or activity of any polypeptide of interest to thereby affect or modulate the biological activity of a polypeptide of interest and counteract side effects of glucocorticoid administration. In some embodiments, methods and compositions are provided for increasing the expression and/or activity of MMPs to counteract the inhibition of one or more MMPs caused by glucocorticoid administration. In some embodiments, a gene therapy is provided for MMP expression. Provided in some embodiments are gene therapy constructs and compositions designed to express one or more MMPs in the presence of a glucocorticoid. In some embodiments, gene therapy constructs and methods are provided to treat, prevent, ameliorate or reverse steroid-induced elevated IOP, increased ECM deposition, steroid glaucoma and other associated effects of ocular glucocorticoid administration.

III.A. Gene Therapy Constructs

In some embodiments the presently disclosed subject matter provides a gene therapy vehicle, delivery system or construct comprising an inducible vector expressing a polypeptide of interest. In some embodiments the inducible vector is designed to treat subjects suffering from untoward effects of steroid administration. In some embodiments a vector of the presently disclosed subject matter is designed to treat subjects suffering from steroid glaucoma. In some embodiments a vector of the presently disclosed subject matter is designed to counteract the downregulation of the expression of one or more genes of interest and/or decreased availability of a polypeptide of interest in a subject receiving steroid treatment. In some embodiments the vector is designed to counteract ECM deposition and/or lower IOP in subjects receiving steroid treatment.

The presently disclosed subject matter provides in some embodiments an inducible vector expressing a polypeptide of interest, e.g. MMP1. In some embodiments, a gene therapy construct is provided wherein the construct increases the levels of the polypeptide of interest, also referred to as a “therapeutic product”, when the agent triggering the disease is present, and stop its mode of action when it is no longer needed. For example, in some embodiments a vector of the presently disclosed subject matter can comprise a steroid response element (SRE). Due to the inducible nature of the SRE, a vector comprising a selected gene and a SRE will express the gene only when exposed to a steroid.

At least one advantage of the inducible vector expressing a polypeptide of interest is that it is active only when the insult-triggered agent is present. Therefore, rather than the coding sequence for the polypeptide of interest being constitutively expressed, it will be expressed only when needed, that is, when the steroid is present. An additional advantage is that an inducible vector expressing a polypeptide of interest can counteract steroid induced down-regulation of the polypeptide of interest during and/or after steroid therapy. By way of example and not limitation, the expression of MMP1 from an inducible vector can prevent or reverse the steroid-induced increase in ECM deposition leading to elevated IOP. In some embodiments application of an inducible vector of the presently disclosed subject matter can help solve the problem with steroid-induced elevated IOP when administering steroids to patients.

In some embodiments the steroid-inducible vector, or steroid-inducible vector system, comprises a coding sequence for a polypeptide of interest (e.g. a MMP1 gene), a minimal promoter and a SRE, wherein the therapeutic gene is under the transcriptional control of the SRE. In some embodiments, the SRE is a glucocorticoid response element (GRE). In some embodiments the vector is an adenovirus vector. In some embodiments the GRE provides for transcription of the coding sequence for the polypeptide of interest in the presence of a glucocorticoid selected from the group comprising dexamethasone, triamcinalone acetonide, prednisolone acetate, and combinations thereof. In some embodiments the polypeptide of interest is MMP1, wherein MMP1 comprises a nucleotide sequence of SEQ ID NO: 3, or a nucleotide sequence substantially identical to SEQ ID NO: 3. In some embodiments MMP1 encodes a MMP1 peptide having an enzymatic activity substantially similar to endogenous MMP1. In some embodiments an expression cassette comprises a MMP1 coding sequence, minimal promoter and SRE. In some embodiments the vector is any vector capable of receiving and employing the expression cassette, as would be known to one of ordinary skill in the art. In some embodiments, the vector is one selected to minimize untoward immunogenic reactions such as inflammation.

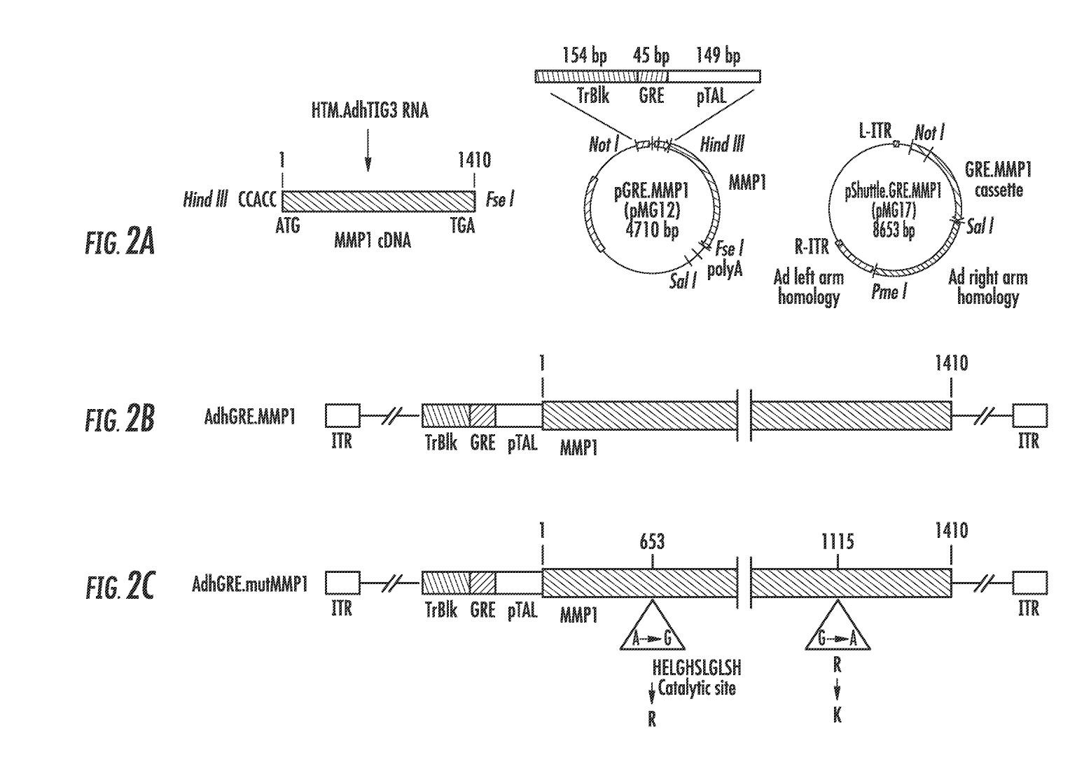

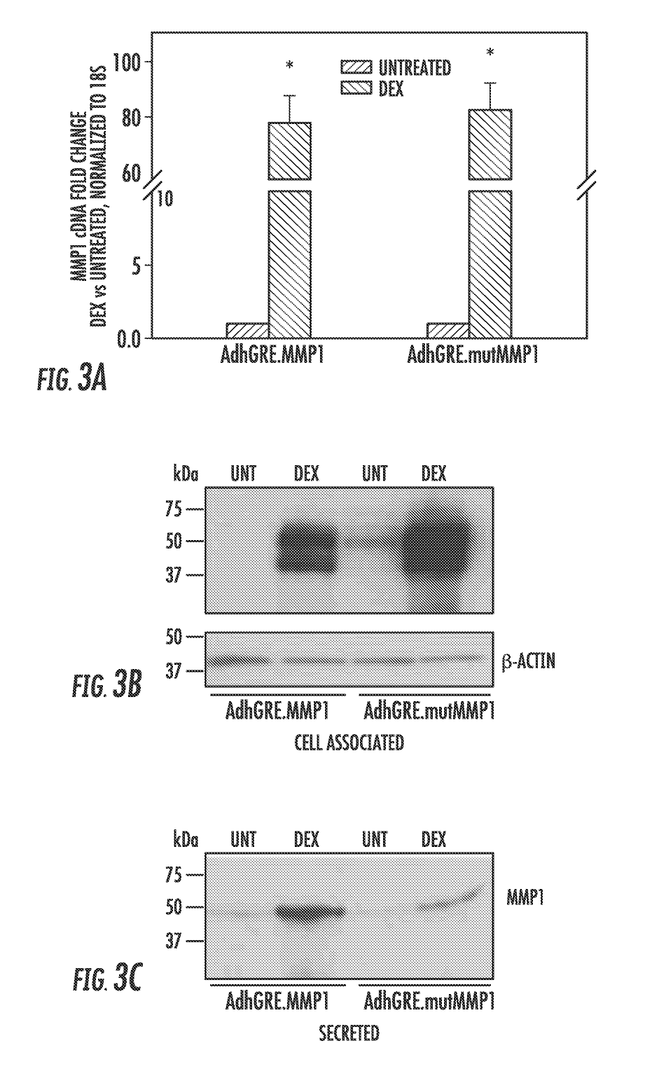

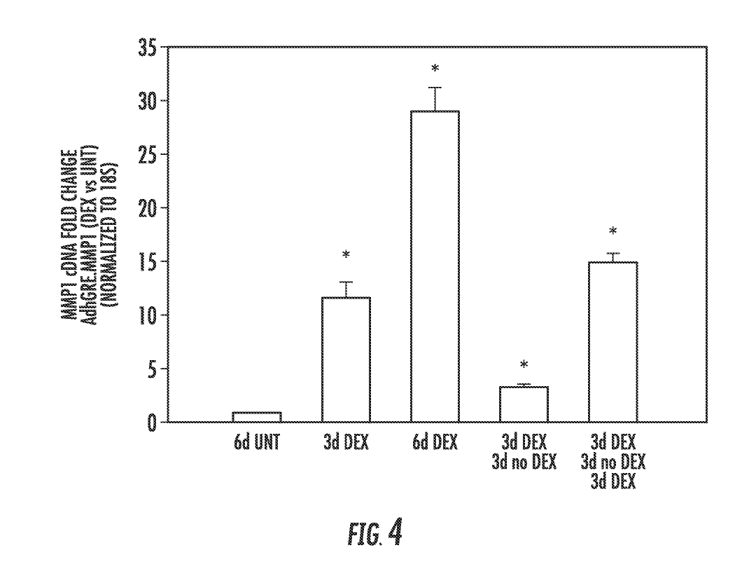

In some embodiments, a gene therapy construct is provided as described in FIGS. 2A-2C. FIGS. 2A-2C are schematic representations of the construction of glucocorticoid inducible virus vectors expressing MMP1. FIG. 2A is a schematic of a glucocorticoid inducible shuttle vector containing the full-coding MMP1 (pMG17) which was generated by first inserting the MMP1 amplified RT from AdhTIG3-infected cells downstream of the TrBlk.GRE.pTAL element of plasmid pGRE-Luc vector (pMG12); this was followed by subcloning the full GRE.MMP1 cassette into pShuttle vector using NotI/SalI enzymes. FIG. 2B is a schematic representation of AdhGRE.MMP1 recombinant virus DNA generated from a plasmid obtained by overlapping recombination of electroporated linear pMG17 DNA into BJ5183-Ad1 cells, which contain the adenovirus backbone vector. FIG. 2C is a schematic representation of AdhGRE.mutMMP1 recombinant virus DNA generated in a similar matter, except that the full coding MMP1 cDNA contained two single-point mutations; one of the mutations is at the catalytic site.

By way of example and not limitation, an inducible gene therapy construct of the presently disclosed subject matter can comprise an expression cassette comprising a nucleotide sequence of SEQ ID NO. 1. The expression cassette of SEQ ID NO.1 comprises and in some embodiments consists of 2,069 base pairs (bp) from the Not I to Sal I restriction sites. The location of features within the expression cassette of SEQ ID NO. 1 are as follows: Not I restriction site, by 1-6; Transcription Blocker (TB), by 7-160; Multiple Cloning Site, by 161-193; Glucocorticoid Response Element (GRE), by 194-238 (SEQ ID NO: 2); Bgl II restriction site, by 239-244; TATA-like promoter (P_{TAL}), by 245-393; Hind III restriction site, by 394-399; Kozak sequence, by 400-404; Human MMP1 coding sequence, by 405-1814 (SEQ ID NO: 3); Fse I restriction site, by 1815-1822; and GRE-Luc sequence (Clontech, Mountain View, Calif., United States of America) including polyA and Sal I site, by 1823-2069.

Thus, in some embodiments glucocorticoid-inducible vectors comprising human recombinant MMP1 cDNA under the control of cis-acting GRE are provided to counteract glucocorticoid down-regulation of MMP1 expression and subsequent effects on the ECM and IOP. The vectors can be designed to increase the expression of MMP1 at the time of glucocorticoid treatment to thereby counteract the glucocorticoid induced down-regulation of MMP1 expression.

III.B. Therapeutic Genes and Peptides

The administration of steroids to subjects can have untoward effects on a number of biological processes. A number of conditions can be associated with the administration of steroids, including conditions related to ocular health. While not being limited to one particular theory, these untoward effects of steroid administration can be attributed to the alteration of the expression of a number of genes, including those in ocular tissues. Indeed, the expression of a number of genes in ocular tissues can be altered by exposure to glucocorticoids, such as in glucocorticoid therapy. Table 1 includes a number of human trabecular meshwork (TM) genes that are susceptible to altered expression in the presence of a steroid in vivo.

TABLE 1
Human Trabecular Meshwork Genes Susceptible to Altered Expression
in the Presence of a Steroid in vivo

| Gene name | Symbol | Basic Function | Accession No. | SEQ ID NO. |
|---|---|---|---|---|
| MMP1 | MMP1 | ECM remodeling | NM_002421 | 3, 4 |
| MMP3 | MMP3 | ECM remodeling | NM_002422 | 5, 6 |
| MMP10 | MMP10 | ECM remodeling | NM_002425 | 7, 8 |
| MMP12 | MMP12 | ECM remodeling | NM_002426 | 9, 10 |
| ADAM10 | ADAM10 | ECM remodeling | NM_001110 | 11, 12 |
| ADAM19 | ADAM19 | ECM remodeling | NM_033274 | 13, 14 |
| ADAMTS28 | ADAMTS28 | ECM remodeling | NM_014265 | 15, 16 |
| ADAMTS1 | ADAMTS1 | ECM remodeling | NM_006988 | 17, 18 |
| ADAMTS3 | ADAMTS3 | ECM remodeling | NM_014243 | 19, 20 |
| ADAMTS5 | ADAMTS5 | ECM remodeling | NM_007038 | 21, 22 |
| Angiopoietin-like factor7/CDT6 | ANGPTL7/CDT6 | Antiangiogenesis/ECM deposit | NM_021146 | 23, 24 |
| Angiopoietin-like factor2 | ANGPTL2 | vascular growth factor | NM_012098 | 25, 26 |
| Angiopoietin 2 | ANGPT2 | vascular growth factor | NM_001147 | 27, 28 |
| Protein disulfide isomerase | PDIA2 | Protein folding | NM_006849 | 29, 30 |
| Protein disulfide isomerase | PDIA5 | Protein folding | NM_006810 | 31, 32 |
| Superoxide dismutase | SOD2 | Destroys superoxide radicals | NM_000636 | 33, 34 |
| Superoxide dismutase | SOD3 | Destroys superoxide radicals | NM_003102 | 35, 36 |
| Tropomyosin | TPM2 | Stabilizes actin filaments | NM_003289 | 37, 38 |
| Aldo-keto reductases | AKR1C1 | Antioxidants | NM_001353 | 39, 40 |
| Aldo-keto reductases | AKR1C3 | Antioxidants | NM_003739 | 41, 42 |
| Aldo-keto reductases | AKR1B10 | Antioxidants | NM_020299 | 43, 44 |
| S100 calcium binding protein | S100A10 | Calcium binding/cytoskelt regul [symbol] | NM_002966 | 45, 46 |
| Calreticulin | CALR | Calcium | NM_004343 | 47, 48 |
| | | homeostasis/chaperone | | |
| Chaperonin containing TCP1 | TCP1 | Protein folding | NM_030752 | 49, 50 |
| Chitinase 3 | CHI3L1 | Cartilage ECM | NM_001276 | 51, 52 |
| Connective tissue growth fact | CTGF | Differentiation of chondrocytes | NM_001901 | 53, 54 |
| Cytochrome P450 | CYP20A1 | Detoxification | NM_177538 | 55, 56 |
| Cytochrome P451 | CYP24A1 | Detoxification | NM_000782 | 57, 58 |
| Heat-shock proteins | HSPB1 | Chaperone | NM_001540 | 59, 60 |
| Heat-shock proteins | HSPA5 | Chaperone | NM_005347 | 61, 62 |
| Insul growth fact | IGF1 | Signaling hormone | NM_001111283 | 63, 64 |
| Insul growth fact | IGF2 | Signaling hormone | NM_001007139 | 65, 66 |
| Insulin-like growth fact bind | IGFBP2 | Bind to IGF, increase its half | NM_000597 | 67, 68 |
| proteins | | life | | |
| Myocilin | MYOC | Stress protein | NM_000261 | 69, 70 |
| Transgelin (smooth muscle22) | TAGLN | Actin crosslinking | NM_001001522 | 71, 72 |
| Thrombomodulin | THBD | Binds thrombin/inhibits clotting | NM_000361 | 73, 74 |
| Thrombospondin* | THBS2 | Cell-cell, cell-matrix interactions | NM_003247 | 75, 76 |
| Apolipoprotein D | APOD | Carrier protein/lipid transport | NM_001647 | 77, 78 |
| α-1-antichymotrypsin (serpin) | SERPINA3 | Serine protease inhibitor | NM_001085 | 79, 80 |
| Cadherin | CDH2 | Calcium depend cell-cell adhes [symbol] | NM_001792 | 81, 82 |
| Cadherin | CDH4 | Calcium depend cell-cell adhes [symbol] | NM_001794 | 83, 84 |
| Cadherin | CDH15 | Calcium depend cell-cell adhes [symbol] | NM_004933 | 85, 86 |
| Fibulin 1 | FBLN1 | Fibrillar ECM/calcium binding | NM_006486 | 87, 88 |
| Pigment epithelium-derived factor | PEDF | Inhibitor of angiogenensis | NM_002615 | 89, 90 |
| Secretogranin II | SCG2 | Neuropeptide precur/secretion | NM_003469 | 91, 92 |
| Serum amyloid A1 | SAA1 | ECM deposits/inflamm [symbol] | NM_000331 | 93, 94 |
| Procollagen C-proteinase | PCOLCE | | NM_002593 | 95, 96 |
| enhancer | | | | |

[symbol]

indicates data missing or illegible when filed

In some embodiments, the methods and compositions of the presently disclosed subject matter employ a gene therapy construct comprising a coding sequence (also referred to herein as a “nucleic acid molecule” or “therapeutic gene”) for a polypeptide of interest (also referred to herein as a “therapeutic polypeptide”). The terms “polypeptide of interest” and “therapeutic polypeptide” are used interchangeably and can refer to peptides whose concentration and/or activity in vivo can be altered by the effects of glucocorticoid exposure on the genes encoding the polypeptides. In some embodiments a coding sequence for a polypeptide of interest corresponds to a gene susceptible to altered expression in the presence of a steroid in vivo. In some embodiments a coding sequence for a polypeptide of interest corresponds to a gene of the TM that is susceptible to altered expression in the presence of a steroid in vivo. In some embodiments, the polypeptide of interest is selected from Table 1. In some embodiments a polypeptide of interest of the presently disclosed subject matter has an ability to modulate and/or ameliorate the effects of glucocorticoid exposure in the trabecular meshwork cells, such as on the expression of genes in the trabecular meshwork cells, and associated morphological and biochemical changes of the outflow tissue.

In some embodiments a gene therapy construct comprises a coding sequence comprising a nucleotide sequence of any of odd numbered SEQ ID NOs: 3-95; or a nucleic acid molecule comprising a nucleotide sequence substantially identical to any of odd numbered SEQ ID NOs: 3-95. In some embodiments, a gene therapy construct of the presently disclosed subject matter comprises a coding sequence comprising a nucleotide sequence that is 70%, 75%, 80%, 85%, 90%, 91%, 92%, 93%, 94%, 95%, 96%, 97%, 98% or 99% identical to any of odd numbered SEQ ID NOs: 3-95.

In some embodiments a gene therapy construct comprises a coding sequence comprising a nucleotide sequence encoding a polypeptide of any of even numbered SEQ ID NOs: 4-96; or a nucleic acid molecule comprising a nucleotide sequence encoding a polypeptide substantially identical to any of even numbered SEQ ID NOs: 4-96. In some embodiments, a gene therapy construct of the presently disclosed subject matter comprises a coding sequence comprising a nucleotide sequence encoding a polypeptide that is 70%, 75%, 80%, 85%, 90%, 91%, 92%, 93%, 94%, 95%, 96%, 97%, 98% or 99% identical to any of even numbered SEQ ID NOs: 4-96.

In one embodiment, a gene therapy construct of the presently disclosed subject matter encodes a polypeptide of interest that modulates ECM deposition. In some embodiments, the polypeptide of interest decreases ECM deposition, and particularly counteracts the increased ECM deposition caused by glucocorticoid administration.

In some embodiments, a gene therapy construct of the presently disclosed subject matter encodes a polypeptide that modulates the aqueous humor drainage. In some embodiments the polypeptide enhances aqueous humor drainage. In some embodiments the enhanced aqueous humor drainage mediated by a polypeptide of interest counteracts glucocorticoid induced impairment of the aqueous humor drainage. In some embodiments, a gene therapy construct of the presently disclosed subject matter encodes a polypeptide that decreases outflow resistance observed in steroid-responder subjects.

In some embodiments, the presently disclosed subject matter provides a gene therapy construct comprising a coding sequence for a MMP. In some embodiments a gene therapy construct of the presently disclosed subject matter can comprise one or more coding sequences for one or more MMPs, including but not limited to MMP1, MMP3, MMP10 and MMP12. See, e.g. Table 1. In some embodiments a gene therapy construct of the presently disclosed subject matter comprises a coding sequence for MMP1. In some embodiments a gene therapy construct is provided that encodes a variant, functional equivalent or mutant MMP polypeptide. In some embodiments, the gene product of a gene therapy construct provided herein comprises a protein having a MMP-like activity or substantially the same biological activity as one or more MMPs, such as but not limited to protease activity.

In some embodiments a gene therapy construct comprises a MMP1 nucleic acid molecule comprising a nucleotide sequence of SEQ ID NO: 3; or a nucleic acid molecule comprising a nucleotide sequence substantially identical of SEQ ID NO: 3. In some embodiments a gene therapy construct comprises a nucleic acid molecule encoding a MMP1 polypeptide comprising an amino acid sequence of SEQ ID NO: 4; a polypeptide comprising an amino acid sequence substantially identical to SEQ ID NO: 4; or a polypeptide that is a biological equivalent of or having a substantially similar biological activity of SEQ ID NO: 4.

Optionally, an MMP polypeptide encoded by a gene therapy construct of the presently disclosed subject matter displays one or more biological properties of a naturally occurring MMP polypeptide. By way of example and not limitation, an MMP1 polypeptide encoded by a gene therapy construct of the presently disclosed subject matter can possess a protease activity, modulate ECM turnover and maintenance, and/or be involved in trabecular meshwork outflow facility. The biological properties of an MMP polypeptide can further be assessed using methods described in Examples herein below.

The presently disclosed subject matter also encompasses MMP polypeptides that are engineered to differ in activity from naturally occurring MMPs. For example, MMP1 can be engineered to more potently inhibit ECM deposition in trabecular meshwork, particularly in the presence of glucocorticoid treatment.

III.B.1. Substantially Identical Nucleic Acids and Polypeptides

The recombinant vectors, therapeutic genes, expression cassettes and/or polypeptides described herein can be variably constructed, for example, by including sequences substantially identical to those described in particular embodiments of the presently disclosed subject matter. As described herein, representative nucleic acids encoding a polypeptide of interest include nucleotide sequences of any one of odd numbered SEQ ID NOs: 3-95. Representative MMP polypeptides can comprise an amino acid sequences of any of even numbered SEQ ID NOs: 4-96. In one embodiment of the presently disclosed subject matter, a recombinant adenovirus can comprise an expression cassette as set forth as SEQ ID NO: 1, or an expression cassette substantially similar to that set forth in SEQ ID NO: 1. Thus, the presently disclosed subject matter can also comprise sequences substantially identical to any of SEQ ID NOs: 1-98, including all known naturally occurring variants.

Nucleic Acids

The term “substantially identical”, as used herein to describe a degree of similarity between nucleotide sequences, refers to two or more sequences that have in one embodiment at least about least 60%, in another embodiment at least about 70%, in another embodiment at least about 80%, in another embodiment at least about 85%, in another embodiment at least about 90%, in another embodiment at least about 91%, in another embodiment at least about 92%, in another embodiment at least about 93%, in another embodiment at least about 94%, in another embodiment at least about 95%, in another embodiment at least about 96%, in another embodiment at least about 97%, in another embodiment at least about 98%, in another embodiment at least about 99%, in another embodiment about 90% to about 99%, and in another embodiment about 95% to about 99% nucleotide identity, when compared and aligned for maximum correspondence, as measured using one of the following sequence comparison algorithms (described herein below under the heading “Comparison of Nucleotide and Amino Acid Sequences”) or by visual inspection. In one embodiment, the substantial identity exists in nucleotide sequences of at least about 100 residues, in another embodiment in nucleotide sequences of at least about 150 residues, and in still another embodiment in nucleotide sequences comprising a full length sequence. The term “full length”, as used herein to refer to a complete open reading frame encoding, for example, a gene of interest polypeptide. The term “full length” also encompasses a non-expressed sequence, for example a promoter or an inverted terminal repeat sequence.

In one aspect, polymorphic sequences can be substantially identical sequences. The term “polymorphic” refers to the occurrence of two or more genetically determined alternative sequences or alleles in a population. An allelic difference can be as small as one base pair.

In another aspect, substantially identical sequences can comprise mutagenized sequences, including sequences comprising silent mutations. A mutation can comprise a single base change.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “‘target” is a test nucleic acid molecule, often found within a heterogeneous population of nucleic acid molecules. A “target sequence” is synonymous with a “test sequence”.

In one embodiment, a nucleotide sequence employed for hybridization studies or assays includes probe sequences that are complementary to or mimic at least an about 14 to 40 nucleotide sequence of a nucleic acid molecule of the presently disclosed subject matter. In one embodiment, probes comprise 14 to 20 nucleotides, or even longer where desired, such as 30, 40, 50, 60, 100, 200, 300, or 500 nucleotides or up to the full length of any one of odd numbered SEQ ID NOs: 1-98. Such fragments can be readily prepared by, for example, chemical synthesis of the fragment, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen, 1993. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook & Russell, 2001, for a description of SSC buffer. Often, a high stringency wash is preceded by a low stringency wash to remove background probe signal. An example of medium stringency wash conditions for a duplex of more than about 100 nucleotides is 15 minutes in 1×SSC at 45° C. An example of low stringency wash for a duplex of more than about 100 nucleotides is 15 minutes in 4× to 6×SSC at 40° C. For short probes (e.g., about 10 to 50 nucleotides), stringent conditions typically involve salt concentrations of less than about 1 M Na⁺ ion, typically about 0.01 to 1M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to identify nucleotide sequences that are substantially identical to reference nucleotide sequences of the presently disclosed subject matter: in one embodiment a probe nucleotide sequence hybridizes to a target nucleotide sequence in 7% sodium dodecyl sulfate (SDS), 0.5 M NaPO₄, 1 mM EDTA at 50° C. followed by washing in 2×SSC, 0.1% SDS at 50° C.; in another embodiment, a probe and target sequence hybridize in 7% SDS, 0.5 M NaPO₄, 1 mM EDTA at 50° C. followed by washing in 1×SSC, 0.1% SDS at 50° C.; in another embodiment, a probe and target sequence hybridize in 7% SDS, 0.5 M NaPO₄, 1 mM EDTA at 50° C. followed by washing in 0.5×SSC, 0.1% SDS at 50° C.; in another embodiment, a probe and target sequence hybridize in 7% SDS, 0.5 M NaPO₄, 1 mM EDTA at 50° C. followed by washing in 0.1×SSC, 0.1% SDS at 50° C.; in another embodiment, a probe and target sequence hybridize in 7% SDS, 0.5 M NaPO₄, 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, or are biologically functional equivalents. These terms are defined further herein below. Nucleic acid molecules that do not hybridize to each other under stringent conditions are still substantially identical if the corresponding proteins are substantially identical. This can occur, for example, when two nucleotide sequences are significantly degenerate as permitted by the genetic code.

The term “conservatively substituted variants” refers to nucleic acid sequences having degenerate codon substitutions wherein the third position of one or more selected (or all) codons is substituted with mixed-base and/or deoxyinosine residues. See Ohtsuka et al., 1985; Batzer et al., 1991; Rossolini et al., 1994.

The term “subsequence” refers to a sequence of nucleic acids that comprises a part of a longer nucleic acid sequence. An exemplary subsequence is a probe, described herein, or a primer. The term “primer” as used herein refers to a contiguous sequence comprising in one embodiment about 8 or more deoxyribonucleotides or ribonucleotides, in another embodiment 10-20 nucleotides, and in yet another embodiment 20-30 nucleotides of a selected nucleic acid molecule. The primers of the presently disclosed subject matter encompass oligonucleotides of sufficient length and appropriate sequence so as to provide initiation of polymerization on a nucleic acid molecule of the presently disclosed subject matter.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

The term “complementary sequences”, as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison set forth above, or is defined as being capable of hybridizing to the nucleic acid segment in question under relatively stringent conditions such as those described herein. A particular example of a complementary nucleic acid segment is an antisense oligonucleotide.

Nucleic acids of the presently disclosed subject matter can be cloned, synthesized, recombinantly altered, mutagenized, or combinations thereof. Standard recombinant DNA and molecular cloning techniques used to isolate nucleic acids are known in the art. Site-specific mutagenesis to create base pair changes, deletions, or small insertions are also known in the art. See e.g., Sambrook & Russell (2001) Molecular Cloning: a Laboratory Manual, 3rd ed. Cold Spring Harbor Laboratory Press, Cold Spring Harbor, N.Y.; Silhavy et al. (1984) Experiments with Gene Fusions. Cold Spring Harbor Laboratory, Cold Spring Harbor, N.Y.; Glover & Hames (1995) DNA Cloning: A Practical Approach, 2nd ed. IRL Press at Oxford University Press, Oxford/New York; Ausubel (1995) Short Protocols in Molecular Biology, 3rd ed. Wiley, New York.

TABLE 2
Functionally Equivalent Codons

| Amino Acids | | | Codons |
|---|---|---|---|
| Alanine | Ala | A | GCA GCC GCG GCU |
| Cysteine | Cys | C | UGC UGU |
| Aspartic Acid | Asp | D | GAC GAU |
| Glumatic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | ACG AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Polypeptides

The term “substantially identical” in the context of two or more polypeptide sequences is measured as polypeptide sequences having in one embodiment at least about 35%, in another embodiment at least about 45%, in another embodiment 45-55%, and in another embodiment 55-65% of identical or functionally equivalent amino acids. In yet another embodiment, “substantially identical” polypeptides will have at least about 70%, in another embodiment at least about 80%, in another embodiment at least about 85%, in another embodiment at least about 90%, in another embodiment at least about 91%, in another embodiment at least about 92%, in another embodiment at least about 93%, in another embodiment at least about 94%, in another embodiment at least about 95%, in another embodiment at least about 96%, in another embodiment at least about 97%, in another embodiment at least about 98%, in another embodiment at least about 99% identical or functionally equivalent amino acids. Methods for determining percent identity are defined herein below under the heading “Comparison of Nucleotide and Amino Acid Sequences”.

Substantially identical polypeptides also encompass two or more polypeptides sharing a conserved three-dimensional structure. Computational methods can be used to compare structural representations, and structural models can be generated and easily tuned to identify similarities around important active sites or ligand binding sites. See Barton, 1998; Saqi et al., 1999; Henikoff et al., 2000; Huang et al., 2000.

Substantially identical proteins also include proteins comprising an amino acid sequence comprising amino acids that are functionally equivalent to amino acids of a reference polypeptide. The term “functionally equivalent” in the context of amino acid sequences is known in the art and is based on the relative similarity of the amino acid side-chain substituents. See Henikoff & Henikoff, 2000. Relevant factors for consideration include side-chain hydrophobicity, hydrophilicity, charge, and size. For example, arginine, lysine, and histidine are all positively charged residues; alanine, glycine, and serine are all of similar size; and phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982). It is known that certain amino acids can be substituted for other amino acids having a similar hydropathic index or score and still retain a similar biological activity. The substitution of amino acids whose hydropathic indices are in one embodiment within ±2 of the original value, in another embodiment within ±1 of the original value, and in yet another embodiment within ±0.5 of the original value are chosen in making changes based upon the hydropathic index.

It is also understood in the art that the substitution of like amino acids can be made effectively on the basis of hydrophilicity. U.S. Pat. No. 4,554,101 describes that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

The substitution of amino acids whose hydrophilicity values are in one embodiment within ±2 of the original value, in another embodiment within ±1 of the original value, and in yet another embodiment within ±0.5 of the original value are chosen in making changes based upon similar hydrophilicity values.

The term “substantially identical” also encompasses polypeptides that are biologically functional equivalents. The term “functional” includes a biological activity of a peptide of the presently disclosed subject matter. By way of example and not limitation, a biological functional equivalent of a MMP polypeptide is a peptide having a protease activity substantially the same as that of a native MMP. Representative methods for assessing MMP polypeptide function are described in the Examples. When used to describe a polypeptide encoded by a gene of interest, the term “functional” refers to any function desirably provided by the gene of interest. The presently disclosed subject matter also provides functional protein fragments of MMP family members or a gene product of interest. Such functional portion need not comprise all or substantially all of the amino acid sequence of a native MMP or polypeptide encoded by a gene of interest.

The presently disclosed subject matter also includes functional polypeptide sequences that are longer sequences than that of a native MMP family members or polypeptides of interest. For example, one or more amino acids can be added to the N-terminus or C-terminus of a therapeutic polypeptide, e.g. MMP1. Methods of preparing such proteins are known in the art.

Comparison of Nucleotide and Amino Acid Sequences

The terms “identical” or percent “identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical” in regards to a nucleotide or polypeptide sequence means that a particular sequence varies from the sequence of a naturally occurring sequence by one or more deletions, substitutions, or additions, the net effect of which is to retain biological activity of a gene, gene product, or sequence of interest.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, for example by the local homology algorithm of Smith & Waterman, 1981, by the homology alignment algorithm of Needleman & Wunsch, 1970, by the search for similarity method of Pearson & Lipman, 1988, by computerized implementations of these algorithms (GAP, BESTFIT, FASTA, and TFASTA in the Wisconsin Genetics Software Package, available from Accelrys Inc., San Diego, Calif., United States of America), or by visual inspection. See generally, Ausubel, 1995.

An exemplary algorithm for determining percent sequence identity and sequence similarity is the BLAST algorithm, which is described in Altschul et al., 1990. Software for performing BLAST analyses is publicly available through the National Center for Biotechnology Information. This algorithm involves first identifying high scoring sequence pairs (HSPS) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPS containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff, 2000.

In addition to calculating percent sequence identity, the BLAST algorithm also performs a statistical analysis of the similarity between two sequences. See e.g., Karlin & Altschul, 1993. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

III.C. Gene Therapy Delivery Systems

The presently disclosed subject matter also provides gene therapy constructs or vectors. The particular vector employed in accordance with the presently disclosed subject matter is not intended to be a limitation of the disclosed and claimed compositions and methods. Thus, any suitable vector, construct or delivery vehicle as would be apparent to those of skill in the art upon a review of the instant disclosure can be used within the scope of the presently disclosed subject matter.

The vector can be a viral vector or a non-viral vector. Suitable viral vectors include adenoviruses, adeno-associated viruses (AAVs), self complementary AAV (scAAV; Buie et al., 2010 Invest Ophthalmol Vis Sci. 51:236-248), retroviruses, pseudotyped retroviruses, herpes viruses, vaccinia viruses, Semiliki forest virus, and baculoviruses. Suitable non-viral vectors comprise plasmids, water-oil emulsions, polethylene imines, dendrimers, micelles, microcapsules, liposomes, and cationic lipids. Polymeric carriers for gene therapy constructs can be used as described in Goldman et al. (1997) Nat Biotechnol 15:462 and U.S. Pat. Nos. 4,551,482 and 5,714,166. Where appropriate, two or more types of vectors can be used together. For example, a plasmid vector can be used in conjunction with liposomes. Provided in some embodiments of the presently disclosed subject matter is the use of an adenovirus, as described further herein below.

Suitable methods for introduction of a gene therapy construct into cells include direct injection into a cell or cell mass, particle-mediated gene transfer, electroporation, DEAE-Dextran transfection, liposome-mediated transfection, viral infection, and combinations thereof. A delivery method is selected based considerations such as the vector type, the toxicity of the encoded gene, the condition or tissue to be treated and the site of administration and/or treatment.

III.C.1. Viral Gene Therapy Vectors

In some embodiments viral vectors of the presently disclosed subject matter can be disabled, e.g. replication-deficient. That is, they lack one or more functional genes required for their replication, which prevents their uncontrolled replication in vivo and avoids undesirable side effects of viral infection. In some embodiments, all of the viral genome is removed except for the minimum genomic elements required to package the viral genome incorporating the therapeutic gene into the viral coat or capsid. For example, in some embodiments it is desirable to delete all the viral genome except the Long Terminal Repeats (LTRs) or Invented Terminal Repeats (ITRs) and a packaging signal. In the cases of adenoviruses, deletions can be made in the E1 region and optionally in one or more of the E2, E3 and/or E4 regions. In the case of retroviruses, genes required for replication, such as env and/or gag/pol can be deleted. Deletion of sequences can be achieved by recombinant approaches, for example, involving digestion with appropriate restriction enzymes, followed by religation. Replication-competent self-limiting or self-destructing viral vectors can also be used.

Nucleic acid constructs of the presently disclosed subject matter can be incorporated into viral genomes by any suitable approach known in the art. In some embodiments, such incorporation can be performed by ligating the construct into an appropriate restriction site in the genome of the virus. Viral genomes can then be packaged into viral coats or capsids by any suitable procedure. In particular, any suitable packaging cell line can be used to generate viral vectors of the presently disclosed subject matter. These packaging lines complement the replication-deficient viral genomes of the presently disclosed subject matter, as they include, typically incorporated into their genomes, the genes which have been deleted from the replication-deficient genome. Thus, the use of packaging lines allows viral vectors of the presently disclosed subject matter to be generated in culture. In some embodiments the vector is an adenoviral vector. By way of example and not limitation, adenovirus titration and determination of infectivity are described in the Examples below. By way of example and not limitation, design and incorporation of nucleic acid constructs and expression cassettes into viral vectors, as well as construction of adenoviral gene therapy constructs of the presently disclosed subject matter is described in the Materials and Methods for Examples 1-7 and in Example 2. By way of example and not limitation, in vitro and in vivo gene expression of adenoviral gene therapy constructs of the presently disclosed subject matter are described in the Examples below.

III.C.2. Plasmid Gene Therapy Vectors

In some embodiments, a therapeutic gene can be encoded by a naked plasmid. The toxicity of plasmid DNA is generally low and large-scale production is relatively easy. Plasmid transfection efficiency in vivo encompasses a multitude of parameters, such as the amount of plasmid, time between plasmid injection and electroporation, temperature during electroporation, and electrode geometry and pulse parameters (field strength, pulse length, pulse sequence, etc.). The methods disclosed herein can be optimized for a particular application by methods known to one of skill in the art, and the presently disclosed subject matter encompasses such variations. See, e.g., Heller et al. (1996) FEBS Lett 389:225-228; Vicat et al. (2000) Hum Gene Ther 11:909-916; Miklavcic et al. (1998) Biophys J 74:2152-2158.

III.C.3. Liposomes

The presently disclosed subject matter also provides for the use of gene therapy constructs comprising liposomes. Liposomes can be prepared by any of a variety of techniques that are known in the art. See, e.g., Betageri et al., 1993 Liposome Drug Delivery Systems, Technomic Publishing, Lancaster; Gregoriadis, ed., 1993 Liposome Technology, CRC Press, Boca Raton, Fla.; Janoff, ed. 1999 Liposomes: Rational Design, M. Dekker, New York, N.Y.; Lasic & Martin, 1995 Stealth Liposomes, CRC Press, Boca Raton, Fla.; Nabel, 1997 “Vectors for Gene Therapy” in Current Protocols in Human Genetics on CD-ROM, John Wiley & Sons, New York, N.Y.; and U.S. Pat. Nos. 4,235,871; 4,551,482; 6,197,333; and 6,132,766. Temperature-sensitive liposomes can also be used, for example THERMOSOMES™ as disclosed in U.S. Pat. No. 6,200,598. Entrapment of an active agent within liposomes of the presently disclosed subject matter can also be carried out using any conventional method in the art. In preparing liposome compositions, stabilizers such as antioxidants and other additives can be used.

Other lipid carriers can also be used in accordance with the presently disclosed subject matter, such as lipid microparticles, micelles, lipid suspensions, and lipid emulsions. See, e.g., Labat-Moleur et al., 1996 Gene Therapy 3:1010-1017; U.S. Pat. Nos. 5,011,634; 6,056,938; 6,217,886; 5,948,767; and 6,210,707.

III.D. Inducible Gene Therapy Vectors

In some instances a continuous un-regulated overexpression of transgene products could result in an unwanted physiological or toxic effect. Thus, in an effort to maximize expression levels of a gene product encoded by a gene therapy vector at a desired site and/or at a desired time, and concomitantly minimize the constitutive expression and/or systemic levels of the same encoded gene product, constructs of the presently disclosed subject matter can comprise an inducible promoter. As disclosed herein, controlled expression of a therapeutic transgene can be achieved by employing an inducible vector. The presently disclosed subject matter provides in some embodiments an inducible vector expressing a therapeutic peptide, e.g. MMP1.

In some embodiments, an insult-induced gene therapy construct is provided that increases the levels of its therapeutic product when the agent triggering the disease is present, and stops its mode of action when it is no longer needed. By way of example and not limitation, an inducible gene therapy construct of the presently disclosed subject matter for treating glaucoma can increase expression of its therapeutic peptide, e.g. MMP1, when the construct is in the presence of a steroid, and stop or substantially decrease expression of its therapeutic gene upon the removal or clearance of the steroid.

At least one advantage of an inducible vector is that it is active only when the insult-triggered agent is present. Therefore, rather than a coding sequence for a polypeptide of interest, e.g. MMP1, being constitutively expressed, it will be expressed only when needed, that is, when a triggering agent, e.g. a steroid, is present. Thus, by way of example and not limitation, an inducible gene therapy construct of the presently disclosed subject matter expressing MMP1 can counteract the down-regulation of the MMP1 enzyme by a steroid administered during steroid therapy. Therefore, in coupling the application of such a gene therapy construct with steroid therapy, the expression of MMP1 from the inducible vector can prevent or reverse steroid-induced increases in ECM deposition leading to elevated IOP. In some embodiments application of an inducible vector of the presently disclosed subject matter can help solve the problem ophthalmologists have with steroid-induced elevated IOP when administering steroids to their patients.

In some embodiments a gene therapy construct of the presently disclosed subject matter can comprise a steroid response element (SRE). Due to the inducible nature of the SRE, a vector comprising a selected gene and a SRE will express the gene only when exposed to a steroid. In some embodiments, the SRE is a glucocorticoid response element (GRE).

In some embodiments, a gene therapy construct of the presently disclosed subject matter can comprise a MMP1 cDNA inserted downstream of 3 tandem copies of a GRE consensus sequence fused to a TATA-like promoter region from the HSV-thymidine kinase gene available in the commercial vector pGRE.Luc (Clontech, Mountain View, Calif., United States of America). The GRE consensus element can comprise a non-perfect palindromic sequence, e.g. SEQ ID NO: 2, which is part of a GC regulatory response unit, which in some cases involves more than one GRE, half-site GREs and/or even negative GREs (Nordeen et al., 1990 Mol. Endocrinol. 4:1866-1873). To reduce and in some cases avoid spurious transcription from upstream sequences, the vector can further comprise a transcription blocker (TrBlk) upstream of the GRE element.

In some embodiments of the presently disclosed subject matter, the GRE promoter comprises a nucleotide sequence of SEQ ID NO: 2 or a nucleotide sequence substantially identical to SEQ ID NO: 2.

In some embodiments a SRE (such as but not limited to a GRE) of the presently disclosed subject matter can be concatamerized or combined with additional response elements, promoters, or elements to amplify transcriptional activity. Alternatively or in addition, a response element or inducible promoter can be combined with an element that acts as an enhancer of mRNA translation.

An inducible vector of the presently disclosed subject matter can further be responsive to non-steroid stimuli that can be used in combined therapy treatments. For example, the mortalin promoter is induced by low doses of ionizing radiation (Sadekova (1997) Int J Radiat Biol 72(6):653-660), the hsp27 promoter is activated by 17β-estradiol and estrogen receptor agonists (Porter et al. (2001) J Mol Endocrinol 26(1):31-42), the HLA-G promoter is induced by arsenite, hsp promoters can be activated by photodynamic therapy (Luna et al. (2000) Cancer Res 60(6):1637-1644). Thus, an inducible vector of the presently disclosed subject matter comprising a SRE or GRE can comprise additional inducible features or additional DNA elements that support combined therapy treatments.

III.E. Pharmaceutical Compositions

The presently disclosed subject matter provides pharmaceutical compositions comprising a gene therapy construct of the presently disclosed subject matter. In some embodiments, a pharmaceutical composition can comprise one or more gene therapy constructs produced in accordance with the presently disclosed subject matter.

III.E.1. Carriers.

In some embodiments a pharmaceutical composition can also contain a pharmaceutically acceptable carrier or adjuvant for administration of the gene therapy construct. In some embodiments, the carrier is pharmaceutically acceptable for use in humans. In some embodiments, the carrier is pharmaceutically acceptable for use in the eye and associated ocular tissues. The carrier or adjuvant desirably should not itself induce the production of antibodies harmful to the individual receiving the composition and should not be toxic. Suitable carriers can be large, slowly metabolized macromolecules such as proteins, polypeptides, liposomes, polysaccharides, polylactic acids, polyglycolic acids, polymeric amino acids, ammo acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonate and benzoates.

Pharmaceutically acceptable carriers in therapeutic compositions can additionally contain liquids such as water, saline, glycerol and ethanol. Additionally, auxiliary substances, such as wetting or emulsifying agents or pH buffering substances, can be present in such compositions. Such carriers enable the pharmaceutical compositions to be formulated for administration to the patient.

The compositions of the presently disclosed subject matter can further comprise a carrier to facilitate composition preparation and administration. Any suitable delivery vehicle or carrier can be used, including but not limited to a microcapsule, for example a microsphere or a nanosphere (Manome et al. (1994) Cancer Res 54:5408-5413; Saltzman & Fung (1997) Adv Drug Deliv Rev 26:209-230), a glycosaminoglycan (U.S. Pat. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a nanosuspension (U.S. Pat. No. 5,858,410), a polymeric micelle or conjugate (Goldman et al. (1997) Cancer Res 57:1447-1451 and U.S. Pat. Nos. 4,551,482, 5,714,166, 5,510,103, 5,490,840, and 5,855,900), and a polysome (U.S. Pat. No. 5,922,545).

III.E.2. Formulation.

Suitable formulations of pharmaceutical compositions of the presently disclosed subject matter include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS in the range of in some embodiments 0.1 to 10 mg/ml, in some embodiments about 2.0 mg/ml; and/or mannitol or another sugar in the range of in some embodiments 10 to 100 mg/ml, in some embodiments about 30 mg/ml; and/or phosphate-buffered saline (PBS). Any other agents conventional in the art having regard to the type of formulation in question can be used. In some embodiments, the carrier is pharmaceutically acceptable. In some embodiments the carrier is pharmaceutically acceptable for use in humans. In some embodiments the carrier is pharmaceutically acceptable for use in the eye and ocular tissue.

Pharmaceutical compositions of the presently disclosed subject matter can have a pH between 5.5 and 8.5, preferably between 6 and 8, and more preferably about 7. The pH can be maintained by the use of a buffer. The composition can be sterile and/or pyrogen free. The composition can be isotonic with respect to humans. Pharmaceutical compositions of the presently disclosed subject matter can be supplied in hermetically-sealed containers.

### IV. Gene Therapy Methods

A therapeutic method according to the presently disclosed subject matter comprises administering to a subject in need thereof a gene therapy construct. The general strategy of gene therapy is the insertion of an introduced non-native sequence of DNA into a cell, tissue or organ of a subject, and in some instances incorporation into the subject's native DNA, in order to facilitate a biological change. Preferably, the gene therapy construct encodes a polypeptide having an ability to elicit a biological response or change in a desired tissue.

In accordance with the presently disclosed subject matter, any gene susceptible to altered expression in the presence of a steroid in vivo, e.g. a TM gene such as one of the MMP genes, can be used as a tool of gene therapy in subjects to treat, prevent or minimize effects associated with glucocorticoid administration. In some embodiments, a gene therapy can be used in conjunction (i.e. before, during, after, or a combination thereof) with glucocorticoid treatments to counteract the side effects of the glucocorticoid treatment on ocular health.

The therapeutic methods of the presently disclosed subject matter are relevant to disorders that are caused by or exacerbated by steroid treatment, or associated with steroid treatment. As disclosed herein, glucocorticoid treatments, including ocular steroid treatments, can lead to alterations in the expression of a number of genes, including TM genes. In some instances the alteration in TM gene expression can affect ocular health as a result of increased ECM and elevated IOP in a substantial number of subjects. One resulting condition is referred to as steroid glaucoma. Accordingly, the disclosed gene therapy constructs can be useful in the treatment of steroid glaucoma. Thus, the presently disclosed subject matter provides therapeutic methods to counteract alterations in TM gene expression and in some instances ECM deposition and elevated IOP in subjects receiving steroid treatment.

The presently disclosed subject matter provides methods of treating steroid glaucoma in a subject in need thereof, the method comprising providing a subject suffering from steroid glaucoma, providing a vector comprising a coding sequence for a polypeptide of interest (e.g., MMP1), a minimal promoter and a SRE, wherein the coding sequence is under the transcriptional control of the SRE, wherein the coding sequence corresponds to a gene susceptible to altered expression in the presence of a steroid in vivo, and administering the vector to the subject, wherein the steroid glaucoma is treated. In some embodiments the polypeptide of interest is MMP1. In some embodiments, the steroid glaucoma comprises elevated IOP. In some embodiments, the elevated IOP is decreased. In some embodiments, the steroid glaucoma comprises increased ECM deposition. In some embodiments, the increased ECM deposition is decreased. In some embodiments, the vector is an adenovirus vector. In some embodiments, the SRE is a GRE. In some embodiments the subject is a mammal. In some embodiments the mammal is a human. In some embodiments a vector of the presently disclosed subject matter is administered to an ocular tissue of the subject by any approach suitable for administration to ocular tissue. In some embodiments the subject is receiving steroid treatment, wherein the steroid is a glucocorticoid selected from the group comprising dexamethasone, triamcinalone acetonide, prednisolone acetate, and combinations thereof.

The presently disclosed subject matter also provides a method of preventing elevated IOP in a subject receiving steroid treatment, the method comprising providing a subject receiving steroid treatment, providing a vector comprising a coding sequence for a polypeptide of interest, a minimal promoter and a SRE, wherein coding sequence is under the transcriptional control of the SRE, wherein the coding sequence corresponds to a gene susceptible to altered expression in the presence of a steroid in vivo, and administering the vector to the subject, wherein elevated IOP in the subject is prevented. In some embodiments, the vector is an adenovirus vector. In some embodiments, the SRE is a GRE. In some embodiments the polypeptide of interest is MMP1. In some embodiments, the subject is a mammal. In some embodiments, the subject is a human. In some embodiments a vector of the presently disclosed subject matter is administered to an ocular tissue of the subject by any approach suitable for administration to ocular tissue. In some embodiments the subject is receiving a steroid treatment, wherein the steroid is a glucocorticoid selected from the group comprising dexamethasone, triamcinalone acetonide, prednisolone acetate, and combinations thereof.

The presently disclosed subject matter also provides a method of reversing elevated IOP in a subject receiving steroid treatment, the method comprising providing a subject receiving steroid treatment, wherein the subject has elevated IOP, providing a vector comprising a coding sequence for a polypeptide of interest, a minimal promoter and a SRE, wherein the coding sequence is under the transcriptional control of the SRE, wherein the coding sequence corresponds to a gene susceptible to altered expression in the presence of a steroid in vivo, and administering the vector to the subject, wherein the elevated IOP in the subject is reversed. In some embodiments, the vector is an adenovirus vector. In some embodiments, the SRE is a GRE. In some embodiments the polypeptide of interest is MMP1. In some embodiments, the subject is a mammal. In some embodiments, the subject is a human. In some embodiments a vector of the presently disclosed subject matter is administered to an ocular tissue of the subject by any approach suitable for administration to ocular tissue. In some embodiments the subject is receiving a steroid treatment, wherein the steroid is a glucocorticoid selected from the group comprising dexamethasone, triamcinalone acetonide, prednisolone acetate, and combinations thereof.

The presently disclosed subject matter also provides an ocular treatment method comprising providing a subject in need of ocular treatment, administering a vector comprising a coding sequence for a polypeptide of interest, a minimal promoter and a SRE, wherein the coding sequence is under the transcriptional control of the SRE, wherein the coding sequence corresponds to a gene susceptible to altered expression in the presence of a steroid in vivo, and administering a steroid, such as but not limited to a glucocorticoid, to an ocular tissue of the subject. In some embodiments the subject in need of ocular treatment comprises a subject suffering from inflammation, ocular inflammation, macular edema, choroidal neovascularization, or any other eye or systemic condition requiring administration of a steroid. In some embodiments, the vector is administered prior to, simultaneously, or after glucocorticoid administration. In some embodiments, the vector is an adenovirus vector. In some embodiments, the SRE is a GRE. In some embodiments, the subject is a mammal. In some embodiments, the mammal is a human. In some embodiments, the glucocorticoid is selected from the group comprising dexamethasone, triamcinalone acetonide, prednisolone acetate, and combinations thereof. In some embodiments, the vector and glucocorticoid are administered to an ocular tissue of the subject by any means suitable for administration to ocular tissue.

The presently disclosed subject matter also provides a method of treating or preventing a condition associated with steroid treatment in a subject, the method comprising providing a subject receiving steroid treatment, providing a vector comprising a coding sequence for a polypeptide of interest, a minimal promoter and a SRE, wherein the coding sequence is under the transcriptional control of the SRE, wherein the coding sequence corresponds to a gene susceptible to altered expression in the presence of a steroid in vivo, and administering the vector to the subject. In some embodiments, the vector is an adenovirus vector. In some embodiments, the SRE is a glucocorticoid response element (GRE). In some embodiments, the polypeptide of interest is MMP1. In some embodiments, the subject is a mammal. In some embodiments, the steroid treatment comprises the administration of a glucocorticoid to the subject, wherein the glucocorticoid is selected from the group consisting of dexamethasone, triamcinalone acetonide, prednisolone acetate, and combinations thereof. In some embodiments, the vector comprises administering the vector to an ocular tissue of the subject. In some embodiments, the vector is administered prior to, simultaneously, or after steroid administration.

IV.A. Subjects

The subject treated in the presently disclosed subject matter is desirably a human subject, although it is to be understood that the principles of the disclosed subject matter indicate that the compositions and methods are effective with respect to invertebrate and to all vertebrate species, including mammals, which are intended to be included in the term “subject”. Moreover, a mammal is understood to include any mammalian species in which treatment of ocular conditions or treatment or prevention of glaucoma is desirable, particularly agricultural and domestic mammalian species.

The methods of the presently disclosed subject matter are particularly useful in the treatment of warm-blooded vertebrates. Thus, the presently disclosed subject matter concerns mammals and birds.

More particularly, provided herein is the treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economical importance to humans. Thus, provided herein is the treatment of livestock, including, but not limited to, domesticated swine (pigs and hogs), ruminants, horses, poultry, and the like.

In some embodiments, the subject to be treated in accordance with the presently disclosed subject matter is a subject in need of ocular treatment. In some embodiments, a subject in need of ocular comprises a subject suffering from ocular inflammation, macular edema, choroidal neovascularization, or combinations thereof.

IV.B. Administration

Suitable methods for administration of a gene therapy construct of the presently disclosed subject matter include but are not limited to intravenous, subcutaneous, or intraocular injection. In some embodiments the gene therapy constructs of the presently disclosed subject matter are administered via sub-Tenon injection or trans-corneal injection. Alternatively, a gene therapy construct can be deposited at a site in need of treatment in any other manner appropriate for the condition to be treated or the target site. For example, any approach for administration suitable for the eye and ocular tissues is within the scope of the presently disclosed subject matter. In some embodiments, the particular mode of administering a therapeutic composition of the presently disclosed subject matter depends on various factors, including the distribution and abundance of cells to be treated, the vector employed, additional tissue- or cell-targeting features of the vector, and mechanisms for metabolism or removal of the vector from its site of administration. For example, given the relative accessibility of the eye a number of administration methods can be employed without departing from the scope of the presently disclosed subject matter, e.g. eye drops.

In some embodiments, the method of administration encompasses features for regionalized vector delivery or accumulation at the site in need of treatment. For treatment of ocular tissue a gene therapy vector can be administered by intraocular injection, or in some embodiments by sub-Tenon injection or trans-corneal injection. See, e.g. Materials and Methods for Examples 9-14. In some embodiments the gene therapy construct can be delivered to the eye using eye drops. In some embodiments, direct administration of the gene therapy construct to the site or tissue of interest, e.g. ocular tissue, can enhance the efficacy of the gene therapy as compared to systemic routes of administration.

IV.C. Dose

An effective dose of a gene therapy composition of the presently disclosed subject matter is administered to a subject in need thereof. The terms “therapeutically effective amount”, “therapeutically effective dose”, “effective amount”, “effective dose” and variations thereof are used interchangeably herein and refer to an amount of a therapeutic composition or gene therapy construct of the presently disclosed subject matter sufficient to produce a measurable response (e.g. decreased ECM deposition and/or decreased IOP in a subject being treated). Actual dosage levels of gene therapy constructs, and in some instances the therapeutic genes expressed by the gene therapy constructs, can be varied so as to administer an amount that is effective to achieve the desired therapeutic response for a particular subject. By way of example and not limitation, in some embodiments the gene therapy constructs can be administered at dose ranging from 5×10⁸ to 1×10¹⁰ virus genomes (vg), which would correspond to 2×10⁸ to 5×10⁹ infectious units (IFU).

In some embodiments, the dosage of a gene therapy construct can be varied to achieve a desired level of MMP1 expression and/or activity in a subject. In some embodiments, a dosage of gene therapy construct of the presently disclosed subject matter can be optimized to treat, prevent or reverse steroid glaucoma in a subject, including but not limited to decreasing ECM deposition and/or IOP in the subject.

In some embodiments, the quantity of a therapeutic composition of the presently disclosed subject matter administered to a subject will depend on a number of factors including but not limited to the subject's size, weight, age, the target tissue or organ, the route of administration, the condition to be treated, and the severity of the condition to be treated. By way of example and not limitation, a pharmaceutical composition of the presently disclosed subject matter can be administered at a rate of approximately 5 to 50 ul/eye. In some embodiments, a pharmaceutical composition of the presently disclosed subject matter can be administered at a rate of approximately 10 to 45 ul/eye, 15 to 40 ul/eye, 20 to 35 ul/eye, or 25 to 30 ul/eye.

In some embodiments the selected dosage level will depend upon the activity of the therapeutic composition, the route of administration, combination with other drugs or treatments, the severity of the condition being treated, and the condition and prior medical history of the subject being treated. However, upon a review of the instant disclosure, it is within the skill of the art to consider these factors in optimizing an appropriate dosage, including for example starting doses of the compound at levels lower than required to achieve the desired therapeutic effect and to gradually increase the dosage until the desired effect is achieved. Moreover, upon review of the instant disclosure one of ordinary skill in the art can tailor the dosages to an individual subject by making appropriate adjustments or variations, as well as evaluation of when and how to make such adjustments or variations, as is routine to those of ordinary skill in the art.

The potency of a therapeutic composition can vary, and therefore a “therapeutically effective” amount can vary. However, using the assay methods described herein below, one skilled in the art can readily assess the potency and efficacy of a gene therapy construct of presently disclosed subject matter and adjust the therapeutic regimen accordingly. For example, MMP1 activity and collagen degradation assays are described in the Examples below. Further, exemplary methods of determining MMP1 expression and effects on ocular health and steroid glaucoma, e.g. IOP and ECM deposition, are described in the Examples below.

### V. Gene Therapy Models

The efficacy of the gene therapy constructs and compositions and methods of gene therapy were tested in a representative non-limiting approach using an in vitro primary culture of human outflow facility cells as a model of human ocular tissue and steroid glaucoma. The efficacy of the inducible gene therapy constructs and compositions and methods of gene therapy were also tested in a representative non-limiting approach ex vivo using perfused human anterior segment organ cultures. See Examples 1-8. Subsequently, the efficacy and safety of the gene therapy constructs and compositions and methods of gene therapy were tested in a representative non-limiting approach using an in vivo ovine model for steroid glaucoma. See Examples 9-14.

### V.I. In Vitro

Briefly, HTM primary cells were generated from trabecular meshwork tissue dissected from residual cornea rims after surgical corneal transplantation and used as a model of human ocular tissue. The HTM cells were grown to 90% confluency and exposed to the recombinant adenoviruses (AdGRE.MMP1 and AdGRE.mutMMP1) in 1 ml serum-free medium. After exposure to the virus for 90 minutes, complete media containing 0.1 μM DEX was added and incubation continued for 3 to 5 days.

Using this in vitro model the experiments showed that glucocorticoid administration to human primary trabecular meshwork cells greatly downregulated endogenous MMP1 expression and activity. Moreover, the administration of gene therapy constructs of the presently disclosed subject matter to the human primary trabecular meshwork cells resulted in a substantial increase in MMP1 expression. The increase in MMP1 expression was also shown to translate to increased MMP1 activity, with the recombinantly expressed MMP1 having substantially similar activity to endogenous MMP1. The results of the studies performed using this in vitro model demonstrate that the gene therapy constructs of the presently disclosed subject matter have the potential to increase MMP expression and counteract the negative effects of glucocorticoid administration on ocular health. These experiments are discussed in further detail in Examples 1-8.

### V.II. Ex Vivo

Three pairs of normal, nonglaucomatous human eyes from donors ages 72 to 74 were obtained from the National Disease Research Interchange (NDR1, Philadelphia, Pa., United States of America) following signed consent of the patients' families. Whole eye globes within 22 to 43 hours of death were dissected at the equator, cleaned and mounted on custom-made perfusion chambers as described previously (Borrás et al., 1999 Gene Ther. 6:515-524; Johnson, D. H., and R. C. Tschumper, 1987 Invest Ophthalmol Vis Sci. 28:945-953). These anterior segments were perfused at constant flow (3 to 6 μl/min) through one of chamber's two cannulas with serum-free high glucose DMEM (Gibco Invitrogen) containing antibiotics. HPLC pumps (MX7900-000, Rheodyne, Rhonert Park, Calif., United States of America) equipped with a 20 μl loop were intercalated between perfusion syringes and chambers to be able to administer virus without injection through the cornea. Outflow facility (flow/pressure in μl/min/mmHg) was calculated from the average of three values obtained from pressure readings recorded at 30 minute intervals. Baseline values were taken just before the steroid treatment and sample delivery.

After obtaining a stable baseline HPLC loops were loaded with AdhGRE.MMP1 (for OS) or virus vehicle (for OD), which were delivered into the eyes by remote control loop injection from a computer. Fresh DEX-media was changed approximately every 36 hours and effluents were collected from the chambers reservoirs at different time points and saved at −20° C. for analysis of secreted proteins. At the end of the experiment, anterior segments were cut in several wedges which were either immersed in 4% paraformaldehyde or RNALATER™ reagent, for immunohistochemistry and transgene expression.

In these organ cultures, the trabecular meshwork maintains its natural architecture and the perfused media flows in a manner that mimics the flow of aqueous humor through the tissue. Organ cultures have also the advantage of their serum-free culture conditions (important for the study of secreted proteins) and the characteristic of maintaining expression of many genes which get downregulated once the cells are placed in standard tissue cultures. Experiments with paired eyes also allow the comparison of vehicle- and vector-injected trabecular meshworks from identical genetic backgrounds. The results with the organ cultures confirmed all findings first observed on the HTM cultured cells. The steroid-regulated increase of recombinant MMP1 was observed at the level of transcription in the dissected tissue and at the level of enzyme secretion in the effluents, showing a further increase with perfusion time. The collagenase activity of the effluents was also found to be greatly increased in the eye injected with the gene therapy vector. The results of the experiments using the ex vivo model strongly supports lowering IOP in vivo. These experiments are discussed in further detail in Examples 1-8.

### V.III. In Vivo Ovine Model

The effectiveness of using Corriedale sheep (Ovis aries) as an animal model for glucocorticosteroid-induced ocular hypertension was recently demonstrated (Gerometta et al., 2009 Invest. Ophthalmol. Vis. Sci. 50:669-673; incorporated herein by reference in its entirety). The IOP of these animals increased approximately 2.5-fold within 2 weeks of topically applying 0.5% prednisolone acetate three times daily. This intraocular pressure elevation occurred with a 100% incidence in the corticosteroid-treated eyes. Following discontinuation of the corticosteroid instillation, the IOP of the treated eyes declined to the baseline values over the course of 1-3 weeks. Similar IOP elevations were obtained in all sheep receiving the corticosteroid, triamcinolone acetonide, which was applied as a single sub-Tenon injection rather than topically.

The 100% incidence of corticosteroid-induced ocular hypertension in Ovis aries, and the docile nature of the animals, which readily submit to manipulations such as those required for in vivo outflow facility measurements, render this species an ideal model for both examining the mechanisms underlying corticosteroid-induced glaucoma and testing possible IOP-lowering agents. Moreover, the ovine physiology appears to be similar in terms of aqueous secretion to that of the human (Gerometta et al., 2005 Exp. Eye Res. 80:307-312), and trabecular meshwork anatomy also appears to be rather similar to primates (Simoens et al., 1996 J. Vet. Med. Sci. 58:977-982; Guyomard et al., 2008 Invest. Ophthalmol. Vis. Sci. 49:5168-5174). Another significant advantage of using an ovine steroid-induced model of IOP elevation is the consistency and robustness of the IOP response as well as the relatively low cost compared with studies in primates. Moreover, the sheep model for corticosteroid-induced ocular hypertension was preferable to other animal models such as rabbit. With the latter, only about 50% of rabbits treated chronically with glucocorticoids such as dexamethasone develop ocular hypertension, and dexamethasone responders are commonly defined as those exhibiting IOP elevations of at least 5 mmHg (Pang et al., 2001 Exp. Eye Res. 73:815-825. In contrast, all treated sheep responded to prednisolone with about 2.5-fold increases in IOP as reported previously (Gerometta et al., 2009 Invest. Ophthalmol. Vis. Sci. 50:669-673), and sub-Tenon injection of a triamcinolone depot was observed to be equally effective as prednisolone in elevating ovine IOP in that all sheep administered triamcinolone exhibited ocular hypertension.

Using the ovine model the gene therapy constructs and compositions of the presently disclosed subject matter were tested for their ability to reduce the corticosteroid-induced ocular hypertension in the eyes of the sheep. Briefly, the sheep were treated with a steroid to increase IOP. Approximately 30 μl of the virus suspension, including an inducible gene therapy vector with or without a therapeutic gene, was injected into the eye using a Hamilton syringe with a 28 G needle. The needles were inserted diagonally through the cornea (a few millimeters inside the limbus) into the anterior chamber without touching the iris. The IOP in the eyes of the sheep was then measured to determine the effect of the gene therapy vectors on IOP.

In summary, no clinical adverse effects were noted in any of the eyes treated with Ad vectors. There were no signs of conjunctival hyperemia, inflammation or irritation. The in vivo experiments using the ovine model demonstrated that single dose of a gene therapy vector carrying an inducible metalloproteinase human gene can both a) protect against the increase in IOP normally produced by corticosteroid instillation in the sheep model, and b) quickly reverse the IOP increase elicited by corticosteroid pretreatments.

### VI. Kits Containing Gene Therapy Constructs and Compositions

In some embodiments of the presently disclosed subject matter, there are provided articles of manufacture and kits containing gene therapy constructs and compositions produced in accordance with the presently disclosed subject matter which can be used, for instance, for therapeutic applications described above. The article of manufacture comprises a container with a label. Suitable containers include, for example, bottles, vials, and test tubes. The containers can be formed from a variety of materials such as glass or plastic. The container holds a composition which includes an active agent that is effective for therapeutic applications, such as described above. The active agent in the composition can comprise one or more gene therapy constructs or compositions of the presently disclosed subject matter. The label on the container indicates that the composition is used for a particular therapy or non-therapeutic application, and can also indicate directions for either in vivo, in vitro, or ex vivo use, such as those described above.

In some embodiments, a kit can comprise compositions for use in treating, preventing or ameliorating steroid glaucoma and/or conditions associated therewith. In some embodiments the kit can comprise an inducible gene therapy construct encoding a MMP. In some embodiments the kit can comprise an inducible gene therapy construct or composition for decreasing IOP and/or decreasing ECM deposition in a subject receiving steroid treatments.

A kit of the presently disclosed subject matter will typically comprise the container described above and one or more other containers comprising materials desirable from a commercial and user standpoint, including buffers, diluents, filters, needles, syringes, eye droppers and package inserts with instructions for use.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Materials and Methods for Examples 1-7

Primary Culture of Human Outflow Facility Cells

To generate primary HTM cells, the trabecular meshwork tissue was dissected from residual cornea rims after surgical corneal transplantation at the University of North Carolina Eye Clinic, Chapel Hill, N.C., United States of America. Trabecular meshworks were isolated from surrounding tissue, cut into small pieces and treated with 1 mg/ml collagenase type IV (Worthington, Lakewood, N.J., United States of America), as previously described (Vittitow et al., Mol. Vis. 2002; 8:32-44). Cells were maintained at 37° C., 7% CO₂ in MEM Richter's modification medium (HyClone, Thermo Fisher Scientific, Waltham, Mass., United States of America) supplemented with 20% FBS and 50 ug/ml gentamicin (Gibco Invitrogen, Carlsbad, Calif., United States of America). At confluency, cells were passed and maintained in the same medium but with 10% FBS (complete medium). All cells were used at passages 4 to 6. These outflow pathway cultures include cells from the three distinct regions of the trabecular meshwork plus cells lining the Schlemm's canal (SC). Because most of the cells in these cultures come from the trabecular meshwork they are commonly referred to as trabecular meshwork cells. The cells used in this study were from a 15-year old Caucasian male (HTM-109), a 2-year old Caucasian female (HTM-95), a 19-year old Caucasian male (HTM-106) and a 54-year old Caucasian female (HTM-140).

Drug Treatments

Drug treatments on the HTM cells were conducted in complete media. HTM cells were grown to pre-confluency and exposed to drugs as follows. Treatment with dexamethasone (DEX; Sigma, St. Louis, Mo., United States of America) was conducted at a final concentration of 0.1 uM. DEX was reconstituted in absolute ethanol at 0.1 mM and diluted 1.000-fold into complete media every 48 to 72 hours for the duration of the experiment. Treatment with triamcinolone acetonide (Kenacort-A; Bristol-Myers Squibb, New York, N.Y., United States of America) was performed at a final concentration of 0.1 mg/ml. Kenacort-A 40 mg/ml suspension was well mixed and diluted into complete medium 400-fold at the time of use. The concentration of 0.1 mg/ml triamcinolone acetonide was chosen because intravitreal injections of 1 mg/ml are widely used in the clinical setting, which would result in a lower concentration of the steroid in the aqueous humor. In addition, the concentration of 0.1 mg/ml has been successfully studied in trabecular meshwork cells (Fan B J et al., Invest Ophthalmol Vis Sci. 2008; 49:1886-1897). Treatment with prednisolone was conducted at a final concentration of 200 uM (80 μg/ml). Prednisolone 21-acetate (Sigma) was reconstituted in ethanol at 200 mM (80 mg/ml) as a suspension and mixed well prior dilution of 1.000-fold into the culture medium. The drugs were exposed to the cells for the period of time described in results. Untreated control dishes received the same volume of absolute ethanol (drug vehicle) under identical conditions.

RNA Extraction, Reverse Transcription, and cDNA Quantification

HTM cells were scraped from tissue culture dishes with guanidine thiocyanate buffer (RLT, Qiagen, Valencia, Calif., United States of America). Total RNA was extracted by loading the solution onto a QIA SHREDDER™ column (Qiagen) and continued by the use of the RNeasy Mini kit with on-column RNase-free DNAse digestion according to manufacturer's recommendations (Qiagen). Purified RNA was eluted in 30 ul RNase-free water and concentration measured with a NanoDrop ND-100 spectrophotometer (Thermo Fisher Scientific). For the tissue, human trabecular meshworks were excised from one week RNAlater (Ambion Applied Biosystems, Austin, Tex., United States of America) immersed anterior segments. One half of the isolated trabecular meshwork tissue was homogenized on 350 μl of RLT and RNA extraction continued as described for the cells. Recoveries were between 1.4 to 2 ug of RNA per human trabecular meshwork.

Reverse transcription (RT) reactions were conducted with 1 μg (HTM cells) or 400 ng (tissue) RNA in a 20 μl total volume of proprietary RT buffer with RNAse inhibitor (High Capacity cDNA kit; Applied Biosystems, ABI, Foster City, Calif., United States of America) following manufacturer's recommendations (25° C. for 10 minutes, 37° C. for 2 hours and 85° C. for 5 seconds). Fluorescently labeled TaqMan probe/primers sets for human MMP1 and 18S RNA were purchased from the ABI TaqMan Gene Expression collection. The human MMP1 probe corresponded to sequences from exons 6 and 7 (Hs00233958_m1, ABI) and the 18S RNA probe corresponded to sequences surrounding position nucleotide 609 (Hs99999901_s1, ABI). Reactions were performed in 20 μl aliquots using TaqMan Universal PCR Master mix No AmpErase UNG, run on an Applied Biosystems 7500 Real-Time PCR System, and analyzed by 7500 System SDS software (ABI). Relative Quantification (RQ) values between treated and untreated samples were calculated by the formula 2^{-ΔΔC}_{T} where C_{T} is the cycle at threshold, ΔC_{T} is C_{T} of the assayed gene minus C_{T} of the endogenous control (18S), and ΔΔC_{T} is the ΔC_{T} of the normalized assayed gene in the treated sample minus the ΔC_{T} of the same gene in the untreated one (calibrator). Because of the high abundance of the 18S rRNA used as the endogenous control and in order to get a linear amplification, RT reactions from treated and untreated samples were diluted 10⁴ times prior to their hybridization to the 18S TaqMan probe.

Protein Extraction, Western Blot Analysis and Protein Quantification

Serum-containing culture medium from treated and untreated HTM cells was collected, cleared of cellular debris by centrifugation at 1,500 rpm for 10 minutes and concentrated 40× with an Amicon Ultra-4 Centrifugal Filter Device Ultracel (10 kDa cutoff; Millipore, Billerica, Mass., United States of America) at 3,500 rpm, 4° C. After medium removal, HTM cells were washed with cold phosphate-buffered saline (PBS) and harvested in 150 ul of cold RIPA buffer (0.15 M NaCl, 0.02 M Tris-HCl pH 8, 1% NP40, 1% sodium deoxycholate, 0.1% SDS) supplemented with 1× protease inhibitor cocktail (Roche Applied Biosciences, Indianapolis, Ind., United States of America). Cells were disrupted with a sonicator (Microson Ultrasonic XL 2000; Misonix, Farmingdale, N.Y., United States of America) equipped with a 2.4 mm microprobe (Misonix) at setting 3 for 5 pulses. The sonicate was then centrifuged at 14,000 g for 20 minutes at 4° C. and supernatants (soluble fraction) collected and stored at −80° C. until use. Serum-free effluents from perfused organ cultures were concentrated 40× in the same manner as media from the cultured cells.

Equivalent volumes from treated and untreated protein extracts, concentrated media or effluents were mixed 1:2 (vol/vol) with loading Laemmli buffer (Bio-Rad, Hercules, Calif., United States of America) containing 5% β-mercaptoethanol and boiled for 5 minutes. Protein samples were separated on a 4-15% SDS-PAGE precast gel (Bio-Rad) and electro-transferred to a PVDF membrane (Bio-Rad). After blocking with 5% nonfat dry milk in 0.01 M Tris, pH 8.0, 0.1% Tween for 1-2 hours at room temperature, membranes were incubated overnight at 4° C. with rabbit anti-human MMP1 (1:1,000, AB8105, Millipore), or goat anti-human collagen type I (1:200, AB758, Millipore) primary antibodies. Membranes were then washed and incubated with anti-rabbit or anti-goat IgG secondary antibodies conjugated to horseradish peroxidase (HRP; 1:5,000; Pierce Thermo Fisher Scientific, Rockford, Ill., United States of America) for 1 hour at room temperature. Immunoreactive bands were visualized by chemiluminescence (ECL plus, GE Healthcare, Piscataway, N.J., United States of America) and exposed to X-ray film (BioMax MR Film, Kodak, Rochester, N.Y., United States of America). To re-probe membranes with other primary antibodies, membranes were stripped in 0.01 M Tris, 0.1% Tween, pH 2.0 for 15 minutes, washed, and neutralized in the same buffer at pH 8.0. For controls, membranes were incubated with mouse monoclonal anti-human β-actin for 1 hour at room temperature (1:5,000, A5441, Sigma) or rabbit anti-human myocilin (1:50, sc-21243, Santa Cruz Biotechnology, Santa Cruz, Calif., United States of America), washed and incubated with HRP-conjugated anti-mouse or anti-rabbit IgG, respectively (1:5,000; Pierce Thermo Fisher Scientific) for 1 hour at room temperature.

Levels of secreted MMP1 in concentrated HTM cultured medium and effluents were determined by ELISA using a human MMP1 ELISA Kit (RayBiotech, Norcross, Ga., United States of America) and following manufacture's recommendations. At the end of incubation, immunoplates were read at 450 nm in a microplate reader (FLUOstar Optima; BMG LABTECH, Cary, N.C., United States of America).

Adenovirus Titration

Physical particles were titered as virus genomes (vg) per ml (vg/ml) by real-time PCR using the MMP1 fluorescent TaqMan primers/probe described above (Hs00233958_m1, ABI). For this, viral DNA was extracted from 5 ul of purified virus using the DNeasy tissue kit (Qiagen) and amplification reactions set in triplicate. A standard curve was generated by amplifying known copy numbers of MMP1 plasmid pMG19 and plotting them against their threshold cycle (C_{T}) values. The number of vg was then determined comparing the C_{T} values of the viral DNA to the standard curve. Viral lots used in this study had concentrations of 3.1×10¹¹ (wild-type) and 4.0×10¹¹ (mutant) vg/ml, respectively.

Viral infectivity (infectious units per ml, IFU/ml) was measured by using the AdenoX Rapid Titer kit (Clontech, Mountain View, Calif., United States of America), which contains an antibody specific to the adenovirus hexon capsid protein produced only in infected cells. QBI-HEK293A cells were seeded in 12-well plates to 90% confluency and infected with serial dilutions (10⁴ to 10⁻⁶) of the adenovirus stock in duplicate wells. At 48 hours, cells were fixed with ice-cold 100% methanol for 10 minutes at −20° C., washed with PBS/1° ABSA, and incubated with a mouse anti-hexon antibody (1:2,000) for 1 hour. Positive brown color spots were detected by incubation with an HRP-conjugated rat anti-mouse (1:1,000, 1 hour 37° C.), washing and developing with 3,3′-Diaminobenzidine (DAB) substrate. Brown spots were counted with a 20× objective in an Olympus IX71 inverted microscope equipped with a DP70 digital camera. Each brown-stained cell corresponds to one infectious viral particle (infectious unit, IFU) and wells from dilutions with ˜50 spots/field were chosen for counting. Quantification of the IFU/ml was done by averaging the number of spots in 3-4 fields per well and applying the formula: brown spots/field×fields/well divided by virus volume used/well (ml)×virus dilution factor. There were 400 fields/well of a 12-well plate. A correction factor for the area of the captured image (1.84×) was obtained by the use of a calibrated slide and introduced in the formula to obtain the final titer. Viral lots used in this study had 1.8×10¹¹ (wild-type) and 2.1×10¹¹ (mutant) IFU/ml, respectively.

Delivery of Recombinant Adenoviruses to HTM Cells

HTM primary cells at passage 4 seeded on 6-well dishes were grown to 90% confluency, washed twice with PBS and exposed to the recombinant adenoviruses (AdGRE.MMP1 and AdGRE.mutMMP1) in 1 ml serum-free medium. After exposure to the virus for 90 minutes, complete media containing 0.1 μM DEX was added and incubation continued for 3 to 5 days. Fresh DEX medium was replaced at 48 to 72 hours intervals as indicated. Viral concentrations were at multiplicity of infection (moi) ranging from 2.3 to 2.6×10³ IFU/cell.

Measurement of MMP1 Activity by Collagen Degradation Assays

To determine the collagenase activity of the human recombinant MMP1 secreted in the media, two assays were performed: the fluorescence resonance energy transfer (FRET) assay, which incorporates the FRET pair labeling technology in a MMP peptide substrate, and the digestion of native rat tail collagen type I measured by gel electrophoresis. Conditioned media from post-infected dishes treated with steroids were cleared of cellular debris and concentrated 40× as indicated above. To activate pro-MMP1 from its latent state, samples were incubated at 37° C. for 3 hours in 1 mM p-Aminophenylmercuric acetate (APMA) (Sellers et al., Biochem J. 1977; 163:303-307) in a total volume of 50 μl. A commercially available purified pro-MMP1 (AnaSpec, Fremont, Calif., United States of America) was used as a positive control.

For the FRET assay, 10 μl of concentrated activated serum-containing media were incubated with the 5-FAM/QXL™520 labeled peptide for 40 minutes at 37° C. following manufacturer's recommendations (SensoLyte® 520 MMP-1 Assay Kit, AnaSpec). Enzyme activity was determined by measuring the fluorescence released upon proteolytic cleavage of the fluorescent peptide in a microplate reader (FLUOstar Optima) using 480/520 nm excitation/emission filters. To test the enzymatic activity against native collagen, 5 μl of activated serum-free media were incubated with 10 μg of native rat tail collagen type I (BD Biosciences, San Jose, Calif., United States of America) for 2 hours at 37° C. in a total volume of 28 μl (Chung L et al., EMBO J. 2004; 23:3020-3030). Half of the reaction volume was analyzed in a 4-15% Tris-HCl PAGE gel (Bio-Rad) at 100 V for 1.5 hours. Gels were subsequently washed with water and stained with Biosafe Coomassie G-250 (Bio-Rad) at 4° C. overnight. Bands were visualized after a final wash, and photographed with a Canon SD850 IS digital camera.

Perfused Human Anterior Segment Organ Cultures

Three pairs of normal, nonglaucomatous human eyes from donors ages 72 to 74 were obtained from the National Disease Research Interchange (NDR1, Philadelphia, Pa., United States of America) following signed consent of the patients' families. All procedures were in accordance with the Tenets of the Declaration of Helsinki. Whole eye globes within 22 to 43 hours of death were dissected at the equator, cleaned and mounted on custom-made perfusion chambers as described previously (Borrás et al., Invest Ophthalmol Vis Sci. 1987; 28:945-953). These anterior segments were perfused at constant flow (3 to 6 μl/min) through one of chamber's two cannulas with serum-free high glucose DMEM (Gibco Invitrogen) containing antibiotics, using a Harvard microinfusion pump (Harvard Bioscience, South Natick, Mass., United States of America). HPLC pumps (MX7900-000, Rheodyne, Rhonert Park, Calif., United States of America) equipped with a 20 μl loop were intercalated between perfusion syringes and chambers to be able to administer virus without injection through the cornea. All pumps were controlled by a custom-made computer program (Infusion Pump Control Program, University of North Carolina Chemistry Department, Electronic Design Facility). Anterior segments were maintained at 37° C., 5% CO₂ and perfused for 24 hours to establish a stable baseline (steady pressure recordings for at least 10 hours). Outflow facility (flow/pressure in μl/min/mmHg) was calculated from the average of three values obtained from pressure readings recorded at 30 minute intervals. Baseline values were taken just before the steroid treatment and sample delivery. The outflow facility at baseline for the eyes used in this study was C=0.29±0.03 (n=6).

After obtaining a stable baseline, the perfusion syringes and eyes anterior chambers were exchanged with fresh media containing 0.1 μM DEX. At the same time, HPLC loops were loaded with AdhGRE.MMP1 (for OS) or virus vehicle (for OD), which were delivered into the eyes by remote control loop injection from the computer. Fresh DEX-media was changed approximately every 36 hours and effluents were collected from the chambers reservoirs at different time points and saved at −20° C. for analysis of secreted proteins. At the end of the experiment, anterior segments were cut in several wedges which were either immersed in 4% paraformaldehyde or RNAlater, for immunohistochemistry and transgene expression.

Immunocytochemistry, Immunohistochemistry and Light Microscopy

Cells were cultured on glass coverslips precoated with poly-D-Lysine, fixed and fluorescently double labeled for the MMP1 and collagen type I proteins. Cells were washed, fixed with 4% paraformaldehyde for 10 minutes, permeabilized with 0.1% Triton X-100/PBS for 10 minutes, washed, and blocked with 2% donkey serum/PBS for 30 minutes. Coverslips were simultaneously incubated with rabbit anti-human MMP1 antibody (1:500, AB8105, Millipore) and goat-anti collagen type I (1:100, AB758, Millipore) for 1 hour at room temperature followed by an additional 1 hour with a mixture of donkey anti-rabbit Alexa Fluor 555 and donkey anti-goat Alexa Fluor 488, respectively (1:400; Molecular Probes, Invitrogen). All antibody solutions were made in 2% donkey serum and three washes were performed between incubation steps. All secondary antibodies were tested for cross-reactivity by incubating coverslips in the absence of the primary antibodies. Cells were counterstained with 4′,6-diamidino-2-phenylindole (DAPI) for 3 minutes before mounting the coverslips with a drop of Fluoromont G (Southern Biotechnology Associates, Birmingham, Aabama, United States of America).

Eyes from pairs #2 and #3 were fixed by immersion in 4% paraformaldehyde in PBS at room temperature overnight. Specimens were then rinsed in distilled water for 10 minutes and transferred to 70% ethanol for delivery to the UNC Histology Core for paraffin embedding. Meridional 10 μm sections from opposite quadrants of each eye were mounted on Superfrost/Premium microscope slides (Thermo Fisher Scientific). For the MMP1 and collagen type I detection, sections were first incubated at 60° C. for 1 hour, deparaffinized with xylene (2×, 7 minutes), rinsed with descending concentrations of ethanol (100% 5 minutes, 95% 4 minutes, 75% 3 minutes) and rehydrated with distilled water for 2 minutes. Heat induced antigen retrieval was achieved by treating the sections with unmasking solution (VectorLabs, Burlingame, Calif., United States of America) for 30 seconds at 125° C. in a decloaking chamber (Biocare Medical, Concord, Calif., United States of America). Slides were then cooled off, washed with PBS, permeabilized with 0.1% Triton X-100/PBS for 10 minutes, washed again, and blocked with 2% donkey serum/PBS for 30 minutes. Tissue sections were incubated with the same MMP1 (1:500, 2 hours at room temperature) and collagen type I (1:200, overnight at 4° C.) primary antibodies used for the cells followed by incubation with donkey anti-rabbit Alexa Fluor 555 and donkey anti-goat Alexa Fluor 488 secondary antibodies (1:200) (Molecular Probes Invitrogen) at room temperature for 1 hour. Sections were mounted with coverslips and Fluoromont G (Southern Biotechnology Associates) and sealed with clear enamel. Fluorescence imaging was conducted with an Olympus IX71 fluorescence microscope and images captured using an Olympus DP70 camera and accompanying software. Images from corresponding viral- and vehicle-treated sections were taken at the same exposure. Digital images were arranged with image analysis software (Photoshop CS; Adobe, Mountain View, Calif., United States of America). Negative controls were run in parallel but were incubated in blocking buffer in place of the primary antibody.

### Example 1

### Effect of Steroids on Endogenous MMP1 Expression

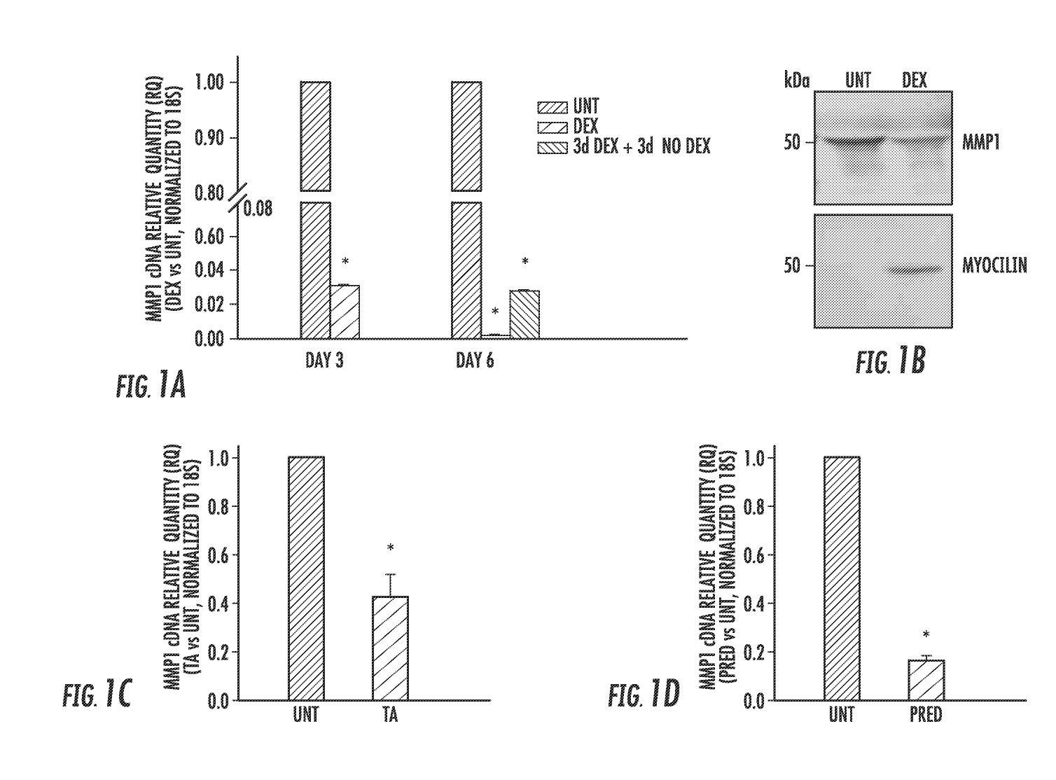

To evaluate the effect of glucocorticoid treatment on MMP1 expression, primary HTM-109 cells were treated with DEX, triamcinolone acetonide and prednisolone, and levels of 18S normalized MMP1 cDNA were measured by real-time TaqMan PCR. Treatment with 0.1 μM DEX in a representative experiment reduced MMP1 expression to RQ values of 0.03±0.0002 at 3 days (n=3, p=7×10⁻⁷) and of 0.002±0.0002 at 6 days (n=3, p=1×10⁻⁶) (FIG. 1A). Removal of DEX from the cultured medium at 3 days prevented the MMP1 reduction observed at day 6 (0.03±0.002, n=6, p=2×1e) (FIG. 1A). Analysis of secreted proteins by western blot conducted in a different experiment showed that MMP1 protein was also reduced in the DEX-treated sample at 5 days while cross-reaction of the stripped blot with myocilin (internal control) was increased (FIG. 1B). These DEX findings were confirmed in four mRNA and two protein additional experiments with similar results. Treatment of the cells with 0.1 mg/ml triamcinolone acetonide and 80 μg/ml prednisolone in a representative experiment reduced MMP1 cDNA levels to 0.42±0.1 (n=3, p=0.013) and 0.16±0.02 (n=3, p=0.002) at 12 and 24 hours, respectively (FIG. 1C). The experiment was repeated once in HTM-140 cell line with similar findings. These results indicate that treatment with glucocorticoids commonly used in a clinical setting reduced the expression of MMP1 in primary HTM cells.

### Example 2

### Design of a Glucocorticoid Inducible Generation of Recombinant Adenovirus Vectors Carrying Wild-type and Mutant MMP1 Genes

To counteract glucocorticoid down-regulation of MMP1 expression and subsequent effects on the ECM and IOP, glucocorticoid-inducible vectors comprising human recombinant MMP1 cDNA under the control of cis-acting GRE were designed. The vectors were designed to increase the expression of MMP1 at the time of glucocorticoid treatment to thereby counteract the glucocorticoid induced down-regulation of MMP1 expression.

Briefly, RNA was reverse transcribed and the full coding MMP1 sequence (1,410 nucleotides; SEQ ID NO: 3) was amplified with restriction sites-ended primers containing a Kozak sequence as indicated herein above in methods. The MMP1 cDNA was inserted downstream of 3 tandem copies of the GRE consensus sequence fused to a TATA-like promoter region from the HSV-thymidine kinase gene available in the commercial vector pGRE.Luc (Clontech, Mountain View, Calif., United States of America). The GRE consensus element consists of a non-perfect palindromic sequence (SEQ ID NO: 2) that is part of a GC regulatory response unit, which in some cases involves more than one GRE, half-site GREs and/or even negative GREs (Nordeen et al., Mol. Endocrinol. 1990; 4:1866-1873). To avoid spurious transcription from upstream sequences, the vector contains a transcription blocker (TrBlk) upstream of the GRE element. This 154 by TrBlk contains a synthetic polyA site and a transcription pause site from the a2 globin gene (Enriquez-Harris et al., EMBO J. 1991; 10:1833-1842). The whole MMP1 expression cassette (TrBlk.GRE.PTAL.MMP1.pA) was then inserted into a shuttle vector to generate the recombinant adenovirus vector (AdhGRE.MMP1) (FIGS. 2A and 2B).

To elaborate further, adenovirus vectors carrying glucocorticoid inducible full coding wild-type and mutant MMP1 cDNAs (AdhGRE.MMP1 and AdhGRE.mutMMP1, respectively) were generated by homologous recombination using the AdEasy Adenoviral Vector System (Stratagene, La Jolla, Calif., United States of America). For the wild-type, the MMP1 cDNA was obtained from RNA extracted from primary HTM cells overexpressing myocilin, which has been shown to increase expression of MMP1 by 26-fold (Borrás T et al., Exp Eye Res. 2006; 82:1002-1010).

A negative functional control vector containing a mutation in the MMP1 active catalytic site was also generated (AdhGRE.mutMMP1; FIG. 2C). For the MMP1 mutant (mutMMP1), the coding sequence was obtained by PCR amplification of plasmid #516 from applicant's HTM1 library (Sellers et al., Biochem J. 1977; 163:303-307) using the same primers, conditions and vector used to amplify and clone wild-type MMP1 (pMG1). Upon sequencing, pMG1 cDNA contained two point mutations at positions 653 and 1115 (position 1 is the A in the ATG initiation of translation codon), which render histidine to arginine and an arginine to lysine amino acid changes, respectively. The first of the two changes affects His 199, which is one of the three essential zinc-binding ligands present in the active site. This change leads to improper folding of the protein and destroys the catalytic activity of MMP1 (Windsor et al., J Biol. Chem. 1994; 269:26201-26207).

Primary HTM-95 cells were infected with AdhTIG3 (Borrás T et al., Exp Eye Res. 2006; 82:1002-1010) at a multiplicity of infection (moi) of 2.6×10⁴ virus genomes/cell (vg/cell), RNA extracted at 72 hours post-infection and RT performed as indicated above. One μl of the RT reaction was amplified using high fidelity Advantage HD polymerase (Clontech, Mountain View, Calif., United States of America) (94° C. 1 minute; 35 cycles: 98° C. 10 seconds, 55° C. 15 seconds, 72° C. 100 seconds; 72° C. 7 minutes), and primers 5′-AAGCTTCCACCATGCACAGCTTTCCTCCACTG-3′ (forward; SEQ ID NO: 97) and 5′-GGCCGGCCTCAATTTTTCCTGCAGTTGA-3′ (reverse; SEQ ID NO: 98). These primers were designed to contain HindIII and FseI sites at their 5′ ends and a CCACC Kozak consensus sequence prior to the MMP1 ATG codon. The amplified 1,424 by DNA fragment was gel purified and cloned into the pCR-blunt II-TOPO plasmid (Invitrogen) (pMG10) for sequence confirmation.

Wild-type (pMG10) and mutant (pMG1) cloning plasmids were then digested with HindIII-FseI, purified, and cloned into a HindIII-FseI predigested pGRE-Luc vector (Clontech, Mountain View, Calif., United States of America) immediately downstream of the transcription blocker (TrBlk), glucocorticoid regulatory element (GRE) and the TATA-like promoter (P_{TAL}) (pMG12 and pMG13, respectively).

To generate recombinant adenoviruses, the MMP1 full expression cassettes (TrBlk.GRE.P_{TAL}.MMP1.pA and TrBlk.GRE.P_{TAL}.mutMMP1.pA) were NotI/SalI digested from vectors pMG12 and pMG13 and inserted at the same restriction sites into the promoterless pShuttle vector (Stratagene) (pMG17 and pMG18). These new vectors were linearized with Pmel and electroporated into BJ5183-Ad1 cells for the recombination with the adenovirus backbone plasmid (pAdEasy1) according to manufacturer's directions. The resultant vectors (pMG19 and pMG20) were amplified in E. coli competent cells XL10-gold (Stratagene), purified, linearized with Pacl and transfected into early-passage QBI-HEK 293A (Qbiogene, Montreal, Canada) for the production of the recombinants (AdhGRE.MMP1 and AdhGRE.mutMMP1). High-titer viral stocks were obtained by propagation in the same cells and purification by double banding CsCl density centrifugation as previously described (Borrás T et al., Gene Ther. 1999; 6:515-524). The collected viral CsCl band was desalted with NAP-5 columns (GE Healthcare, Piscataway, N.J., United States of America) equilibrated with virus vehicle (0.01 M Tris pH 7.4, 1 mM MgCl₂, 10% glycerol), aliquoted and saved at −80° C.

### Example 3

### DEX Regulated Induction of Viral Transferred MMP1

To evaluate the expression levels of recombinant MMP1 in response to DEX treatment, 80% confluent HTM-109 cells were infected with either AdhGRE.MMP1 or AdhGRE.mutMMP1 (moi 5,000 and 6,600 vg/cell, respectively) for 5 days in the presence or absence of 0.1 μM DEX. Results from a representative experiment showed that the normalized level of MMP1 mRNA in the wild-type infected, DEX treated samples was 79.2±9.8-fold of the infected untreated controls (n=3, p=5×10⁻⁶). The MMP1 expression of the mutant infected DEX treated cells was 83.2±10.1-fold of the infected untreated controls (n=3, p=1×10⁻⁶). This high value of expression by the mutant MMP1 virus was expected since the MMP1 mutations were not designed to affect gene transcription (FIG. 3A). The experiment was repeated once in with similar findings.

To determine the levels of recombinant MMP1 protein in HTM cells infected with AdhGRE.MMP1, immunoblotting analysis was carried out for both conditioned media and cell lysates. The results showed that infected cells treated with DEX produced high levels of recombinant MMP1 protein both in the cell associated and secreted fractions as compared to infected, untreated cells (FIG. 3B). Additional quantification of secreted MMP1 levels after AdhGRE.MMP1 infection was determined by ELISA. At 5 days post-infection, recombinant MMP1 levels DEX treated HTM cells were 6,491±169 ng/ml (n=2) compared to 282±32 ng/ml (n=2) in infected untreated cells, respectively, which correspond to a 23-fold induction upon DEX treatment. Repeated experiments, also including infection with the mutated virus confirmed these findings. They further revealed that the level of secreted protein in the AdhGRE.mutMMP1 infected cells was reduced 8.0±2.4 times (n=3), suggesting that the mutated protein might be either secretion impaired and/or subjected to easier degradation than the wild-type. Taken together, the results indicate that cells treated with DEX can induce high expression of MMP1 in cells infected with the adenovirus vector, both at mRNA and protein levels.

### Example 4

### Sequential on and Off Regulation of Delivered MMP1 by Dexamethasone

Once a gene is delivered, one of the goals of using an inducible vector is to be able to turn its expression off and back on again when the corticosteroid stimuli is reapplied. To test the ability of AdhGRE.MMP1 to express the gene under those conditions, HTM-109 cells were infected with 5.1×10³ vg/cell and treated them with 0.1 μM DEX. In parallel wells the corticosteroid was either: removed at 3 days, left for an additional 3 days, or added again at day 6. Results of MMP1 cDNA levels from a representative experiment are shown in FIG. 4. DEX induction for 3 days increased the expression of recombinant MMP1 as expected to 11.7±1.4-fold (n=3, p=2×10⁻⁵). Removal of DEX from the media reversed the induction to 3.4±0.29-fold (n=3, p=0.0002) which was 11.7% of the dish that received DEX continuously for the 6 days (29.1±2.0-fold, n=3, p=5×10⁻⁶). Re-induction with DEX for another 3 days restored the levels of MMP1 close to the original level (14.8±1.2-fold, n=3, p=0.0003). Two more experiments confirmed the findings. These results indicate that during periods of DEX absence, the vector does not induce overexpression of the recombinant gene. This is desirable because overexpression under normal physiological conditions could be damaging for the cells and tissues.

### Example 5

### Collagenase Activity of the HTM Adenovirus-infected Culture Medium

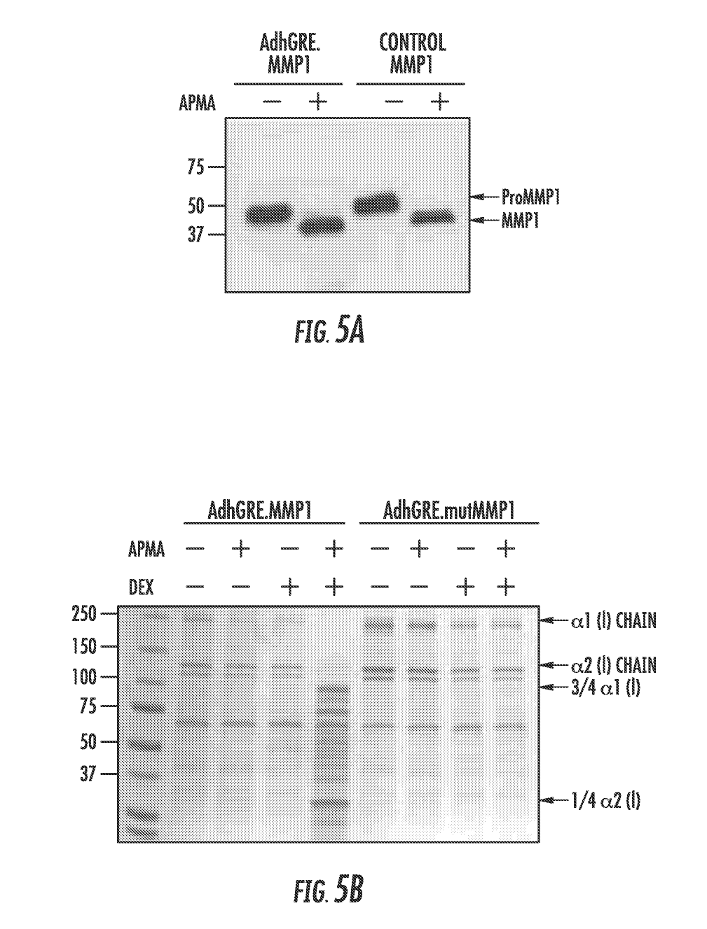

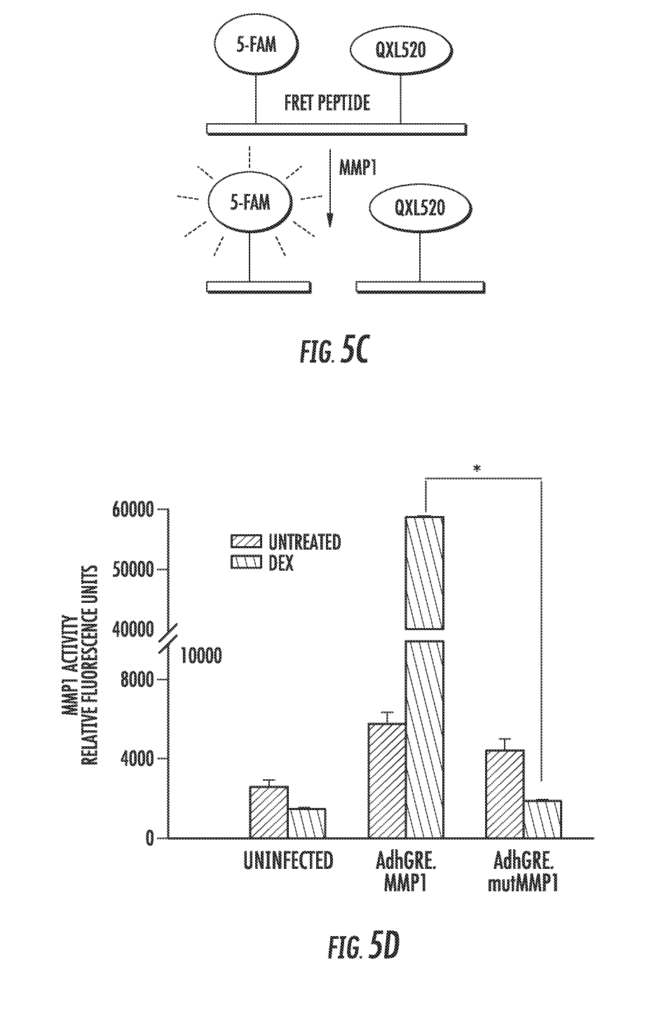

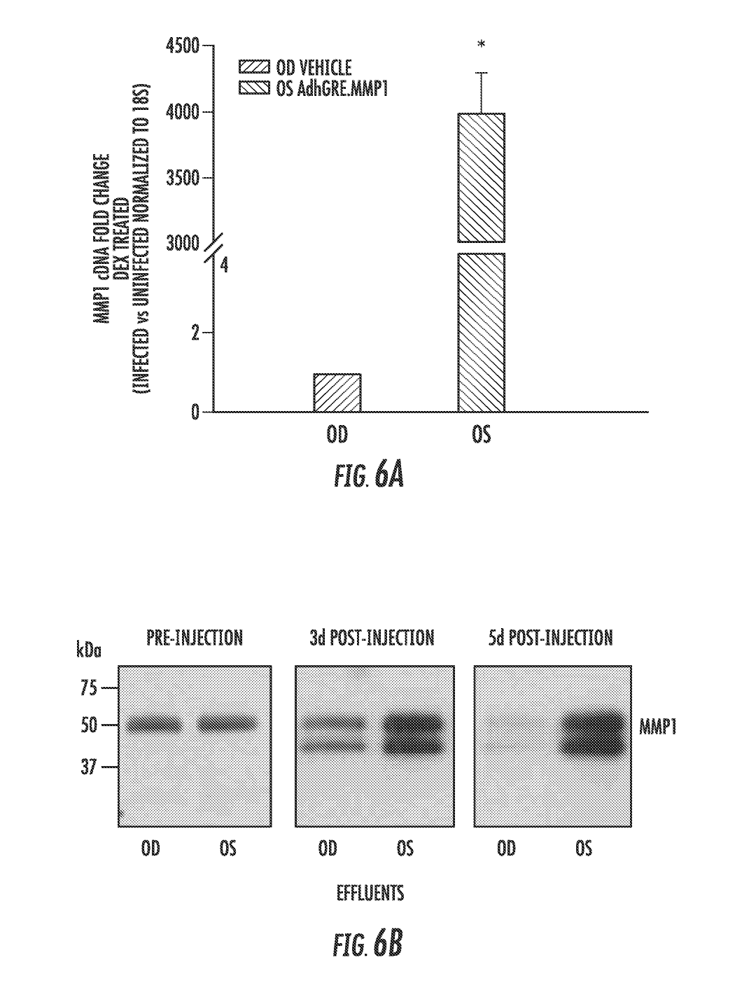

To determine the enzymatic activity of the recombinant MMP1 protein and its ability to degrade collagen, collagen breakdown of the steroid-treated infected cells media was measured using two independent assays. The first assay measured the ability of the DEX-induced infected media to degrade native rat collagen type I by gel electrophoresis. HTM-109 cells at 80-90% confluency in 6-well dishes were infected with AdhGRE.MMP1 and AdhGRE.mutMMP1 (moi 5,000 and 6,600 vg/cell, respectively) and treated with DEX for 5-7 days (n=3). Serum was removed for the last three days of the experiment and media collected for western blot with MMP1 antibody as indicated in methods. Incubation of the concentrated media with APMA for 3 hours resulted in the switch resulted in the switch of the pro-MMP1 band (51 kDa) to the lower, active form of the human MMP1 (41 kDa) (FIG. 5A). Incubation of 25 ng of purified pro-MMP1 (AnaSpec) was used in parallel as a positive control (FIG. 5A).

Activated media from the AdhGRE.MMP1 infected dishes treated with DEX digested native collagen type I into smaller fragments (FIG. 5B, lane 5). The digested products are the ¾ and the ¼ fragments of the α1 and α2 chains respectively and correspond to those previously defined as cleaved by MMP1 (Chung et al., EMBO J. 2004; 23:3020-3030). In contrast, medium from cells infected with AdhGRE.mutMMP1 and overexpressing MMP1 did not exhibit MMP1 enzymatic activity and was unable to cleave collagen (FIG. 5B, lane 9), consistent with the expression of a recombinant mutant MMP1 protein lacking an active catalytic site. In the absence of DEX, the sensitivity of the gel assay was not sufficient to detect collagen digestion by the secreted wild-type (FIG. 5B, lane 3), which was nonetheless observed by the high sensitivity fluorescence assay (FIGS. 5C and 5D).

The second assay measured the potential of the conditioned media to degrade a fluorescently labeled MMP1 substrate FRET peptide (AnaSpec). HTM-109 cells at 80-90% confluency in 6-well dishes were infected with AdhGRE.MMP1 and AdhGRE.mutMMP1 (moi 5,000 and 6,600 vg/cell respectively) and treated with DEX or left untreated for 5 days (n=3). Media was cleared of cellular debris, concentrated 40×, and treated with APMA as indicated in the Materials and Methods for Examples 1-7 above. Equivalent aliquots of treated and controls were incubated with the labeled collagen FRET peptide and the released fluorescence of the cleaved peptide was read in the fluorophotometer (FIG. 5C). The average of three independent experiments is shown in FIG. 5D. Relative fluorescence units of the media of uninfected dishes were 2,719±292 and 1,607±63 for the untreated and DEX-treated cells, respectively. The media from wild-type (AdhGRE.MMP1) infected dishes showed 5,712±550 in the untreated and 59,013±148 in that of DEX-treated cells. In contrast, the media from cells infected with AdhGRE.mutMMP1 gave 4,384±512 and 1,945±101 in the untreated and DEX-treated samples respectively. The statistical comparison between the DEX treated wild-type and mutant was highly significant (p=1×10⁹) (FIG. 5D). Validation of equivalent cell number in treated and untreated dishes was performed in one experiment by measuring intracellular lactate dehydrogenase (LDH) levels (0.96 vs 1.27 OD492/ml in uninfected dishes, 1.29 vs 1.24 OD492/ml and 1.30 vs 1.23 OD492/ml in wild-type and mutant virus infected respectively) using a LDH assay kit (Promega). Altogether, these results indicate that only the recombinant virus with the GRE element driving the wild-type MMP1 secretes the active collagenase when exposed to the glucocorticoid. In addition, the data confirms that not only MMP1 mRNA and protein (FIGS. 1A and B, above), but also MMP1 activity is downregulated by DEX (FIG. 5D). Together these data indicate that the recombinant MMP1 produced by the AdhGRE.MMP1 virus is able to cleave collagen type I in vitro.

### Example 6

### Local Effect of MMP1 Overexpression on Primary HTM Cells

The effect of overexpressing MMP1 in situ was assessed by immunocytochemistry. Primary HTM-106 cells grown in coverslips were treated with 0.1 μM DEX and infected with 2.5×10³ vg/cell of AdhGRE.MMP1. Localization of collagen type I and MMP1 were detected at 48 hours post-infection by double labeling with the corresponding primary and secondary antibodies. In particular, after 48 hours cells were fixed and incubated simultaneously with human anti-MMP1 and anti-collagen type I antibodies followed by fluorescently-tagged Alexa Fluor 555 (for MMP1 in red) and Alexa Fluor 488 (for collagen type I in green) antibodies. Cells were counterstained with DAPI (in blue). Individual cells overexpressing MMP1 (infected by the virus) exhibited lower levels of collagen type I than those not infected. Conversely, cells with lower MMP1 expression exhibited a higher staining intensity for collagen type I. These results indicate that the recombinant MMP1 has a local effect on the collagen of HTM cells. In addition, some of the recombinant MMP1 appeared associated with the cell nuclei.

### Example 7

### Effect of MMP1 Overexpression on Perfused Human Anterior Segments

To best mimic an in vivo situation, the delivery and DEX induction of the AdhGRE.MMP1 vector to the intact human trabecular meshwork (TM) in the perfused organ culture system was assessed. The expression levels of MMP1 were assessed and the ability of the secreted MMP1 to degrade collagen by the FRET assay and immunohistochemistry were measured. Three eye pairs from non glaucomatous post-mortem donors were perfused at constant flow and treated with 0.1 μM DEX as indicated in methods. The HPLC valve connected to one eye was loaded with 6.2×10⁹ vg of AdhGRE.MMP1 at the time of DEX treatment (t=0) (eye pair #1) or twice (t=0 and t=24 h) (eye pairs #2 and #3). The HPLC connected to the contralateral eye was loaded with virus vehicle at the same times.

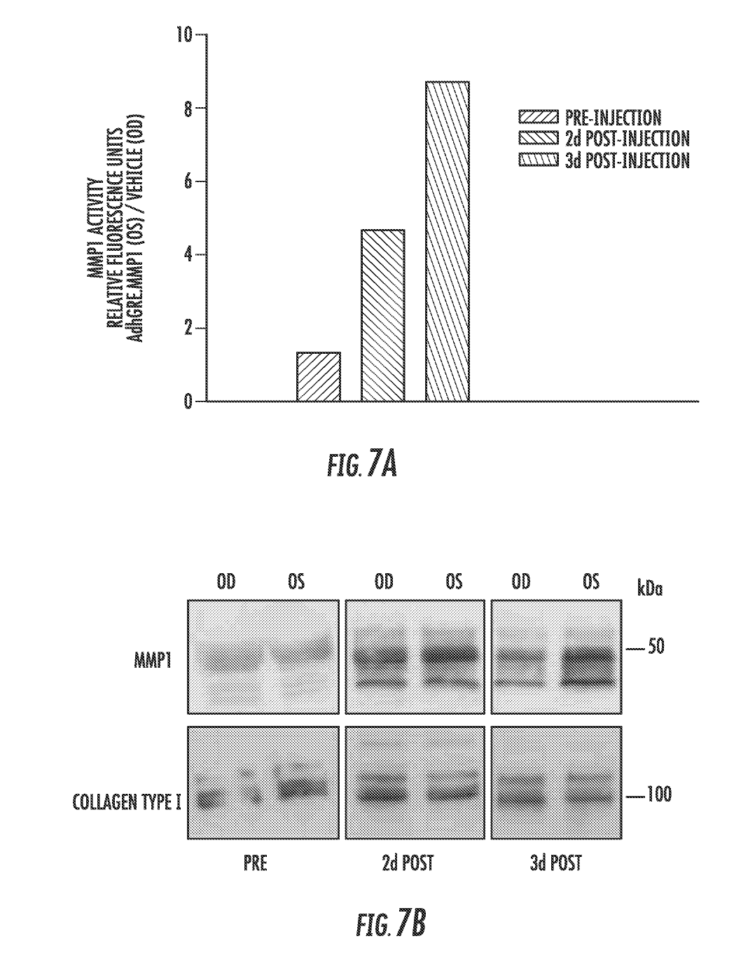

Examination of the delivered MMP1 cDNA in the dissected tissue showed that the mRNA level in the viral injected eye was increased 3,977±220-fold over that of the vehicle injected one (eye #1, n=3, p=1×10⁻⁸) (FIG. 6A). Equivalent aliquots from 40× concentrated effluents from the same eye pair analyzed by western blot showed similar MMP1 band intensities at pre-injection time, while the intensity increased considerably in the eye injected with AdhGRE.MMP1 at 3 and 5 post-injection days (FIG. 6B). Both pro-MMP1 and MMP1 were observed after perfusion with DEX. Quantification of MMP1 levels in these effluents was determined by ELISA at dilution of 1:1,000, each in duplicate. At pre-injection, MMP1 levels were similar in the effluent of both eyes (310 and 317 ng/ml, respectively). At 3 days post-injection, MMP1 levels in the effluent of the AdhGRE.MMP1 injected eye increased 2.4-fold over those in the vehicle-injected (409 vs 168 ng/ml). At 5 days, MMP1 levels in the AdhGRE.MMP1 injected eye reached 4.9-fold over those in the vehicle-injected eye (842 vs 143 ng/ml).

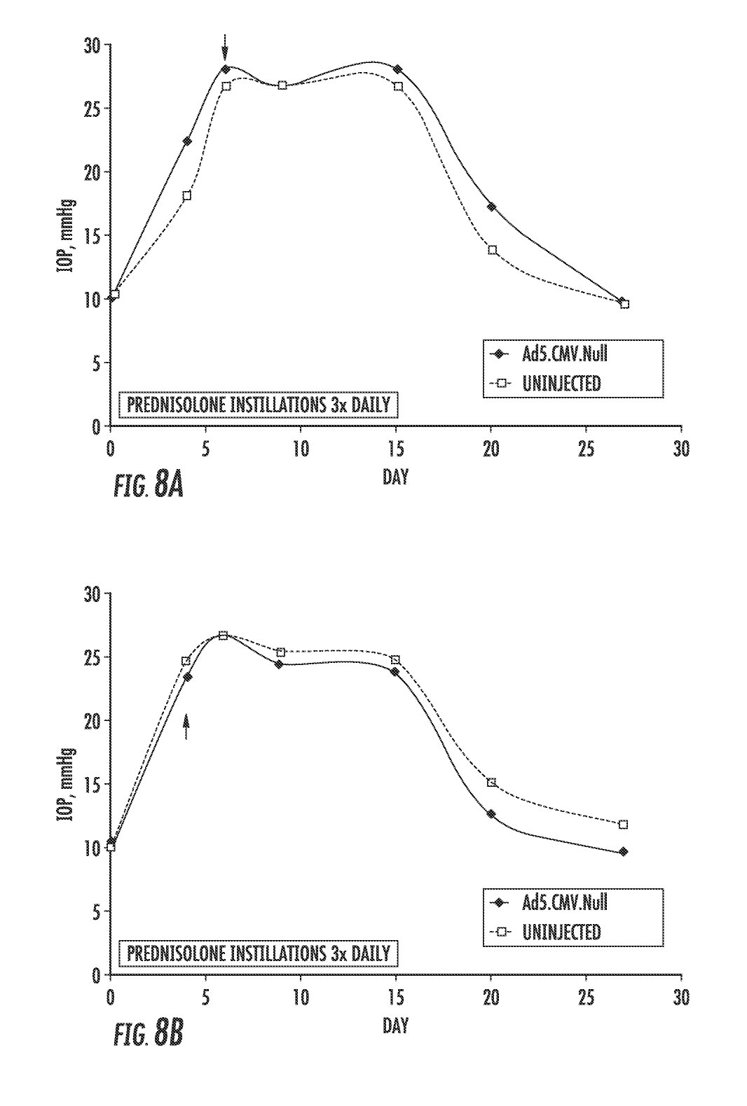

Activity of the MMP1 enzyme by two different assays is shown in FIG. 7. The FRET assay was performed on eye pairs #2 and #3. The ratio of relative fluorescence units in viral- and vehicle-injected eyes (OS/OD) effluents from pair #2 was 1.3-fold at pre-injection time and reached 4.7- and 8.7-fold at 2 and 3 days post-injection respectively (FIG. 7A). For the second pair (#3), the ratio was 1.3-fold at pre-injection time and reached 2.8- and 20.0-fold at 1 and 2 days post-injection, respectively. Concentrated effluents from eye pair #1 were run on PAGE gels, electroblotted, and sequentially probed with MMP1 and collagen type I antibodies as described in the Materials and Methods for Examples 1-7 above. Results in FIG. 7B showed that the intensity of MMP1 protein bands was higher in the effluent of the viral-injected eye than in that of the vehicle-injected control. Conversely, collagen type I bands appeared to be more intense in the vehicle-injected than in the virus-injected eye, concomitant with the presence of a higher activity of the collagenase. Together, these results indicate that the vector successfully enters the cell of the trabecular meshwork tissue, is overexpressed in the presence of DEX, and secretes active MMP1 into the effluent media.

At 60 hours post-injection, the average of the percent changes of outflow facility from baseline of the three eye pairs treated with AdhGRE.MMP1 increased 17.9%±5.9% μl/min/mmHg over that of the vehicle-treated eyes. These preliminary results, showing an increase in outflow facility with overexpression of MMP1, were an indication of this vector's potential for a physiological effect in lowering IOP.

The increase in MMP1 and decrease in collagen could also be observed by immunohistochemistry of the trabecular meshwork tissue by MMP1/collagen type I double-labeling of sections from different quadrants in eye pairs #2 and #3. Eye pairs from non glaucomatous donors were perfused to stable baseline with DMEM and followed by media exchange containing 0.1 μM DEX in both eyes (t=0). Eyes were injected twice through an HPLC loop and perfusion continued in DMEM/DEX media. One eye (OD) received virus vehicle (t=0 and t=24 hours) while the contralateral eye (OS) received 6.2×10⁹ vg per dose of AdhGRE.MMP1 at the same time points. At t=6 days, anterior segments were fixed and embedded in paraffin. Immunohistochemistry was conducted by double labeling with human anti-MMP1 and anti-collagen type I followed by fluorescently-tagged Alexa Fluor 555 (for MMP1 in red) and Alexa Fluor 488 (for collagen type I in green). Tissues were counterstained with DAPI (in blue). In regions of the trabecular meshwork where the MMP1 was intensively stained, collagen type I staining was very faint, especially on the inner wall of the SC and in the juxtacanalicular (JCT) region. The architecture and cell number of the trabecular meshwork tissue was not detrimentally affected by the infection with the virus and subsequent overexpression of MMP1. All regions of the outflow tissue appeared healthy and conserved the canonical layered trabecular meshwork structure and a well-formed SC.

### Example 8

### Discussion of Examples 1-7

One of the significant side effects of corticosteroid therapy is the induction of ocular hypertension, that if untreated would result in the development of steroid-induced glaucoma (Clark et al., Exp Eye Res. 2009; 88:752-759; Jones et al., Curr Opin Ophthalmol. 2006; 17:163-167). To address this unwanted clinical effect at the molecular level the, gene therapy vectors were developed for the potential treatment of steroid-induced glaucoma.

These results first showed that human primary trabecular meshwork cells treated with corticosteroids greatly downregulated MMP1, a metalloproteinase shown to be involved in the ECM turnover of the trabecular meshwork. The three steroids tested here, DEX, triamcinolone acetonide (Kenacort-A), and prednisolone acetate, are widely used in the clinic setting. In particular, the use of intravitreal Kenacort-A has become very popular for the treatment of macular edema and choroidal neovascularization and as a result of this heavy use more patients are developing elevated IOP (Jonas et al., Ophthalmology. 2005; 112:593-598). At the transcriptional level, these experiments showed a downregulation of MMP1 of 500-fold on DEX-treated cells for 6 days, and of 2.4- and 6.2-fold on cells treated with the other two steroids at shorter time periods. The lower transcription of MMP1 resulted in decreased levels of secreted MMP1 protein, which supports that having a downregulated MMP1 protein has detrimental consequences for trabecular meshwork function. Thus, to address the MMP1 deficiency that occurs during a steroid administration episode, a delivery vector with an inducible cassette upstream of the cDNA encoding the MMP1 was designed and engineered. In a representative embodiment the cassette included a GRE element to respond to the steroid, a basal promoter, and an upstream blocker to avoid the generation of other, non specific transcripts.

When primary cells of the human trabecular meshwork were transduced with this vector (AdhGRE.MMP1) they increased their expression of MMP1 mRNA 757-fold in the presence of DEX but not in its absence. This is an indication that in a clinical setting the vector would be active only during a steroid treatment. Desirably, the cycle of induction/non induction was carried over more than once in the same cells. That is, cells transduced with the vector over expressed MMP1 under the first exposure to DEX, returned to basal level when the steroid was removed and overexpressed the enzyme again when exposed to DEX for a second time. This cycled induction/silencing of the vector activity is of value in cases where a patient would require separate steroid administrations. Because some gene therapy vectors have been shown to remain intracellularly (as episomes), and to have the ability of expressing their transgene for as long as five years (Rivera et al., Blood. 2005; 105:1424-1430), having this inducible vector can mean in some embodiments that a single dose would be sufficient, and that further doses would not need to be reapplied when a next steroid treatment is required. During the vector expression term, its transgene DNA will be present in the eye, albeit latent during periods when steroids are not being administered.

An extensive characterization of the MMP1 protein produced by this vector in human primary HTM cells and intact tissue showed that the recombinant enzyme seems to have the same characteristics as the endogenous MMP1. In the primary HTM cells, the protein is secreted as a pro-enzyme which is cleaved and activated by APMA, a thiol-blocking reagent, known to activate latent pro-collagenases, which are enzyme-inhibitor complexes (Sellers et al., Biochem J. 1977; 163:303-307). After incubation with APMA, and using an anti-human MMP1 antibody which detects pro-MMP1 and MMP1, the shift from the 51 kDa pro-MMP1 to the lower 41 kDa active form of the human enzyme was observed. Interestingly, the intracellular MMP1 associated with cultured cell extracts contained both forms of the enzyme, pro-MMP1 and MMP1. Regarding the determination of the recombinant enzyme functional activity, these results showed that the liberated active secreted MMP1 retained its full ability to degrade collagen type I. This activity was measured with a classic assay using exogenous native rat collagen and with a state of the art FRET technology assay using a fluorescently labeled MMP peptide substrate. The specificity of these results was shown by comparing the expression behavior of the wild-type MMP1 adenovirus with that of a parallel control carrying an identical inducible cassette but with a mutant MMP1 cDNA. This mutant contained a point mutation in the cDNA region encoding the catalytic site of the MMP1 which theoretically would produce an inactive enzyme. It was found that the levels of mutant MMP1 mRNA and protein were similar to those produced by the wild-type vector. However, the recombinant mutant protein was unable to degrade collagen in both assays. Together these findings demonstrate that the enzyme produced by the wild-type recombinant vector has the specific ability to degrade components of the trabecular meshwork's ECM. Although the main role attributed to MMP1 is that of degradation of the ECM, this enzyme could perform yet unidentified intracellular functions in the human trabecular meshwork. The observation that some of the recombinant enzyme appears associated with the nuclei is intriguing and raises the possibility that MMP1 could also play a role in other cell functions, as it has been shown in other cell types (Limb et al., Am J. Pathol. 2005; 166:1555-1563).

The MMP1 encoded by the AdhGRE.MMP1 vector was also induced by steroids in a model of perfused human anterior segments from post-mortem donors. In these organ cultures, the trabecular meshwork maintains its natural architecture and the perfused media flows in a manner that mimics the flow of aqueous humor through the tissue. Organ cultures have also the advantage of their serum-free culture conditions (helpful for the study of secreted proteins) and the characteristic of maintaining expression of many genes which get downregulated once the cells are placed in standard tissue cultures. Also, experiments with paired eyes allow the comparison of vehicle- and vector-injected trabecular meshworks from identical genetic backgrounds. The results with the organ cultures confirmed all findings first observed on the HTM cultured cells. The steroid-regulated increase of recombinant MMP1 was observed at the level of transcription in the dissected tissue and at the level of enzyme secretion in the effluents, showing a further increase with perfusion time. Interestingly, at pre-DEX perfusion the trabecular meshwork secreted only the pro-MMP1 form of the protein, while after perfusion with DEX both vector and vehicle-injected eyes secreted the latent and the active form. The collagen activity of the effluents measured by FRET was also found to be greatly increased in the eye injected with the gene therapy vector. Double-labeling immunohistochemistry showed that the intensely stained regions of the delivered MMP1 overlapped with the weak staining of collagen type I. To a lesser extent, western blots also revealed overall lower levels of collagen type I on the eyes with higher MMP1. Lastly, although the number of eyes used in this study was not sufficient to assess a significant change in outflow facility, an increasing trend on the eyes overexpressing MMP1 was observed. These results support lowering IOP in vivo, which is demonstrated in a large animal model of steroid-induced hypertension (see Examples 9-14).

In summary, Examples 1-7 provide a representative novel gene therapy strategy for the treatment of steroid-induced glaucoma in accordance with the presently disclosed subject matter. The vector design and engineering showed that an ECM remodeling enzyme, MMP1, can be induced and silenced by the presence or absence of the steroid. The enzyme produced by the vector under steroid conditions is similar, if not identical, to the endogenous enzyme, and retains the activity to degrade the collagen type I MMP1 substrate. The overproduction of the enzyme counteracts its downregulation by corticosteroids. These findings are further strengthened by the ability of this gene transfer vector to reduce elevated IOP in a model of steroid ocular hypertension in sheep (see Examples 9-14).

### Materials and Methods for Examples 9-14

Animals

All animal experiments were performed in accordance with the Association for Research in Vision and Ophthalmology (ARVO) guidelines. A total of 18 healthy (female) sheep (Corriedale breed) between 12 and 24 months of age, and weighing 35 to 40 kg, were selected from a local ranch in Corrientes, Argentina for this study. The eyes and general health of the animals were considered normal by an ophthalmologist and a veterinarian, respectively. Sheep were tagged for individual identification on their ear lobes and herded from pasture whenever it was necessary to 1) topically instill prednisolone, 2) inject a sub-Tenon depot of triamcinolone acetonide, 3) inject adenoviral vectors carrying the MMP1 transgene intracamerally via the cornea, or 4) measure IOP by applanation tonometry. To apply prednisolone, the sheep were guided into a funnel corral ending in a loose-fitting yoke. This arrangement allowed movement and holding of the head by one person while another instilled the drops. To measure IOP with a Perkins tonometer, the sheep were also guided into the funnel corral and then into the neck yoke. For the sub-Tenon injection of triamcinolone, and the trans-corneal injections of adenoviral vectors, sheep were anesthetized topically. Between all procedures, the sheep were free to pasture.

Prednisolone Instillation Protocol

In those sheep eyes in which prednisolone was used to induce ocular hypertension, the following general protocol was applied. After determining the baseline measurement of IOP over the course of several days, two drops of 0.5% prednisolone acetate (Ultracortenol; Novartis Ophthalmics, Hettlingen, Switzerland) was topically instilled 3-times daily at 7 AM, 2 PM, and 7 PM for durations that lasted for 10-15 days depending on the experiment. In some experiments, prednisolone was instilled bilaterally; in others only one eye received the corticosteroid.

Sub-Tenon Injection of Triamcinolone in Topically Anesthetized Sheep Eyes

In those sheep in which triamcinolone was used to induce ocular hypertension, the following general protocol was applied. Two drops of proparacaine (0.5%) were topically instilled to the ocular surface. Then, a single 1-ml injection of sterile triamcinolone acetonide (40 mg/ml, or 4%; Bristol-Myers Squibb Co., Princeton, N.J., United States of America) was administered via sub-Tenon injection using a 30 G needle inserted 5 mm from the limbus.

For the injections, the distance from the limbus was determined from the width of a 5 mm×30 mm Schirmer strip that was held between the limbus and the injection site. The conjunctiva at the injection site was grasped with fine forceps and an initial oblique conjunctival puncture was made with the needle bevel facing upwards. The needle was then pushed deeper (continuing at an oblique angle) to create another puncture in Tenon's capsule, in such a manner that the Tenon's puncture did not underlie the conjunctival puncture. The needle was pushed through Tenon's capsule until the tip reached the sclera (as determined by feel). Care was taken to avoid puncturing the sclera itself. Also, since Tenon's capsule consists of several layers, care was also taken to administer a sub-Tenon (to create a juxta-scleral depot), and not an intra-Tenon's injection. Immediately following the injection the needle was rapidly withdrawn. The volume injected (1 ml) yielded a characteristic 180°-240° quasi-donut-shaped bolus of fluid around the limbus. Each injected eye then received 2 drops of TOBREX® (tobramycin ophthalmic solution, 0.3%, Alcon Laboratories, Inc., Forth Worth, Tex., United States of America).

Measurement of IOP of Conscious Sheep with the Handheld Perkins Applanation Tonometer

Animals were led to a funnel corral and their heads were suitably oriented within a neck yoke to enable an ophthalmologist to measure IOP with the Perkins tonometer. Before the IOP measurement, 2 drops of topical 0.5% proparacaine (Alcon Argentina) followed by 2 drops of 0.25% fluorescein were instilled. Two sets of measurements were taken on each eye alternating first one eye and then the other. All IOP measurements were taken between 2 PM and 4 PM every 2 or 3 days. The Perkins tonometry readings were converted to mmHg as described in detail previously (Kass et al., Arch Ophthalmol. 2002; 120:701-713).

Intraocular Injection of Adenoviral Vectors into the Anterior Chamber of Sheep

Details of the design, construction, characterization and titration of the steroid-inducible MMP1 adenoviruses are provided in Example 2, above. At the time of viral administration, an EPPENDORF™ tube containing the frozen virus suspension was thawed in the field as the sheep were immobilized within a narrow passage ending in a yoke. Two drops of topical proparacaine 0.5% (Alcon, Argentina) were instilled on the eyes as an anesthetic. Following this 30 μl of the virus suspension was injected into the eye using a Hamilton syringe with a 28 G needle. The needles were inserted diagonally through the cornea (a few millimeters inside the limbus) into the anterior chamber without touching the iris. The injection procedure took less than 30 seconds.

Data Analysis

The significance of experimentally elicited changes in IOP were analyzed using Student's t-test as either paired or unpaired data, with a=0.05 chosen as the level of significance. There are instances in which the two eyes from the same animal can react equally to a treatment; in which case paired analysis can be used. On the other hand, there is evidence that fellow eyes are not identical; in which case unpaired tests should be used. To avoid uncertainties, both tests were used in these experiments.

### Example 9

### Administration of Corticosteroids

The IOP in both eyes of the normal sheep used in this study was measured prior to any treatment to establish the baseline values. The measured Perkins tonometry readings and the equivalent IOP as determined from a calibration curve indicated baseline pressures between about 9-11 mmHg, values similar to those obtained previously (Gerometta et al., Invest. Ophthalmol. Vis. Sci. 2009; 50:669-673).

The present experiments were designed to determine if the intracameral administration of adenoviral vectors carrying an active human MMP1 transgene could both 1) prevent the IOP elevation in a sheep model for glucocorticosteroid-induced ocular hypertension, and 2) reduce the elevation in IOP after its establishment by pretreatments of corticosteroid. Prednisolone was administered as one of the IOP-elevating agents, as used previously (Gerometta et al., Invest. Ophthalmol. Vis. Sci. 2009; 50:669-673). This corticosteroid was administered by thrice-daily topical instillations. With this agent, IOP will remain elevated for as long as the instillation regimen is maintained (Gerometta et al., Invest. Ophthalmol. Vis. Sci. 2009; 50:669-673). The second corticosteroid used in the present study was triamcinolone, which was administered as a single sub-Tenon injection. The advantage of the latter is its less tedious application, because the agent is only introduced once, and subsequent daily administrations are avoided. A disadvantage is that it is difficult to accurately assess as to when the administered triamcinolone depot has been depleted. In experiments described in this study, the triamcinolone depots appeared to subsist for periods of about 2-3 weeks as determined by the IOP, as discussed below.

### Example 10

### Hypotensive Effect of A Single Dose Of Glucocorticoid-Inducible MMP1 Adenovirus On Prednisolone-Induced Ocular Hypertension

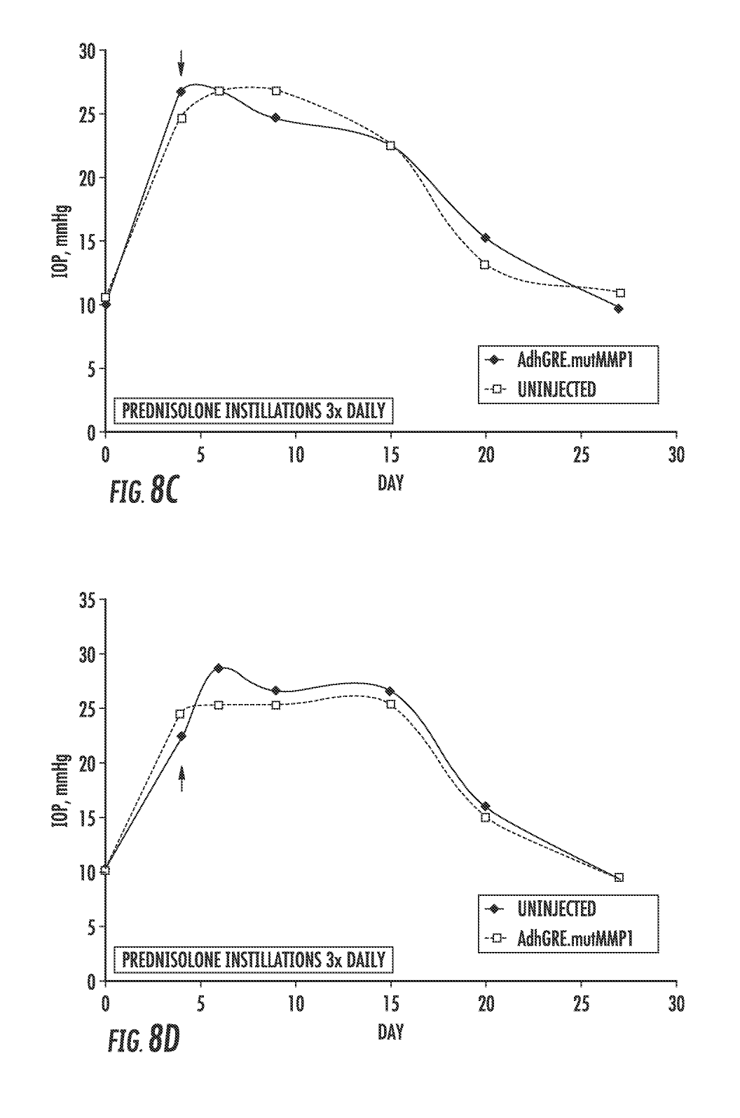

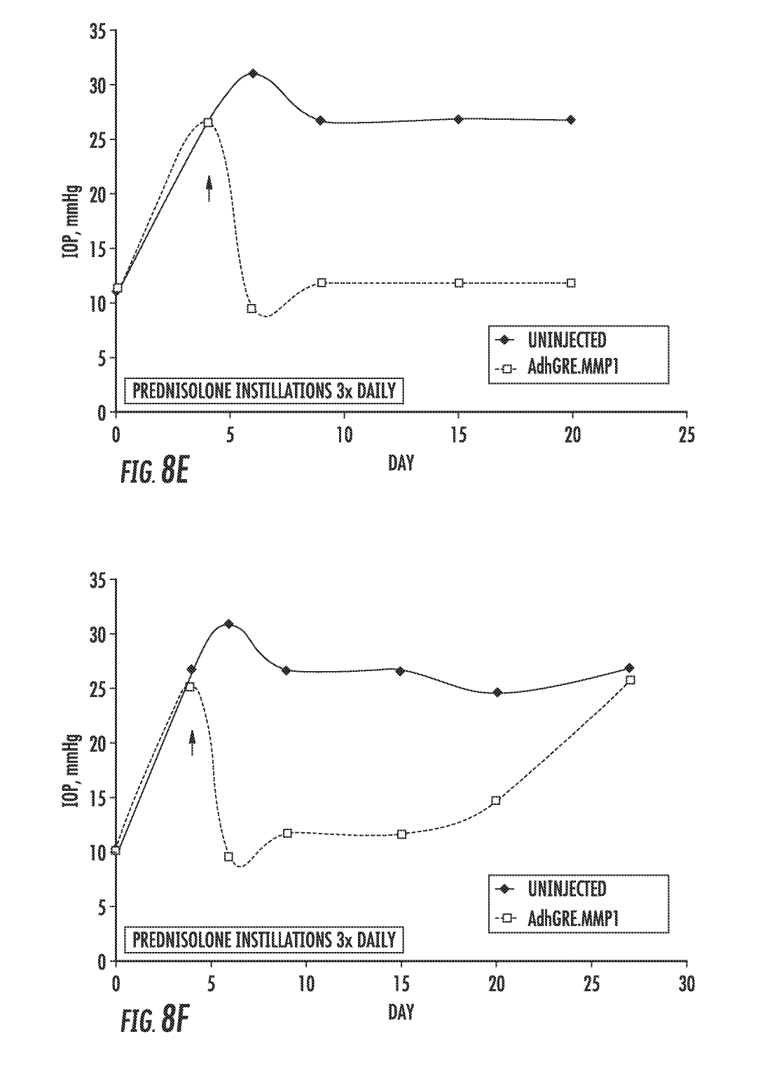

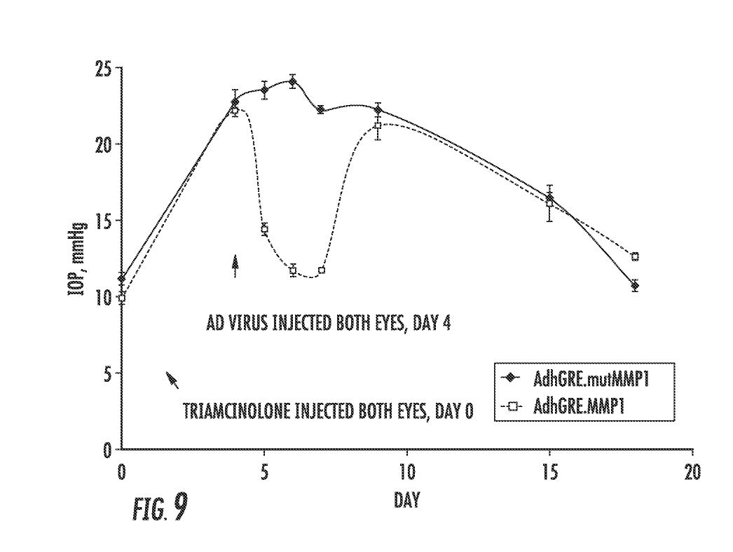

In the first set of experiments (FIGS. 8A-8F), six normal sheep were treated topically with prednisolone three times a day in both eyes beginning on Day 0, Subsequently, as IOP increased, one eye of each sheep was injected with either of 3 adenoviral (Ad) vectors. The active Ad vector (AdhGRE.MMP1) carried the wild-type MMP1 transgene. MMP1 was chosen as it encodes for a well-known TM enzyme that breaks down ECM components. The other 2 vectors (used as controls) included a null Ad vector (without transgene; Ad5.CMV.Null) and a vector carrying a mutated transgene with an inactive catalytic site (AdhGRE.mutMMP1). These vectors also carried the inducible GRE element so that transgene expression would only be activated in the presence of steroids. Among the 6 sheep, two were selected to receive one of the 3 Ad vectors that were prepared. Three to five days after the Ad vector injection, the elevated IOP was reduced in eyes receiving the MMP1 transgene but not in any of the two control eyes.

In the 2 eyes receiving the active MMP1 transgene (FIGS. 8E and 8F), IOP returned to normal levels for at least 15 days. In one sheep, IOP was followed for 20 days (FIG. 8E), while in the second one, the IOP was continuously monitored until Day 27 (FIG. 8F). At this point, the IOP increased to a level identical to that of the fellow eye not exposed to the Ad vector, suggesting that either the induced MMP1 activity was transitory, or overwhelmed by the continuous daily prednisolone applications.

No clinical adverse effects were noted in any of the eyes treated with Ad vectors. There were no signs of conjunctival hyperemia, inflammation or irritation. Likewise, the cornea remained clear without signs of edema in response to the viral injection. Presumably the viral dose administered to the sheep, 5 to 6×10⁹ vg corresponding to approximately 2.5 to 3×10⁹ infectious units, was not sufficient to trigger inflammation in this species.

### Example 11

### Hypotensive Effect of A Single Dose Of Glucocorticoid-Inducible MMP1 Adenovirus On Triamcinolone-Induced Ocular Hypertension

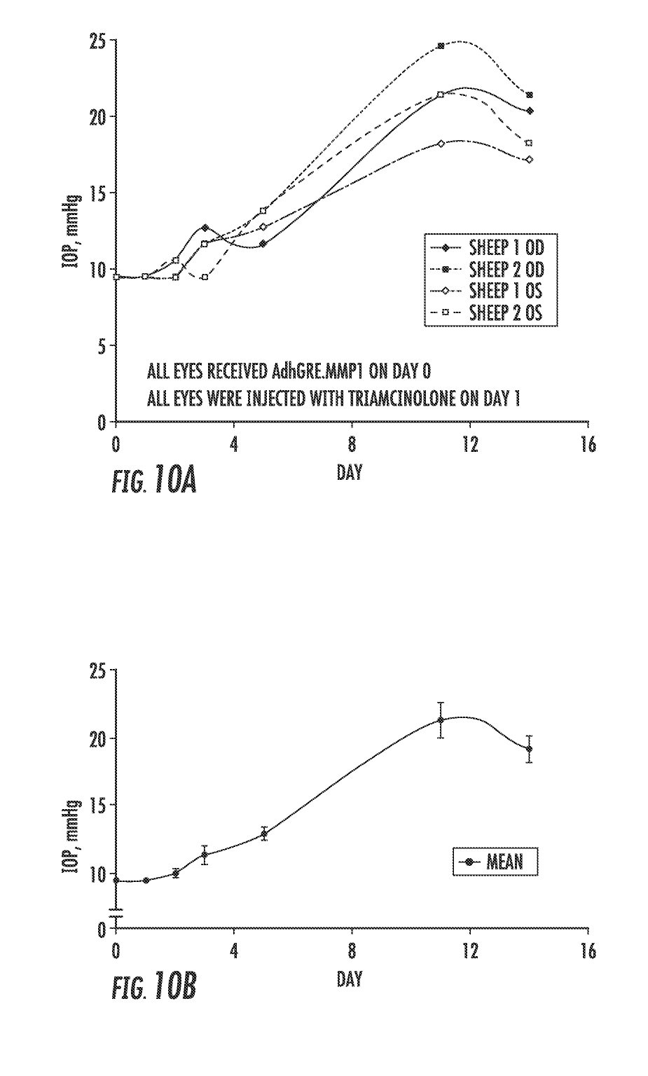

Triamcinolone was used as the IOP-elevating agent in the second set of experiments (FIG. 9). For this, 4 normal sheep received bilaterally sub-Tenon injections of triamcinolone on Day 0, which caused the IOP to approximately double within 4 days. IOP increased from 11.0±0.3 to 22.6±0.8 mmHg (n=4) in the eyes to which the Ad vectors carrying the mutated MMP1 transgene were then injected; and from 9.7±0.2 to 22.1±0.2 mmHg (n=4) in the eyes to which the Ad vectors carrying the active MMP1 transgene were then injected (P<3×10⁻⁴, as paired data for these IOP increases between days 0 and 4). After recording the IOP on Day 4, both eyes of each animal were injected with the respective vectors. On Days 6 and 7 (or 2 and 3 days after the virus injections), the IOPs of the eyes receiving the active form of the transgene were significantly lower than that of the follow eye (P<2×10⁻⁵, as paired data). For example, on Day 7, the IOP of the eye injected with the Ad with the mutated form of MMP1 was 22.1±0.2 mmHg (n=4), while that of the fellow eye receiving the active form was 11.6±0.4 mmHg (n=4). Thereafter, the IOP of the latter eye increased to a level nearly identical to that of the fellow eye by Day 9 (P>0.15, as paired data). The reason for this increase was not determined. It is not clear as to why the apparent transitory MMP1 activity persisted for at least 15 days in the case of the experiments shown in FIGS. 8A-8F, and only 3 days in the case of the experiments shown in FIG. 9. In the experiments shown in FIG. 9, the triamcinolone depot appeared to be nearly consumed by Day 18, as judged by the IOP measurements. On this day, the IOP's of the fellow eyes were 10.5±0.4 mmHg (n=4) and 12.4±0.2 mmHg (n=4).

In tandem with the experiments shown in FIG. 9, three other sheep were analyzed in parallel at the same time to check the effect of injecting null Ad vectors (without transgene) on the elevated IOP induced by triamcinolone (IOP plots not shown). For this test, each sheep unilaterally received a single sub-Tenon injection of triamcinolone on Day 0. With 2 sheep the injections were made in the right eye, while the left eye was used for the third sheep. On Day 4, the IOP of the control eyes was 10.9±0.7 mmHg (n=3) and 25.2±0.4 mmHg (n=3) in the eyes administered triamcinolone (P<3×10⁻⁴, as either paired or unpaired data). After taking these readings, the triamcinolone-treated eyes were injected with null Ad vectors. Three days later, the IOP's were 9.4±0.0 mmHg (n=3) and 23.1±0.4 (n=3) for the control and steroid-treated eyes, respectively. The IOP of the latter was significantly lower statistically (P<0.04, as paired data) three days after receiving the null Ad, yet it was clear that the measured pressure difference was meager and the IOP remained 2.5-fold higher than the control fellow eye. In contrast, in eyes receiving active MMP1 transgene, marked hypotensive effects can be measured within 24 hours of Ad vector injection, with reversions to the baseline IOP within 2-3 days (FIGS. 8A-8F and 9).

### Example 12

### Protective Effect of A Single Dose Of Glucocorticoid-Inducible MMP1 Adenovirus Injected Prior To Triamcinolone Treatment

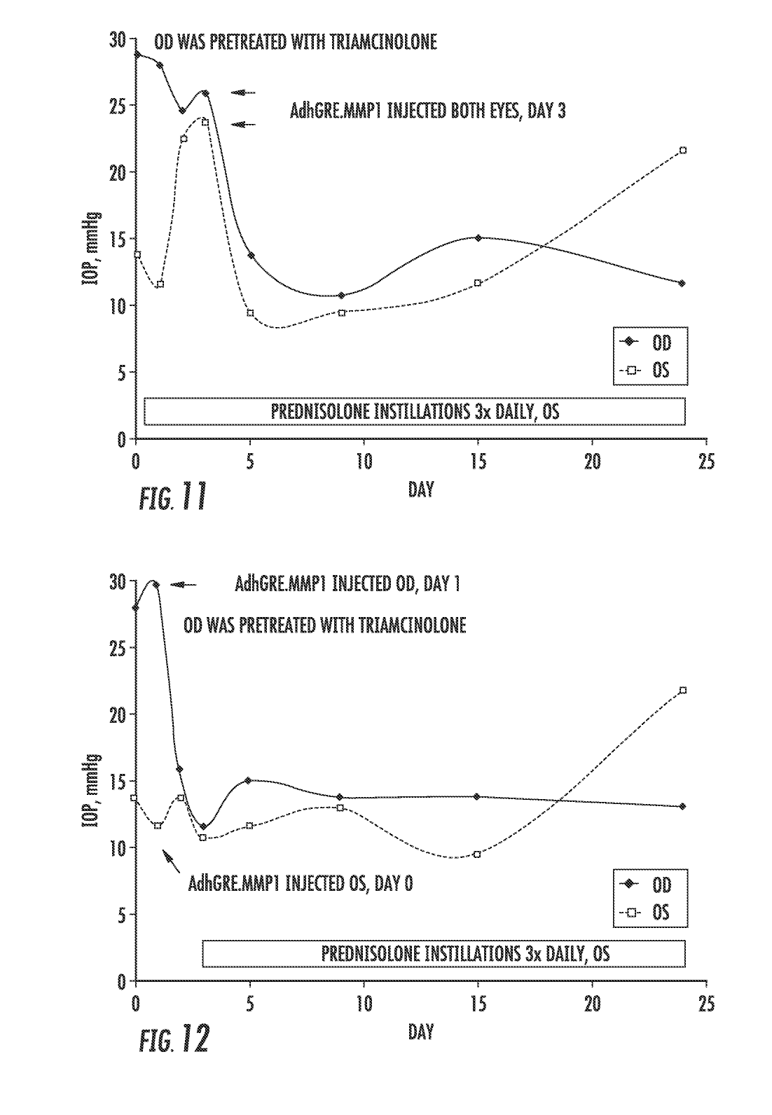

This protocol tested for preventive effects from pretreatments with MMP1 transgene on the elevated IOP induced by the triamcinolone depot. In these experiments, two sheep were bilaterally injected with Ad virus carrying the active transgene on Day 0, and received sub-Tenon injections of triamcinolone in both eyes on Day 1 (FIGS. 10A and 10B). In these 4 eyes, IOP remained near normal levels until at least Day 5, at which point IOP was 12.9±0.5 (n=4). The Day 5 IOP value was significantly higher than the 9.4 ±0.1 mm Hg (n=4) measured on Day 0 (P<0.005, as paired data). At Days 11 and 14, IOP readings were 21.3±1.3 (n=4) and 19.1±1.0 (n=4), respectively. These latter values were significantly higher than that at Day 5 (P<0.005, as paired data). The increase in IOP between Day 5 and Day 14 occurred with a time frame within which the triamcinolone depot likely subsisted. These results suggest that the active transgene offered protection against triamcinolone administration for at least 3 days. A protective effect is evident because the injections of triamcinolone in the control eye evoke a doubling of IOP within 4 days (FIG. 9).

### Example 13

### Hypotensive Effects Of A Single Dose Of Glucocorticoid-Inducible MMP1 Adenovirus On Sheep Simultaneously Administered Triamcinolone In One Eye And Prednisolone In The Other

Additional experiments (employing 2 separate protocols, each on one sheep) tested the effects of the injection of Ad virus carrying active MMP1 transgene on the IOP of contralateral eyes treated with unilateral triamcinolone and unilateral prednisolone. In the first protocol of this set, one eye of a sheep was administered triamcinolone via a sub-Tenon injection two weeks before the start of IOP measurements. The point at which IOP measurements were initiated was designated “Day 0” of the experiment. Eye OD, which had received the triamcinolone exhibited an IOP about twice that of eye OS (FIG. 11). After recording these measurements, thrice daily instillations of prednisolone were begun on eye OS. Three-days later, when the IOP's of both eyes were nearly identical, Ad virus carrying active MMP1 transgene was bilaterally injected into the anterior chambers of the eyes. Subsequently, IOP declined in tandem in the fellow eyes (FIG. 11). At the point of virus injection, IOP of eye OD had been elevated for 17 days; after virus injection, IOP was reduced up to Day 24. It is also possible that triamcinolone lost its effectiveness, or the depot was gradually depleted, between Days 15 and 24 (FIG. 11). In eye OS, between Days 15 and 24, IOP gradually increased presumably because of a diminished effect from the injected MMP1 transgene in the face of continuous daily prednisolone instillations.

In the second protocol, eye OS of a sheep was injected with Ad virus carrying the active transgene on Day 0, and the thrice-daily instillations of prednisolone were begun on Day 3 (FIG. 12). The contralateral OD eye was pretreated with a sub-Tenon injection of triamcinolone two weeks before the initiation of IOP measurements. On Day 0, IOP measurements were initiated, and eye OD exhibited an IOP about twice that of eye OS (FIG. 12), indicating a typical response to corticosteroid administration. On Day 1, eye OD was injected with Ad virus carrying active MMP1 transgene from the same lot as that given to eye OS; this injection resulted in an ocular hypotensive effect within 24 hours in the right eye. Thereafter, IOP of eye OD remained low due to active protection from the MMP1 transgene and/or from a loss of effect of triamcinolone. The fellow OS eye appeared to have received a protective effect from the MMP1 transgene administration up to at least Day 15; thereafter, IOP gradually increased due to the continuous prednisolone instillations (FIG. 12).

### Example 14

### Discussion of Examples 9-13

Sheep are docile and compliant animals that are particularly well suited for in vivo experiments such as those done in Examples 9-13. Moreover, the ovine physiology appears to be similar in terms of aqueous secretion to that of the human (Gerometta et al., Exp. Eye Res. 2005; 80:307-312), and trabecular meshwork anatomy also appears to be rather similar to primates (Simoens P. et al., J. Vet. Med. Sci. 1996; 58:977-982; Guyomard J L et al., Invest. Ophthalmol. Vis. Sci. 2008; 49:5168-5174). One advantage of using an ovine steroid-induced model of IOP elevation is the consistency and robustness of the IOP response as well as the relatively low cost compared with studies in primates. Moreover, the sheep model for corticosteroid-induced ocular hypertension was preferable to other animal models such as rabbit. With the latter, only about 50% of rabbits treated chronically with glucocorticoids such as dexamethasone develop ocular hypertension, and dexamethasone responders are commonly defined as those exhibiting IOP elevations of at least 5 mmHg (Pang et al., Exp. Eye Res. 2001; 73:815-825). In contrast, all treated sheep responded to prednisolone with about 2.5-fold increases in IOP as reported previously (Gerometta et al., Invest. Ophthalmol. Vis. Sci. 2009; 50:669-673), and sub-Tenon injection of a triamcinolone depot was observed to be equally effective as prednisolone in elevating ovine IOP in that all sheep administered triamcinolone exhibited ocular hypertension.

In the present study, triamcinolone was used for convenience to avoid the necessity of applying prednisolone 3-times daily as the sole method for elevating IOP in all protocols. The triamcinolone acetonide preparation of the drug is minimally water soluble and is injected into sub-Tenon's space as a suspension. In this form, the lowered water solubility contributes to the formation of a relatively long-lasting depot, from which drug delivery into the eye occurs via the sclera (Jermak C M et al., Surv. Ophthalmol. 2007; 52:503-522; Mora et al., Curr. Eye Res. 2005; 30:355-361). However, as discussed by Robinson et al., many studies have observed intra-subject variability in intraocular drug levels following sub-conjunctival injection (Robinson M R et al., Exp. Eye Res. 2006; 82:479-487). These authors have suggested that a number of factors might influence drug release from the depot, and intra-ocular entry, including differences in conjunctival lymphatic and capillary blood flow, in scleral thickness and choroidal flow, and differences in the geometry of the depot, in itself (Robinson et al., Exp. Eye Res. 2006; 82:479-487). Presumably, the complex release kinetics of sub-Tenon triamcinolone might account for the varied degree and duration of the IOP elevations that were obtained in our protocols. For example, the IOP of the triamcinolone-treated eye is above 25 mmHg on Day 15 in both FIGS. 11 and 12, whereas in FIG. 9, an IOP of less than 20 mmHg was observed 15 days after sub-Tenon drug injection. Nevertheless, the MMP1 transgene was tested at the plateau of the elevated IOP to confirm the utility of this approach in reversing the induced ocular hypertension, which was an aim of this study.

In this work, it was determined that a single dose of a gene therapy vector carrying an inducible human metalloproteinase 1 gene (MMP1) could both 1) temporarily prevent the increase in IOP normally produced by glucocorticosteroid instillations in the sheep model, and 2) reverse the IOP increase previously induced by the glucocorticosteroids.

Cataract formation was not observed in any of the animals used in these experiments. In the case of the triamcinolone depot, the drug appeared to subsist in the eye for about 14-20 days, as judged by the IOP measurements.

Putatively, the triamcinolone depot might be a more effective experimental approach (compared to topical instillations) for elevating IOP since the drug is continuously present in the eye until the depot is totally dissipated. In contrast, there were 7-12 hour intervals between the administrations of the prednisolone drops. This difference in technique might explain the current observations that the MMP1 activity induced by the Ad vector preparations seems to have persisted for at least 15 days in the case of the prednisolone experiments shown in FIGS. 8A-8F, and only 3 days in the case of the triamcinolone experiments shown in FIG. 9.

The experiments in Examples 9-13 demonstrate that steroid glaucoma can treated with an inducible over-expression of extracellular matrix modulator genes. Given common characteristics between steroid glaucoma and POAG, this therapy should have general applicability to numerous species including humans.

### REFERENCES

Altschul et al. (1990) J Mol Biol 215: 403-410.
Ausubel (1995) Short Protocols in Molecular Biology, 3rd ed. Wiley, New York.
Barton (1998) Acta Crystallogr D Biol Crystallogr 54:1139-1146.
Batzer et al. (1991) Nuc Acids Res 19:5081.
Betageri et al., 1993 Liposome Drug Delivery Systems, Technomic Publishing, Lancaster.
Borrás T, Rowlette L L, Erzurum S C, Epstein D L. Adenoviral reporter gene transfer to the human trabecular meshwork does not alter aqueous humor outflow. relevance for potential gene therapy of glaucoma. Gene Ther. 1999; 6:515-524.
Buie L K, Rasmussen C A, Porterfield E C, et al. Self-complementary AAV virus (scAAV) safe and long-term gene transfer in the trabecular meshwork of living rats and monkeys. Invest Ophthalmol Vis Sci. 2010; 51:236-248.
Clark A F. AL-3789: A novel ophthalmic angiostatic steroid. Expert Opin. Investig. Drugs 1997; 6:1867-1877.
Clark A F. Mechanism of action of the angiostatic cortisene anecortave acetate. Surv. Ophthalmol. 2007; 52 Suppl 1:S26-34.
Gerometta R, Podos S M, Danias J, Candia O A. Steroid-induced ocular hypertension in normal sheep. Invest. Ophthalmol. Vis. Sci. 2009; 50:669-673.
Gerometta R M, Malgor L A, Vilalta E, Leiva J, Candia O A. Cl-concentrations of bovine, porcine and ovine aqueous humor are higher than in plasma. Exp. Eye Res. 2005; 80:307-312.
Gillies M C, Kuzniarz M, Craig J, Ball M, Luo W, Simpson J M. Intravitreal triamcinolone-induced elevated intraocular pressure is associated with the development of posterior subcapsular cataract. Ophthalmology 2005; 112:139-143.
Glover & Hames (1995) DNA Cloning: A Practical Approach, 2nd ed. IRL Press at Oxford University Press, Oxford/New York.
Goldman et al. (1997) Cancer Res 57:1447-1451.
Goldman et al. (1997) Nat Biotechnol 15:462.
Gregoriadis, ed., 1993 Liposome Technology, CRC Press, Boca Raton, Fla.
Guyomard J L, Rosolen S G, Paques M, et al. A low-cost and simple imaging technique of the anterior and posterior segments: Eye fundus, ciliary bodies, iridocorneal angle. Invest. Ophthalmol. Vis. Sci. 2008; 49:5168-5174.
Heller et al. (1996) FEBS Lett 389:225-228.
Henikoff & Henikoff (2000) Adv Protein Chem 54:73-97
Henikoff et al. (2000) Electrophoresis 21(9):1700-1706.
Huang et al. (2000) Pac Symp Biocomput 230-241.
Janoff, ed. 1999 Liposomes: Rational Design, M. Dekker, New York, N.Y.
Jermak C M, Dellacroce J T, Heffez J, Peyman G A. Triamcinolone acetonide in ocular therapeutics. Surv. Ophthalmol. 2007; 52:503-522.
Jones R, 3rd, Rhee D J. Corticosteroid-induced ocular hypertension and glaucoma: A brief review and update of the literature. Curr. Opin. Ophthalmol. 2006; 17:163-167.
Karlin & Altschul (1993) Proc Natl Acad Sci USA 90:5873-5887.
Kersey J P, Broadway D C. Corticosteroid-induced glaucoma: A review of the literature. Eye 2006; 20:407-416.
Kyte et al. (1982) J Mol Biol 157:105.
Labat-Moleur et al., 1996 Gene Therapy 3:1010-1017.
Lasic & Martin, 1995 Stealth Liposomes, CRC Press, Boca Raton, Fla.
Luna et al. (2000) Cancer Res 60(6):1637-1644.
Manome et al. (1994) Cancer Res 54:5408-5413.
McLean J. Use of ACTH and cortisone. Trans Am Ophthalmol Soc 1950; 48:293-296.
Miklavcic et al. (1998) Biophys J 74:2152-2158.
Mora P, Eperon S, Felt-Baeyens O, et al. Trans-scleral diffusion of triamcinolone acetonide. Curr. Eye Res. 2005; 30:355-361.
Nabel, 1997 “Vectors for Gene Therapy” in Current Protocols in Human Genetics on CD-ROM, John Wiley & Sons, New York, N.Y.
Needleman & Wunsch (1970) J Mol Biol 48:443.
Ohtsuka et al. (1985) J Biol Chem 260:2605-2608.
Pang I H, Moll H, McLaughlin M A, et al. Ocular hypotensive and aqueous outflow-enhancing effects of AL-3037A (sodium ferri ethylenediaminetetraacetate). Exp. Eye Res. 2001; 73:815-825.
Pearson & Lipman (1988) Proc Natl Acad Sci USA 85:2444-2448.
Porter et al. (2001) J Mol Endocrinol 26(1):31-42.
Robinson M R, Lee S S, Kim H, et al. A rabbit model for assessing the ocular barriers to the transscleral delivery of triamcinolone acetonide. Exp. Eye Res. 2006; 82:479-487.
Rossolini et al. (1994) Mol Cell Probes 8:91-98.
Sadekova (1997) Int J Radiat Biol 72(6):653-660.
Saltzman & Fung (1997) Adv Drug Deliv Rev 26:209-230.
Sambrook & Russell (2001) Molecular Cloning: a Laboratory Manual, 3rd ed. Cold Spring Harbor Laboratory Press, Cold Spring Harbor, N. Y.
Saqi et al. (1999) Bioinformatics 15(6):521-522.
Silhavy et al. (1984) Experiments with Gene Fusions. Cold Spring Harbor Laboratory, Cold Spring Harbor, N.Y.
Simoens P, DeGeest J P, Lauwers H. Comparative morphology of the pectinate ligaments of domestic mammals, as observed under the dissecting microscope and the scanning electron microscope. J. Vet. Med. Sci. 1996; 58:977-982.
Smith & Waterman (1981) Adv Appl Math 2:482.
U.S. Pat. No. 4,235,871.
U.S. Pat. No. 4,551,482
U.S. Pat. No. 4,554,101.
U.S. Pat. No. 5,011,634.
U.S. Pat. No. 5,490,840.
U.S. Pat. No. 5,510,103.
U.S. Pat. No. 5,651,991.
U.S. Pat. No. 5,688,931.
U.S. Pat. No. 5,714,166.
U.S. Pat. No. 5,786,387.
U.S. Pat. No. 5,855,900.
U.S. Pat. No. 5,858,410.
U.S. Pat. No. 5,922,356.
U.S. Pat. No. 5,922,545.
U.S. Pat. No. 5,948,767.
U.S. Pat. No. 5,994,392.
U.S. Pat. No. 6,056,938.
U.S. Pat. No. 6,106,866.
U.S. Pat. No. 6,132,766.
U.S. Pat. No. 6,197,333.
U.S. Pat. No. 6,200,598.
U.S. Pat. No. 6,210,707.
U.S. Pat. No. 6,217,886.
Vicat et al. (2000) Hum Gene Ther 11:909-916.

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A steroid-inducible vector comprising a coding sequence for a polypeptide of interest, a minimal promoter and a steroid response element (SRE), wherein the coding sequence is under the transcriptional control of the SRE, wherein the coding sequence corresponds to a gene susceptible to altered expression in the presence of a steroid in vivo.

2. The steroid-inducible vector of claim 1, wherein the vector is an adenovirus vector.

3. The steroid-inducible vector of claim 1, wherein the SRE is a glucocorticoid response element (GRE).

4. The steroid-inducible vector of claim 3, wherein the GRE increases transcription of the coding sequence in the presence of a steroid selected from the group consisting of dexamethasone, triamcinalone acetonide, prednisolone acetate, and combinations thereof.

5. The steroid-inducible vector of claim 1, wherein the polypeptide of interest is MMP1.

6. The steroid-inducible vector of claim 5, wherein the coding sequence for MMP1 comprises a nucleotide sequence of SEQ ID NO: 3, or a nucleotide sequence 95% identical to SEQ ID NO: 3.

7. The steroid-inducible vector of claim 6, wherein the MMP1 polypeptide comprises an amino acid sequence of SEQ ID NO: 4, or an amino acid sequence 95% identical to SEQ ID NO: 4.

8. A method of treating steroid glaucoma in a subject in need thereof, the method comprising:
i) providing a subject suffering from steroid glaucoma;
ii) providing a vector comprising a coding sequence for a polypeptide of interest, a minimal promoter and a SRE, wherein the coding sequence is under the transcriptional control of the SRE, wherein the coding sequence corresponds to a gene susceptible to altered expression in the presence of a steroid in vivo; and
iii) administering the vector to the subject, wherein the steroid glaucoma is treated.

9. The method of claim 8, wherein the steroid glaucoma comprises elevated intraocular pressure (IOP).

10. The method of claim 9, wherein the elevated IOP is decreased.

11. The method of claim 8, wherein the steroid glaucoma comprises increased extracellular matrix (ECM) deposition.

12. The method of claim 11, wherein the ECM deposition is decreased.

13. The method of claim 8, wherein the vector is an adenovirus vector.

14. The method of claim 8, wherein the SRE is a glucocorticoid response element (GRE).

15. The method of claim 8, wherein the polypeptide of interest is MMP1.

16. The method of claim 8, wherein administering the vector comprises administering the vector to an ocular tissue of the subject.

17. The method of claim 8, wherein the subject is a mammal.

18. The method of claim 8, wherein the subject is receiving a steroid treatment, wherein the steroid is a glucocorticoid selected from the group consisting of dexamethasone, triamcinalone acetonide, prednisolone acetate, and combinations thereof.

19. A method of preventing elevated intraocular pressure (IOP) in a subject receiving steroid treatment, the method comprising:
i) providing a subject receiving steroid treatment;
ii) providing a vector comprising a coding sequence for a polypeptide of interest, a minimal promoter and a SRE, wherein coding sequence is under the transcriptional control of the SRE, wherein the coding sequence corresponds to a gene susceptible to altered expression in the presence of a steroid in vivo; and
iii) administering the vector to the subject, wherein elevated IOP in the subject is prevented.

20. The method of claim 19, wherein the vector is an adenovirus vector.

21. The method of claim 19, wherein the SRE is a glucocorticoid response element (GRE).

22. The steroid-inducible vector of claim 19, wherein the polypeptide of interest is MMP1.

23. The method of claim 19, wherein the subject is a mammal.

24. The method of claim 19, wherein the steroid treatment comprises the administration of a glucocorticoid to the subject, wherein the glucocorticoid is selected from the group consisting of dexamethasone, triamcinalone acetonide, prednisolone acetate, and combinations thereof.

25. The method of claim 19, wherein administering the vector comprises administering the vector to an ocular tissue of the subject.

26. A method of reversing elevated intraocular pressure (IOP) in a subject receiving steroid treatment, the method comprising:
i) providing a subject receiving steroid treatment, wherein the subject has elevated IOP;
ii) providing a vector comprising a coding sequence for a polypeptide of interest, a minimal promoter and a SRE, wherein the coding sequence is under the transcriptional control of the SRE, wherein the coding sequence corresponds to a gene susceptible to altered expression in the presence of a steroid in vivo; and
iii) administering the vector to the subject, wherein the elevated IOP in the subject is reversed.

27. The method of claim 26, wherein the vector is an adenovirus vector.

28. The method of claim 26, wherein the SRE is a glucocorticoid response element (GRE).

29. The steroid-inducible vector of claim 26, wherein the polypeptide of interest is MMP1.

30. The method of claim 26, wherein the subject is a mammal.

31. The method of claim 26, wherein the steroid treatment comprises the administration of a glucocorticoid to the subject, wherein the glucocorticoid is selected from the group consisting of dexamethasone, triamcinalone acetonide, prednisolone acetate, and combinations thereof.

32. The method of claim 26, wherein administering the vector comprises administering the vector to an ocular tissue of the subject.

33. An steroid treatment method comprising:
i) providing a subject in need of steroid treatment;
ii) administering a vector comprising a coding sequence for a polypeptide of interest, a minimal promoter and a SRE, wherein the coding sequence is under the transcriptional control of the SRE, wherein the coding sequence corresponds to a gene susceptible to altered expression in the presence of a steroid in vivo; and
iii) administering a steroid to the subject.

34. The method of claim 33, wherein the subject in need of steroid treatment comprises a subject suffering from inflammation, ocular inflammation, macular edema, choroidal neovascularization, or any other eye or systemic condition requiring administration of a steroid.

35. The method of claim 33, wherein the vector is administered prior to, simultaneously, or after steroid administration.

36. The method of claim 33, wherein the vector is an adenovirus vector.

37. The method of claim 33, wherein the SRE is a glucocorticoid response element (GRE).

38. The method of claim 33, wherein the polypeptide of interest is MMP1.

39. The method of claim 33, wherein the subject is a mammal.

40. The method of claim 33, wherein the steroid is selected from the group consisting of dexamethasone, triamcinalone acetonide, prednisolone acetate, and combinations thereof.

41. A method of treating or preventing a condition associated with steroid treatment in a subject, the method comprising:
i) providing a subject receiving steroid treatment;
ii) providing a vector comprising a coding sequence for a polypeptide of interest, a minimal promoter and a SRE, wherein the coding sequence is under the transcriptional control of the SRE, wherein the coding sequence corresponds to a gene susceptible to altered expression in the presence of a steroid in vivo; and
iii) administering the vector to the subject.

42. The method of claim 41, wherein the vector is an adenovirus vector.

43. The method of claim 41, wherein the SRE is a glucocorticoid response element (GRE).

44. The method of claim 41, wherein the polypeptide of interest is MMP1.

45. The method of claim 41, wherein the subject is a mammal.

46. The method of claim 41, wherein the steroid treatment comprises the administration of a glucocorticoid to the subject, wherein the glucocorticoid is selected from the group consisting of dexamethasone, triamcinalone acetonide, prednisolone acetate, and combinations thereof.

47. The method of claim 41, wherein administering the vector comprises administering the vector to an ocular tissue of the subject.

48. The method of claim 41, wherein the vector is administered prior to, simultaneously, or after steroid administration.

49. A composition comprising the steroid-inducible vector of claim 1 and a pharmaceutically acceptable carrier.


SEQUENCE LISTING (98 sequences)

SEQ ID NO: 1 - DNA, 2069 bp, Artificial Sequence
  expression cassette
ggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataggtaccgagctcttacgcgtgctagcggtacattttgttctagaacaaaatgtaccggtacattttgttctagatctgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagaagcttccaccatgcacagctttcctccactgctgctgctgctgttctggggtgtggtgtctcacagcttcccagcgactctagaaacacaagagcaagatgtggacttagtccagaaatacctggaaaaatactacaacctgaagaatgatgggaggcaagttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgaggggaaccctcgctgggagcaaacacatctgacctacaggattgaaaattacacgccagatttgccaagagcagatgtggaccatgccattgagaaagccttccaactctggagtaatgtcacacctctgacattcaccaaggtctctgagggtcaagcagacatcatgatatcttttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcggctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcgtgtgacagtaagctaacctttgatgctataactacgattcggggagaagtgatgttctttaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaggacatctacagctcctttggcttccctagaactgtgaagcatatcgatgctgctctttctgaggaaaacactggaaaaacctacttctttgttgctaacaaatactggaggtatgatgaatataaacgatctatggatccaggttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaattgaggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccg

SEQ ID NO: 2 - DNA, 45 bp, Homo sapiens
ggtacattttgttctagaacaaaatgtaccggtacattttgttct

SEQ ID NO: 3 - DNA, 1410 bp, Homo sapiens
atgcacagctttcctccactgctgctgctgctgttctggggtgtggtgtctcacagcttcccagcgactctagaaacacaagagcaagatgtggacttagtccagaaatacctggaaaaatactacaacctgaagaatgatgggaggcaagttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgaggggaaccctcgctgggagcaaacacatctgacctacaggattgaaaattacacgccagatttgccaagagcagatgtggaccatgccattgagaaagccttccaactctggagtaatgtcacacctctgacattcaccaaggtctctgagggtcaagcagacatcatgatatcttttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcggctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcgtgtgacagtaagctaacctttgatgctataactacgattcggggagaagtgatgttctttaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaggacatctacagctcctttggcttccctagaactgtgaagcatatcgatgctgctctttctgaggaaaacactggaaaaacctacttctttgttgctaacaaatactggaggtatgatgaatataaacgatctatggatccaggttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaattga

SEQ ID NO: 4 - protein, 469 aa, Homo sapiens
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 5 - DNA, 1828 bp, Homo sapiens
ctacaaggaggcaggcaagacagcaaggcatagagacaacatagagctaagtaaagccagtggaaatgaagagtcttccaatcctactgttgctgtgcgtggcagtttgctcagcctatccattggatggagctgcaaggggtgaggacaccagcatgaaccttgttcagaaatatctagaaaactactacgacctcaaaaaagatgtgaaacagtttgttaggagaaaggacagtggtcctgttgttaaaaaaatccgagaaatgcagaagttccttggattggaggtgacggggaagctggactccgacactctggaggtgatgcgcaagcccaggtgtggagttcctgatgttggtcacttcagaacctttcctggcatcccgaagtggaggaaaacccaccttacatacaggattgtgaattatacaccagatttgccaaaagatgctgttgattctgctgttgagaaagctctgaaagtctgggaagaggtgactccactcacattctccaggctgtatgaaggagaggctgatataatgatctcttttgcagttagagaacatggagacttttacccttttgatggacctggaaatgttttggcccatgcctatgcccctgggccagggattaatggagatgcccactttgatgatgatgaacaatggacaaaggatacaacagggaccaatttatttctcgttgctgctcatgaaattggccactccctgggtctctttcactcagccaacactgaagctttgatgtacccactctatcactcactcacagacctgactcggttccgcctgtctcaagatgatataaatggcattcagtccctctatggacctccccctgactcccctgagacccccctggtacccacggaacctgtccctccagaacctgggacgccagccaactgtgatcctgctttgtcctttgatgctgtcagcactctgaggggagaaatcctgatctttaaagacaggcacttttggcgcaaatccctcaggaagcttgaacctgaattgcatttgatctcttcattttggccatctcttccttcaggcgtggatgccgcatatgaagttactagcaaggacctcgttttcatttttaaaggaaatcaattctgggctatcagaggaaatgaggtacgagctggatacccaagaggcatccacaccctaggtttccctccaaccgtgaggaaaatcgatgcagccatttctgataaggaaaagaacaaaacatatttctttgtagaggacaaatactggagatttgatgagaagagaaattccatggagccaggctttcccaagcaaatagctgaagactttccagggattgactcaaagattgatgctgtttttgaagaatttgggttcttttatttctttactggatcttcacagttggagtttgacccaaatgcaaagaaagtgacacacactttgaagagtaacagctggcttaattgttgaaagagatatgtagaaggcacaatatgggcactttaaatgaagctaataattcttcacctaagtctctgtgaattgaaatgttcgttttctcctgcctgtgctgtgactcgagtcacactcaagggaacttgagcgtgaatctgtatcttgccggtcatttttatgttattacagggcattcaaatgggctgctgcttagcttgcaccttgtcacatagagtgatctttcccaagagaaggggaagcactcgtgtgcaacagacaagtgactgtatctgtgtagactatttgcttatttaataaagacgatttgtcagttattttatctt

SEQ ID NO: 6 - protein, 477 aa, Homo sapiens
MKSLPILLLLCVAVCSAYPLDGAARGEDTSMNLVQKYLENYYDLKKDVKQFVRRKDSGPVVKKIREMQKFLGLEVTGKLDSDTLEVMRKPRCGVPDVGHFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPVPPEPGTPANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 7 - DNA, 1743 bp, Homo sapiens
aaagaaggtaagggcagtgagaatgatgcatcttgcattccttgtgctgttgtgtctgccagtctgctctgcctatcctctgagtggggcagcaaaagaggaggactccaacaaggatcttgcccagcaatacctagaaaagtactacaacctcgaaaaggatgtgaaacagtttagaagaaaggacagtaatctcattgttaaaaaaatccaaggaatgcagaagttccttgggttggaggtgacagggaagctagacactgacactctggaggtgatgcgcaagcccaggtgtggagttcctgacgttggtcacttcagctcctttcctggcatgccgaagtggaggaaaacccaccttacatacaggattgtgaattatacaccagatttgccaagagatgctgttgattctgccattgagaaagctctgaaagtctgggaagaggtgactccactcacattctccaggctgtatgaaggagaggctgatataatgatctctttcgcagttaaagaacatggagacttttactcttttgatggcccaggacacagtttggctcatgcctacccacctggacctgggctttatggagatattcactttgatgatgatgaaaaatggacagaagatgcatcaggcaccaatttattcctcgttgctgctcatgaacttggccactccctggggctctttcactcagccaacactgaagctttgatgtacccactctacaactcattcacagagctcgcccagttccgcctttcgcaagatgatgtgaatggcattcagtctctctacggacctccccctgcctctactgaggaacccctggtgcccacaaaatctgttccttcgggatctgagatgccagccaagtgtgatcctgctttgtccttcgatgccatcagcactctgaggggagaatatctgttctttaaagacagatatttttggcgaagatcccactggaaccctgaacctgaatttcatttgatttctgcattttggccctctcttccatcatatttggatgctgcatatgaagttaacagcagggacaccgtttttatttttaaaggaaatgagttctgggccatcagaggaaatgaggtacaagcaggttatccaagaggcatccataccctgggttttcctccaaccataaggaaaattgatgcagctgtttctgacaaggaaaagaagaaaacatacttctttgcagcggacaaatactggagatttgatgaaaatagccagtccatggagcaaggcttccctagactaatagctgatgactttccaggagttgagcctaaggttgatgctgtattacaggcatttggatttttctacttcttcagtggatcatcacagtttgagtttgaccccaatgccaggatggtgacacacatattaaagagtaacagctggttacattgctaggcgagatagggggaagacagatatgggtgtttttaataaatctaataattattcatctaatgtattatgagccaaaatggttaatttttcctgcatgttctgtgactgaagaagatgagccttgcagatatctgcatgtgtcatgaagaatgtttctggaattcttcacttgcttttgaattgcactgaacagaattaagaaatactcatgtgcaataggtgagagaatgtattttcatagatgtgttattacttcctcaataaaaagttttattttgggcctgttcctt

SEQ ID NO: 8 - protein, 476 aa, Homo sapiens
MMHLAFLVLLCLPVCSAYPLSGAAKEEDSNKDLAQQYLEKYYNLEKDVKQFRRKDSNLIVKKIQGMQKFLGLEVTGKLDTDTLEVMRKPRCGVPDVGHFSSFPGMPKWRKTHLTYRIVNYTPDLPRDAVDSAIEKALKVWEEVTPLTFSRLYEGEADIMISFAVKEHGDFYSFDGPGHSLAHAYPPGPGLYGDIHFDDDEKWTEDASGTNLFLVAAHELGHSLGLFHSANTEALMYPLYNSFTELAQFRLSQDDVNGIQSLYGPPPASTEEPLVPTKSVPSGSEMPAKCDPALSFDAISTLRGEYLFFKDRYFWRRSHWNPEPEFHLISAFWPSLPSYLDAAYEVNSRDTVFIFKGNEFWAIRGNEVQAGYPRGIHTLGFPPTIRKIDAAVSDKEKKKTYFFAADKYWRFDENSQSMEQGFPRLIADDFPGVEPKVDAVLQAFGFFYFFSGSSQFEFDPNARMVTHILKSNSWLHC

SEQ ID NO: 9 - DNA, 1877 bp, Homo sapiens
cagaacccggactaagggctatataaagaggaacagttcaggaacttaggctagaaaggaacacagtaaactgaattgatccgtttagaagtttacaatgaagtttcttctaatactgctcctgcaggccactgcttctggagctcttcccctgaacagctctacaagcctggaaaaaaataatgtgctatttggtgaaagatacttagaaaaattttatggccttgagataaacaaacttccagtgacaaaaatgaaatatagtggaaacttaatgaaggaaaaaatccaagaaatgcagcacttcttgggtctgaaagtgaccgggcaactggacacatctaccctggagatgatgcacgcacctcgatgtggagtccccgatgtccatcatttcagggaaatgccaggggggcccgtatggaggaaacattatatcacctacagaatcaataattacacacctgacatgaaccgtgaggatgttgactacgcaatccggaaagctttccaagtatggagtaatgttacccccttgaaattcagcaagattaacacaggcatggctgacattttggtggtttttgcccgtggagctcatggagacttccatgcttttgatggcaaaggtggaatcctagcccatgcttttggacctggatctggcattggaggggatgcacatttcgatgaggacgaattctggactacacattcaggaggcacaaacttgttcctcactgctgttcacgagattggccattccttaggtcttggccattctagtgatccaaaggccgtaatgttccccacctacaaatatgttgacatcaacacatttcgcctctctgctgatgacatacgtggcattcagtccctgtatggagacccaaaagagaaccaacgcttgccaaatcctgacaattcagaaccagctctctgtgaccccaatttgagttttgatgctgtcactaccgtgggaaataagatctttttcttcaaagacaggttcttctggctgaaggtttctgagagaccaaagaccagtgttaatttaatttcttccttatggccaaccttgccatctggcattgaagctgcttatgaaattgaagccagaaatcaagtttttctttttaaagatgacaaatactggttaattagcaatttaagaccagagccaaattatcccaagagcatacattcttttggttttcctaactttgtgaaaaaaattgatgcagctgtttttaacccacgtttttataggacctacttctttgtagataaccagtattggaggtatgatgaaaggagacagatgatggaccctggttatcccaaactgattaccaagaacttccaaggaatcgggcctaaaattgatgcagtcttctactctaaaaacaaatactactatttcttccaaggatctaaccaatttgaatatgacttcctactccaacgtatcaccaaaacactgaaaagcaatagctggtttggttgttagaaatggtgtaattaatggtttttgttagttcacttcagcttaataagtatttattgcatatttgctatgtcctcagtgtaccactacttagagatatgtatcataaaaataaaatctgtaaaccataggtaatgattatataaaatacataatatttttcaattttgaaaactctaattgtccattcttgcttgactctactattaagtttgaaaatagttaccttcaaaggccaagagaattctatttgaagcatgctctgtaagttgcttcctaacatccttggactgagaaattatacttacttctggcataactaaaattaagtatatatattttggctcaaataaaattgaaaaaaaaatca

SEQ ID NO: 10 - protein, 470 aa, Homo sapiens
MKFLLILLLQATASGALPLNSSTSLEKNNVLFGERYLEKFYGLEINKLPVTKMKYSGNLMKEKIQEMQHFLGLKVTGQLDTSTLEMMHAPRCGVPDVHHFREMPGGPVWRKHYITYRINNYTPDMNREDVDYAIRKAFQVWSNVTPLKFSKINTGMADILVVFARGAHGDFHAFDGKGGILAHAFGPGSGIGGDAHFDEDEFWTTHSGGTNLFLTAVHEIGHSLGLGHSSDPKAVMFPTYKYVDINTFRLSADDIRGIQSLYGDPKENQRLPNPDNSEPALCDPNLSFDAVTTVGNKIFFFKDRFFWLKVSERPKTSVNLISSLWPTLPSGIEAAYEIEARNQVFLFKDDKYWLISNLRPEPNYPKSIHSFGFPNFVKKIDAAVFNPRFYRTYFFVDNQYWRYDERRQMMDPGYPKLITKNFQGIGPKIDAVFYSKNKYYYFFQGSNQFEYDFLLQRITKTLKSNSWFGC

SEQ ID NO: 11 - DNA, 3927 bp, Homo sapiens
gcggcggcaggcctagcagcacgggaaccgtcccccgcgcgcatgcgcgcgcccctgaagcgcctgggggacgggtatgggcgggaggtaggggcgcggctccgcgtgccagttgggtgcccgcgcgtcacgtggtgaggaaggaggcggaggtctgagtttcgaaggagggggggagagaagagggaacgagcaagggaaggaaagcggggaaaggaggaaggaaacgaacgagggggagggaggtccctgttttggaggagctaggagcgttgccggcccctgaagtggagcgagagggaggtgcttcgccgtttctcctgccaggggaggtcccggcttcccgtggaggctccggaccaagccccttcagcttctccctccggatcgatgtgctgctgttaacccgtgaggaggcggcggcggcggcagcggcagcggaagatggtgttgctgagagtgttaattctgctcctctcctgggcggcggggatgggaggtcagtatgggaatcctttaaataaatatatcagacattatgaaggattatcttacaatgtggattcattacaccaaaaacaccagcgtgccaaaagagcagtctcacatgaagaccaatttttacgtctagatttccatgcccatggaagacatttcaacctacgaatgaagagggacacttcccttttcagtgatgaatttaaagtagaaacatcaaataaagtacttgattatgatacctctcatatttacactggacatatttatggtgaagaaggaagttttagccatgggtctgttattgatggaagatttgaaggattcatccagactcgtggtggcacattttatgttgagccagcagagagatatattaaagaccgaactctgccatttcactctgtcatttatcatgaagatgatattaactatccccataaatacggtcctcaggggggctgtgcagatcattcagtatttgaaagaatgaggaaataccagatgactggtgtagaggaagtaacacagatacctcaagaagaacatgctgctaatggtccagaacttctgaggaaaaaacgtacaacttcagctgaaaaaaatacttgtcagctttatattcagactgatcatttgttctttaaatattacggaacacgagaagctgtgattgcccagatatccagtcatgttaaagcgattgatacaatttaccagaccacagacttctccggaatccgtaacatcagtttcatggtgaaacgcataagaatcaatacaactgctgatgagaaggaccctacaaatcctttccgtttcccaaatattggtgtggagaagtttctggaattgaattctgagcagaatcatgatgactactgtttggcctatgtcttcacagaccgagattttgatgatggcgtacttggtctggcttgggttggagcaccttcaggaagctctggaggaatatgtgaaaaaagtaaactctattcagatggtaagaagaagtccttaaacactggaattattactgttcagaactatgggtctcatgtacctcccaaagtctctcacattacttttgctcacgaagttggacataactttggatccccacatgattctggaacagagtgcacaccaggagaatctaagaatttgggtcaaaaagaaaatggcaattacatcatgtatgcaagagcaacatctggggacaaacttaacaacaataaattctcactctgtagtattagaaatataagccaagttcttgagaagaagagaaacaactgttttgttgaatctggccaacctatttgtggaaatggaatggtagaacaaggtgaagaatgtgattgtggctatagtgaccagtgtaaagatgaatgctgcttcgatgcaaatcaaccagagggaagaaaatgcaaactgaaacctgggaaacagtgcagtccaagtcaaggtccttgttgtacagcacagtgtgcattcaagtcaaagtctgagaagtgtcgggatgattcagactgtgcaagggaaggaatatgtaatggcttcacagctctctgcccagcatctgaccctaaaccaaacttcacagactgtaataggcatacacaagtgtgcattaatgggcaatgtgcaggttctatctgtgagaaatatggcttagaggagtgtacgtgtgccagttctgatggcaaagatgataaagaattatgccatgtatgctgtatgaagaaaatggacccatcaacttgtgccagtacagggtctgtgcagtggagtaggcacttcagtggtcgaaccatcaccctgcaacctggatccccttgcaacgattttagaggttactgtgatgttttcatgcggtgcagattagtagatgctgatggtcctctagctaggcttaaaaaagcaatttttagtccagagctctatgaaaacattgctgaatggattgtggctcattggtgggcagtattacttatgggaattgctctgatcatgctaatggctggatttattaagatatgcagtgttcatactccaagtagtaatccaaagttgcctcctcctaaaccacttccaggcactttaaagaggaggagacctccacagcccattcagcaaccccagcgtcagcggccccgagagagttatcaaatgggacacatgagacgctaactgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgcattttcttcacttgcaggcaaacttggctctcaataaacttttaccacaaattgaaataaatatatttttttcaactgccaatcaaggctaggaggctcgaccacctcaacattggagacatcacttgccaatgtacataccttgttatatgcagacatgtatttcttacgtacactgtacttctgtgtgcaattgtaaacagaaattgcaatatggatgtttctttgtattataaaatttttccgctcttaattaaaaattactgtttaattgacatactcaggataacagagaatggtggtattcagtggtccaggattctgtaatgctttacacaggcagttttgaaatgaaaatcaatttacctttctgttacgatggagttggttttgatactcattttttctttatcacatggctgctacgggcacaagtgactatactgaagaacacagttaagtgttgtgcaaactggacatagcagcacatactacttcagagttcatgatgtagatgtctggtttctgcttacgtcttttaaactttctaattcaattccatttttcaattaataggtgaaattttattcatgctttgatagaaattatgtcaatgaaatgattctttttatttgtagcctacttatttgtgtttttcatatatctgaaatatgctaattatgttttctgtctgatatggaaaagaaaagctgtgtctttatcaaaatatttaaacggttttttcagcatatcatcactgatcattggtaaccactaaagatgagtaatttgcttaagtagtagttaaaattgtagataggccttctgacattttttttcctaaaatttttaacagcattgaaggtgaaacagcacaatgtcccattccaaatttatttttgaaacagatgtaaataattggcattttaaagag

SEQ ID NO: 12 - protein, 748 aa, Homo sapiens
MVLLRVLILLLSWAAGMGGQYGNPLNKYIRHYEGLSYNVDSLHQKHQRAKRAVSHEDQFLRLDFHAHGRHFNLRMKRDTSLFSDEFKVETSNKVLDYDTSHIYTGHIYGEEGSFSHGSVIDGRFEGFIQTRGGTFYVEPAERYIKDRTLPFHSVIYHEDDINYPHKYGPQGGCADHSVFERMRKYQMTGVEEVTQIPQEEHAANGPELLRKKRTTSAEKNTCQLYIQTDHLFFKYYGTREAVIAQISSHVKAIDTIYQTTDFSGIRNISFMVKRIRINTTADEKDPTNPFRFPNIGVEKFLELNSEQNHDDYCLAYVFTDRDFDDGVLGLAWVGAPSGSSGGICEKSKLYSDGKKKSLNTGIITVQNYGSHVPPKVSHITFAHEVGHNFGSPHDSGTECTPGESKNLGQKENGNYIMYARATSGDKLNNNKFSLCSIRNISQVLEKKRNNCFVESGQPICGNGMVEQGEECDCGYSDQCKDECCFDANQPEGRKCKLKPGKQCSPSQGPCCTAQCAFKSKSEKCRDDSDCAREGICNGFTALCPASDPKPNFTDCNRHTQVCINGQCAGSICEKYGLEECTCASSDGKDDKELCHVCCMKKMDPSTCASTGSVQWSRHFSGRTITLQPGSPCNDFRGYCDVFMRCRLVDADGPLARLKKAIFSPELYENIAEWIVAHWWAVLLMGIALIMLMAGFIKICSVHTPSSNPKLPPPKPLPGTLKRRRPPQPIQQPQRQRPRESYQMGHMRR

SEQ ID NO: 13 - DNA, 6466 bp, Homo sapiens
gccgggcagtgggcaggtatggctgagggcgtgtgagcgccgagcgctaagggccgccgccaccatgccagggggcgcaggcgccgcccggctctgcttgctggcgtttgccctgcagcccctccggccgcgggcggcgcgggagcctggatggacaagaggaagtgaggaaggcagccccaagctgcagcatgaacttatcatacctcagtggaagacttcagaaagccccgtgagagaaaagcatccactcaaagctgagctcagggtaatggctgaggggcgagaactgatcctggacctggagaagaatgagcaactttttgctccttcctacacagaaacccattatacttcaagtggtaaccctcaaaccaccacacggaaattggaggatcactgcttttaccacggcacggtgagggagacagaactgtccagcgtcacgctcagcacttgccgaggaattagaggactgattacggtgagcagcaacctcagctacgtcatcgagcccctccctgacagcaagggccaacaccttatttacagatctgaacatctcaagccgcccccgggaaactgtgggttcgagcactccaagcccaccaccagggactgggctcttcagtttacacaacagaccaagaagcgacctcgcaggatgaaaagggaagatttaaactccatgaagtatgtggagctttacctcgtggctgattatttagagtttcagaagaatcgacgagaccaggacgccaccaaacacaagctcatagagatcgccaactatgttgataagttttaccgatccttgaacatccggattgctctcgtgggcttggaagtgtggacccacgggaacatgtgtgaagtttcagagaatccatattctaccctctggtcctttctcagttggaggcgcaagctgcttgcccagaagtaccatgacaacgcccaattaatcacgggcatgtccttccacggcaccaccatcggcctggcccccctcatggccatgtgctctgtgtaccagtctggaggagtcaacatggaccactccgagaatgccattggcgtggctgccaccatggcccacgagatgggccacaactttggcatgacccatgattctgcagattgctgctcggccagtgcggctgatggtgggtgcatcatggcagctgccactgggcacccctttcccaaagtgttcaatggatgcaacaggagggagctggacaggtatctgcagtcaggtggtggaatgtgtctctccaacatgccagacaccaggatgttgtatggaggccggaggtgtgggaacgggtatctggaagatggggaagagtgtgactgtggagaagaagaggaatgtaacaacccctgctgcaatgcctctaattgtaccctgaggccgggggcggagtgtgctcacggctcctgctgccaccagtgtaagctgttggctcctgggaccctgtgccgcgagcaggccaggcagtgtgacctcccggagttctgtacgggcaagtctccccactgccctaccaacttctaccagatggatggtaccccctgtgagggcggccaggcctactgctacaacggcatgtgcctcacctaccaggagcagtgccagcagctgtggggacccggagcccgacctgcccctgacctctgcttcgagaaggtgaatgtggcaggagacacctttggaaactgtggaaaggacatgaatggtgaacacaggaagtgcaacatgagagatgcgaagtgtgggaagatccagtgtcagagctctgaggcccggcccctggagtccaacgcggtgcccattgacaccactatcatcatgaatgggaggcagatccagtgccggggcacccacgtctaccgaggtcctgaggaggagggtgacatgctggacccagggctggtgatgactggaaccaagtgtggctacaaccatatttgctttgaggggcagtgcaggaacacctccttctttgaaactgaaggctgtgggaagaagtgcaatggccatggggtctgtaacaacaaccagaactgccactgcctgccgggctgggccccgcccttctgcaacacaccgggccacgggggcagtatcgacagtgggcctatgccccctgagagtgtgggtcctgtggtagctggagtgttggtggccatcttggtgctggcggtcctcatgctgatgtactactgctgcagacagaacaacaaactaggccaactcaagccctcagctctcccttccaagctgaggcaacagttcagttgtcccttcagggtttctcagaacagcgggactggtcatgccaacccaactttcaagctgcagacgccccagggcaagcgaaaggtgatcaacactccggaaatcctgcggaagccctcccagcctcctccccggccccctccagattatctgcgtggtgggtccccacctgcaccactgccagctcacctgagcagggctgctaggaactccccagggcccgggtctcaaatagagaggacggagtcgtccaggaggcctcctccaagccggccaattccccccgcaccaaattgcatcgtttcccaggacttctccaggcctcggccgccccagaaggcactcccggcaaacccagtgccaggccgcaggagcctccccaggccaggaggtgcatccccactgcggccccctggtgctggccctcagcagtcccggcctctggcagcacttgccccaaagtttccagaatacagatcacagagggctggagggatgattagctcgaaaatctagacctgtccaaggggcttctccctttccttgagctctctggacactgcagaggacccatggccatggaaccctgaagaagcatgtctggccgcctctgagctcctcccaccctcctccaggaacctccacatctccaaaaatctccctgttgactcagtgcctcctcggcttccttggaagcccagagggactatgatctgatggcctctaggtgttgttttgtgcaatatacagccccaggtagggaggggagagtatgaggagggtgactggcagcttctcctccagactcctagccccgaggtgctgatggagatgctcaaggccagcaagcccctcaggccagcacttcgcttgcagaagccatccattcactcctggggtgcagggcacgcaagagagcttcccattgcttctgctctcctcagaggtcccgggctggatggaggctggtacttacccaccccttttagcttttagggattaaggaagggtcaagccagccactgctgtggccctgcccagggcttggttgagggaacggcttctggctgtatggctgcatgtgacaagccacgtcccctcccacctctccccaaacccctgcatccctgtattcacacgggtcactctgactcagacaggtactattcgtaggcagtgtagacagcaggaggagcaccgggcttgggcttcctctgagccgtgatgccaaaggttgcgactcctgactctggataatttttagttgctctttgttttctctgccgcactttcctggtgccccacgcttttctctcttccttcccctctcattctccctctaatgtgtggtgctttggtgagcaaaccctcagcagtcctgaccttcgggtgaccaggtgcttgtgacctacaagtcagagtcctctctcacagtcggccactggatttccctcactggctctcaggagtgtgaccagagtagacttggggcatggccattggggtcatatgtttatttttcattgtgttttgtgacctcagcagggtgggggtcttcctccttactctaagctaaatctaggtgaggtttccccttagggagcccagctatttacaaagtacacacgagggagcaggctggtcattgacttcgggctggaccgttgccctctgagcagagaacagacccatttctgggagctgcccgagatcactggagaaggcagccagcagcagctgcactggaacagtcagagcagggagcctcttcctcaacccagctttttgtcattcacttccttttgttctctctctggtcactgcccttacctgaccctcacagaaagagagctctgagcaggtgagggggtctgcggtggctcctgtcttccctgcagcagggaaggagggccgtgtggtgctttgctagataggacggtttttgcaaagcacctggagatgtttgctgggagatagactcccactccacaaaggtgctgggtggctctccggacaggagctggcctgactctcactcctctgaggctttcctggggcctcctcccatcctgccatgagcaattgtttgctcttgaaaacctcactgcaaggctgaggctgagcttctgattcaccaccccagggcctccttatagttctctgcacacaataggtgcttcttggatgttcttgggtttggaaataagtggaaaatacgggatgtacccctgggggaaaagcctgggttgggtttagaaagatctcaggaaaatgagtttctcttccctcagggtggctgtgatacaggttccccatgtccttgccgtgggtcatccttgctgtgggtcatccttgctgtggagatccattccccacctttcctgtggcccaaccttttatttaaatgtgctaccctctgcctcaaggcttggttcctggaaagtaaaggtgaaaacatcccctttcacccctctgcaaaacaaacaagcaacatcctcaaaacccaaccccatgcctcacagagcttcctgtggcttctccagcctttctccctcacatcaggaggtagatagctctgaaatgacagcgccacagccatagtgactgcatgagccatctgaacctgcagtccaccctccctggaaccacaccagaaagagacctgggttgtcgttttcttgctttttgttttgttttgttttattattttcatatcacctccatcccataaagttgtactgtgaactggaagatggtggaatgttttggaatttgatagactttcggcaaccagttctactaatgcttcactcctggctctgttcagggaggctgcccaggaggaagactggccattatgcatccccttttctttccagtgcccagtatgctgttttgaggtgtcaaatacaaataaatctgggcttagggaaggagagaccttattccaaagcacgattgcagaaggggaaagggaatattgcaaaagggagaggaaggggccttatgggaatagtgaaaaggctcagaccgaccgatggcaagatctgcaagcgtctcaaagcccaggcagaaaaggacttttcttttattggaagaagtaaacatggctagaaagaaccacgttcagggaatgacgttgtgcccagcctttttttttttttttttttttttgtctccaggggaggggctgtttgctggctcaggctgaggatggcccaaagtccagggtctggtggggaggagggaagcttaactcaagtttgggttagtgagttagcaagctctttgtgcagatggggatgtaggtaaatctttttaaaagtgaaattaacctcctgccaattttacaacccaagaatttttttttaagggccttggagccatctctaaaacaaacctcaagggatttagtgccctgtctccctgtctctagaagccttagcctgggcacctggctcaatcttgtaactgcctgctagccatagattcctttcagccttgctgacttctccctataaaagtaaagcctttttctgccccagctctgagacacttgcagatcttaaggtctgagacttgctgattttctggttggagtgtttttttgtattgccatagtcccttccccctgaagcaatagcccctccccacctcctgcaatacgcctttccaatctttattggaagtctctccctgcctacttcctaatttattcttatttgacagagggtatggaagacttgcaatttgaaaactggggaccagttccaaagtcagtaattgtgttaaccacgtgtataacagctctgctggacacccaagaaagccatgggaacgccaactggaaaggtccccttccccaggggagcctgcgaaggagaggttctgtagaatccaagcccacatttccaaagtcacccccaacgcgtcctctcacaccgtccactgtgcgtttgtatgtgtctgggatccagggcaatgtgaattttctttttatttgggagattgttcacggaaaacagatcttcttctctcttgtccacctattaattgtttacaatatttgtacatctatgcaaaatacttgaatgggccatggtgccttttttccttgttagtatttaattaaaaatgaattgtttgtcatttgcaatgtta

SEQ ID NO: 14 - protein, 918 aa, Homo sapiens
MPGGAGAARLCLLAFALQPLRPRAAREPGWTRGSEEGSPKLQHELIIPQWKTSESPVREKHPLKAELRVMAEGRELILDLEKNEQLFAPSYTETHYTSSGNPQTTTRKLEDHCFYHGTVRETELSSVTLSTCRGIRGLITVSSNLSYVIEPLPDSKGQHLIYRSEHLKPPPGNCGFEHSKPTTRDWALQFTQQTKKRPRRMKREDLNSMKYVELYLVADYLEFQKNRRDQDATKHKLIEIANYVDKFYRSLNIRIALVGLEVWTHGNMCEVSENPYSTLWSFLSWRRKLLAQKYHDNAQLITGMSFHGTTIGLAPLMAMCSVYQSGGVNMDHSENAIGVAATMAHEMGHNFGMTHDSADCCSASAADGGCIMAAATGHPFPKVFNGCNRRELDRYLQSGGGMCLSNMPDTRMLYGGRRCGNGYLEDGEECDCGEEEECNNPCCNASNCTLRPGAECAHGSCCHQCKLLAPGTLCREQARQCDLPEFCTGKSPHCPTNFYQMDGTPCEGGQAYCYNGMCLTYQEQCQQLWGPGARPAPDLCFEKVNVAGDTFGNCGKDMNGEHRKCNMRDAKCGKIQCQSSEARPLESNAVPIDTTIIMNGRQIQCRGTHVYRGPEEEGDMLDPGLVMTGTKCGYNHICFEGQCRNTSFFETEGCGKKCNGHGVCNNNQNCHCLPGWAPPFCNTPGHGGSIDSGPMPPESVGPVVAGVLVAILVLAVLMLMYYCCRQNNKLGQLKPSALPSKLRQQFSCPFRVSQNSGTGHANPTFKLQTPQGKRKVINTPEILRKPSQPPPRPPPDYLRGGSPPAPLPAHLSRAARNSPGPGSQIERTESSRRPPPSRPIPPAPNCIVSQDFSRPRPPQKALPANPVPGRRSLPRPGGASPLRPPGAGPQQSRPLAALAPKFPEYRSQRAGGMISSKI

SEQ ID NO: 15 - DNA, 3220 bp, Homo sapiens
tcactggagaggaggcagggacagacccagcagcacccacctgagcgagaagagcagacaccgtgctcctggaatcacccagcatgttgcaaggtctcctgccagtcagtctcctcctctctgttgcagtaagtgctataaaagaactccctggggtgaagaagtatgaagtggtttatcctataagacttcatccactgcataaaagagaggccaaagagccagagcaacaggaacaatttgaaactgaattaaagtataaaatgacaattaatggaaaaattgcagtgctttatttgaaaaaaaacaagaacctccttgcaccaggctacacggaaacatattataattccactggaaaggagatcaccacaagcccacaaattatggatgattgttattatcaaggacatattcttaatgaaaaggtttctgacgctagcatcagcacatgtaggggtctaaggggctacttcagtcagggggatcaaagatactttattgaacctttaagccccatacatcgggatggacaggagcatgcactcttcaagtataaccctgatgaaaagaattatgacagcacctgtgggatggatggtgtgttgtgggcccacgatttgcagcagaacattgccctacctgccaccaaactagtaaaattgaaagacaggaaggttcaggaacatgagaaatacatagaatattatttggtcctggataatggtgagtttaaaaggtacaatgagaatcaagatgagatcagaaagagggtatttgagatggctaattatgtcaacatgctttataaaaagctcaatactcatgtggccttagttggtatggaaatctggactgacaaggataagataaagataaccccaaatgcaagcttcaccttggagaatttttctaaatggagggggagtgttctctcaagaagaaagcgtcatgatattgctcagttaatcacagcaacagaacttgctggaacgactgtgggtcttgcatttatgtctacaatgtgttctccttattctgttggcgttgttcaggaccacagcgataatcttcttagagttgcagggacaatggcacatgaaatgggccacaactttggaatgtttcatgacgactattcttgcaagtgtccttctacaatatgtgtgatggacaaagcactgagcttctatatacccacagacttcagttcctgcagccgtctcagctatgacaagttttttgaagataaattatcaaattgcctctttaatgctccattgcctacagatatcatatccactccaatttgtgggaaccagttggtggaaatgggagaggactgtgattgtgggacatctgaggaatgtaccaatatttgctgtgatgctaagacatgtaaaatcaaagcaacttttcaatgtgcattaggagaatgttgtgaaaaatgccaatttaaaaaggctgggatggtgtgcagaccagcaaaagatgagtgcgacctgcctgaaatgtgtaatggtaaatctggtaattgtcctgatgatagattccaagtcaatggcttcccttgccatcacgggaagggccactgcttgatggggacatgccccacactgcaggagcagtgcacagagctgtggggaccaggaactgaggttgcagataagtcatgttacaacaggaatgaaggtgggtcaaagtacgggtactgtcgcagagtggatgacacactcattccctgcaaagcaaatgataccatgtgtgggaagttgttctgtcaaggtgggtcggataatttgccctggaaaggacggatagtgactttcctgacatgtaaaacatttgatcctgaagacacaagtcaagaaataggcatggtggccaatggaactaagtgtggcgataacaaggtttgcattaatgcagaatgtgtggatattgagaaagcctacaaatcaaccaattgctcatctaagtgcaaaggacatgctgtgtgtgaccatgagctccagtgtcaatgtgaggaaggatggatccctcccgactgcgatgactcctcagtggtcttccacttctccattgtggttggggtgctgttcccaatggcggtcatttttgtggtggttgctatggtaatccggcaccagagctccagagaaaagcagaagaaagatcagaggccactatctaccactggcaccaggccacacaaacagaagaggaaaccccagatggtaaaggctgttcaaccccaagagatgagtcagatgaagccccatgtgtatgatctgccagtagaaggcaatgagcccccagcctcttttcataaagacacaaacgcacttccccctactgttttcaaggataatccagtgtctacacctaaggactcaaatccaaaagcatgaagcaacagctaagcaagaactaatggctaaattatcaacttggaaaactggaaaatctggatggcagagaaatatactatctatctcaccagtatttgctctcgactcaagaaggttaacattttctgattcatgttagactttgaagagactaaagaaaattttcaagaggaacatatgcctgagaacctttgcatgaatttaaaatttcaattatccattcttataagaaggaagatgattgtaaagaaatatctccgaagttaaaatctgtaataggaattgattcattctctaatgaaaacaaaacataaaaacatcacactaatcttggaggaataagaaaaattgtacatccattaaatgtacaattgattgcaacatcttgattgttttaaccattaacttgtcaaattacaatcacagttaagaaaatgatgtaaaattctgttttgtggatctctttcctagattagcttctgaaatcattattagctatatcatttgaggttttctacaatttggtataactaagaatttaaaaatgttttatcatatatatttgtataattaattactggcatggttaaagtggttttcactttttaaatggagaaaatttcagttaaattaataggataaaccaggttgcgaactggtgacctgtaggccatgtttgcactgcaaatatatttggtctgaatgatattgatattggacacatagtacttttacatgttttgaatgtattgctaatatttaaaaattgagagatcttgcataaacaatagattcccagctttgtcaga

SEQ ID NO: 16 - protein, 775 aa, Homo sapiens
MLQGLLPVSLLLSVAVSAIKELPGVKKYEVVYPIRLHPLHKREAKEPEQQEQFETELKYKMTINGKIAVLYLKKNKNLLAPGYTETYYNSTGKEITTSPQIMDDCYYQGHILNEKVSDASISTCRGLRGYFSQGDQRYFIEPLSPIHRDGQEHALFKYNPDEKNYDSTCGMDGVLWAHDLQQNIALPATKLVKLKDRKVQEHEKYIEYYLVLDNGEFKRYNENQDEIRKRVFEMANYVNMLYKKLNTHVALVGMEIWTDKDKIKITPNASFTLENFSKWRGSVLSRRKRHDIAQLITATELAGTTVGLAFMSTMCSPYSVGVVQDHSDNLLRVAGTMAHEMGHNFGMFHDDYSCKCPSTICVMDKALSFYIPTDFSSCSRLSYDKFFEDKLSNCLFNAPLPTDIISTPICGNQLVEMGEDCDCGTSEECTNICCDAKTCKIKATFQCALGECCEKCQFKKAGMVCRPAKDECDLPEMCNGKSGNCPDDRFQVNGFPCHHGKGHCLMGTCPTLQEQCTELWGPGTEVADKSCYNRNEGGSKYGYCRRVDDTLIPCKANDTMCGKLFCQGGSDNLPWKGRIVTFLTCKTFDPEDTSQEIGMVANGTKCGDNKVCINAECVDIEKAYKSTNCSSKCKGHAVCDHELQCQCEEGWIPPDCDDSSVVFHFSIVVGVLFPMAVIFVVVAMVIRHQSSREKQKKDQRPLSTTGTRPHKQKRKPQMVKAVQPQEMSQMKPHVYDLPVEGNEPPASFHKDTNALPPTVFKDNPVSTPKDSNPKA

SEQ ID NO: 17 - DNA, 4670 bp, Homo sapiens
gcactcgctggaaagcggctccgagccaggggctattgcaaagccagggtgcgctaccggacggagaggggagagccctgagcagagtgagcaacatcgcagccaaggcggaggccgaagaggggcgccaggcaccaatctccgcgttgcctcagccccggaggcgccccagagcgcttcttgtcccagcagagccactctgcctgcgcctgcctctcagtgtctccaactttgcgctggaagaaaaacttcccgcgcgccggcagaactgcagcgcctccttttagtgactccgggagcttcggctgtagccggctctgcgcgcccttccaacgaataatagaaattgttaattttaacaatccagagcaggccaacgaggctttgctctcccgacccgaactaaaggtccctcgctccgtgcgctgctacgagcggtgtctcctggggctccaatgcagcgagctgtgcccgaggggttcggaaggcgcaagctgggcagcgacatggggaacgcggagcgggctccggggtctcggagctttgggcccgtacccacgctgctgctgctcgccgcggcgctactggccgtgtcggacgcactcgggcgcccctccgaggaggacgaggagctagtggtgccggagctggagcgcgccccgggacacgggaccacgcgcctccgcctgcacgcctttgaccagcagctggatctggagctgcggcccgacagcagctttttggcgcccggcttcacgctccagaacgtggggcgcaaatccgggtccgagacgccgcttccggaaaccgacctggcgcactgcttctactccggcaccgtgaatggcgatcccagctcggctgccgccctcagcctctgcgagggcgtgcgcggcgccttctacctgctgggggaggcgtatttcatccagccgctgcccgccgccagcgagcgcctcgccaccgccgccccaggggagaagccgccggcaccactacagttccacctcctgcggcggaatcggcagggcgacgtcggcggcacgtgcggggtcgtggacgacgagccccggccgactgggaaagcggagaccgaagacgaggacgaagggactgagggcgaggacgaaggggctcagtggtcgccgcaggacccggcactgcaaggcgtaggacagcccacaggaactggaagcataagaaagaagcgatttgtgtccagtcaccgctatgtggaaaccatgcttgtggcagaccagtcgatggcagaattccacggcagtggtctaaagcattaccttctcacgttgttttcggtggcagccagattgtacaaacaccccagcattcgtaattcagttagcctggtggtggtgaagatcttggtcatccacgatgaacagaaggggccggaagtgacctccaatgctgccctcactctgcggaacttttgcaactggcagaagcagcacaacccacccagtgaccgggatgcagagcactatgacacagcaattcttttcaccagacaggacttgtgtgggtcccagacatgtgatactcttgggatggctgatgttggaactgtgtgtgatccgagcagaagctgctccgtcatagaagatgatggtttacaagctgccttcaccacagcccatgaattaggccacgtgtttaacatgccacatgatgatgcaaagcagtgtgccagccttaatggtgtgaaccaggattcccacatgatggcgtcaatgctttccaacctggaccacagccagccttggtctccttgcagtgcctacatgattacatcatttctggataatggtcatggggaatgtttgatggacaagcctcagaatcccatacagctcccaggcgatctccctggcacctcgtacgatgccaaccggcagtgccagtttacatttggggaggactccaaacactgccccgatgcagccagcacatgtagcaccttgtggtgtaccggcacctctggtggggtgctggtgtgtcaaaccaaacacttcccgtgggcggatggcaccagctgtggagaagggaaatggtgtatcaacggcaagtgtgtgaacaaaaccgacagaaagcattttgatacgccttttcatggaagctggggaatgtgggggccttggggagactgttcgagaacgtgcggtggaggagtccagtacacgatgagggaatgtgacaacccagtcccaaagaatggagggaagtactgtgaaggcaaacgagtgcgctacagatcctgtaaccttgaggactgtccagacaataatggaaaaacctttagagaggaacaatgtgaagcacacaacgagttttcaaaagcttcctttgggagtgggcctgcggtggaatggattcccaagtacgctggcgtctcaccaaaggacaggtgcaagctcatctgccaagccaaaggcattggctacttcttcgttttgcagcccaaggttgtagatggtactccatgtagcccagattccacctctgtctgtgtgcaaggacagtgtgtaaaagctggttgtgatcgcatcatagactccaaaaagaagtttgataaatgtggtgtttgcgggggaaatggatctacttgtaaaaaaatatcaggatcagttactagtgcaaaacctggatatcatgatatcatcacaattccaactggagccaccaacatcgaagtgaaacagcggaaccagaggggatccaggaacaatggcagctttcttgccatcaaagctgctgatggcacatatattcttaatggtgactacactttgtccaccttagagcaagacattatgtacaaaggtgttgtcttgaggtacagcggctcctctgcggcattggaaagaattcgcagctttagccctctcaaagagcccttgaccatccaggttcttactgtgggcaatgcccttcgacctaaaattaaatacacctacttcgtaaagaagaagaaggaatctttcaatgctatccccactttttcagcatgggtcattgaagagtggggcgaatgttctaagtcatgtgaattgggttggcagagaagactggtagaatgccgagacattaatggacagcctgcttccgagtgtgcaaaggaagtgaagccagccagcaccagaccttgtgcagaccatccctgcccccagtggcagctgggggagtggtcatcatgttctaagacctgtgggaagggttacaaaaaaagaagcttgaagtgtctgtcccatgatggaggggtgttatctcatgagagctgtgatcctttaaagaaacctaaacatttcatagacttttgcacaatggcagaatgcagttaagtggtttaagtggtgttagctttgagggcaaggcaaagtgaggaagggctggtgcagggaaagcaagaaggctggagggatccagcgtatcttgccagtaaccagtgaggtgtatcagtaaggtgggattatgggggtagatagaaaaggagttgaatcatcagagtaaactgccagttgcaaatttgataggatagttagtgaggattattaacctctgagcagtgatatagcataataaagccccgggcattattattattatttcttttgttacatctattacaagtttagaaaaaacaaagcaattgtcaaaaaaagttagaactattacaacccctgtttcctggtacttatcaaatacttagtatcatgggggttgggaaatgaaaagtaggagaaaagtgagattttactaagacctgttttactttacctcactaacaatggggggagaaaggagtacaaataggatctttgaccagcactgtttatggctgctatggtttcagagaatgtttatacattatttctaccgagaattaaaacttcagattgttcaacatgagagaaaggctcagcaacgtgaaataacgcaaatggcttcctctttccttttttggaccatctcagtctttatttgtgtaattcattttgaggaaaaaacaactccatgtatttattcaagtgcattaaagtctacaatggaaaaaaagcagtgaagcattagatgctggtaaaagctagaggagacacaatgagcttagtacctccaacttcctttctttcctaccatgtaaccctgctttgggaatatggatgtaaagaagtaacttgtgtctcatgaaaatcagtacaatcacacaaggaggatgaaacgccggaacaaaaatgaggtgtgtagaacagggtcccacaggtttggggacattgagatcacttgtcttgtggtggggaggctgctgaggggtagcaggtccatctccagcagctggtccaacagtcgtatcctggtgaatgtctgttcagctcttctgtgagaatatgattttttccatatgtatatagtaaaatatgttactataaattacatgtactttataagtattggtttgggtgttccttccaagaaggactatagttagtaataaatgcctataataacatatttatttttatacatttatttctaatgaaaaaaacttttaaattatatcgcttttgtggaagtgcatataaaatagagtatttatacaatatatgttactagaaataaaagaacacttttggaaaaaaaaaaaaaaaaaaaaa

SEQ ID NO: 18 - protein, 967 aa, Homo sapiens
MQRAVPEGFGRRKLGSDMGNAERAPGSRSFGPVPTLLLLAAALLAVSDALGRPSEEDEELVVPELERAPGHGTTRLRLHAFDQQLDLELRPDSSFLAPGFTLQNVGRKSGSETPLPETDLAHCFYSGTVNGDPSSAAALSLCEGVRGAFYLLGEAYFIQPLPAASERLATAAPGEKPPAPLQFHLLRRNRQGDVGGTCGVVDDEPRPTGKAETEDEDEGTEGEDEGAQWSPQDPALQGVGQPTGTGSIRKKRFVSSHRYVETMLVADQSMAEFHGSGLKHYLLTLFSVAARLYKHPSIRNSVSLVVVKILVIHDEQKGPEVTSNAALTLRNFCNWQKQHNPPSDRDAEHYDTAILFTRQDLCGSQTCDTLGMADVGTVCDPSRSCSVIEDDGLQAAFTTAHELGHVFNMPHDDAKQCASLNGVNQDSHMMASMLSNLDHSQPWSPCSAYMITSFLDNGHGECLMDKPQNPIQLPGDLPGTSYDANRQCQFTFGEDSKHCPDAASTCSTLWCTGTSGGVLVCQTKHFPWADGTSCGEGKWCINGKCVNKTDRKHFDTPFHGSWGMWGPWGDCSRTCGGGVQYTMRECDNPVPKNGGKYCEGKRVRYRSCNLEDCPDNNGKTFREEQCEAHNEFSKASFGSGPAVEWIPKYAGVSPKDRCKLICQAKGIGYFFVLQPKVVDGTPCSPDSTSVCVQGQCVKAGCDRIIDSKKKFDKCGVCGGNGSTCKKISGSVTSAKPGYHDIITIPTGATNIEVKQRNQRGSRNNGSFLAIKAADGTYILNGDYTLSTLEQDIMYKGVVLRYSGSSAALERIRSFSPLKEPLTIQVLTVGNALRPKIKYTYFVKKKKESFNAIPTFSAWVIEEWGECSKSCELGWQRRLVECRDINGQPASECAKEVKPASTRPCADHPCPQWQLGEWSSCSKTCGKGYKKRSLKCLSHDGGVLSHESCDPLKKPKHFIDFCTMAECS

SEQ ID NO: 19 - DNA, 5821 bp, Homo sapiens
gctttgcccagtagttggaaagtgaactcgactcgtgatggttctcctgtcactttggttgatagcagccgctctggtagaggttaggacttcagctgatggacaagctggtaatgaagaaatggtgcaaatagatttaccaataaagagatatagagagtatgagctggtgactccagtcagcacaaatctagaaggacgctatctctcccatactctttctgcgagtcacaaaaagaggtcagcgagggacgtgtcttccaaccctgagcagttgttctttaacatcacggcatttggaaaagattttcatctgcgactaaagcccaacactcaactagtagctcctggggctgttgtggagtggcatgagacatctctggtgcctgggaatataaccgatcccattaacaaccatcaaccaggaagtgctacgtatagaatccggaaaacagagcctttgcagactaactgtgcttatgttggtgacatcgtggacattccaggaacctctgttgccatcagcaactgtgatggtctggctggaatgataaaaagtgataatgaagagtatttcattgaacccttggaaagaggtaaacagatggaggaagaaaaaggaaggattcatgttgtctacaagagatcagctgtagaacaggctcccatagacatgtccaaagacttccactacagagagtcggacctggaaggccttgatgatctaggtactgtttatggcaacatccaccagcagctgaatgaaacaatgagacgccgcagacacgcgggagaaaacgattacaatatcgaggtactgctgggagtggatgactctgtggtccgtttccatggcaaagagcacgtccaaaactacctcctgaccctaatgaacattgtgaatgaaatttaccatgatgagtccctcggagtgcatataaatgtggtcctggtgcgcatgataatgctgggatatgcaaagtccatcagcctcatagaaaggggaaacccatccagaagcttggagaatgtgtgtcgctgggcgtcccaacagcaaagatctgatctcaaccactctgaacaccatgaccatgcaatttttttaaccaggcaagactttggacctgctggaatgcaaggatatgctccagtcaccggcatgtgtcatccagtgagaagttgtaccctgaatcatgaggatggtttttcatctgcttttgtagtagcccatgaaacgggccatgtgttgggaatggagcatgatggacaaggcaacaggtgtggtgatgagactgctatgggaagtgtcatggctcccttggtacaagcagcattccatcgttaccactggtcccgatgcagtggtcaagaactgaaaagatatatccattcctatgactgtctccttgatgacccttttgatcatgattggcctaaactcccagaacttcctggaatcaattattctatggatgagcaatgtcgttttgattttggtgttggctataaaatgtgcaccgcgttccgaacctttgacccatgtaaacagctgtggtgtagccatcctgataatccctacttttgtaagactaaaaagggacctccacttgatgggactgaatgtgctgctggaaaatggtgctataagggtcattgcatgtggaagaatgctaatcagcaaaaacaagatggcaattgggggtcatggactaaatttggctcctgttctcggacatgtggaactggtgttcgtttcagaacacgccagtgcaataatcccatgcccatcaatggtggtcaggattgtcctggtgttaattttgagtaccagctttgtaacacagaagaatgccaaaaacactttgaggacttcagagcacagcagtgtcagcagcgaaactcccactttgaataccagaataccaaacaccactggttgccatatgaacatcctgaccccaagaaaagatgccacctttactgtcagtccaaggagactggagatgttgcttacatgaaacaactggtgcatgatggaacgcactgttcttacaaagatccatatagcatatgtgtgcgaggagagtgtgtgaaagtgggctgtgataaagaaattggttctaataaggttgaggataagtgtggtgtctgtggaggagataattcccactgccgaaccgtgaaggggacatttaccagaactcccaggaagcttgggtaccttaagatgtttgatataccccctggggctagacatgtgttaatccaagaagacgaggcttctcctcatattcttgctattaagaaccaggctacaggccattatattttaaatggcaaaggggaggaagccaagtcgcggaccttcatagatcttggtgtggagtgggattataacattgaagatgacattgaaagtcttcacaccgatggacctttacatgatcctgttattgttttgattatacctcaagaaaatgatacccgctctagcctgacatataagtacatcatccatgaagactctgtacctacaatcaacagcaacaatgtcatccaggaagaattagatacttttgagtgggctttgaagagctggtctcagtgttccaaaccctgtggtggaggtttccagtacactaaatatggatgccgtaggaaaagtgataataaaatggtccatcgcagcttctgtgaggccaacaaaaagccgaaacctattagacgaatgtgcaatattcaagagtgtacacatccactctgggtagcagaagaatgggaacactgcaccaaaacctgtggaagttctggctatcagcttcgcactgtacgctgccttcagccactccttgatggcaccaaccgctctgtgcacagcaaatactgcatgggtgaccgtcccgagagccgccggccctgtaacagagtgccctgccctgcacagtggaaaacaggaccctggagtgagtgttcagtgacctgcggtgaaggaacggaggtgaggcaggtcctctgcagggctggggaccactgtgatggtgaaaagcctgagtcggtcagagcctgtcaactgcctccttgtaatgatgaaccatgtttgggagacaagtccatattctgtcaaatggaagtgttggcacgatactgctccataccaggttataacaagttatgttgtgagtcctgcagcaagcgcagtagcaccctgccaccaccataccttctagaagctgctgaaactcatgatgatgtcatctctaaccctagtgacctccctagatctctagtgatgcctacatctttggttccttatcattcagagacccctgcaaagaagatgtctttgagtagcatctcttcagtgggaggtccaaatgcatatgctgctttcaggccaaacagtaaacctgatggtgctaatttacgccagaggagtgctcagcaagcaggaagtaagactgtgagactggtcaccgtaccatcctccccacccaccaagagggtccacctcagttcagcttcacaaatggctgctgcttccttctttgcagccagtgattcaataggtgcttcttctcaggcaagaacctcaaagaaagatggaaagatcattgacaacagacgtccgacaagatcatccaccttagaaagatgagaaagtgaaccaaaaaggctagaaaccagaggaaaacctggacaacctctctcttcccatggtgcatatgcttgtttaaagtggaaatctctatagatcgtcagctcattttatctgtaattggaagaacagaaagtgctggctcactttctagttgctttcatcctccttttgttctgcattgactcatttaccagaattcattggaagaaatcaccaaagattattacaaaagaaaaatatgttgctaagattgtgttggtcgctctctgaagcagaaaagggactggaaccaattgtgcatatcagctgactttttgtttgttttagaaaagttacagtaaaaattaaaaagagataccaatggtttacactttaacaagaaattttggatatggaacaaagaattcttagacttgtattcctatttatctatattagaaatattgtatgagcaaatttgcagctgttgtgtaaatactgtatattgcaaaaatcagtattattttaagagatgtgttctcaaatgattgtttactatattacatttctggatgttctaggtgcctgtcgttgagtattgccttgtttgacattctataggttaattttcaaagcagagtattacaaaagagaagttagaattacagctactgacaatataaagggttttgttgaatcaacaatgtgatacgtaaattatagaaaaagaaaagaaacacaaaagctatagatatacagatatcagcttacctattgccttctatacttataatttaaaggattggtgtcttagtacacttgtggtcacagggatcaacgaatagtaaataatgaactcgtgcaagacaaaactgaaaccctctttccaggacctcagtaggcaccgttgaggtgtcctttgtttttgtgtgtgtgtgttcttttttaattttcgcattgttgacagatacaaacagttatactcaatgtactgtaataatcgcaaaggaaaaagttttgggataacttatttgtatgttggtagctgagaaaaatatcatcagtctagaattgatatttgagtatagtagagctttggggctttgaaggcaggttcaagaaagcatatgtcgatggttgagatatttattttccatatggttcatgttcaaatgttcacaaccacaatgcatctgactgcaataatgtgctaataatttatgtcagtagtcaccttgctcacagcaaagccagaaatgctctctccagggagtagatgtaaagtacttgtacatagaattcagaactgaagatatttattaaaagttgattttttttcttgatagtatttttatgtactaaatatttacactaatatcaattacatattttggtaaactagagagacataattagagatgcatgctttgttctgtgcatagagacctttaagcaaactactacagccaactcaaaagctaaaactgaacaaatttgatgttatgcaaacatcttgcatttttagtagttgatattaagttgatgacttgtttcccttcaaggaaacattaaattgtatggactcagctagctgttcaatgaaattgtgaattagaaacatttttaaaagtttttgaaagagataagtgcatcatgaattacatgtacatgagaggagatagtgatatcagcataatgattttgaggtcagtacctgagctgtctaaaaatatattatacaaactaaaatgtagatgaattaacctctcaaagcacagaatgtgcaagaacttttgcattttaatcgttgtaaactaacagcttaaactattgactctatacctctaaagaattgctgctactttgtgcaagaactttgaaggtcaaattaggcaaattccagatagtaaaacaatccctaagccttaagtcttttttttttcctaaaaattcccatagaataaaattctctctagtttacttgtgtgtgcatacatctcatccacaggggaagataaagatggtcacacaaacagtttccataaagatgtacatattcattatacttctgacctttgggctttcttttctactaagctaaaaattcctttttatcaaagtgtacactactgatgctgtttgttgtactgagagcacgtaccaataaaaatgttaacaaaatat

SEQ ID NO: 20 - protein, 1205 aa, Homo sapiens
MVLLSLWLIAAALVEVRTSADGQAGNEEMVQIDLPIKRYREYELVTPVSTNLEGRYLSHTLSASHKKRSARDVSSNPEQLFFNITAFGKDFHLRLKPNTQLVAPGAVVEWHETSLVPGNITDPINNHQPGSATYRIRKTEPLQTNCAYVGDIVDIPGTSVAISNCDGLAGMIKSDNEEYFIEPLERGKQMEEEKGRIHVVYKRSAVEQAPIDMSKDFHYRESDLEGLDDLGTVYGNIHQQLNETMRRRRHAGENDYNIEVLLGVDDSVVRFHGKEHVQNYLLTLMNIVNEIYHDESLGVHINVVLVRMIMLGYAKSISLIERGNPSRSLENVCRWASQQQRSDLNHSEHHDHAIFLTRQDFGPAGMQGYAPVTGMCHPVRSCTLNHEDGFSSAFVVAHETGHVLGMEHDGQGNRCGDETAMGSVMAPLVQAAFHRYHWSRCSGQELKRYIHSYDCLLDDPFDHDWPKLPELPGINYSMDEQCRFDFGVGYKMCTAFRTFDPCKQLWCSHPDNPYFCKTKKGPPLDGTECAAGKWCYKGHCMWKNANQQKQDGNWGSWTKFGSCSRTCGTGVRFRTRQCNNPMPINGGQDCPGVNFEYQLCNTEECQKHFEDFRAQQCQQRNSHFEYQNTKHHWLPYEHPDPKKRCHLYCQSKETGDVAYMKQLVHDGTHCSYKDPYSICVRGECVKVGCDKEIGSNKVEDKCGVCGGDNSHCRTVKGTFTRTPRKLGYLKMFDIPPGARHVLIQEDEASPHILAIKNQATGHYILNGKGEEAKSRTFIDLGVEWDYNIEDDIESLHTDGPLHDPVIVLIIPQENDTRSSLTYKYIIHEDSVPTINSNNVIQEELDTFEWALKSWSQCSKPCGGGFQYTKYGCRRKSDNKMVHRSFCEANKKPKPIRRMCNIQECTHPLWVAEEWEHCTKTCGSSGYQLRTVRCLQPLLDGTNRSVHSKYCMGDRPESRRPCNRVPCPAQWKTGPWSECSVTCGEGTEVRQVLCRAGDHCDGEKPESVRACQLPPCNDEPCLGDKSIFCQMEVLARYCSIPGYNKLCCESCSKRSSTLPPPYLLEAAETHDDVISNPSDLPRSLVMPTSLVPYHSETPAKKMSLSSISSVGGPNAYAAFRPNSKPDGANLRQRSAQQAGSKTVRLVTVPSSPPTKRVHLSSASQMAAASFFAASDSIGASSQARTSKKDGKIIDNRRPTRSSTLER

SEQ ID NO: 21 - DNA, 9663 bp, Homo sapiens
ataaattcattgttccacctcctcgcatcttcacagcgctcgcgctgctctcggcgctcgcagctgccgactggggatgacggcgggcaggaggagaccgcagccgaagggacacagacacgccgcttcaccagctcgcctcaggctgcccccctgcatttttgttttaatttttacggctttttcccctctctttcttcccttcctcctggtcccagcagagccaaggaaacccacaaaataagaaaggaagtgggccccggagcttggaacctccacagccggcttgtccagcgcagcgcgggggcgggaggctgcgcgcaccagttgccagcccggtgcgcggtacctttccttacttttcttgaaacagcgatcgtgcctgcatttggtggttttttggtttttgtttttttccttttcccgtatttgctgaatctccactatccgactttttttttttaatcttttctttccccccccccccaccccacctctttctggagcacgaatccaaacattttcccaagcaacaaagaaaagttcgcacgctggcaccgcagcccggacaggctggcgctgctgccgggcccccctccctccgacacttgactcaatcctgcaagcaagtgtgtgtgtgtccccatcccccgccccgttaacttcatagcaaataacaaatacccataaagtcccagtcgcgcagcccctccccgcgggcagcgcactatgctgctcgggtgggcgtccctgctgctgtgcgcgttccgcctgcccctggccgcggtcggccccgccgcgacacctgcccaggataaagccgggcagcctccgactgctgcagcagccgcccagccccgccggcggcagggggaggaggtgcaggagcgagccgagcctcccggccacccgcaccccctggcgcagcggcgcaggagcaaggggctggtgcagaacatcgaccaactctactccggcggcggcaaggtgggctacctcgtctacgcgggcggccggaggttcctcttggacctggagcgagatggttcggtgggcattgctggcttcgtgcccgcaggaggcgggacgagtgcgccctggcgccaccggagccactgcttctatcggggcacagtggacggtagtccccgctctctggctgtctttgacctctgtgggggtctcgacggcttcttcgcggtcaagcacgcgcgctacaccctaaagccactgctgcgcggaccctgggcggaggaagaaaaggggcgcgtgtacggggatgggtccgcacggatcctgcacgtctacacccgcgagggcttcagcttcgaggccctgccgccgcgcgccagctgcgaaacccccgcgtccacaccggaggcccacgagcatgctccggcgcacagcaacccgagcggacgcgcagcactggcctcgcagctcttggaccagtccgctctctcgcccgctgggggctcaggaccgcagacgtggtggcggcggcggcgccgctccatctcccgggcccgccaggtggagctgcttctggtggctgacgcgtccatggcgcggttgtatggccggggcctgcagcattacctgctgaccctggcctccatcgccaataggctgtacagccatgctagcatcgagaaccacatccgcctggccgtggtgaaggtggtggtgctaggcgacaaggacaagagcctggaagtgagcaagaacgctgccaccacactcaagaacttttgcaagtggcagcaccaacacaaccagctgggagatgaccatgaggagcactacgatgcagctatcctgtttactcgggaggatttatgtgggcatcattcatgtgacaccctgggaatggcagacgttgggaccatatgttctccagagcgcagctgtgctgtgattgaagacgatggcctccacgcagccttcactgtggctcacgaaatcggacatttacttggcctctcccatgacgattccaaattctgtgaagagacctttggttccacagaagataagcgcttaatgtcttccatccttaccagcattgatgcatctaagccctggtccaaatgcacttcagccaccatcacagaattcctggatgatggccatggtaactgtttgctggacctaccacgaaagcagatcctgggccccgaagaactcccaggacagacctacgatgccacccagcagtgcaacctgacattcgggcctgagtactccgtgtgtcccggcatggatgtctgtgctcgcctgtggtgtgctgtggtacgccagggccagatggtctgtctgaccaagaagctgcctgcggtggaagggacgccttgtggaaaggggagaatctgcctgcagggcaaatgtgtggacaaaaccaagaaaaaatattattcaacgtcaagccatggcaactggggatcttggggatcctggggccagtgttctcgctcatgtggaggaggagtgcagtttgcctatcgtcactgtaataaccctgctcccagaaacaacggacgctactgcacagggaagagggccatctaccgctcctgcagtctcatgccctgcccacccaatggtaaatcatttcgtcatgaacagtgtgaggccaaaaatggctatcagtctgatgcaaaaggagtcaaaacttttgtggaatgggttcccaaatatgcaggtgtcctgccagcggatgtgtgcaagctgacctgcagagccaagggcactggctactatgtggtattttctccaaaggtgaccgatggcactgaatgtaggctgtacagtaattccgtctgcgtccgggggaagtgtgtgagaactggctgtgacggcatcattggctcaaagctgcagtatgacaagtgcggagtatgtggaggagacaactccagctgtacaaagattgttggaacctttaataagaaaagtaagggttacactgacgtggtgaggattcctgaaggggcaacccacataaaagttcgacagttcaaagccaaagaccagactagattcactgcctatttagccctgaaaaagaaaaacggtgagtaccttatcaatggaaagtacatgatctccacttcagagactatcattgacatcaatggaacagtcatgaactatagcggttggagccacagggatgacttcctgcatggcatgggctactctgccacgaaggaaattctaatagtgcagattcttgcaacagaccccactaaaccattagatgtccgttatagcttttttgttcccaagaagtccactccaaaagtaaactctgtcactagtcatggcagcaataaagtgggatcacacacttcgcagccgcagtgggtcacgggcccatggctcgcctgctctaggacctgtgacacaggttggcacaccagaacggtgcagtgccaggatggaaaccggaagttagcaaaaggatgtcctctctcccaaaggccttctgcgtttaagcaatgcttgttgaagaaatgttagcctgtggttatgatcttatgcacaaagataactggaggattcagcactgatgcagtcgtggtgaacaggaggtctacctaacgcacagaaagtcatgcttcagtgacattgtcaacaggagtccaattatgggcagaatctgctctctgtgaccaaaagaggatgtgcactgcttcacgtgacagtggtgaccttgcaatatagaaaaacttgggagttattgaacatcccctgggcttacaagaaacactgatgaatgtaaaatcaggggacatttgaagatggcagaactgtctcccccttgtcacctacctctgatagaatgtctttaatggtatcataatcattttcacccataatacacagtagcttcttcttactgtttgtaaatacattctcccttggtatgtcactttatatcccctggttctattaaaatatccatatatatttctataaaaaaagtgtttgaccaaagtaggtctgcagctatttcaacttccttccgtttccagaaagagctgtggatattttactggaaattaagaacttgctgctgttttaataagatgtagtatattttctgactacaggagataaaatttcagtcaaaaaaccattttgacagcaagtatcttctgagaaattttgaaaagtaaatagatctcagtgtatctagtcacttaaatacatacacgggttcatttacttaaacctttgactgcctgtatttttttcaggtagctagccaaattaatgcataatttcagatgtagaagtagggtttgcgtgtgtgtgtgtgatcatactcaagagtctaaaaactagtttccttgtgttggaaatttaaaaggaaaaaaatcgtatttcactgtgttttcaatttatattttcacaactactttctctctccagagctttcatctgatatctcacaatgtatgatatacgtacaaaacacacagcaagttttctatcatgtccaacacattcaacactggtatacctcctaccagcaagcctttaaaatgcatttgtgtttgcttatttgttttgttcaagggttcagtaagacctacaatgttttgtatttcttgacttattttattagaaacattaaagatcacttggtagttagccacattgagaagtggttatcattgttaatgtggttaatgccaaaaagtggttaatattaataagactgtttccacaccataggcaataatttcttaatttaaaaaatctaagtatattcctattgtactaaatatttttcccaactggaaagcacttgattgtacccgtaagtgtttgagtgatgacatgtgatgattttcagaaagttgttgtttttgtttccatagcctgtttaagtaggttgtaagtttgaatagttagacatggaaattattttataagcacacacctaaagatatctttttagatgataaaatgtacacccccccatcaccaacctcacaacttagaaaatctaagttgtttgatttctttgggatttcttttgttgtgaaacactgcaaagccaatttttctttataaaaattcatagtaatcctgccaaatgtgcctattgttaaagatttgcatgtgaagatcttagggaaccactgtttgagttctacaagctcatgagagtttatttttattataagatgtttttaatataaaagaattatgtaactgatcactatattacatcatttcagtgggccaggaaaatagatgtcttgctgttttcagtattttcttaagaaattgcttttaaaacaaataattgttttacaaaaccaataattatcctttgaattttcatagactgactttgcttttgacgtagaaattttttttctcaataaattatcactttgagaaatgaggcctgtacaaggctgataacctatatgtgatggagatcacccaatgccaagggcagaaagcaaacctagttaaataggtgagaaaaaaaataataatcccagtgccatttgtctgtgcaaagagaattaggagagaggttaatgttacttttttccattttggaaataattttaatcaagtaactcaaatgtgacaaaatttatttttattttttgtggttatattcccaacaacattaaaaaatactcgaggcataaatgtagttgtctcctactctgcttctcttactatactcatacatttttaatatggtttatcaatgattcatgtttccctcaaatagtgatggtttacacctgtcatggaaacaatcctagagagctcagagcaattaaaccactattccatgcttttaagtagttttctccacctttttcttatgagtctcactagattgactgaggaatgtatgtctaaattcctggagaagatgatatggattggaaactgaaattcagagaaatggagtgttcaatagataccacgaattgtgaacaaagggaaaattctatacaactcaatctaagtcagtccactttgacttcgtactgtctttcacctttccattgttgcatcttgaattttttaaaatgtctagaattcaggatgctaggggctacttctttaaaaaaaaaaaaaaaaaagaattcgtctgaaaatgctcaggtttgtaagaatctaatctcacttacataactaagcactccataataagttttattaagtacaaagggagccagaaaaaatgacatttatttcttctagatcagaaaaatttaaattaagccctgccttgctgtttagaaatatgtgggcattgttataatttattcaataaatttatgttcctttgccttcctgtggaaacagttttatcccactaaactaggaattaggggataaatcacaaacaaaaaaaaagttgcagcactgaaaaaaagtaatttattgtttttgcaactggtatgtgaatttgtgtgataaaattatttattcttatttaacaaaaatatgttcaaatttttctatatttaaaatgttttgctgttgtcctactttttaatttatgcttcatgtttgtgtataaagtacacttttacactttgtgagtttacataatatacagcactggttgcttttgtatttttttacagaaagctttctgtgtgaagcaggtgtatatgtatatattcctcatgtattcttattctgatactatcatttttctttccaaggaaattttaatctgtcatgaccaatagtgttcattacttgtgcctatgataataggttttttacatcacattaacactattttttccaagtcacaaataagaaaaacacttattcaatgaaacaaggtgcaagttttaaatttgggtacacaaatagcctagaagcttcctacagacgctaagacacagccaataatcagatcctttcacttcatcgagaaacttggacaagtcgatattgatgtattagatgaaagttgtctacacacaacttctgagggatacaaacgataataaaaccaaatgttgtctgtttctcctttagaaacacctcctaaaattaatatcatttagtctctagtgtctgtaggattctacagatgagcacaaatagattgggtttgtataacaaatgctaatagtcataactgtttctacaaatatggggtgtccattaagagaatgtgatgttttcctactgctgttgaatcccatggggtgattataggacttgaaataggcagagtcacctctgatgacatcagcttgcctctgtgatttcacagtctgatcctggcaacaagacaaagcacccttggacacacagccaatctctggttgtgatatttccccattgattccttccttgttaacaaggtcattttaatggttcaggtgaggacagcagccagattcaaagtccagaatttgtgctgttacatagagttcacactgtcaaataacattgaatttaataatgatcaaatttttctagtagtctttggcagagtgtataatctcattggcatgattggtgaatattactaatctctttataatgaaagatgctttacaaataccttatatttgctaacatttcaaaactactaaataaatgaaatagccatgtgtacagaaatggtcatttaaagctttaatagaaccaaattcaagacaatgtatcatttagacacacagaaaaggaacttgtatgttttccctattatttttctcatttgccaacaatctatagttttaggttatcaaacagatagatcaacttaactggctagtacattgaaaaatcttcctaagaatcctttgttagcataatctatagagataatttctcaaattatatcatcatgatgcatataaactctataatgtataattgtgtttcatttatttaatgtatgagaacatattgaaatacaaaaccatgcattagccaaaaaattggaatacaggtagtgttcagatcagcaaaacattcagtctggtaaatgcctgcctggggctatgatatcattctcaatgcaggttttatggaaaaactaaaagaatatgttgttagatgatgttggttttgaaaaaaaaaagacattaacatacacattagttagcccagttaattgcattctactaatatagttgcacattagcaataattttgctgtctctggtctttattttgtggcttcaactaactggaccatgtggactgtaaaggtcaaatggaaaaaacgagcagtggcccctcatcctgtaaggtactgctacatcagagtgacctaaaagtctaacactgtgaggaaaactgtgatttgtaggaaaaaaaaaaaaaacaaataaaaaacagggcatgctttttaatttttttccactttcctttggcacacccaatgaacaattctaatttttattgaggtgctaacatctttcgtgaccgactgtcaaatgtggtatttttgagttactatttttctacatgattttacagtttgcaagaaagacctctaagctttgtgtcacggtagggcacaacttgatactcaaaatttgaaaaataagcacatccaatgattgttttgaccaacagtggtcagtgacgtaaactgcatgtgcatctgaggacatttaaggggtcattaaaatttgaggagcatcaggccggagtagcagacttttagatgagtcatatttcagcattcactaagtcctcagcattccattcaaactgtcgtgtatatttggcctgattttttttcaagctttgcaataatttatgttattggtaaacacttggtgactatatctcagccttttctttaacaactcacaatatattagaaacacgtctacctatactgagagtatatttacaatagaagaacatactgtatgtgactttgtaaagctagacttttgattaagaaatatataatctctggatgctatttttgcattatacactcaggcacaacgtaaaccttgatggctcatcttgctacaattacgagttgaaaaacactacttacgtatttgtatgacctattagtcagaggaaatcatacatatgctttgtaaatagactttgcagataactaaatagactgaagaaatatgttgcatttgatagaagcaattgcataaatatttggtttctatattagagtctgtgagtaaagtcaagtaataaacctaagtaggtataacagatttttaaaccttgaaacttgctttgatggtagagaaaatcattgaagatttacatactgtatataagatgtaaaatgtacgctgcttattaccctcaattttccagaagcaatggtatataatgcagttgaaaaaccaaaaatcttggaaaactaagacgggtcttgtttaaaatgtctctcagctttggcaaccttcaaatcttaatcaactatttaaagcattactgtgtcttgtagcctgcattccacaacagctctgttattcaggtaaaagacttgaactgagccgtttgggacctatactgtaatattttcattgaggaacaatatcctattttgtaaagcatttccctatgtgtgactttaaactgtaaaattaaacactgcttttgtgggttcagtgggcataataaatataaattgtaaactaggttaaagta

SEQ ID NO: 22 - protein, 930 aa, Homo sapiens
MLLGWASLLLCAFRLPLAAVGPAATPAQDKAGQPPTAAAAAQPRRRQGEEVQERAEPPGHPHPLAQRRRSKGLVQNIDQLYSGGGKVGYLVYAGGRRFLLDLERDGSVGIAGFVPAGGGTSAPWRHRSHCFYRGTVDGSPRSLAVFDLCGGLDGFFAVKHARYTLKPLLRGPWAEEEKGRVYGDGSARILHVYTREGFSFEALPPRASCETPASTPEAHEHAPAHSNPSGRAALASQLLDQSALSPAGGSGPQTWWRRRRRSISRARQVELLLVADASMARLYGRGLQHYLLTLASIANRLYSHASIENHIRLAVVKVVVLGDKDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEETFGSTEDKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRICLQGKCVDKTKKKYYSTSSHGNWGSWGSWGQCSRSCGGGVQFAYRHCNNPAPRNNGRYCTGKRAIYRSCSLMPCPPNGKSFRHEQCEAKNGYQSDAKGVKTFVEWVPKYAGVLPADVCKLTCRAKGTGYYVVFSPKVTDGTECRLYSNSVCVRGKCVRTGCDGIIGSKLQYDKCGVCGGDNSSCTKIVGTFNKKSKGYTDVVRIPEGATHIKVRQFKAKDQTRFTAYLALKKKNGEYLINGKYMISTSETIIDINGTVMNYSGWSHRDDFLHGMGYSATKEILIVQILATDPTKPLDVRYSFFVPKKSTPKVNSVTSHGSNKVGSHTSQPQWVTGPWLACSRTCDTGWHTRTVQCQDGNRKLAKGCPLSQRPSAFKQCLLKKC

SEQ ID NO: 23 - DNA, 2255 bp, Homo sapiens
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtgaaaaaaaaaaaaaaaaaa

SEQ ID NO: 24 - protein, 346 aa, Homo sapiens
MLKKPLSAVTWLCIFIVAFVSHPAWLQKLSKHKTPAQPQLKAANCCEEVKELKAQVANLSSLLSELNKKQERDWVSVVMQVMELESNSKRMESRLTDAESKYSEMNNQIDIMQLQAAQTVTQTSADAIYDCSSLYQKNYRISGVYKLPPDDFLGSPELEVFCDMETSGGGWTIIQRRKSGLVSFYRDWKQYKQGFGSIRGDFWLGNEHIHRLSRQPTRLRVEMEDWEGNLRYAEYSHFVLGNELNSYRLFLGNYTGNVGNDALQYHNNTAFSTKDKDNDNCLDKCAQLRKGGYWYNCCTDSNLNGVYYRLGEHNKHLDGITWYGWHGSTYSLKRVEMKIRPEDFKP

SEQ ID NO: 25 - DNA, 3572 bp, Homo sapiens
gcctttctggggcctgggggatcctcttgcactggtgggtggagagaagcgcctgcagccaaccagggtcaggctgtgctcacagtttcctctggcggcatgtaaaggctccacaaaggagttgggagttcaaatgaggctgctgcggacggcctgaggatggaccccaagccctggacctgccgagcgtggcactgaggcagcggctgacgctactgtgagggaaagaaggttgtgagcagccccgcaggacccctggccagccctggccccagcctctgccggagccctctgtggaggcagagccagtggagcccagtgaggcagggctgcttggcagccaccggcctgcaactcaggaacccctccagaggccatggacaggctgccccgctgacggccagggtgaagcatgtgaggagccgccccggagccaagcaggagggaagaggctttcatagattctattcacaaagaataaccaccattttgcaaggaccatgaggccactgtgcgtgacatgctggtggctcggactgctggctgccatgggagctgttgcaggccaggaggacggttttgagggcactgaggagggctcgccaagagagttcatttacctaaacaggtacaagcgggcgggcgagtcccaggacaagtgcacctacaccttcattgtgccccagcagcgggtcacgggtgccatctgcgtcaactccaaggagcctgaggtgcttctggagaaccgagtgcataagcaggagctagagctgctcaacaatgagctgctcaagcagaagcggcagatcgagacgctgcagcagctggtggaggtggacggcggcattgtgagcgaggtgaagctgctgcgcaaggagagccgcaacatgaactcgcgggtcacgcagctctacatgcagctcctgcacgagatcatccgcaagcgggacaacgcgttggagctctcccagctggagaacaggatcctgaaccagacagccgacatgctgcagctggccagcaagtacaaggacctggagcacaagtaccagcacctggccacactggcccacaaccaatcagagatcatcgcgcagcttgaggagcactgccagagggtgccctcggccaggcccgtcccccagccaccccccgctgccccgccccgggtctaccaaccacccacctacaaccgcatcatcaaccagatctctaccaacgagatccagagtgaccagaacctgaaggtgctgccaccccctctgcccactatgcccactctcaccagcctcccatcttccaccgacaagccgtcgggcccatggagagactgcctgcaggccctggaggatggccacgacaccagctccatctacctggtgaagccggagaacaccaaccgcctcatgcaggtgtggtgcgaccagagacacgaccccgggggctggaccgtcatccagagacgcctggatggctctgttaacttcttcaggaactgggagacgtacaagcaagggtttgggaacattgacggcgaatactggctgggcctggagaacatttactggctgacgaaccaaggcaactacaaactcctggtgaccatggaggactggtccggccgcaaagtctttgcagaatacgccagtttccgcctggaacctgagagcgagtattataagctgcggctggggcgctaccatggcaatgcgggtgactcctttacatggcacaacggcaagcagttcaccaccctggacagagatcatgatgtctacacaggaaactgtgcccactaccagaagggaggctggtggtataacgcctgtgcccactccaacctcaacggggtctggtaccgcgggggccattaccggagccgctaccaggacggagtctactgggctgagttccgaggaggctcttactcactcaagaaagtggtgatgatgatccgaccgaaccccaacaccttccactaagccagctccccctcctgacctctcgtggccattgccaggagcccaccctggtcacgctggccacagcacaaagaacaactcctcaccagttcatcctgaggctgggaggaccgggatgctggattctgttttccgaagtcactgcagcggatgatggaactgaatcgatacggtgttttctgtccctcctactttccttcacaccagacagcccctcatgtctccaggacaggacaggactacagacaactctttctttaaataaattaagtctctacaataaaaacacaactgcaaagtaccttcataatatacatgtgtatgagcctcccttgtgcacgtatgtgtataccacatatatatgcatttagatatacatcacatgtgatatatctagatccatatataggtttgccttagatacctaaatacacatatattcagttctcagatgttgaagctgtcaccagcagctttgctcttaggagaaaagcatttcattagtgttgtattacttgagtctaagggtagatcacagactgtgtggtctcaactgaaaggatcacccttggcatctgtgtgcctggattcttccagaatgtctacaatgctaatctctcacatagaggttcccagcttcttaagaaccccttttggcacctaatcaaatttcaaaatccctccccccacattttcatacttttccccattctcaggacttttcaccatccatcacccacttatcccttcatttgacaccattcattaagtgccttctgtgtgtcagtccctggccactcactgcagttcaaggccccctttccgctctgctgtactcctcgcctacctactccttgccttttctgtcgcacagccccttctttccaggcgagattcctcagcttctgagtaggaaacactccgggctccaggtttctggttgggaagggaaggccaggccaaaagctccaccggccgtatagataatgtactcgcagttttgtatcttccattcatactttaacctacaggtcatttgagtcttcacacaaataataacctatctggccaggagaattatctcagaacagaagtcatcagatcatcagagcccccagatggctacagaccagagattccacgctctcaggctgactagagtccgcatctcatctccaaactacacttccctggagaacaagtgccacaaaaatgaaaacaggccacttctcaggagttgaataatcaggggtcaccggaccccttggttgatgcactgcagcatggtggctttctgagtcctgttggccaccaagtgtcagcctcagcactcccgggactattgccaagaaggggcaagggatgagtcaagaaggtgagacccttcccggtgggcacgtgggccaggctgtgtgagatgttggatgtttggtactgtccatgtctgggtgtgtgcctattacctcagcatttctcacaaagtgtaccatgtagcatgttttgtgtatataaaagggagggtttttttaaaaatatattcccagattatccttgtaatgacacgaatctgcaataaaagccatcagtgct

SEQ ID NO: 26 - protein, 493 aa, Homo sapiens
MRPLCVTCWWLGLLAAMGAVAGQEDGFEGTEEGSPREFIYLNRYKRAGESQDKCTYTFIVPQQRVTGAICVNSKEPEVLLENRVHKQELELLNNELLKQKRQIETLQQLVEVDGGIVSEVKLLRKESRNMNSRVTQLYMQLLHEIIRKRDNALELSQLENRILNQTADMLQLASKYKDLEHKYQHLATLAHNQSEIIAQLEEHCQRVPSARPVPQPPPAAPPRVYQPPTYNRIINQISTNEIQSDQNLKVLPPPLPTMPTLTSLPSSTDKPSGPWRDCLQALEDGHDTSSIYLVKPENTNRLMQVWCDQRHDPGGWTVIQRRLDGSVNFFRNWETYKQGFGNIDGEYWLGLENIYWLTNQGNYKLLVTMEDWSGRKVFAEYASFRLEPESEYYKLRLGRYHGNAGDSFTWHNGKQFTTLDRDHDVYTGNCAHYQKGGWWYNACAHSNLNGVWYRGGHYRSRYQDGVYWAEFRGGSYSLKKVVMMIRPNPNTFH

SEQ ID NO: 27 - DNA, 5270 bp, Homo sapiens
aaagtgattgattcggatactgacactgtaggatctggggagagaggaacaaaggaccgtgaaagctgctctgtaaaagctgacacagccctcccaagtgagcaggactgttcttcccactgcaatctgacagtttactgcatgcctggagagaacacagcagtaaaaaccaggtttgctactggaaaaagaggaaagagaagactttcattgacggacccagccatggcagcgtagcagccctgcgttttagacggcagcagctcgggactctggacgtgtgtttgccctcaagtttgctaagctgctggtttattactgaagaaagaatgtggcagattgttttctttactctgagctgtgatcttgtcttggccgcagcctataacaactttcggaagagcatggacagcataggaaagaagcaatatcaggtccagcatgggtcctgcagctacactttcctcctgccagagatggacaactgccgctcttcctccagcccctacgtgtccaatgctgtgcagagggacgcgccgctcgaatacgatgactcggtgcagaggctgcaagtgctggagaacatcatggaaaacaacactcagtggctaatgaagcttgagaattatatccaggacaacatgaagaaagaaatggtagagatacagcagaatgcagtacagaaccagacggctgtgatgatagaaatagggacaaacctgttgaaccaaacagcggagcaaacgcggaagttaactgatgtggaagcccaagtattaaatcagaccacgagacttgaacttcagctcttggaacactccctctcgacaaacaaattggaaaaacagattttggaccagaccagtgaaataaacaaattgcaagataagaacagtttcctagaaaagaaggtgctagctatggaagacaagcacatcatccaactacagtcaataaaagaagagaaagatcagctacaggtgttagtatccaagcaaaattccatcattgaagaactagaaaaaaaaatagtgactgccacggtgaataattcagttcttcagaagcagcaacatgatctcatggagacagttaataacttactgactatgatgtccacatcaaactcagctaaggaccccactgttgctaaagaagaacaaatcagcttcagagactgtgctgaagtattcaaatcaggacacaccacgaatggcatctacacgttaacattccctaattctacagaagagatcaaggcctactgtgacatggaagctggaggaggcgggtggacaattattcagcgacgtgaggatggcagcgttgattttcagaggacttggaaagaatataaagtgggatttggtaacccttcaggagaatattggctgggaaatgagtttgtttcgcaactgactaatcagcaacgctatgtgcttaaaatacaccttaaagactgggaagggaatgaggcttactcattgtatgaacatttctatctctcaagtgaagaactcaattataggattcaccttaaaggacttacagggacagccggcaaaataagcagcatcagccaaccaggaaatgattttagcacaaaggatggagacaacgacaaatgtatttgcaaatgttcacaaatgctaacaggaggctggtggtttgatgcatgtggtccttccaacttgaacggaatgtactatccacagaggcagaacacaaataagttcaacggcattaaatggtactactggaaaggctcaggctattcgctcaaggccacaaccatgatgatccgaccagcagatttctaaacatcccagtccacctgaggaactgtctcgaactattttcaaagacttaagcccagtgcactgaaagtcacggctgcgcactgtgtcctcttccaccacagagggcgtgtgctcggtgctgacgggacccacatgctccagattagagcctgtaaactttatcacttaaacttgcatcacttaacggaccaaagcaagaccctaaacatccataattgtgattagacagaacacctatgcaaagatgaacccgaggctgagaatcagactgacagtttacagacgctgctgtcacaaccaagaatgttatgtgcaagtttatcagtaaataactggaaaacagaacacttatgttatacaatacagatcatcttggaactgcattcttctgagcactgtttatacactgtgtaaatacccatatgtcctgaattcaccatcactatcacaattaaaaggaagaaaaaaactctctaagccataaaaagacatattcagggatattctgagaaggggttactagaagtttaatatttggaaaaacagttagtgcatttttactccatctcttaggtgctttaaatttttatttcaaaaacagcgtatttacatttatgttgacagcttagttataagttaatgctcaaatacgtatttcaaatttatatggtagaaacttccagaatctctgaaattatcaacagaaacgtgccattttagtttatatgcagaccgtactatttttttctgcctgattgttaaatatgaaggtatttttagtaattaaatataacttattaggggatatgcctatgtttaacttttatgataatatttacaattttataatttgtttccaaaagacctaattgtgccttgtgataaggaaacttcttacttttaatgatgaggaaaattatacatttcattctatgacaaagaaactttactatcttctcactattctaaaacagaggtctgttttctttcctagtaagatatatttttatagaactagactacaatttaatttctggttgagaaaagccttctatttaagaaatttacaaagctatatgtctcaagattcacccttaaatttacttaaggaaaaaaataattgacactagtaagtttttttatgtcaatcagcaaactgaaaaaaaaaaaagggtttcaaagtgcaaaaacaaaatctgatgttcataatatatttaaatatttaccaaaaatttgagaacacagggctgggcgcagtggctcacacctataatcccagtacattggtaggcaaggtgggcagatcacctgaggtcaggagttcaagaccagcctggacaacatggtgaaaccctgtctctactaaataatacaaaaattagccaggcgtgctggcgggcacctgtaatcccagctactcgggaggctgaggcagggagaattgcttgcaccagggaggtagaggttgcagtgagccaagatcgcaccactgcactccagccggggcaacagagcaagactccatctcaaaaaaaaaaaaaaaaaaagaaagaaaagaaaatttgagaacacagctttatactcgggactacaaaaccataaactcctggagttttaactccttttgaaattttcatagtacaattaatactaatgaacatttgtgtaaagctttataatttaaaggcaatttctcatatattcttttctgaatcatttgcaaggaagttcagagtccagtctgtaactagcatctactatatgtctgtcttcaccttacagtgttctaccattattttttctttattccatttcaaaatctaatttattttaccccaacttctccccaccacttgacgtagttttagaacacacaggtgttgctacatatttggagtcaatgatggactctggcaaagtcaaggctctgttttatttccaccaaggtgcacttttccaacaactatttaactagttaagaacctccctatcttagaactgtatctactttatatttaagaaggttttatgaattcaacaacggtatcatggccttgtatcaagttgaaaaacaactgaaaataagaaaatttcacagcctcgaaagacaacaacaagtttctaggatatctcaatgacaagagtgatggatacttaggtagggaaacgctaatgcaggaaaaactggcaacaacacaatttatatcaattctctttgtaggcaggtgataaaaaattcaaggacaaatctcattatgtcattgtgcatcatatataatctcttatgagcgagaatggggggaatttgtgtttttactttacacttcaattccttacacggtatttcaaacaaacagttttgctgagaggagcttttgtctctccttaagaaaatgtttataaagctgaaaggaaatcaaacagtaatcttaaaaatgaaaacaaaacaacccaacaacctagataactacagtgatcagggagcacagttcaactccttgttatgttttagtcatatggcctactcaaacagctaaataacaacaccagtggcagataaaaatcaccatttatctttcagctattaatcttttgaatgaataaactgtgacaaacaaattaacatttttgaacatgaaaggcaacttctgcacaatcctgtatccaagcaaactttaaattatccacttaattattacttaatcttaaaaaaaattagaacccagaacttttcaatgaagcatttgaaagttgaagtggaatttaggaaagccataaaaatataaatactgttatcacagcaccagcaagccataatctttatacctatcagttctatttctattaacagtaaaaacattaagcaagatataagactacctgcccaagaattcagtcttttttcatttttgtttttctcagttctgaggatgttaatcgtcaaattttctttggactgcattcctcactactttttgcacaatggtctcacgttctcacatttgttctcgcgaataaattgataaaaggtgttaagttctgtgaatgtctttttaattatgggcataattgtgcttgactggataaaaacttaagtccacccttatgtttataataatttcttgagaacagcaaactgcatttaccatcgtaaaacaacatctgacttacgggagctgcagggaagtggtgagacagttcgaacggctcctcagaaatccagtgacccaattctaaagaccatagcacctgcaagtgacacaacaagcagatttattatacatttattagccttagcaggcaataaaccaagaatcactttgaagacacagcaaaaagtgatacactccgcagatctgaaatagatgtgttctcagacaacaaagtcccttcagaatcttcatgttgcataaatgttatgaatattaataaaaagttgattgagaaaaa

SEQ ID NO: 28 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 29 - DNA, 1726 bp, Homo sapiens
cttggcttctggcccccttatctgcccccgccccacgcgccctggcagcaccatgagccgccagcttctgcctgtactgctgctgctgctgctcagggcttcgtgcccatggggtcaggaacagggagcgaggagcccctcggaggagcctccagaggaggaaatccccaaggaggatgggatcttggtgctgagccgccacaccctgggcctggccctgcgggagcaccctgccctgctggtggaattctatgccccgtggtgtgggcactgccaggccctggcccccgagtacagcaaggcagctgccgtgctcgcggccgagtcaatggtggtcacgctggccaaggtggatgggcccgcgcagcgcgagctggctgaggagtttggtgtgacggagtaccctacgctcaagttcttccgcaatgggaaccgcacgcacccggaggagtacacaggaccacgggacgctgagggcattgccgagtggctgcgacggcgggtggggcccagtgccatgcggctggaggacgaggcggccgcccaggcgctgatcggtggccgggacctagtggtcattggcttcttccaggacctgcaggacgaggacgtggccaccttcttggccttggcccaggacgccctggacatgacctttggcctcacagaccggccgcggctctttcagcagtttggcctcaccaaggacactgtggttctcttcaagaagtttgatgaggggcgggcagacttccccgtggacgaggagcttggcctggacctgggggatctgtcgcgcttcctggtcacacacagcatgcgcctggtcacggagttcaacagccagacgtctgccaagatcttcgcggccaggatcctcaaccacctgctgctgtttgtcaaccagacgctggctgcgcaccgggagctcctagcgggctttggggaggcagctccccgcttccgggggcaggtgctgttcgtggtggtggacgtggcggccgacaatgagcacgtgctgcagtactttggactcaaggctgaggcagcccccactctgcgcttggtcaaccttgaaaccactaagaagtatgcgcctgtggatgggggccctgtcaccgcagcgtccatcactgctttctgccatgcagtcctcaacggccaagtcaagccctatctcctgagccaggagataccccctgattgggatcagcggccagttaagaccctcgtgggcaagaattttgagcaggtggcttttgacgaaaccaagaatgtgtttgtcaagttctatgccccgtggtgcacccactgcaaggagatggcccctgcctgggaggcattggctgagaagtaccaagaccacgaggacatcatcattgctgagctggatgccacggccaacgagctggatgccttcgctgtgcacggcttccctactctcaagtacttcccagcagggccaggtcggaaggtgattgaatacaaaagcaccagggacctggagactttctccaagttcctggacaacgggggcgtgctgcccacggaggagcccccggaggagccagcagccccgttcccggagccaccggccaactccactatggggtccaaggaggaactgtagctgcccccgtgtcacccccgccatcactgctggacaggagccacccccttgggtaccagagggagctgtgcattgtgaataaagagtgagcttggt

SEQ ID NO: 30 - protein, 525 aa, Homo sapiens
MSRQLLPVLLLLLLRASCPWGQEQGARSPSEEPPEEEIPKEDGILVLSRHTLGLALREHPALLVEFYAPWCGHCQALAPEYSKAAAVLAAESMVVTLAKVDGPAQRELAEEFGVTEYPTLKFFRNGNRTHPEEYTGPRDAEGIAEWLRRRVGPSAMRLEDEAAAQALIGGRDLVVIGFFQDLQDEDVATFLALAQDALDMTFGLTDRPRLFQQFGLTKDTVVLFKKFDEGRADFPVDEELGLDLGDLSRFLVTHSMRLVTEFNSQTSAKIFAARILNHLLLFVNQTLAAHRELLAGFGEAAPRFRGQVLFVVVDVAADNEHVLQYFGLKAEAAPTLRLVNLETTKKYAPVDGGPVTAASITAFCHAVLNGQVKPYLLSQEIPPDWDQRPVKTLVGKNFEQVAFDETKNVFVKFYAPWCTHCKEMAPAWEALAEKYQDHEDIIIAELDATANELDAFAVHGFPTLKYFPAGPGRKVIEYKSTRDLETFSKFLDNGGVLPTEEPPEEPAAPFPEPPANSTMGSKEEL

SEQ ID NO: 31 - DNA, 1873 bp, Homo sapiens
ctgcgcatgctttggcagacgtggcaccgggaactcggaggcggggagcggctgggaagtggccgtggtggttggccgcggtggagctagcaggcgggcgggcgggagcgggcgccggagtggagaaaggagccagcggtgggcagcgctgctgggatggcgcgggccgggccggcgtggctgctgctggcaatctgggtggtcctgccatcatggctgtcctctgcaaaggtctcctcgctcattgagagaatctctgaccccaaggacttgaaaaaactgctcagaacccggaataatgtactggtgctttactccaaatctgaggtggcagctgaaaatcatctcaggttactgtccacagtggcccaggcggtgaaaggacaagggaccatctgctgggtggactgtggtgatgcagagagtagaaaattgtgcaagaagatgaaagttgacctgagcccgaaggacaaaaaggttgaattattccattaccaggatggtgcatttcatactgaatataaccgagctgtgacatttaagtccatagtggcctttttgaaggatccaaaagggcccccactgtgggaggaagatcctggagccaaagatgttgtccaccttgacagtgaaaaggacttcagacggctcctgaagaaggaagagaagccgctcctgatcatgttttatgccccctggtgcagcatgtgcaagaggatgatgccgcatttccagaaggctgcgactcagctgcgaggccacgccgtgctggccgggatgaatgtctactcctctgaatttgaaaacatcaaggaggagtacagcgtgcgcggcttccccaccatctgctattttgagaaaggacggttcttgttccagtatgacaactatgggtccacagctgaggacattgtggagtggctgaagaatccgcagccgccacagccccaggtccctgagactccctgggcagatgagggcggctccgtttatcacctgaccgatgaagactttgaccagtttgtgaaggaacactcctctgtcctcgtcatgttccacgccccatggtgtggccactgtaagaaaatgaagccggagtttgagaaggcagcagaagccctccatggagaagcggatagctctggtgtccttgcagctgtcgatgccactgtcaacaaggccctggcagaaagattccacatctcagagtttcctacgttgaagtattttaagaatggagagaaatacgcagtgcctgtgctcaggacaaagaagaagtttctcgagtggatgcaaaaccctgaggcccccccgcccccagagcccacgtgggaagagcagcagacaagcgtgttgcacctggtgggggacaacttccgggagaccctgaagaagaagaaacacaccttggtcatgttctacgccccttggtgcccacactgtaagaaggtcattccgcactttactgctactgctgatgccttcaaagatgaccgaaagattgcctgtgccgctgttgactgtgtcaaagacaagaaccaagacctgtgccagcaggaggcggtcaagggctaccccactttccactactaccactatgggaagttcgcagaaaagtatgacagcgaccgcacagaattgggatttaccaattatattcgagccctccgggagggagaccatgaaagactagggaaaaagaaggaagagttataattcctgcctcagaaaaagcttttccattacactgtgaatgatacctgttttgttgtttctgaatttccacatgttctgaagacaaattttttatagccgcttatggccattttgtacaattttgaaataaaattaaaccatttattaaaaaaaaaaa

SEQ ID NO: 32 - protein, 519 aa, Homo sapiens
MARAGPAWLLLAIWVVLPSWLSSAKVSSLIERISDPKDLKKLLRTRNNVLVLYSKSEVAAENHLRLLSTVAQAVKGQGTICWVDCGDAESRKLCKKMKVDLSPKDKKVELFHYQDGAFHTEYNRAVTFKSIVAFLKDPKGPPLWEEDPGAKDVVHLDSEKDFRRLLKKEEKPLLIMFYAPWCSMCKRMMPHFQKAATQLRGHAVLAGMNVYSSEFENIKEEYSVRGFPTICYFEKGRFLFQYDNYGSTAEDIVEWLKNPQPPQPQVPETPWADEGGSVYHLTDEDFDQFVKEHSSVLVMFHAPWCGHCKKMKPEFEKAAEALHGEADSSGVLAAVDATVNKALAERFHISEFPTLKYFKNGEKYAVPVLRTKKKFLEWMQNPEAPPPPEPTWEEQQTSVLHLVGDNFRETLKKKKHTLVMFYAPWCPHCKKVIPHFTATADAFKDDRKIACAAVDCVKDKNQDLCQQEAVKGYPTFHYYHYGKFAEKYDSDRTELGFTNYIRALREGDHERLGKKKEEL

SEQ ID NO: 33 - DNA, 1593 bp, Homo sapiens
gcggtgcccttgcggcgcagctggggtcgcggccctgctccccgcgctttcttaaggcccgcgggcggcgcaggagcggcactcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgagtatgttaagctctttatgactgtttttgtagtggtatagagtactgcagaatacagtaagctgctctattgtagcatttcttgatgttgcttagtcacttatttcataaacaacttaatgttctgaataatttcttactaaacattttgttattgggcaagtgattgaaaatagtaaatgctttgtgtgattgaatctgattggacattttcttcagagagctaaattacaattgtcatttataaaaccatcaaaaatattccatccatatactttggggacttgtagggatgcctttctagtcctattctattgcagttatagaaaatctagtcttttgccccagttacttaaaaataaaatattaacactttcccaagggaaacactcggctttctatagaaaattgcactttttgtcgagtaatcctctgcagtgatacttctggtagatgtcacccagtggtttttgttaggtcaaatgttcctgtatagtttttgcaaatagagctgtatactgtttaaatgtagcaggtgaactgaactggggtttgctcacctgcacagtaaaggcaaacttcaacagcaaaactgcaaaaaggtggtttttgcagtaggagaaaggaggatgtttatttgcagggcgccaagcaaggagaattgggcagctcatgcttgagacccaatctccatgatgacctacaagctagagtatttaaaggcagtggtaaatttcaggaaagcagaagtt

SEQ ID NO: 34 - protein, 222 aa, Homo sapiens
MLSRAVCGTSRQLAPVLGYLGSRQKHSLPDLPYDYGALEPHINAQIMQLHHSKHHAAYVNNLNVTEEKYQEALAKGDVTAQIALQPALKFNGGGHINHSIFWTNLSPNGGGEPKGELLEAIKRDFGSFDKFKEKLTAASVGVQGSGWGWLGFNKERGHLQIAACPNQDPLQGTTGLIPLLGIDVWEHAYYLQYKNVRPDYLKAIWNVINWENVTERYMACKK

SEQ ID NO: 35 - DNA, 1546 bp, Homo sapiens
ggggaggtctggcctgcttttcctccctgaactggcccaatgactggctccctcacgctgaccactcctctgggctggcctcctgcactcgcgctaacagcccaggctccagggacagcctgcgttcctgggctggctgggtgcagctctcttttcaggagagaaagctctcttggaggagctggaaaggtgcccgactccagccatgctggcgctactgtgttcctgcctgctcctggcagccggtgcctcggacgcctggacgggcgaggactcggcggagcccaactctgactcggcggagtggatccgagacatgtacgccaaggtcacggagatctggcaggaggtcatgcagcggcgggacgacgacggcgcgctccacgccgcctgccaggtgcagccgtcggccacgctggacgccgcgcagccccgggtgaccggcgtcgtcctcttccggcagcttgcgccccgcgccaagctcgacgccttcttcgccctggagggcttcccgaccgagccgaacagctccagccgcgccatccacgtgcaccagttcggggacctgagccagggctgcgagtccaccgggccccactacaacccgctggccgtgccgcacccgcagcacccgggcgacttcggcaacttcgcggtccgcgacggcagcctctggaggtaccgcgccggcctggccgcctcgctcgcgggcccgcactccatcgtgggccgggccgtggtcgtccacgctggcgaggacgacctgggccgcggcggcaaccaggccagcgtggagaacgggaacgcgggccggcggctggcctgctgcgtggtgggcgtgtgcgggcccgggctctgggagcgccaggcgcgggagcactcagagcgcaagaagcggcggcgcgagagcgagtgcaaggccgcctgagcgcggcccccacccggcggcggccagggacccccgaggcccccctctgcctttgagcttctcctctgctccaacagacaccctccactctgaggtctcaccttcgcctttgctgaagtctccccgcagccctctccacccagaggtctccctataccgagacccaccatccttccatcctgaggaccgccccaaccctcggagccccccactcagtaggtctgaaggcctccatttgtaccgaaacaccccgctcacgctgacagcctcctaggctccctgaggtacctttccacccagaccctccttccccaccccataagccctgagactcccgcctttgacctgacgatcttcccccttcccgccttcaggttcctcctaggcgctcagaggccgctctggggggttgcctcgagtccccccacccctccccacccaccaccgctcccgcggcaagccagcccgtgcaacggaagccaggccaactgccccgcgtcttcagctgtttcgcatccaccgccaccccactgagagctgctcctttgggggaatgtttggcaacctttgtgttacagattaaaaattcagcaattcagtaaaaaaaaaaaaaaaaaa

SEQ ID NO: 36 - protein, 240 aa, Homo sapiens
MLALLCSCLLLAAGASDAWTGEDSAEPNSDSAEWIRDMYAKVTEIWQEVMQRRDDDGALHAACQVQPSATLDAAQPRVTGVVLFRQLAPRAKLDAFFALEGFPTEPNSSSRAIHVHQFGDLSQGCESTGPHYNPLAVPHPQHPGDFGNFAVRDGSLWRYRAGLAASLAGPHSIVGRAVVVHAGEDDLGRGGNQASVENGNAGRRLACCVVGVCGPGLWERQAREHSERKKRRRESECKAA

SEQ ID NO: 37 - DNA, 1327 bp, Homo sapiens
gcggccgcaccccccggccgggccgtgcttctgcccctacaaggtttgggccgaggtgggggagggtcctggttgccggccccgcccggtccctccccgccttttaggcgcccgcgtggccgggacgtcccagtcccgctccgtcctcctcgcctgccaccggtgcacccagtccgctcacccagcccagtccgtccggtcctcaccgcctgccggccggcccaccccccaccgcagccatggacgccatcaagaagaagatgcagatgctgaagctggacaaggagaacgccatcgaccgcgccgagcaggccgaagccgacaagaagcaagctgaggaccgctgcaagcagctggaggaggagcagcaggccctccagaagaagctgaaggggacagaggatgaggtggaaaagtattctgaatccgtgaaggaggcccaggagaaactggagcaggccgagaagaaggccactgatgctgaggcagatgtggcctccctgaaccgccgcattcagctggttgaggaggagctggaccgggcccaggagcgcctggctacagccctgcagaagctggaggaggccgagaaggcggctgatgagagcgagagaggaatgaaggtcatcgaaaaccgggccatgaaggatgaggagaagatggaactgcaggagatgcagctgaaggaggccaagcacatcgctgaggattcagaccgcaaatatgaagaggtggccaggaagctggtgatcctggaaggagagctggagcgctcggaggagagggctgaggtggccgagagtaaatgtggggacctagaggaggagctgaaaattgttaccaacaacttgaaatccctggaggcccaggcggacaagtattccaccaaagaagataaatatgaagaggagatcaaactgttggaggagaagctgaaggaggctgagacccgagcagagtttgccgagaggtctgtggcaaagttggagaaaaccatcgatgacctagaagatgaagtctatgcccagaagatgaagtacaaggccattagcgaggaactggacaacgcactcaatgacatcacctccctctgagccccacgccagcgtggccacctcagctctcttctctcctctcctttccattctctctatggggaggggagcaggcaggaggagcagaaattgccaacattgcacagccaggctgggagcagcctagggagagcccccatcatgcccaccacccactctggcactggcttcatcctttacctatccccttccaccctcctttgctgcttaataaattctgaacttggtctcca

SEQ ID NO: 38 - protein, 284 aa, Homo sapiens
MDAIKKKMQMLKLDKENAIDRAEQAEADKKQAEDRCKQLEEEQQALQKKLKGTEDEVEKYSESVKEAQEKLEQAEKKATDAEADVASLNRRIQLVEEELDRAQERLATALQKLEEAEKAADESERGMKVIENRAMKDEEKMELQEMQLKEAKHIAEDSDRKYEEVARKLVILEGELERSEERAEVAESKCGDLEEELKIVTNNLKSLEAQADKYSTKEDKYEEEIKLLEEKLKEAETRAEFAERSVAKLEKTIDDLEDEVYAQKMKYKAISEELDNALNDITSL

SEQ ID NO: 39 - DNA, 1384 bp, Homo sapiens
gtaagaaacggttgaactggatgcaatttttatcacagcttgtgtaagactgcctctgtccctcctctcacatgccattggttaaccagcagacagtgtgctcaggggcgttgccagctcattgctcttatagcctgtgagggaggaagaaagaaacatttgccagccaggctagtgacagaaatggattcgaaatatcagtgtgtgaagctgaatgatggtcacttcatgcctgtcctgggatttggcacctatgcgcctgcagaggttcctaaaagtaaagctttagaggccaccaaattggcaattgaagctggcttccgccatattgattctgctcatttatacaataatgaggagcaggttggactggccatccgaagcaagattgcagatggcagtgtgaagagagaagacatattctacacttcaaagctttggtgcaattcccatcgaccagagttggtccgaccagccttggaaaggtcactgaaaaatcttcaattggattatgttgacctctaccttattcattttccagtgtctgtaaagccaggtgaggaagtgatcccaaaagatgaaaatggaaaaatactatttgacacagtggatctctgtgccacatgggaggccgtggagaagtgtaaagatgcaggattggccaagtccatcggggtgtccaacttcaaccgcaggcagctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccaccgagaagaaccatgggtggacccgaactccccggtgctcttggaggacccagtcctttgtgccttggcaaaaaagcacaagcgaaccccagccctgattgccctgcgctaccagctacagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaacatggagggcattgcatgaggtctgccagaaggccctgcgtgtggatggtgacacagaggatggctctatgctggtgactggacacatcgcctctggttaaatctctcctgcttggtgatttcagcaagctacagcaaagcccattggccagaaaggaaagacaataattttgttttttcattttgaaaaaattaaatgctctctcctaaagattcttcacctaaaaaa

SEQ ID NO: 40 - protein, 323 aa, Homo sapiens
MDSKYQCVKLNDGHFMPVLGFGTYAPAEVPKSKALEATKLAIEAGFRHIDSAHLYNNEEQVGLAIRSKIADGSVKREDIFYTSKLWCNSHRPELVRPALERSLKNLQLDYVDLYLIHFPVSVKPGEEVIPKDENGKILFDTVDLCATWEAVEKCKDAGLAKSIGVSNFNRRQLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDIFAGPPNYPFSDEY

SEQ ID NO: 41 - DNA, 1224 bp, Homo sapiens
gcccattgtttttgtaatctctgaggagaagcagcagcaaacatttgctagtcagacaagtgacagggaatggattccaaacaccagtgtgtaaagctaaatgatggccacttcatgcctgtattgggatttggcacctatgcacctccagaggttccgagaagtaaagctttggaggtcacaaaattagcaatagaagctgggttccgccatatagattctgctcatttatacaataatgaggagcaggttggactggccatccgaagcaagattgcagatggcagtgtgaagagagaagacatattctacacttcaaagctttggtccacttttcatcgaccagagttggtccgaccagccttggaaaactcactgaagaaagctcaattggactatgttgacctctatcttattcattctccaatgtctctaaagccaggtgaggaactttcaccaacagatgaaaatggaaaagtaatatttgacatagtggatctctgtaccacctgggaggccatggagaagtgtaaggatgcaggattggccaagtccattggggtgtcaaacttcaaccgcaggcagctggagatgatcctcaacaagccaggactcaagtacaagcctgtctgcaaccaggtagaatgtcatccgtatttcaaccggagtaaattgctagatttctgcaagtcgaaagatattgttctggttgcctatagtgctctgggatctcaacgagacaaacgatgggtggacccgaactccccggtgctcttggaggacccagtcctttgtgccttggcaaaaaagcacaagcgaaccccagccctgattgccctgcgctaccagctgcagcgtggggttgtggtcctggccaagagctacaatgagcagcgcatcagacagaacgtgcaggtttttgagttccagttgactgcagaggacatgaaagccatagatggcctagacagaaatctccactattttaacagtgatagttttgctagccaccctaattatccatattcagatgaatattaacatggagggctttgcctgatgtctaccagaagccctgtgtgtggatggtgacgcagaggacgtctctatgccggtgactggacatatcacctctacttaaatccgtcctgtttagcgacttcagtcaactacagctgagtccataggccagaaagacaataaatttttatcattttgaaataa

SEQ ID NO: 42 - protein, 323 aa, Homo sapiens
MDSKHQCVKLNDGHFMPVLGFGTYAPPEVPRSKALEVTKLAIEAGFRHIDSAHLYNNEEQVGLAIRSKIADGSVKREDIFYTSKLWSTFHRPELVRPALENSLKKAQLDYVDLYLIHSPMSLKPGEELSPTDENGKVIFDIVDLCTTWEAMEKCKDAGLAKSIGVSNFNRRQLEMILNKPGLKYKPVCNQVECHPYFNRSKLLDFCKSKDIVLVAYSALGSQRDKRWVDPNSPVLLEDPVLCALAKKHKRTPALIALRYQLQRGVVVLAKSYNEQRIRQNVQVFEFQLTAEDMKAIDGLDRNLHYFNSDSFASHPNYPYSDEY

SEQ ID NO: 43 - DNA, 1610 bp, Homo sapiens
acagtagctcacacctgtaatcccagcactttggaaggccgaggtgggcggatcacctgagctcaggagtttgagaccagcctgtctctactaacaatataaaaattagctgggagtcacggtgggcgcctgtaatcccagctactcgggaggctgaggcaggagaattgcttgaacccaggagacagaggttgtagtgagctgagatcgcaccactgcactctagccttggcaacagtgcaagactgtctcaaaaacagcaacagagagcaggacgtgagacttctacctgctcactcagaatcatttctgcaccaaccatggccacgtttgtggagctcagtaccaaagccaagatgcccattgtgggcctgggcacttggaagtctcctcttggcaaagtgaaagaagcagtgaaggtggccattgatgcaggatatcggcacattgactgtgcctatgtctatcagaatgaacatgaagtgggggaagccatccaagagaagatccaagagaaggctgtgaagcgggaggacctgttcatcgtcagcaagttgtggcccactttctttgagagaccccttgtgaggaaagcctttgagaagaccctcaaggacctgaagctgagctatctggacgtctatcttattcactggccacagggattcaagtctggggatgaccttttccccaaagatgataaaggtaatgccatcggtggaaaagcaacgttcttggatgcctgggaggccatggaggagctggtggatgaggggctggtgaaagcccttggggtctccaatttcagccacttccagatcgagaagctcttgaacaaacctggactgaaatataaaccagtgactaaccaggttgagtgtcacccatacctcacacaggagaaactgatccagtactgccactccaagggcatcaccgttacggcctacagccccctgggctctccggatagaccttgggccaagccagaagacccttccctgctggaggatcccaagattaaggagattgctgcaaagcacaaaaaaaccgcagcccaggttctgatccgtttccatatccagaggaatgtgattgtcatccccaagtctgtgacaccagcacgcattgttgagaacattcaggtctttgactttaaattgagtgatgaggagatggcaaccatactcagcttcaacagaaactggagggcctgtaacgtgttgcaatcctctcatttggaagactatcccttcaatgcagaatattgaggttgaatctcctggtgagattatacaggagattctctttcttcgctgaagtgtgactacctccactcatgtcccattttagccaagcttatttaagatcacagtgaacttagtcctgttatagacgagaatcgaggtgctgttttagacatttatttctgtatgttcaactaggatcagaatatcacagaaaagcatggcttgaataaggaaatgacaattttttccacttatctgatcagaacaaatgtttattaagcatcagaaactctgccaacactgaggatgtaaagatcaataaaaaaaataataatcataaccaacaaaaaaaaaaaaaaa

SEQ ID NO: 44 - protein, 316 aa, Homo sapiens
MATFVELSTKAKMPIVGLGTWKSPLGKVKEAVKVAIDAGYRHIDCAYVYQNEHEVGEAIQEKIQEKAVKREDLFIVSKLWPTFFERPLVRKAFEKTLKDLKLSYLDVYLIHWPQGFKSGDDLFPKDDKGNAIGGKATFLDAWEAMEELVDEGLVKALGVSNFSHFQIEKLLNKPGLKYKPVTNQVECHPYLTQEKLIQYCHSKGITVTAYSPLGSPDRPWAKPEDPSLLEDPKIKEIAAKHKKTAAQVLIRFHIQRNVIVIPKSVTPARIVENIQVFDFKLSDEEMATILSFNRNWRACNVLQSSHLEDYPFNAEY

SEQ ID NO: 45 - DNA, 1069 bp, Homo sapiens
aagtaattcctagacccgtaggtggccgcagagccggttacctctggttctgcgccagcgtgccccacccgcaggacggccgggttctttgatttgtacactttctaaaaccaaacccgagaggaagggcaggctcagggtggggatgccctgaaatattcgagagcaggaccgtttctactgaagagaagtttacaagaacgctctgtctggggcgggcgaggcctctgcgaggcgggtccgggagcgagggcagggcgtgggccgcgcgcccggggtcgggggagtcgggggcaggaagagggggaggagacagggctgggggagcgccctgccgagcgcccgccaggctcctcccgctcccgcgccgcctccctctacccacccgccgcacgtactaaggaaggcgcacagcccgccgcgctcgcctctccgccccgcgtccagctcgcccagctcgcccagcgtccgccgcgcctcggccaaggcttcaacggaccacaccaaaatgccatctcaaatggaacacgccatggaaaccatgatgtttacatttcacaaattcgctggggataaaggctacttaacaaaggaggacctgagagtactcatggaaaaggagttccctggatttttggaaaatcaaaaagaccctctggctgtggacaaaataatgaaggacctggaccagtgtagagatggcaaagtgggcttccagagcttcttttccctaattgcgggcctcaccattgcatgcaatgactattttgtagtacacatgaagcagaagggaaagaagtaggcagaaatgagcagttcgctcctccctgataagagttgtcccaaagggtcgcttaaggaatctgccccacagcttcccccatagaaggatttcatgagcagatcaggacacttagcaaatgtaaaaataaaatctaactctcatttgacaagcagagaaagaaaagttaaataccagataagcttttgatttttgtattgtttgcatccccttgccctcaataaataaagttcttttttagttccaaatttgaaaaaaaaaaaaaaa

SEQ ID NO: 46 - protein, 97 aa, Homo sapiens
MPSQMEHAMETMMFTFHKFAGDKGYLTKEDLRVLMEKEFPGFLENQKDPLAVDKIMKDLDQCRDGKVGFQSFFSLIAGLTIACNDYFVVHMKQKGKK

SEQ ID NO: 47 - DNA, 1929 bp, Homo sapiens
gcggcgtccgtccgtactgcagagccgctgccggagggtcgttttaaagggcccgcgcgttgccgccccctcggcccgccatgctgctatccgtgccgctgctgctcggcctcctcggcctggccgtcgccgagcctgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtagagaggcctgcctccagggctggactgaggcctgagcgctcctgccgcagagctggccgcgccaaataatgtctctgtgagactcgagaactttcatttttttccaggctggttcggatttggggtggattttggttttgttcccctcctccactctcccccaccccctccccgcccttttttttttttttttttaaactggtattttatctttgattctccttcagccctcacccctggttctcatctttcttgatcaacatcttttcttgcctctgtccccttctctcatctcttagctcccctccaacctggggggcagtggtgtggagaagccacaggcctgagatttcatctgctctccttcctggagcccagaggagggcagcagaagggggtggtgtctccaaccccccagcactgaggaagaacggggctcttctcatttcacccctccctttctcccctgcccccaggactgggccacttctgggtggggcagtgggtcccagattggctcacactgagaatgtaagaactacaaacaaaatttctattaaattaaattttgtgtctccaaaaaaaaaaaaaaaaaa

SEQ ID NO: 48 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 49 - DNA, 2463 bp, Homo sapiens
gtcctgtttctctccctgttgtccctgcctctttttccttcccgccgtgccccgcggccgggccggggcagccgggaagcgggtggggtggtgtgttacccagtagctcctgggacatcgctcgggtacgctccacgccgtcgcagccactgctgtggtcgccggtcggccgaggggccgcgatactggttgcccgcggtgtaagcagaattcgacgtgtatcgctgccgtcaagatggaggggcctttgtccgtgttcggtgaccgcagcactggggaaacgatccgctcccaaaacgttatggctgcagcttcgattgccaatattgtaaaaagttctcttggtccagttggcttggataaaatgttggtggatgatattggtgatgtaaccattactaacgatggtgcaaccatcctgaagttactggaggtagaacatcctgcagctaaagttctttgtgagctggctgatctgcaagacaaagaagttggagatggaactacttcagtggttattattgcagcagaactcctaaaaaatgcagatgaattagtcaaacagaaaattcatcccacatcagttattagtggctatcgacttgcttgcaaggaagcagtgcgttatatcaatgaaaacctaattgttaacacagatgaactgggaagagattgcctgattaatgctgctaagacatccatgtcttccaaaatcattggaataaatggtgatttctttgctaacatggtagtagatgctgtacttgctattaaatacacagacataagaggccagccacgctatccagtcaactctgttaatattttgaaagcccatgggagaagtcaaatggagagtatgctcatcagtggctatgcactcaactgtgtggtgggatcccagggcatgcccaagagaatcgtaaatgcaaaaattgcttgccttgacttcagcctgcaaaaaacaaaaatgaagcttggtgtacaggtggtcattacagaccctgaaaaactggaccaaattagacagagagaatcagatatcaccaaggagagaattcagaagatcctggcaactggtgccaatgttattctaaccactggtggaattgatgatatgtgtctgaagtattttgtggaggctggtgctatggcagttagaagagttttaaaaagggaccttaaacgcattgccaaagcttctggagcaactattctgtcaaccctggccaatttggaaggtgaagaaacttttgaagctgcaatgttgggacaggcagaagaagtggtacaggagagaatttgtgatgatgagctgatcttaatcaaaaatactaaggctcgtacgtctgcatcgattatcttacgtggggcaaatgatttcatgtgtgatgagatggagcgctctttacatgatgcactttgtgtagtgaagagagttttggagtcaaaatctgtggttcccggtgggggtgctgtagaagcagccctttccatataccttgaaaactatgcaaccagcatggggtctcgggaacagcttgcgattgcagagtttgcaagatcacttcttgttattcccaatacactagcagttaatgctgcccaggactccacagatctggttgcaaaattaagagcttttcataatgaggcccaggttaacccagaacgtaaaaatctaaaatggattggtcttgatttgagcaatggtaaacctcgagacaacaaacaagcaggggtgtttgaaccaaccatagttaaagttaagagtttgaaatttgcaacagaagctgcaatcaccattcttcgaattgatgatcttattaaattacatccagaaagtaaagatgataaacatggaagttatgaagatgctgttcactctggagcccttaatgattgatctgatgttccttttatttataacaatgttaaatgcaattgtcttgtaccttgagttgagtattacacattaaagtaaagtacaagctgtaaacttgggtttttgtgatgtaggaaatggtttccatctgtactttggtcctctgatttcacatattgcaacctagtactttattagtttaaaaagaaattgaggttgttcaaagtttaagcaattcattctctctgaacacacattgctattcccatcccacccccaatgcacagggctgcaacaccacgacttctgcccattctctccagtgtgtgtaacagggtcacaagaattcgacagccagatgctccaagagggtggcccaaggctatagcccctccttcaatattgacctaacgggggagaaaagatttagattgtttattcttctgtggacacagtttaaaatcttaaacttgtctttttcctcttaatgtatcagcatgctaccctttcaaactcaaattttcattttaactgcttaggaataaatttacacctttgtgaaaattcaaaaaaaaaaa

SEQ ID NO: 50 - protein, 556 aa, Homo sapiens
MEGPLSVFGDRSTGETIRSQNVMAAASIANIVKSSLGPVGLDKMLVDDIGDVTITNDGATILKLLEVEHPAAKVLCELADLQDKEVGDGTTSVVIIAAELLKNADELVKQKIHPTSVISGYRLACKEAVRYINENLIVNTDELGRDCLINAAKTSMSSKIIGINGDFFANMVVDAVLAIKYTDIRGQPRYPVNSVNILKAHGRSQMESMLISGYALNCVVGSQGMPKRIVNAKIACLDFSLQKTKMKLGVQVVITDPEKLDQIRQRESDITKERIQKILATGANVILTTGGIDDMCLKYFVEAGAMAVRRVLKRDLKRIAKASGATILSTLANLEGEETFEAAMLGQAEEVVQERICDDELILIKNTKARTSASIILRGANDFMCDEMERSLHDALCVVKRVLESKSVVPGGGAVEAALSIYLENYATSMGSREQLAIAEFARSLLVIPNTLAVNAAQDSTDLVAKLRAFHNEAQVNPERKNLKWIGLDLSNGKPRDNKQAGVFEPTIVKVKSLKFATEAAITILRIDDLIKLHPESKDDKHGSYEDAVHSGALND

SEQ ID NO: 51 - DNA, 1867 bp, Homo sapiens
cacatagctcagttcccataaaagggctggtttgccgcgtcggggagtggagtgggacaggtatataaaggaagtacagggcctggggaagaggccctgtctaggtagctggcaccaggagccgtgggcaagggaagaggccacaccctgccctgctctgctgcagccagaatgggtgtgaaggcgtctcaaacaggctttgtggtcctggtgctgctccagtgctgctctgcatacaaactggtctgctactacaccagctggtcccagtaccgggaaggcgatgggagctgcttcccagatgcccttgaccgcttcctctgtacccacatcatctacagctttgccaatataagcaacgatcacatcgacacctgggagtggaatgatgtgacgctctacggcatgctcaacacactcaagaacaggaaccccaacctgaagactctcttgtctgtcggaggatggaactttgggtctcaaagattttccaagatagcctccaacacccagagtcgccggactttcatcaagtcagtaccgccatttctgcgcacccatggctttgatgggctggaccttgcctggctctaccctggacggagagacaaacagcattttaccaccctaatcaaggaaatgaaggccgaatttataaaggaagcccagccagggaaaaagcagctcctgctcagcgcagcactgtctgcggggaaggtcaccattgacagcagctatgacattgccaagatatcccaacacctggatttcattagcatcatgacctacgattttcatggagcctggcgtgggaccacaggccatcacagtcccctgttccgaggtcaggaggatgcaagtcctgacagattcagcaacactgactatgctgtggggtacatgttgaggctgggggctcctgccagtaagctggtgatgggcatccccaccttcgggaggagcttcactctggcttcttctgagactggtgttggagccccaatctcaggaccgggaattccaggccggttcaccaaggaggcagggacccttgcctactatgagatctgtgacttcctccgcggagccacagtccatagaatcctcggccagcaggtcccctatgccaccaagggcaaccagtgggtaggatacgacgaccaggaaagcgtcaaaagcaaggtgcagtacctgaaggacaggcagctggcgggcgccatggtatgggccctggacctggatgacttccagggctccttctgcggccaggatctgcgcttccctctcaccaatgccatcaaggatgcactcgctgcaacgtagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgattaatggaaatgtttacagatccccaagcctggcaagggaatttcttcaactccctgccccccagccctccttatcaaaggacaccattttggcaagctctatcaccaaggagccaaacatcctacaagacacagtgaccatactaattataccccctgcaaagcccagcttgaaaccttcacttaggaacgtaatcgtgtcccctatcctacttccccttcctaattccacagctgctcaataaagtacaagagcttaacagtgaaaaaaaaaaaaaaaaaaaaaaaaaaaaaa

SEQ ID NO: 52 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRILGQQVPYATKGNQWVGYDDQESVKSKVQYLKDRQLAGAMVWALDLDDFQGSFCGQDLRFPLTNAIKDALAAT

SEQ ID NO: 53 - DNA, 2358 bp, Homo sapiens
aaactcacacaacaactcttccccgctgagaggagacagccagtgcgactccaccctccagctcgacggcagccgccccggccgacagccccgagacgacagcccggcgcgtcccggtccccacctccgaccaccgccagcgctccaggccccgccgctccccgctcgccgccaccgcgccctccgctccgcccgcagtgccaaccatgaccgccgccagtatgggccccgtccgcgtcgccttcgtggtcctcctcgccctctgcagccggccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccctgcgacccgcacaagggcctcttctgtgacttcggctccccggccaaccgcaagatcggcgtgtgcaccgccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcggcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcaaccccagacactggtttgaagaatgttaagacttgacagtggaactacattagtacacagcaccagaatgtatattaaggtgtggctttaggagcagtgggagggtaccagcagaaaggttagtatcatcagatagcatcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaatattgtgtgtgtgtgtgtgtgtgtatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaaaggggaggcatcagtgtccttggcaggctgatttctaggtaggaaatgtggtagcctcacttttaatgaacaaatggcctttattaaaaactgagtgactctatatagctgatcagttttttcacctggaagcatttgtttctactttgatatgactgtttttcggacagtttatttgttgagagtgtgaccaaaagttacatgtttgcacctttctagttgaaaataaagtgtatattttttctataaaaaaaaaaaaaaaaa

SEQ ID NO: 54 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 55 - DNA, 10943 bp, Homo sapiens
ttttttttgtagagactggggtctcactatgttgcccacgctggtctcaaactcctgggctcaagcgatcctcccgcttcagcctcccaaggtgctgggattacaggtggagccaccgcgcccggcatcgataatctgaatctctaacgagctcccgggcgacgttgatgcagctggtaccgggaccactcttcgaaaatcatcggtttagaggttttaattctgaatccattgcgaaaactgttgggcggacagagcactactaactcctaacttttctcagaaagctgggctctgctttcgccagcaacccggtccgaagtcgcgggcatattctgtctgaaatcgtgtgcaccgaaatccccgccttgcggtggaggctggcgctaggcggcctcagcctcggcctgctgcgctcaggaacccgcgccccggctcctcggcgatccattgctctttcctctggcgccggccgcaggcctcggtcacgcccccagcggcccgttggtttccgggtcccgcggggtgcccccgcccacacgctatgccttaaattgggccaggctgaggcgctgctgctggagcggccgatccgagacgtggctccctgggcggcagaaccatgttggacttcgcgatcttcgccgttaccttcttgctggcgttggtgggagccgtgctctacctctatccggcttccagacaagctgcaggaattccagggattactccaactgaagaaaaagatggtaatcttccagatattgtgaatagtggaagtttgcatgagttcctggttaatttgcatgagagatatgggcctgtggtctccttctggtttggcaggcgcctcgtggttagtttgggcactgttgatgtactgaagcagcatatcaatcccaataagacatcggacccttttgaaaccatgctgaagtcattattaaggtatcaatctggtggtggcagtgtgagtgaaaaccacatgaggaaaaaattgtatgaaaatggtgtgactgattctctgaagagtaactttgccctcctcctaaagctttcagaagaattattagataaatggctctcctacccagagacccagcacgtgcccctcagccagcatatgcttggttttgctatgaagtctgttacacagatggtaatgggtagtacatttgaagatgatcaggaagtcattcgcttccagaagaatcatggcacagtttggtctgagattggaaaaggctttctagatgggtcacttgataaaaacatgactcggaaaaaacaatatgaagatgccctcatgcaactggagtctgttttaaggaacatcataaaagaacgaaaaggaaggaacttcagtcaacatattttcattgactccttagtacaagggaaccttaatgaccaacagatcctagaagacagtatgatattttctctggccagttgcataataactgcaaaattgtgtacctgggcaatctgttttttaaccacctctgaagaagttcaaaaaaaattatatgaagagataaaccaagtttttggaaatggtcctgttactccagagaaaattgagcagctcagatattgtcagcatgtgctttgtgaaactgttcgaactgccaaactgactccagtttctgcccagcttcaagatattgaaggaaaaattgaccgatttattattcctagagagaccctcgtcctttatgcccttggtgtggtacttcaggatcctaatacttggccatctccacacaagtttgatccagatcggtttgatgatgaattagtaatgaaaactttttcctcacttggattctcaggcacacaggagtgtccagagttgaggtttgcatatatggtgaccacagtacttcttagtgtattggtgaagagactgcacctactttctgtggagggacaggttattgaaacaaagtatgaactggtaacatcatcaagggaagaagcttggatcactgtctcaaagagatattaaaattttatacatttaaaatcattgttaaattgattgaggaaaacaaccatttaaaaaaaatctatgttgaatccttttataaaccagtatcactttgtaatataaacacctatttgtacttaattttgtaaatttggatttttatatatcatattttcttaattcattgtacacatttgacttactgcacagtatattgatcattttaatgggaaactttagctttctactttttatttttgttttttcactttctatgccattatttttgtattctttttcttagtgtgagctctaaaatcaatgttcttgaaaaagaaattattttgcagaagttggggaatcatgtttgttgaatatgtataaaatagaaacataggctgggcgcggtggctcacacctgtaatccctacactttgggaggctgaggcaggtggatcacctgaggtccagagtttgagaccagtctggccaacatgatgaaaccccatctctactaaaaatacaaaacattggccgggagtggtggctcatgcctgtaatcccagcactttgggatgctgaggcgggtggatcacctgaggtcaggagtttgcgaccagcctggccaacatgatgaaaccctgtctctactaaaaatacaaaaaaattggctgggtgtggtggcccacacctgtaatcccagcactttgggaggtcgaggcgggtggatcacctgaggtccgaagttcgaggccagcctggccaacaggatgaaaccctgtctctattaaaaatacagaaaattggccgggtgcggtggctcacccctgtaatcccagtactttgggaggctgaggcgggtggagcacctgaggtcaggaattcgagatcagcctggccaacatggtgaaaccccatctctactgaaaaacacacacaaaaaaattagctgggcatggtggcacatgcctgtaatcccagctactcaggaggctgaggcaggagaatcatttgaacctgggaggcggagcttgcagtgagccgagattgcacccctgcactccagcctgggccacagagcaagactctgtctcaagaaaaacaaaaaaaaagataaatggacaaaagacatgaacaaacagcttttataggatagtatggctaacaaacttttagaaaagtgtttagtctcactagtaagtaacaaaatattggggcagggcacagtggctcatgcgtataatcctagaactttggaaggccaaagctggtggattgcttgagcccaggagtttgagaccagcccaggcaacattgggagaccctgcctctacaaatatacaaaaaaattactgggcatggtgacacacacctgtagtcccagctgttctgaaggctgaggtgggaggatcacttgagaccaggagaggtcaaggctgcagtgatctgtgatcacaccactgcactccagcctgggcaacagagcaaaaccctgcctttaaaaaaacaaaaaacaaaaaaggccgggctcggtggctcatgcctataatactttgggaggccgaggcaggtggatcaattgaggtcaggtgttcgagaccagccaggccaacttggtgaaaccccgtctctactaaaaatacaaaaaaaaaaaaaaaaaattagccagatgtggtggtgcacatttgtaatcccagctactcaggaggctgaggcaggataatcgcttgaacctgggaggcagaggttgcagtgagccaagatcgcaccactgcactccagcctgggtgtcgagtgaaactcattctcaaaaaaaaaaaaaaaaggaggtggggagagctatttcaagaatattttaagtaggctgggcacggtagctcatgcctgttatcctaacactttgggaagccgacatgggcagatcacgaggtcaagagatttgagaccatcctggccaacatggtgaaaccccatctctactaaaaatacaaaaaattatctggacatgctggcgtgcgcctgtagtctcagctacttgggaggctgaggcaggagaatcgcttgaacctgggaggcggaggttgcagtgagctgatgttgtgctagtgcactccagcctgggcgacagggctgagaccttgtctcaaaaaaaaaaggagtatcttaagtgtctttcaaaattacactttagcacttataaattttatatataatactttactgatagcattgtatattagtccaacttttttggctacactatttgtgaaggattcctttttttctttaagacagccactgtaggaaatatgccaagtacagatagtgaggaaagaaaggaagaaaaatgataattgtctgcaagtgggcttatttttaacactgctgaatttcatttgtaattttactttcaagaatatactctggtagactgaggcgggaggatcacttgagcccaggagttggaggcttcagtgagctgtgatcacaccactgccctcagccaaacctgagtgataaagtgagaccctatctcttaaaaaaaaaaatccctttgttgttgagttattttatgtcatttattggtaaatacctaacattttagacctagtcattaccagttaacatagaatttaattggatgcagtctgtatttttgaaaaaaaaaattttttttggaataacttaaattctaaaactttggccgggcgtgatggctcatgcctgtaatcccagcactttgggaggccgaggcaggcagatcacctgaggtcaggcgttcaagaccagcctgaccaacatggagaaaccccgtctctactaaaaatacaaaattagccaggcttggtggtgcatgcctgtaataccagctactcgggaggctgaagcaggagaactgcttgaacctgggaggcagaggttgcggtgagccaagatcgcgccattgcactccagcctgggcaacaagagtgaaactccgtctcaaaaaaaaaagaaaaattctaaaactttaagtgaaatttattatcccttcccacttattttctgtggttttgtgtgtatagagcatcttcctttgttacccaagtacctcgaatgggttggaaactggcggtagaaggttagagtagccaattgatatgtaaaccaagtgtcagatcagatgagatgaaagaggtggagtttaaagcaagtgagatatgataaacaatgatgaaagcaaaaatagggcagttgaaccagagtcagccagttggcataactgccaataaatgaggcagtgtagaataactaataatgattggaatcaaggaaaagagagcatagaggaatcagctaaattagtatgttttatccatagttacatgactaacaaatgattaaaaagaagttattctctagataattttttaaataaaagtactgttaatatataaactaagttattcattcactgtttttcttcacagtaagaatgacaaaaatctgtgttctttgtacttatcaagtgactcaaattttggccacttcactgtcactgatggcttatagaatgattctagcaggactcaagttattgaactatcaaatgggtttttaattgatattgaagtaagtaagttttgtagcttttggagtttttaaaaactacttcagtgattttgatttatattctttttgatagttcagaagtgtattgttaaaaaacataagcatggccgggcacagtggctcacacctgtaatcccagctctttgggaggccaaggcgggtaaatcacctgaggttgggagtttgagaccagcctgaccaacatggagaaaccctgtctctaccaaaaatacaaaattagccgggcatgatggagcatccctgtaatcctagctacttgagacgctgaggcaggaaaattgcttgaacccgtgaggcagaggttgcgatgagccgagatcgtgccattgcactccagcctgggcaataagagcaaaactccgtctcagaaaaaaaaaaaaaaaaaaaaaacggccaggcgaggtggctcacacctgtaatcccagcactttgggaggctgaggcgggcagatcacctgaagtcaggagttcaagaccagcctgaccaacatggagaaacttcatctctactaaaaatacaaaattagccgggcgtggtggcacatgcctttaatcccagctactggggaggctgaaacaggagaatcgcttgaacccagtaggggaaggttgtggtgagccaagattgcaccattgcactccagcctgggcaagaagagtgaaactctatccaaaaaaaaaaaaaaaccatacgtacataactttttttctttttcttttctttctttcttttcttttcttttttttttttgaggcacagtcttgctttgttgaccaagctggagtgcattggcatgatcacagcttactgtagcctccacctcctgggctccagcgatctcacctcagcctctagaatagctgggactacatatgcatgccaccatgcccagctgtttttttgtttgcttgtttttggttttgtttttttgagacagggtctccctgcgtcgcccagtcttgtctcaaactcctaggctcaagcgatcctcccacctgggcctcccaaagtgttgagattgcaggtgtgagccacggcacctggtccataaaacatttttttatgaactaaagagctctctcaaacttgtgctaacaaaatttaaggcaatattaattctgtgcatgtgtttgggaaaattaaaaaaacttacatagtgcttatttggcccacaaaagtacagttccattttttatataaatagattctacctaacttataacccctatgtcaaaatttgtttatcttaaaatagtttgtataaaaagtctttactgctttattatgaaataataatgtttattgtagaaaaatctaggaaaacacaaaaatgtcaaactcagttaacagtcatagtttcactatttgaagttaaccactgttaagattcttttttttttttttttttttttgttttgagactgagtcccgctgtctattgcccaggctggagtgccatggcaccatctcggctcactgcaacctctgcttcccaggttcaagtgatcctcccgcctcagcctcccaagtagctgggattgcaaacatgtgccaccacacctggctaatttttgtgtttttagtacagatggggtttcaccatgttggccaggctggcctcgaactcctcaacctcaagtagtctgcccaccttgacctcccaaagtgctgggattacaggcatggggcaccatgcccagccccactgttaagattctaatgtaggccgggcacagtgactcatgcctataactcaagcactttgggagttcaaggcggccagattgcttgagcttttagaagtttgagaccagcctgggcaacgtggcaaaagcttgtttctataaaaaaatataaaaacttagccagccttggtggcaccacctgcagtcccaactattcaggaggctgaggcgagaggatggcttgagctcaggaagtcaaggctacagtgagctgtggtagtgccactgcactccctcatggttgacagagcaagaccctgtctcaaaagattctaatatagatactctttttatgcatatgtaatttatatatattatacaaaatattatttgcatttaacatattctgaaccaatagtcttttctacaagcagaacattagtattcttgtcactctgaatgtaggcacagatttttgtcattctttatcttttttgtgtgtgtgtgacagagtctcactgtcaccaggctggaatgcagtggcgtgatctcggctcactgcaacctctgcctcccaggttcaagcgattctcttgcctcagccttttgagtaactggggttacaggcgcgtgccatcacacccagctcatttttgtatttttagtagagatggggttttaccgtgttggtcaggctggtcctgaactcttgaccttgtgatctgcccaactcagcctcccagagtgctgggattacaagcatgagccaccgcgctcggtccctttctttttatttatttatttattttgagatggagtctcgctgtgtcacccaggctggagtgtggtggcacgatcttggctcactgcaacccctgcctcccaggttcaagcaattctcctgcctcagcctcctgagtagctgagactacgggtatgtgccactacactcagctaatttttgtatttttttttagtagagacagggtttcaccatgttggccaggctggtcttgaactcctaacctcaggtgattcacaccccttggcctcctgaagtgctgggattacaggcatgagccactgtgcccggccatttttttgtgtttttagtggagacaggttttcaccatgttggccagtagtgtatttagttatttaaggagggctcaatcaacaggtttaaatggtgtgctattcataattattgcaaaaataggccgggcctgtaattccagcactttgggaggccaaggcaggtgaatcacttgaggccaggagtttgagatcagcctggccaacgtggtgaaactccatctctactaaaaataccaaaagtagcgggcgtggtgacaggtgcctctaatcccagctactcgggaggctgaggcaggagaatcgcttgaacccaagagtcggaggttgcagtgagccgagatcgcgccattgcactccagtgtgggtgacgagcgaaaatccgtctcaaccaagaaaaaagaaaaaaagaaaaagaaaacttaactggcctttggggcacatgcctgtaatcccagcttcttgggaggctgaggcatgagaaaaaaaataaacctgggaggtggaggttgcagtgagttgagattgtgccactgcactccagcttaggcaataaaacaagactgtctcaaaaaaaaaaaaaaaaaaaacttattgagagaataatataccactaatatttaaagaaatttttggctggtgccgtggctcacacctgtaatcccagcactttgggagtccaaagtgggcgtataacttgaggtcaggagttcaagaacagcctggccaacatggggaaaccgcatctctactaaaaatacaaaaattagtgggtgtggaggcgggcgaatgtaatcccagctagttgagaggctgaggcaggagaatgtctgggaggtggaggttgcagtgtgccgagatcgcaccactgtactccagcctaggtgacagagtgagactccataggtctcagccagactctggaggtctcagcccaggcttgagtgcagtggcatgatctgggctcactacaacctccatctccccagttcaagcgattctcctgcctcagcctcccgagtagctgcgactacaggcgagtgcctccatgcccagctaattttttgtatttttagtagagacggggtttcaccatgttagccaggatggtcttgatctcctgaccttgtgatctgtccaccgcggcttcccaaagtgctgggattacaggtgtgagccacagcgcccggccaaaaaaaggaatttttaagaggaaaaagaatgctaccaacctaaacacatttctgtgactgtttatatttttccctgttccacatacatacatttttacatagtacgttcattgcagcatgagttacttttcacttaataaattttaaacattttccagctgggtgtggtggctcatgcctgtaatcccaacacttggagaggccaagtcaggcttattgggtgagtccaggtgtttgagactagcctaggcaacatggcgaaactgcatctctacaaaaaatacaaaaattagccaggtgtgctggcgcacacctgtagtcccaactactcaggaggctgaggtgggaggattgcttgagcccaggaggtagaggttgcagtgagccatgatcatgccactgcactccagcctaggtgacaaagcacaaccctcaaataaataaataaatacatgttaaacatatacctttattacttcatttttgtaagagcttcagggaataggcttttttaaagggaatagacttatagatcaattttaatatttgacttatagaattttatgacactaaatggataattgtaaagtttgagttgggacccattttttaagacattcaggctataaacaaggtaataaatcacactcttccagttctactcccaatttaattggtttacagttcggtggctgtctttttttttttttttttttttttttgagaccgagtctccctctgtcaccaggctggagtgcagtggtgcaatctcagctcactgcaacctccccctctcaggttcaagtaattctcctgcctcagcctgccgagtagctgggactacagtcgcacgccaaccacccccagctaatttttttatttttagtagagacaggatttcaccatgtttgccaggatggtctcgatctcttgtgctcataatctacctacctcggcctcccaaagtgctcggattacagacgtgagccactgcgcccggcccgttggcttgctttctagatactgggaatagatcttgttatagtatgtgataaatagatcttgttatagtatgtgataaaatgccatgatcaaataaattacttatatcctgtttggtgatttttagaaaaacatatttatctacatctaaaatacttattttaattttaaacacttgatggtatgaaatgaaattttgttactttaccattgtaactatgtcataaagtcataaagtacctgtccccaaaaaggcaaccagtaaatatttttgagtaaatgaatgaaatctttattgccggtgttctgtgcacatagaataaaggaaattattaatgtta

SEQ ID NO: 56 - protein, 462 aa, Homo sapiens
MLDFAIFAVTFLLALVGAVLYLYPASRQAAGIPGITPTEEKDGNLPDIVNSGSLHEFLVNLHERYGPVVSFWFGRRLVVSLGTVDVLKQHINPNKTSDPFETMLKSLLRYQSGGGSVSENHMRKKLYENGVTDSLKSNFALLLKLSEELLDKWLSYPETQHVPLSQHMLGFAMKSVTQMVMGSTFEDDQEVIRFQKNHGTVWSEIGKGFLDGSLDKNMTRKKQYEDALMQLESVLRNIIKERKGRNFSQHIFIDSLVQGNLNDQQILEDSMIFSLASCIITAKLCTWAICFLTTSEEVQKKLYEEINQVFGNGPVTPEKIEQLRYCQHVLCETVRTAKLTPVSAQLQDIEGKIDRFIIPRETLVLYALGVVLQDPNTWPSPHKFDPDRFDDELVMKTFSSLGFSGTQECPELRFAYMVTTVLLSVLVKRLHLLSVEGQVIETKYELVTSSREEAWITVSKRY

SEQ ID NO: 57 - DNA, 3287 bp, Homo sapiens
gacaggaggaaacgcagcgccagcagcatctcatctaccctccttgacacctccccgtggctccagccagaccctagaggtcagccttgcggaccaacaggaggactcccagctttcccttttcaagaggtccccagacaccggccaccctcttccagcccctgcggccagtgcaaggaggcaccaatgctctgaggctgtcgcgtggtgcagcgtcgagcatcctcgccgaggtcctttctgctgcctgtcccgcctcaccccgctccatcacaccagctggccctctttgcttccttttcccagaatcgttaagccccgactcccactagcacctcgtaccaacctcgccccaccccatcctcctgccttcccgcgctccggtgtcccccgctgccatgagctcccccatcagcaagagccgctcgcttgccgccttcctgcagcagctgcgcagtccgaggcagcccccgagactggtgacatctacggcgtacacgtcccctcagccgcgagaggtgccagtctgcccgctgacagctggtggcgagactcagaacgcggccgccctgccgggccccaccagctggccactgctgggcagcctgctgcagattctctggaaagggggtctcaagaaacagcacgacaccctggtggagtaccacaagaagtatggcaagattttccgcatgaagttgggttcctttgagtcggtgcacctgggctcgccatgcctgctggaagcgctgtaccgcaccgagagcgcgtacccgcagcggctggagatcaaaccgtggaaggcctatcgcgactaccgcaaagaaggctacgggctgctgatcctggaaggggaagactggcagcgggtccggagtgcctttcaaaagaaactaatgaaaccaggggaagtgatgaagctggacaacaaaatcaatgaggtcttggccgattttatgggcagaatagatgagctctgtgatgaaagaggccacgttgaagacttgtacagcgaactgaacaaatggtcgtttgaaagtatctgcctcgtgttgtatgagaagagatttgggcttctccagaagaatgcaggggatgaagctgtgaacttcatcatggccatcaaaacaatgatgagcacgtttgggaggatgatggtcactccagtcgagctgcacaagagcctcaacaccaaggtctggcaggaccacactctggcctgggacaccattttcaaatcagtcaaagcttgtatcgacaaccggttagagaagtattctcagcagcctagtgcagatttcctttgtgacatttatcaccagaatcggctttcaaagaaagaattgtatgctgctgtcacagagctccagctggctgcggtggaaacgacagcaaacagtctaatgtggattctctacaatttatcccgtaatccccaagtgcaacaaaagcttcttaaggaaattcaaagtgtattacctgagaatcaggtgccacgggcagaagatttgaggaatatgccgtatttaaaagcctgtctgaaagaatctatgaggcttacgccgagtgtaccatttacaactcggactcttgacaaggcaacagttctgggtgaatatgctttacccaaaggaacagtgctcatgctaaatacccaggtgttgggatccagtgaagacaattttgaagattcaagtcagtttagacctgaacgttggcttcaggagaaggaaaaaattaatccttttgcgcatcttccatttggcgttggaaaaagaatgtgcattggtcgccgattagcagagcttcaactgcatttggctctttgttggattgtccgcaaatacgacatccaggccacagacaatgagcctgttgagatgctacactcaggcaccctggtgcccagccgggaactccccatcgcgttttgccagcgataatacgcctcagatggtggtatttgctaacatcatatccaactcagggaagcggactgagtgctgggatccaaggcattctacagggttcactgctggtttacacttcacctgtgtcagcaccatcttcaggtgcttagaatggcctgggagcctgttctgtcttgcatcttccatgacatgaaagggaggctggcacttgtcagtcaggtagaggttacaaaccgtttcaggccctgcctaccacattcactgtttgaatctttaattcccaagaataagtttacatttcacaatgaatgacctacaacagctaaattttctggggctgggagtaatactgacaatccatttactgtagctctgcttaatgtactacttaggaaaatgtccctgcttaataatgtaagccaagctaaatgatggttaaagttatcaggcctcccatgaaattgcgttcttcctgcattgaaataaaaacattattgggaaactagagaacacctctatttttaaaaggactttaacgaagtcaaacaacttataagactagtgattcactggggcattattttgttagaggaccttaaaattgtttattttttaaatgtgattcctttatggcattagggtaaagatgaagcaataatttttaaattgtgtatgtgcatatgaagcacagacatgcatgtgtgtgtgtgtctgtgtgtgtgtgtccgtgtatgtgtgtgtgggttctaatggtaatttgcctcagtcatttttttaatatttgcagtacttgatttaggatctgtggtgcagggcaatgtttcaaagtttagtcacagcttaaaaacattcagtgtgactttaatattataaaatgatttcccatgccataatttttctgtctattaaatgggacaagtgtaaagcatgcaaaagttagagatctgttatataacatttgttttgtgatttgaactcctaggaaaaatatgatttcataaatgtaaaatgcacagaaatgcatgcaatacttataagacttaaaaattgtgtttacagatggtttatttgtgcatatttttactactgcttttcctaaatgcatactgtatataattctgtgtatttgataaatatttcttcctacattatatttttagaatatttcagaaatatacatttatgtctttatattgtaataaatatgtacatatctaggtatatgctttctctctgctgtgaaattatttttagaattataaattcacgtcttgtcagatttcatctgtataccttcaaattctctgaaagtaaaaataaaagtttttaaatattaaaaaaaaaaaaaaaaaaaaa

SEQ ID NO: 58 - protein, 514 aa, Homo sapiens
MSSPISKSRSLAAFLQQLRSPRQPPRLVTSTAYTSPQPREVPVCPLTAGGETQNAAALPGPTSWPLLGSLLQILWKGGLKKQHDTLVEYHKKYGKIFRMKLGSFESVHLGSPCLLEALYRTESAYPQRLEIKPWKAYRDYRKEGYGLLILEGEDWQRVRSAFQKKLMKPGEVMKLDNKINEVLADFMGRIDELCDERGHVEDLYSELNKWSFESICLVLYEKRFGLLQKNAGDEAVNFIMAIKTMMSTFGRMMVTPVELHKSLNTKVWQDHTLAWDTIFKSVKACIDNRLEKYSQQPSADFLCDIYHQNRLSKKELYAAVTELQLAAVETTANSLMWILYNLSRNPQVQQKLLKEIQSVLPENQVPRAEDLRNMPYLKACLKESMRLTPSVPFTTRTLDKATVLGEYALPKGTVLMLNTQVLGSSEDNFEDSSQFRPERWLQEKEKINPFAHLPFGVGKRMCIGRRLAELQLHLALCWIVRKYDIQATDNEPVEMLHSGTLVPSRELPIAFCQR

SEQ ID NO: 59 - DNA, 914 bp, Homo sapiens
gcatggggaggggcggccctcaaacgggtcattgccattaatagagacctcaaacaccgcctgctaaaaatacccgactggaggagcataaaagcgcagccgagcccagcgccccgcacttttctgagcagacgtccagagcagagtcagccagcatgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaatacacgctgccccccggtgtggaccccacccaagtttcctcctccctgtcccctgagggcacactgaccgtggaggcccccatgcccaagctagccacgcagtccaacgagatcaccatcccagtcaccttcgagtcgcgggcccagcttgggggcccagaagctgcaaaatccgatgagactgccgccaagtaaagccttagcccggatgcccacccctgctgccgccactggctgtgcctcccccgccacctgtgtgttcttttgatacatttatcttctgtttttctcaaataaagttcaaagcaaccacctgtcaaaaaaaaaaaaaaaaaa

SEQ ID NO: 60 - protein, 205 aa, Homo sapiens
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK

SEQ ID NO: 61 - DNA, 3965 bp, Homo sapiens
gggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctggcaagatgaagctctccctggtggccgcgatgctgctgctgctcagcgcggcgcgggccgaggaggaggacaagaaggaggacgtgggcacggtggtcggcatcgacctggggaccacctactcctgcgtcggcgtgttcaagaacggccgcgtggagatcatcgccaacgatcagggcaaccgcatcacgccgtcctatgtcgccttcactcctgaaggggaacgtctgattggcgatgccgccaagaaccagctcacctccaaccccgagaacacggtctttgacgccaagcggctcatcggccgcacgtggaatgacccgtctgtgcagcaggacatcaagttcttgccgttcaaggtggttgaaaagaaaactaaaccatacattcaagttgatattggaggtgggcaaacaaagacatttgctcctgaagaaatttctgccatggttctcactaaaatgaaagaaaccgctgaggcttatttgggaaagaaggttacccatgcagttgttactgtaccagcctattttaatgatgcccaacgccaagcaaccaaagacgctggaactattgctggcctaaatgttatgaggatcatcaacgagcctacggcagctgctattgcttatggcctggataagagggagggggagaagaacatcctggtgtttgacctgggtggcggaaccttcgatgtgtctcttctcaccattgacaatggtgtcttcgaagttgtggccactaatggagatactcatctgggtggagaagactttgaccagcgtgtcatggaacacttcatcaaactgtacaaaaagaagacgggcaaagatgtcaggaaagacaatagagctgtgcagaaactccggcgcgaggtagaaaaggccaaacgggccctgtcttctcagcatcaagcaagaattgaaattgagtccttctatgaaggagaagacttttctgagaccctgactcgggccaaatttgaagagctcaacatggatctgttccggtctactatgaagcccgtccagaaagtgttggaagattctgatttgaagaagtctgatattgatgaaattgttcttgttggtggctcgactcgaattccaaagattcagcaactggttaaagagttcttcaatggcaaggaaccatcccgtggcataaacccagatgaagctgtagcgtatggtgctgctgtccaggctggtgtgctctctggtgatcaagatacaggtgacctggtactgcttgatgtatgtccccttacacttggtattgaaactgtgggaggtgtcatgaccaaactgattccaaggaacacagtggtgcctaccaagaagtctcagatcttttctacagcttctgataatcaaccaactgttacaatcaaggtctatgaaggtgaaagacccctgacaaaagacaatcatcttctgggtacatttgatctgactggaattcctcctgctcctcgtggggtcccacagattgaagtcacctttgagatagatgtgaatggtattcttcgagtgacagctgaagacaagggtacagggaacaaaaataagatcacaatcaccaatgaccagaatcgcctgacacctgaagaaatcgaaaggatggttaatgatgctgagaagtttgctgaggaagacaaaaagctcaaggagcgcattgatactagaaatgagttggaaagctatgcctattctctaaagaatcagattggagataaagaaaagctgggaggtaaactttcctctgaagataaggagaccatggaaaaagctgtagaagaaaagattgaatggctggaaagccaccaagatgctgacattgaagacttcaaagctaagaagaaggaactggaagaaattgttcaaccaattatcagcaaactctatggaagtgcaggccctcccccaactggtgaagaggatacagcagaaaaagatgagttgtagacactgatctgctagtgctgtaatattgtaaatactggactcaggaacttttgttaggaaaaaattgaaagaacttaagtctcgaatgtaattggaatcttcacctcagagtggagttgaaactgctatagcctaagcggctgtttactgcttttcattagcagttgctcacatgtctttgggtgggggggagaagaagaattggccatcttaaaaagcgggtaaaaaacctgggttagggtgtgtgttcaccttcaaaatgttctatttaacaactgggtcatgtgcatctggtgtaggaagttttttctaccataagtgacaccaataaatgtttgttatttacactggtctaatgtttgtgagaagcttctaattagatcaattacttattttaggaaatttaagactagatactcgtgtgtggggtgaggggagggagtatttggtatgttgggataaggaaacacttctatttaatgcttccagggatttttttttttttttttaaccctcctgggcccaagtgatccttccacctcagtctcccagctaattgagaccacaggcttgttaccaccatgctcggcttttgcattaatctaagaaaaggggagagaagttaatccacatctttactcaggcaaggggcatttcacagtgcccaagagtggggttttcttgaacatacttggtttcctatttccccttatctttctaaaactgcctttctggtggctttttttaaaattattactaatgatgcttttatagctgcttggattctctgagaaatgatggggagtgagtgatcactggtattaactttatacacttggatttcatttgtaactttaggatgtaaaggtatattgtgaaccctagctgtgtcagaatctccatccctgaaatttctcattagtggtactggggtgggatcttggatggtgacattgaaactacactaaatcccctcactatgaatgggttgttaaaggcaatggtttgtgtcaaaactggtttaggattacttagattgtgttcctgaagaaaagagtccaggtaaatggtatgatcaataaaggacaggctggtgctaacataaaatccaatattgtaatcctagcactttgggaggccaaggcgggtggatcacaaggtcaagagatagagaccatctttgccaacatggtgaaactccatctctactgaaaatacaaaaattagctgggcgtggtagtgcaagctgaaggctgaggcaggagaatcactcgaacccgggaggcagaggttgcagtgagccgagatcacaccactgtactccagcccggcactccagcctggcgacaagagtgagactccacctcaaaaaaaaaaaaaagaatccaatactgcccaaggataggtattttatagatgggcaactggctgaaaggttaattctctagggctagtagaactggatcccaacaccaaactcttaattagacctaggcctcagctgcactgcccgaaaagcatttgggcagaccctgagcagaatactggtctcaggccaagcccaatacagccattaaagatgacctacagtgctgtgtaccctggggcaatagggttaaatggtagttagcaactagggctagtcttcccttacctcaaaggctctcactaccgtggaccacctagtctgtaactctttctgaggagctgttactgaatattaaaaagatagacttcaactatg

SEQ ID NO: 62 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 63 - DNA, 7370 bp, Homo sapiens
ttttgtagataaatgtgaggattttctctaaatccctcttctgtttgctaaatctcactgtcactgctaaattcagagcagatagagcctgcgcaatggaataaagtcctcaaaattgaaatgtgacattgctctcaacatctcccatctctctggatttctttttgcttcattattcctgctaaccaattcattttcagactttgtacttcagaagcaatgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaagtatcagcccccatctaccaacaagaacacgaagtctcagagaaggaaaggaagtacatttgaagaacgcaagtagagggagtgcaggaaacaagaactacaggatgtaggaagaccctcctgaggagtgaagagtgacatgccaccgcaggatcctttgctctgcacgagttacctgttaaactttggaacacctaccaaaaaataagtttgataacatttaaaagatgggcgtttcccccaatgaaatacacaagtaaacattccaacattgtctttaggagtgatttgcaccttgcaaaaatggtcctggagttggtagattgctgttgatcttttatcaataatgttctatagaaaagaaaaaaaaaatatatatatatatatatcttagtccctgcctctcaagagccacaaatgcatgggtgttgtatagatccagttgcactaaattcctctctgaatcttggctgctggagccattcattcagcaaccttgtctaagtggtttatgaattgtttccttatttgcacttctttctacacaactcgggctgtttgttttacagtgtctgataatcttgttagtctatacccaccacctcccttcataacctttatatttgccgaatttggcctcctcaaaagcagcagcaagtcgtcaagaagcacaccaattctaacccacaagattccatctgtggcatttgtaccaaatataagttggatgcattttattttagacacaaagctttatttttccacatcatgcttacaaaaaagaataatgcaaatagttgcaactttgaggccaatcatttttaggcatatgttttaaacatagaaagtttcttcaactcaaaagagttccttcaaatgatgagttaatgtgcaacctaattagtaactttcctctttttattttttccatatagagcactatgtaaatttagcatatcaattatacaggatatatcaaacagtatgtaaaactctgttttttagtataatggtgctattttgtagtttgttatatgaaagagtctggccaaaacggtaatacgtgaaagcaaaacaataggggaagcctggagccaaagatgacacaaggggaagggtactgaaaacaccatccatttgggaaagaaggcaaagtccccccagttatgccttccaagaggaacttcagacacaaaagtccactgatgcaaattggactggcgagtccagagaggaaactgtggaatggaaaaagcagaaggctaggaattttagcagtcctggtttctttttctcatggaagaaatgaacatctgccagctgtgtcatggactcaccactgtgtgaccttgggcaagtcacttcacctctctgtgcctcagtttcctcatctgcaaaatgggggcaatatgtcatctacctacctcaaaggggtggtataaggtttaaaaagataaagattcagattttttttaccctgggttgctgtaagggtgcaacatcagggcgcttgagttgctgagatgcaaggaattctataaataacccattcatagcatagctagagattggtgaattgaatgctcctgacatctcagttcttgtcagtgaagctatccaaataactggccaactagttgttaaaagctaacagctcaatctcttaaaacacttttcaaaatatgtgggaagcatttgattttcaatttgattttgaattctgcatttggttttatgaatacaaagataagtgaaaagagagaaaggaaaagaaaaaggagaaaaacaaagagatttctaccagtgaaaggggaattaattactctttgttagcactcactgactcttctatgcagttactacatatctagtaaaacctcgtttaatactataaataatattctattcattttgaaaaacacaatgattccttcttttctaggcaatataaggaaagtgatccaaaatttgaaatattaaaataatatctaataaaaagtcacaaagttatcttctttaacaaactttactcttattcttagctgtatatacatttttttaaaagtttgttaaaatatgcttgactagagtttccagttgaaaggcaaaaacttccatcacaacaagaaatttcccatgcctgctcagaagggtagcccctagctctctgtgaatgtgttttatccattcaactgaaaattggtatcaagaaagtccactggttagtgtactagtccatcatagcctagaaaatgatccctatctgcagatcaagattttctcattagaacaatgaattatccagcattcagatctttctagtcaccttagaactttttggttaaaagtacccaggcttgattatttcatgcaaattctatattttacattcttggaaagtctatatgaaaaacaaaaataacatcttcagtttttctcccactgggtcacctcaaggatcagaggccaggaaaaaaaaaaaaaagactccctggatctctgaatatatgcaaaaagaaggccccatttagtggagccagcaatcctgttcagtcaacaagtattttaactctcagtccaacattatttgaattgagcacctcaagcatgcttagcaatgttctaatcactatggacagatgtaaaagaaactatacatcatttttgccctctgcctgttttccagacatacaggttctgtggaataagatactggactcctcttcccaagatggcacttctttttatttcttgtccccagtgtgtaccttttaaaattattccctctcaacaaaactttataggcagtcttctgcagacttaacgtgttttctgtcatagttagatgtgataattctaagagtgtctatgacttatttccttcacttaattctatccacagtcaaaaatcccccaaggaggaaagctgaaagatgcactgccatattatctttcttaactttttccaacacataatcctctccaactggattataaataaattgaaaataactcattataccaattcactattttattttttaatgaattaaaactagaaaacaaattgatgcaaaccctggaagtcagttgattactatatactacagcagaatgactcagatttcatagaaaggagcaaccaaaatgtcacaacccaaaactttacaagctttgcttcagaattagattgctttataattcttgaatgaggcaatttcaagatatttgtaaaagaacagtaaacattggtaagaatgagctttcaactcataggcttatttccaatttaattgaccatactggatacttaggtcaaatttctgttctctcttccccaaataatattaaagtattatttgaactttttaagatgaggcagttcccctgaaaaagttaatgcagctctccatcagaatccactcttctagggatatgaaaatctcttaacacccaccctacatacacagacacacacacacacacacacacacacacacacacacacattcaccctaaggatccaatggaatactgaaaagaaatcacttccttgaaaattttattaaaaaacaaacaaacaaacaaaaagcctgtccacccttgagaatccttcctctccttggaacgtcaatgtttgtgtagatgaaaccatctcatgctctgtggctccagggtttctgttactattttatgcacttgggagaaggcttagaataaaagatgtagcacattttgctttcccatttattgtttggccagctatgccaatgtggtgctattgtttctttaagaaagtacttgactaaaaaaaaaagaaaaaaagaaaaaaaagaaagcatagacatatttttttaaagtataaaaacaacaattctatagatagatggcttaataaaatagcattaggtctatctagccaccaccacctttcaactttttatcactcacaagtagtgtactgttcaccaaattgtgaatttgggggtgcaggggcaggagttggaaattttttaaagttagaaggctccattgttttgttggctctcaaacttagcaaaattagcaatatattatccaatcttctgaacttgatcaagagcatggagaataaacgcgggaaaaaagatcttataggcaaatagaagaatttaaaagataagtaagttccttattgatttttgtgcactctgctctaaaacagatattcagcaagtggagaaaataagaacaaagagaaaaaatacatagatttacctgcaaaaaatagcttctgccaaatcccccttgggtattctttggcatttactggtttatagaagacattctcccttcacccagacatctcaaagagcagtagctctcatgaaaagcaatcactgatctcatttgggaaatgttggaaagtatttccttatgagatgggggttatctactgataaagaaagaatttatgagaaattgttgaaagagatggctaacaatctgtgaagattttttgtttcttgtttttgttttttttttttttttactttatacagtctttatgaatttcttaatgttcaaaatgacttggttcttttcttctttttttatatcagaatgaggaataataagttaaacccacatagactctttaaaactataggctagatagaaatgtatgtttgacttgttgaagctataatcagactatttaaaatgttttgctatttttaatcttaaaagattgtgctaatttattagagcagaacctgtttggctctcctcagaagaaagaatctttccattcaaatcacatggctttccaccaatattttcaaaagataaatctgatttatgcaatggcatcatttattttaaaacagaagaattgtgaaagtttatgcccctcccttgcaaagaccataaagtccagatctggtaggggggcaacaacaaaaggaaaatgttgttgattcttggttttggattttgttttgttttcaatgctagtgtttaatcctgtagtacatatttgcttattgctattttaatattttataagaccttcctgttaggtattagaaagtgatacatagatatcttttttgtgtaatttctatttaaaaaagagagaagactgtcagaagctttaagtgcatatggtacaggataaagatatcaatttaaataaccaattcctatctggaacaatgcttttgttttttaaagaaacctctcacagataagacagaggcccaggggatttttgaagctgtctttattctgcccccatcccaacccagcccttattattttagtatctgcctcagaattttatagagggctgaccaagctgaaactctagaattaaaggaacctcactgaaaacatatatttcacgtgttccctctttttttttttcctttttgtgagatggggtctcgcactgtcccccaggctggagtgcagtggcatgatctcggctcactgcaacctccacctcctgggtttaagcgattctcctgcctcagcctcctgagtagctgggattacaggcacccaccactatgcccggctaattttttggatttttaatagagacggggttttaccatgttggccaggttggtctcaaactcctgaccttgtgatttgcccgcctcagcctcccaaattgctgggattacaggcatgagccaccacaccctgcccatgtgttccctcttaatgtatgattacatggatcttaaacatgatccttctctcctcattcttcaactatctttgatggggtctttcaaggggaaaaaaatccaagcttttttaaagtaaaaaaaaaaaaagagaggacacaaaaccaaatgttactgctcaactgaaatatgagttaagatggagacagagtttctcctaataaccggagctgaattacctttcactttcaaaaacatgaccttccacaatccttagaatctgcctttttttatattactgaggcctaaaagtaaacattactcattttattttgcccaaaatgcactgatgtaaagtaggaaaaataaaaacagagctctaaaatccctttcaagccacccattgaccccactcaccaactcatagcaaagtcacttctgttaatcccttaatctgattttgtttggatatttatcttgtacccgctgctaaacacactgcaggagggactctgaaacctcaagctgtctacttacatcttttatctgtgtctgtgtatcatgaaaatgtctattcaaaatatcaaaacctttcaaatatcacgcagcttatattcagtttacataaaggccccaaataccatgtcagatctttttggtaaaagagttaatgaactatgagaattgggattacatcatgtattttgcctcatgtatttttatcacacttataggccaagtgtgataaataaacttacagacactgaattaatttcccctgctactttgaaaccagaaaataatgactggccattcgttacatctgtcttagttgaaaagcatattttttattaaattaattctgattgtatttgaaattattattcaattcacttatggcagaggaatatcaatcctaatgacttctaaaaatgtaactaattgaatcattatcttacatttactgtttaataagcatattttgaaaatgtatggctagagtgtcataataaaatggtatatctttctttagtaattacattaaaattagtcatgtttgattaattagttc

SEQ ID NO: 64 - protein, 158 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGSTFEERK

SEQ ID NO: 65 - DNA, 5156 bp, Homo sapiens
ccgctaatgtaccatgccctggtgctggaaagtgcctgagccagctgccccagcggcctcagcactaccaagttggcacaaagctccccaaattcggaggggctcagggaaacgagtggaggggatgaggaggtgaggggtaaacccatcatttcagttggcatttgagcaggtgccatgctcagcggagatgaggctctcccatctgtaggggccgtattaacatgcacactctaaaagtgcccttcgtttctccagcctcagctttgtccctctcctcctccacgtcaacctggccagagggtctggacgccacagccagggcaccccctgctttggtggtgactgctaatattggccaggccggcggatcatcgtccaggcagtttcggcagagagccttgggcaccagtgactccccggtcctctttatccactgtccaggagctgcggggactgcgcagggactagagtacaggggccgaagagtcaccaccgagcttgtgtgggaggaggtggattccagcccccagccccagggctctgaatcgctgccagctcagccccctgcccagcctgccccacagcctgagccccagcaggccagagagcccagtcctgaggtgagctgctgtggcctgtggcccaggcgaccccagcgctcccagaactgaggctggcagccagccccagcctcagccccaactgcgaggcagagagacaccaatgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggcctgcctgccctcctgcgtgcccgccggggtcacgtgctcgccaaggagctcgaggcgttcagggaggccaaacgtcaccgtcccctgattgctctacccacccaagaccccgcccacgggggcgcccccccagagatggccagcaatcggaagtgagcaaaactgccgcaagtctgcagcccggcgccaccatcctgcagcctcctcctgaccacggacgtttccatcaggttccatcccgaaaatctctcggttccacgtccccctggggcttctcctgacccagtccccgtgccccgcctccccgaaacaggctactctcctcggccccctccatcgggctgaggaagcacagcagcatcttcaaacatgtacaaaatcgattggctttaaacacccttcacataccctccccccaaattatccccaattatccccacacataaaaaatcaaaacattaaactaacccccttcccccccccccacaacaaccctcttaaaactaattggctttttagaaacaccccacaaaagctcagaaattggctttaaaaaaaacaaccaccaaaaaaaatcaattggctaaaaaaaaaaagtattaaaaacgaattggctgagaaacaattggcaaaataaaggaatttggcactccccacccccctctttctcttctcccttggactttgagtcaaattggcctggacttgagtccctgaaccagcaaagagaaaagaaggaccccagaaatcacaggtgggcacgtcgctgctaccgccatctcccttctcacgggaattttcagggtaaactggccatccgaaaatagcaacaacccagactggctcctcactcccttttccatcactaaaaatcacagagcagtcagagggacccagtaagaccaaaggaggggaggacagagcatgaaaaccaaaatccatgcaaatgaaatgtaattggcacgaccctcacccccaaatcttacatctcaattcccatcctaaaaagcactcatactttatgcatccccgcagctacacacacacaacacacagcacacgcatgaacacagcacacacacgagcacagcacacacacaaacgcacagcacacacagcacacagatgagcacacagcacacacacaaacgcacagcacacacacgcacacacatgcacacacagcacacaaacgcacggcacacacacgcacacacatgcacacacagcacacacacaaacgcacagcacacacaaacgcacagcacacacgcacacacagcacacacacgagcacacagcacacaaacgcacagcacacgcacacacatgcacacacagcacacacactagcacacagcacacacacaaagacacagcacacacatgcacacacagcacacacacgcgaacacagcacacacgaacacagcacacacagcacacacacaaacacagcacacacatgcacacagcacacgcacacacagcacacacatgaacacagcacacagcacacacatgcacacacagcacacacgcatgcacagcacacatgaacacagcacacacacaaacacacagcacacacatgcacacacagcacacacactcatgcgcagcacatacatgaacacagctcacagcacacaaacacgcagcacacacgttgcacacgcaagcacccacctgcacacacacatgcgcacacacacgcacacccccacaaaattggatgaaaacaataagcatatctaagcaactacgatatctgtatggatcaggccaaagtcccgctaagattctccaatgttttcatggtctgagccccgctcctgttcccatctccactgcccctcggccctgtctgtgccctgcctctcagaggagggggctcagatggtgcggcctgagtgtgcggccggcggcatttgggatacacccgtagggtgggcggggtgtgtcccaggcctaattccatctttccaccatgacagagatgcccttgtgaggctggcctccttggcgcctgtccccacggcccccgcagcgtgagccacgatgctccccataccccacccattcccgatacaccttacttactgtgtgttggcccagccagagtgaggaaggagtttggccacattggagatggcggtagctgagcagacatgcccccacgagtagcctgactccctggtgtgctcctggaaggaagatcttggggacccccccaccggagcacacctagggatcatctttgcccgtctcctggggaccccccaagaaatgtggagtcctcgggggccgtgcactgatgcggggagtgtgggaagtctggcggttggaggggtgggtggggggcagtgggggctgggcggggggagttctggggtaggaagtggtcccgggagattttggatggaaaagtcaggaggattgacagcagacttgcagaattacatagagaaattaggaacccccaaatttcatgtcaattgatctattccccctctttgtttcttggggcatttttccttttttttttttttttgttttttttttacccctccttagctttatgcgctcagaaaccaaattaaaccccccccccatgtaacaggggggcagtgacaaaagcaagaacgcacgaagccagcctggagaccaccacgtcctgccccccgccatttatcgccctgattggattttgtttttcatctgtccctgttgcttgggttgagttgagggtggagcctcctggggggcactggccactgagcccccttggagaagtcagaggggagtggagaaggccactgtccggcctggcttctggggacagtggctggtccccagaagtcctgagggcggaggggggggttgggcagggtctcctcaggtgtcaggagggtgctcggaggccacaggagggggctcctggctggcctgaggctggccggaggggaaggggctagcaggtgtgtaaacagagggttccatcaggctggggcagggtggccgccttccgcacacttgaggaaccctcccctctccctcggtgacatcttgcccgcccctcagcaccctgccttgtctccaggaggtccgaagctctgtgggacctcttgggggcaaggtggggtgaggccggggagtagggaggtcaggcgggtctgagcccacagagcaggagagctgccaggtctgcccatcgaccaggttgcttgggccccggagcccacgggtctggtgatgccatagcagccaccaccgcggcgcctagggctgcggcagggactcggcctctgggaggtttacctcgcccccacttgtgcccccagctcagcccccctgcacgcagcccgactagcagtctagaggcctgaggcttctgggtcctggtgacggggctggcatgaccccgggggtcgtccatgccagtccgcctcagtcgcagagggtccctcggcaagcgccctgtgagtgggccattcggaacattggacagaagcccaaagagccaaattgtcacaattgtggaacccacattggcctgagatccaaaacgcttcgaggcaccccaaattacctgcccattcgtcaggacacccacccacccagtgttatattctgcctcgccggagtgggtgttcccgggggcacttgccgaccagccccttgcgtccccaggtttgcagctctcccctgggccactaaccatcctggcccgggctgcctgtctgacctccgtgcctagtcgtggctctccatcttgtctcctccccgtgtccccaatgtcttcagtggggggccccctcttgggtcccctcctctgccatcacctgaagacccccacgccaaacactgaatgtcacctgtgcctgccgcctcggtccaccttgcggcccgtgtttgactcaactcaactcctttaacgctaatatttccggcaaaatcccatgcttgggttttgtctttaaccttgtaacgcttgcaatcccaataaagcattaaaagtcatgaaaaaaaaaaaaaaaaaa

SEQ ID NO: 66 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 67 - DNA, 1439 bp, Homo sapiens
tgcggcggcgagggaggaggaagaagcggaggaggcggctcccgcgctcgcagggccgtgccacctgcccgcccgcccgctcgctcgctcgcccgccgcgccgcgctgccgaccgccagcatgctgccgagagtgggctgccccgcgctgccgctgccgccgccgccgctgctgccgctgctgccgctgctgctgctgctactgggcgcgagtggcggcggcggcggggcgcgcgcggaggtgctgttccgctgcccgccctgcacacccgagcgcctggccgcctgcgggcccccgccggttgcgccgcccgccgcggtggccgcagtggccggaggcgcccgcatgccatgcgcggagctcgtccgggagccgggctgcggctgctgctcggtgtgcgcccggctggagggcgaggcgtgcggcgtctacaccccgcgctgcggccaggggctgcgctgctatccccacccgggctccgagctgcccctgcaggcgctggtcatgggcgagggcacttgtgagaagcgccgggacgccgagtatggcgccagcccggagcaggttgcagacaatggcgatgaccactcagaaggaggcctggtggagaaccacgtggacagcaccatgaacatgttgggcgggggaggcagtgctggccggaagcccctcaagtcgggtatgaaggagctggccgtgttccgggagaaggtcactgagcagcaccggcagatgggcaagggtggcaagcatcaccttggcctggaggagcccaagaagctgcgaccaccccctgccaggactccctgccaacaggaactggaccaggtcctggagcggatctccaccatgcgccttccggatgagcggggccctctggagcacctctactccctgcacatccccaactgtgacaagcatggcctgtacaacctcaaacagtgcaagatgtctctgaacgggcagcgtggggagtgctggtgtgtgaaccccaacaccgggaagctgatccagggagcccccaccatccggggggaccccgagtgtcatctcttctacaatgagcagcaggaggctcgcggggtgcacacccagcggatgcagtagaccgcagccagccggtgcctggcgcccctgccccccgcccctctccaaacaccggcagaaaacggagagtgcttgggtggtgggtgctggaggattttccagttctgacacacgtatttatatttggaaagagaccagcaccgagctcggcacctccccggcctctctcttcccagctgcagatgccacacctgctccttcttgctttccccgggggaggaagggggttgtggtcggggagctggggtacaggtttggggagggggaagagaaatttttatttttgaacccctgtgtcccttttgcataagattaaaggaaggaaaagtaaa

SEQ ID NO: 68 - protein, 328 aa, Homo sapiens
MLPRVGCPALPLPPPPLLPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 69 - DNA, 2061 bp, Homo sapiens
gaagcctcaccaagcctctgcaatgaggttcttctgtgcacgttgctgcagctttgggcctgagatgccagctgtccagctgctgcttctggcctgcctggtgtgggatgtgggggccaggacagctcagctcaggaaggccaatgaccagagtggccgatgccagtataccttcagtgtggccagtcccaatgaatccagctgcccagagcagagccaggccatgtcagtcatccataacttacagagagacagcagcacccaacgcttagacctggaggccaccaaagctcgactcagctccctggagagcctcctccaccaattgaccttggaccaggctgccaggccccaggagacccaggaggggctgcagagggagctgggcaccctgaggcgggagcgggaccagctggaaacccaaaccagagagttggagactgcctacagcaacctcctccgagacaagtcagttctggaggaagagaagaagcgactaaggcaagaaaatgagaatctggccaggaggttggaaagcagcagccaggaggtagcaaggctgagaaggggccagtgtccccagacccgagacactgctcgggctgtgccaccaggctccagagaagtttctacgtggaatttggacactttggccttccaggaactgaagtccgagctaactgaagttcctgcttcccgaattttgaaggagagcccatctggctatctcaggagtggagagggagacaccggatgtggagaactagtttgggtaggagagcctctcacgctgagaacagcagaaacaattactggcaagtatggtgtgtggatgcgagaccccaagcccacctacccctacacccaggagaccacgtggagaatcgacacagttggcacggatgtccgccaggtttttgagtatgacctcatcagccagtttatgcagggctacccttctaaggttcacatactgcctaggccactggaaagcacgggtgctgtggtgtactcggggagcctctatttccagggcgctgagtccagaactgtcataagatatgagctgaataccgagacagtgaaggctgagaaggaaatccctggagctggctaccacggacagttcccgtattcttggggtggctacacggacattgacttggctgtggatgaagcaggcctctgggtcatttacagcaccgatgaggccaaaggtgccattgtcctctccaaactgaacccagagaatctggaactcgaacaaacctgggagacaaacatccgtaagcagtcagtcgccaatgccttcatcatctgtggcaccttgtacaccgtcagcagctacacctcagcagatgctaccgtcaactttgcttatgacacaggcacaggtatcagcaagaccctgaccatcccattcaagaaccgctataagtacagcagcatgattgactacaaccccctggagaagaagctctttgcctgggacaacttgaacatggtcacttatgacatcaagctctccaagatgtgaaaagcctccaagctgtacaggcaatggcagaaggagatgctcagggctcctggggggagcaggctgaagggagagccagccagccagggcccaggcagctttgactgctttccaagttttcattaatccagaaggatgaacatggtcaccatctaactattcaggaattgtagtctgagggcgtagacaatttcatataataaatatcctttatcttctgtcagcatttatgggatgtttaatgacatagttcaagttttcttgtgatttggggcaaaagctgtaaggcataatagtttcttcctgaaaaccattgctcttgcatgttacatggttaccacaagccacaataaaaagcataacttctaaaggaagcagaatagctcctctggccagcatcgaatataagtaagatgcatttactacagttggcttctaatgcttcagatagaatacagttgggtctcacataaccctttacattgtgaaataaaattttcttacccaaaaaaaaaaaaaaaaaa

SEQ ID NO: 70 - protein, 392 aa, Homo sapiens
MRFFCARCCSFGPEMPAVQLLLLACLVWDVGARTAQLRKANDQSGRCQYTFSVASPNESSCPEQSQAMSVIHNLQRDSSTQRLDLEATKARLSSLESLLHQLTLDQAARPQETQEGLQRELGTLRRERDQLETQTRELETAYSNLLRDKSVLEEEKKRLRQENENLARRLESSSQEVARLRRGQCPQTRDTARAVPPGSREVSTWNLDTLAFQELKSELTEVPASRILKESPSGYLRSGEGDTGCGELVWVGEPLTLRTAETITGKYGVWMRDPKPTYPYTQETTWRIDTVGTDVRQVFEYDLISQFMQGYPSKVHILPRPLESTGAVVYSGSLYFQGAESRTVIRYELNTETVKAEKEIPGAGYHGQFPYSWGGYTDIDLAVDEAGLWVIY

SEQ ID NO: 71 - DNA, 1574 bp, Homo sapiens
tcaccacggcggcagccctttaaacccctcacccagccagcgccccatcctgtctgtccgaacccagacacaagtcttcactccttcctgcgagccctgaggaagccttgtgagtgcattggctggggcttggagggaagttgggctggagctggacaggagcagtgggtgcatttcaggcaggctctcctgaggtcccaggcgccagctccagctccctggctagggaaacccaccctctcagtcagcatgggggcccaagctccaggcagggtgggctggatcactagcgtcctggatctctctcagactgggcagccccgggctcattgaaatgccccggatgacttggctagtgcagaggaattgatggaaaccaccggggtgagagggaggctccccatctcagccagccacatccacaaggtgtgtgtaagggtgcaggcgccggccggttaggccaaggctctactgtctgttgcccctccaggagaacttccaaggagctttccccagacatggccaacaagggtccttcctatggcatgagccgcgaagtgcagtccaaaatcgagaagaagtatgacgaggagctggaggagcggctggtggagtggatcatagtgcagtgtggccctgatgtgggccgcccagaccgtgggcgcttgggcttccaggtctggctgaagaatggcgtgattctgagcaagctggtgaacagcctgtaccctgatggctccaagccggtgaaggtgcccgagaacccaccctccatggtcttcaagcagatggagcaggtggctcagttcctgaaggcggctgaggactatggggtcatcaagactgacatgttccagactgttgacctctttgaaggcaaagacatggcagcagtgcagaggaccctgatggctttgggcagcttggcagtgaccaagaatgatgggcactaccgtggagatcccaactggtttatgaagaaagcgcaggagcataagagggaattcacagagagccagctgcaggagggaaagcatgtcattggccttcagatgggcagcaacagaggggcctcccaggccggcatgacaggctacggacgacctcggcagatcatcagttagagcggagagggctagccctgagcccggccctcccccagctccttggctgcagccatcccgcttagcctgcctcacccacacccgtgtggtaccttcagccctggccaagctttgaggctctgtcactgagcaatggtaactgcacctgggcagctcctccctgtgcccccagcctcagcccaacttcttacccgaaagcatcactgccttggcccctccctcccggctgcccccatcacctctactgtctcctccctgggctaagcaggggagaagcgggctgggggtagcctggatgtgggccaagtccactgtcctccttggcggcaaaagcccattgaagaagaaccagcccagcctgccccctatcttgtcctggaatatttttggggttggaactcaaaaaaaaaaaaaaaaaatcaatcttttctcaaaaaaaaaaaaaaaaaa

SEQ ID NO: 72 - protein, 201 aa, Homo sapiens
MANKGPSYGMSREVQSKIEKKYDEELEERLVEWIIVQCGPDVGRPDRGRLGFQVWLKNGVILSKLVNSLYPDGSKPVKVPENPPSMVFKQMEQVAQFLKAAEDYGVIKTDMFQTVDLFEGKDMAAVQRTLMALGSLAVTKNDGHYRGDPNWFMKKAQEHKREFTESQLQEGKHVIGLQMGSNRGASQAGMTGYGRPRQIIS

SEQ ID NO: 73 - DNA, 4048 bp, Homo sapiens
ggctgcctcgcaggggctgcgcgcagcggcaagaagtgtctgggctgggacggacaggagaggctgtcgccatcggcgtcctgtgcccctctgctccggcacggccctgtcgcagtgcccgcgctttccccggcgcctgcacgcggcgcgcctgggtaacatgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgcccgccacattggcaccgactgtgactccggcaaggtggacggtggcgacagcggctctggcgagcccccgcccagcccgacgcccggctccaccttgactcctccggccgtggggctcgtgcattcgggcttgctcataggcatctccatcgcgagcctgtgcctggtggtggcgcttttggcgctcctctgccacctgcgcaagaagcagggcgccgccagggccaagatggagtacaagtgcgcggccccttccaaggaggtagtgctgcagcacgtgcggaccgagcggacgccgcagagactctgagcggcctccgtccaggagcctggctccgtccaggagcctgtgcctcctcacccccagctttgctaccaaagcaccttagctggcattacagctggagaagaccctccccgcaccccccaagctgttttcttctattccatggctaactggcgagggggtgattagagggaggagaatgagcctcggcctcttccgtgacgtcactggaccactgggcaatgatggcaattttgtaacgaagacacagactgcgatttgtcccaggtcctcactaccgggcgcaggagggtgagcgttattggtcggcagccttctgggcagaccttgacctcgtgggctagggatgactaaaatatttattttttttaagtatttaggtttttgtttgtttcctttgttcttacctgtatgtctccagtatccactttgcacagctctccggtctctctctctctacaaactcccacttgtcatgtgacaggtaaactatcttggtgaatttttttttcctagccctctcacatttatgaagcaagccccacttattccccattcttcctagttttctcctcccaggaactgggccaactcacctgagtcaccctacctgtgcctgaccctacttcttttgctcttagctgtctgctcagacagaacccctacatgaaacagaaacaaaaacactaaaaataaaaatggccatttgctttttcaccagatttgctaatttatcctgaaatttcagattcccagagcaaaataattttaaacaaaggttgagatgtaaaaggtattaaattgatgttgctggactgtcatagaaattacacccaaagaggtatttatctttacttttaaacagtgagcctgaattttgttgctgttttgatttgtactgaaaaatggtaattgttgctaatcttcttatgcaatttccttttttgttattattacttatttttgacagtgttgaaaatgttcagaaggttgctctagattgagagaagagacaaacacctcccaggagacagttcaagaaagcttcaaactgcatgattcatgccaattagcaattgactgtcactgttccttgtcactggtagaccaaaataaaaccagctctactggtcttgtggaattgggagcttgggaatggatcctggaggatgcccaattagggcctagccttaatcaggtcctcagagaatttctaccatttcagagaggccttttggaatgtggcccctgaacaagaattggaagctgccctgcccatgggagctggttagaaatgcagaatcctaggctccaccccatccagttcatgagaatctatatttaacaagatctgcagggggtgtgtctgctcagtaatttgaggacaaccattccagactgcttccaattttctggaatacatgaaatatagatcagttataagtagcaggccaagtcaggcccttattttcaagaaactgaggaattttctttgtgtagctttgctctttggtagaaaaggctaggtacacagctctagacactgccacacagggtctgcaaggtctttggttcagctaagctaggaatgaaatcctgcttcagtgtatggaaataaatgtatcatagaaatgtaacttttgtaagacaaaggttttcctcttctattttgtaaactcaaaatatttgtacatagttatttatttattggagataatctagaacacaggcaaaatccttgcttatgacatcacttgtacaaaataaacaaataacaatgtgctctcgggttgtgtgtctgttcacttttcctccctcagtgccctcattttatgtcattaaatggggctcacaaaccatgcaaatgctatgagatgcatggagggctgccctgtaccccagcacttgtgttgtctggtggtggcaccatctctgattttcaaagctttttccagaggctattattttcactgtagaatgatttcatgctatctctgtgtgcacaaatatttattttctttctgtaaccataacaacttcatatatgaggacttgtgtctctgtgcttttaaatgcataaatgcattataggatcatttgttggaatgaattaaataaacccttcctggggcatctggcgaatcccaaaaaaaaaaaaaaaaa

SEQ ID NO: 74 - protein, 575 aa, Homo sapiens
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHSGLLIGISIASLCLVVALLALLCHLRKKQGAARAKMEYKCAAPSKEVVLQHVRTERTPQRL

SEQ ID NO: 75 - DNA, 5826 bp, Homo sapiens
gaggaggagacggcatccagtacagaggggctggacttggacccctgcagcagccctgcacaggagaagcggcatataaagccgcgctgcccgggagccgctcggccacgtccaccggagcatcctgcactgcagggccggtctctcgctccagcagagcctgcgcctttctgactcggtccggaacactgaaaccagtcatcactgcatctttttggcaaaccaggagctcagctgcaggaggcaggatggtctggaggctggtcctgctggctctgtgggtgtggcccagcacgcaagctggtcaccaggacaaagacacgaccttcgaccttttcagtatcagcaacatcaaccgcaagaccattggcgccaagcagttccgcgggcccgaccccggcgtgccggcttaccgcttcgtgcgctttgactacatcccaccggtgaacgcagatgacctcagcaagatcaccaagatcatgcggcagaaggagggcttcttcctcacggcccagctcaagcaggacggcaagtccaggggcacgctgttggctctggagggccccggtctctcccagaggcagttcgagatcgtctccaacggccccgcggacacgctggatctcacctactggattgacggcacccggcatgtggtctccctggaggacgtcggcctggctgactcgcagtggaagaacgtcaccgtgcaggtggctggcgagacctacagcttgcacgtgggctgcgacctcatagacagcttcgctctggacgagcccttctacgagcacctgcaggcggaaaagagccggatgtacgtggccaaaggctctgccagagagagtcacttcaggggtttgcttcagaacgtccacctagtgtttgaaaactctgtggaagatattctaagcaagaagggttgccagcaaggccagggagctgagatcaacgccatcagtgagaacacagagacgctgcgcctgggtccgcatgtcaccaccgagtacgtgggccccagctcggagaggaggcccgaggtgtgcgaacgctcgtgcgaggagctgggaaacatggtccaggagctctcggggctccacgtcctcgtgaaccagctcagcgagaacctcaagagagtgtcgaatgataaccagtttctctgggagctcattggtggccctcctaagacaaggaacatgtcagcttgctggcaggatggccggttctttgcggaaaatgaaacgtgggtggtggacagctgcaccacgtgtacctgcaagaaatttaaaaccatttgccaccaaatcacctgcccgcctgcaacctgcgccagtccatcctttgtggaaggcgaatgctgcccttcctgcctccactcggtggacggtgaggagggctggtctccgtgggcagagtggacccagtgctccgtgacgtgtggctctgggacccagcagagaggccggtcctgtgacgtcaccagcaacacctgcttggggccctccatccagacacgggcttgcagtctgagcaagtgtgacacccgcatccggcaggacggcggctggagccactggtcaccttggtcttcatgctctgtgacctgtggagttggcaatatcacacgcatccgtctctgcaactccccagtgccccagatggggggcaagaattgcaaagggagtggccgggagaccaaagcctgccagggcgccccatgcccaatcgatggccgctggagcccctggtccccgtggtcggcctgcactgtcacctgtgccggtgggatccgggagcgcacccgggtctgcaacagccctgagcctcagtacggagggaaggcctgcgtgggggatgtgcaggagcgtcagatgtgcaacaagaggagctgccccgtggatggctgtttatccaacccctgcttcccgggagcccagtgcagcagcttccccgatgggtcctggtcatgcggctcctgccctgtgggcttcttgggcaatggcacccactgtgaggacctggacgagtgtgccctggtccccgacatctgcttctccaccagcaaggtgcctcgctgtgtcaacactcagcctggcttccactgcctgccctgcccgccccgatacagagggaaccagcccgtcggggtcggcctggaagcagccaagacggaaaagcaagtgtgtgagcccgaaaacccatgcaaggacaagacacacaactgccacaagcacgcggagtgcatctacctgggccacttcagcgaccccatgtacaagtgcgagtgccagacaggctacgcgggcgacgggctcatctgcggggaggactcggacctggacggctggcccaacctcaatctggtctgcgccaccaacgccacctaccactgcatcaaggataactgcccccatctgccaaattctgggcaggaagactttgacaaggacgggattggcgatgcctgtgatgatgacgatgacaatgacggtgtgaccgatgagaaggacaactgccagctcctcttcaatccccgccaggctgactatgacaaggatgaggttggggaccgctgtgacaactgcccttacgtgcacaaccctgcccagatcgacacagacaacaatggagagggtgacgcctgctccgtggacattgatggggacgatgtcttcaatgaacgagacaattgtccctacgtctacaacactgaccagagggacacggatggtgacggtgtgggggatcactgtgacaactgccccctggtgcacaaccctgaccagaccgacgtggacaatgaccttgttggggaccagtgtgacaacaacgaggacatagatgacgacggccaccagaacaaccaggacaactgcccctacatctccaacgccaaccaggctgaccatgacagagacggccagggcgacgcctgtgaccctgatgatgacaacgatggcgtccccgatgacagggacaactgccggcttgtgttcaacccagaccaggaggacttggacggtgatggacggggtgatatttgtaaagatgattttgacaatgacaacatcccagatattgatgatgtgtgtcctgaaaacaatgccatcagtgagacagacttcaggaacttccagatggtccccttggatcccaaagggaccacccaaattgatcccaactgggtcattcgccatcaaggcaaggagctggttcagacagccaactcggaccccggcatcgctgtaggttttgacgagtttgggtctgtggacttcagtggcacattctacgtaaacactgaccgggacgacgactatgccggcttcgtctttggttaccagtcaagcagccgcttctatgtggtgatgtggaagcaggtgacgcagacctactgggaggaccagcccacgcgggcctatggctactccggcgtgtccctcaaggtggtgaactccaccacggggacgggcgagcacctgaggaacgcgctgtggcacacggggaacacgccggggcaggtgcgaaccttatggcacgaccccaggaacattggctggaaggactacacggcctataggtggcacctgactcacaggcccaagactggctacatcagagtcttagtgcatgaaggaaaacaggtcatggcagactcaggacctatctatgaccaaacctacgctggcgggcggctgggtctatttgtcttctctcaagaaatggtctatttctcagacctcaagtacgaatgcagagatatttaaacaagatttgctgcatttccggcaatgccctgtgcatgccatggtccctagacacctcagttcattgtggtccttgtggcttctctctctagcagcacctcctgtcccttgaccttaactctgatggttcttcacctcctgccagcaaccccaaacccaagtgccttcagaggataaatatcaatggaactcagagatgaacatctaacccactagaggaaaccagtttggtgatatatgagactttatgtggagtgaaaattgggcatgccattacattgctttttcttgtttgtttaaaaagaatgacgtttacatataaaatgtaattacttattgtatttatgtgtatatggagttgaagggaatactgtgcataagccattatgataaattaagcatgaaaaatattgctgaactacttttggtgcttaaagttgtcactattcttgaattagagttgctctacaatgacacacaaatcccattaaataaattataaacaagggtcaattcaaatttgaagtaatgttttagtaaggagagattagaagacaacaggcatagcaaatgacataagctaccgattaactaatcggaacatgtaaaacagttacaaaaataaacgaactctcctcttgtcctacaatgaaagccctcatgtgcagtagagatgcagtttcatcaaagaacaaacatccttgcaaatgggtgtgacgcggttccagatgtggatttggcaaaacctcatttaagtaaaaggttagcagagcaaagtgcggtgctttagctgctgcttgtgccgctgtggcgtcggggaggctcctgcctgagcttccttccccagctttgctgcctgagaggaaccagagcagacgcacaggccggaaaaggcgcatctaacgcgtatctaggctttggtaactgcggacaagttgcttttacctgatttgatgatacatttcattaaggttccagttataaatattttgttaatatttattaagtgactatagaatgcaactccatttaccagtaacttattttaaatatgcctagtaacacatatgtagtataatttctagaaacaaacatctaataagtatataatcctgtgaaaatatgaggcttgataatattaggttgtcacgatgaagcatgctagaagctgtaacagaatacatagagaataatgaggagtttatgatggaaccttaaatatataatgttgccagcgattttagttcaatatttgttactgttatctatctgctgtatatggaattcttttaattcaaacgctgaaaagaatcagcatttagtcttgccaggcacacccaataatcagtcatgtgtaatatgcacaagtttgtttttgtttttgttttttttgttggttggtttgtttttttgctttaagttgcatgatctttctgcaggaaatagtcactcatcccactccacataaggggtttagtaagagaagtctgtctgtctgatgatggatagggggcaaatctttttcccctttctgttaatagtcatcacatttctatgccaaacaggaacaatccataactttagtcttaatgtacacattgcattttgataaaattaattttgttgtttcctttgaggttgatcgttgtgttgttgttttgctgcactttttacttttttgcgtgtggagctgtattcccgagaccaacgaagcgttgggatacttcattaaatgtagcgactgtcaacagcgtgcaggttttctgtttctgtgttgtggggtcaaccgtacaatggtgtgggagtgacgatgatgtgaatatttagaatgtaccatattttttgtaaattatttatgtttttctaaacaaatttatcgtataggttgatgaaacgtcatgtgttttgccaaagactgtaaatatttatttatgtgttcacatggtcaaaatttcaccactgaaaccctgcacttagctagaacctcatttttaaagattaacaacaggaaataaattgtaaaaaaggttttctatacatgaaaaaaaaaaaaaaaaaa

SEQ ID NO: 76 - protein, 1172 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGSCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

SEQ ID NO: 77 - DNA, 1148 bp, Homo sapiens
tctctctcgcacacatacccacacacacacacacacacacacacgcgcgcgcgaaaacaatatctcatttcttcttcagggagcagctgtgaaggaaatcgggggaggaggatggacacaacatcccatctttgtgtttcgatacagactaagcttttaggccaaccctcctgactggatgggggcggcgggcgtggcatgcatgaaaagtaaacatcagagacctgaagaagcttataaaatagcttgggagaggccagtcaccaagacaggcatctcaaatcggctgattctgcatctggaaactgccttcatcttgaaagaaaagctccaggtcccttctccagccacccagccccaagatggtgatgctgctgctgctgctttccgcactggctggcctcttcggtgcggcagagggacaagcatttcatcttgggaagtgccccaatcctccggtgcaggagaattttgacgtgaataagtatctcggaagatggtacgaaattgagaagatcccaacaacctttgagaatggacgctgcatccaggccaactactcactaatggaaaacggaaagatcaaagtgttaaaccaggagttgagagctgatggaactgtgaatcaaatcgaaggtgaagccaccccagttaacctcacagagcctgccaagctggaagttaagttttcctggtttatgccatcggcaccgtactggatcctggccaccgactatgagaactatgccctcgtgtattcctgtacctgcatcatccaactttttcacgtggattttgcttggatcttggcaagaaaccctaatctccctccagaaacagtggactctctaaaaaatatcctgacttctaataacattgatgtcaagaaaatgacggtcacagaccaggtgaactgccccaagctctcgtaaccaggttctacagggaggctgcacccactccatgttacttctgcttcgctttcccctaccccccccccataaagacaaaccaatcaaccacgacaaaggaagttgacctgaacatgtaaccatgccctaccctgttaccttgctagctgcaaaataaacttgttgctgacctgctgtgctcgcagtagattccaagttaaaaaaaaaaaaaaaaaa

SEQ ID NO: 78 - protein, 189 aa, Homo sapiens
MVMLLLLLSALAGLFGAAEGQAFHLGKCPNPPVQENFDVNKYLGRWYEIEKIPTTFENGRCIQANYSLMENGKIKVLNQELRADGTVNQIEGEATPVNLTEPAKLEVKFSWFMPSAPYWILATDYENYALVYSCTCIIQLFHVDFAWILARNPNLPPETVDSLKNILTSNNIDVKKMTVTDQVNCPKLS

SEQ ID NO: 79 - DNA, 1629 bp, Homo sapiens
attcatgaaaatccactactccagacagacggctttggaatccaccagctacatccagctccctgaggcagagttgagaatggagagaatgttacctctcctggctctggggctcttggcggctgggttctgccctgctgtcctctgccaccctaacagcccacttgacgaggagaatctgacccaggagaaccaagaccgagggacacacgtggacctcggattagcctccgccaacgtggacttcgctttcagcctgtacaagcagttagtcctgaaggcccctgataagaatgtcatcttctccccactgagcatctccaccgccttggccttcctgtctctgggggcccataataccaccctgacagagattctcaaaggcctcaagttcaacctcacggagacttctgaggcagaaattcaccagagcttccagcacctcctgcgcaccctcaatcagtccagcgatgagctgcagctgagtatgggaaatgccatgtttgtcaaagagcaactcagtctgctggacaggttcacggaggatgccaagaggctgtatggctccgaggcctttgccactgactttcaggactcagctgcagctaagaagctcatcaacgactacgtgaagaatggaactagggggaaaatcacagatctgatcaaggaccttgactcgcagacaatgatggtcctggtgaattacatcttctttaaagccaaatgggagatgccctttgacccccaagatactcatcagtcaaggttctacttgagcaagaaaaagtgggtaatggtgcccatgatgagtttgcatcacctgactataccttacttccgggacgaggagctgtcctgcaccgtggtggagctgaagtacacaggcaatgccagcgcactcttcatcctccctgatcaagacaagatggaggaagtggaagccatgctgctcccagagaccctgaagcggtggagagactctctggagttcagagagataggtgagctctacctgccaaagttttccatctcgagggactataacctgaacgacatacttctccagctgggcattgaggaagccttcaccagcaaggctgacctgtcagggatcacaggggccaggaacctagcagtctcccaggtggtccataaggctgtgcttgatgtatttgaggagggcacagaagcatctgctgccacagcagtcaaaatcaccctcctttctgcattagtggagacaaggaccattgtgcgtttcaacaggcccttcctgatgatcattgtccctacagacacccagaacatcttcttcatgagcaaagtcaccaatcccaagcaagcctagagcttgccatcaagcagtggggctctcagtaaggaacttggaatgcaagctggatgcctgggtctctgggcacagcctggcccctgtgcaccgagtggccatggcatgtgtggccctgtctgcttatccttggaaggtgacagcgattccctgtgtagctctcacatgcacaggggcccatggactcttcagtctggagggtcctgggcctcctgacagcaataaataatttcgttggaaaaaaaaaaaaaaaaaaaaaaaaaaaaaaaaaaaaaaaa

SEQ ID NO: 80 - protein, 423 aa, Homo sapiens
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 81 - DNA, 4380 bp, Homo sapiens
ggggagcgccatccgctccacttccacctccacatcctccaccggccaaggtccccgccgctgcatccctcgcggcttccgctgcgctccgggccggagccgagccgcctgcgctgccacagcagccgcctccacacactcgcagacgctcacacgctctccctccctgttcccccgccccctccccagctccttgatctctgggtctgttttattactcctggtgcgagtcccgcggactccgcggcccgctatttgtcatcagctcgctctccattggcggggagcggagagcagcgaagaagggggtggggaggggaggggaagggaagggggtggaaactgcctggagccgtttctccgcgccgctgttggtgctgccgctgcctcctcctcctccgccgccgccgccgccgccgccgcctcctccggctcttcgctcggcccctctccgcctccatgtgccggatagcgggagcgctgcggaccctgctgccgctgctggcggccctgcttcaggcgtctgtagaggcttctggtgaaatcgcattatgcaagactggatttcctgaagatgtttacagtgcagtcttatcgaaggatgtgcatgaaggacagcctcttctcaatgtgaagtttagcaactgcaatggaaaaagaaaagtacaatatgagagcagtgagcctgcagattttaaggtggatgaagatggcatggtgtatgccgtgagaagctttccactctcttctgagcatgccaagttcctgatatatgcccaagacaaagagacccaggaaaagtggcaagtggcagtaaaattgagcctgaagccaaccttaactgaggagtcagtgaaggagtcagcagaagttgaagaaatagtgttcccaagacaattcagtaagcacagtggccacctacaaaggcagaagagagactgggtcatccctccaatcaacttgccagaaaactccaggggaccttttcctcaagagcttgtcaggatcaggtctgatagagataaaaacctttcactgcggtacagtgtaactgggccaggagctgaccagcctccaactggtatcttcattatcaaccccatctcgggtcagctgtcggtgacaaagcccctggatcgcgagcagatagcccggtttcatttgagggcacatgcagtagatattaatggaaatcaagtggagaaccccattgacattgtcatcaatgttattgacatgaatgacaacagacctgagttcttacaccaggtttggaatgggacagttcctgagggatcaaagcctggaacatatgtgatgaccgtaacagcaattgatgctgacgatcccaatgccctcaatgggatgttgaggtacagaatcgtgtctcaggctccaagcaccccttcacccaacatgtttacaatcaacaatgagactggtgacatcatcacagtggcagctggacttgatcgagaaaaagtgcaacagtatacgttaataattcaagctacagacatggaaggcaatcccacatatggcctttcaaacacagccacggccgtcatcacagtgacagatgtcaatgacaatcctccagagtttactgccatgacgttttatggtgaagttcctgagaacagggtagacatcatagtagctaatctaactgtgaccgataaggatcaaccccatacaccagcctggaacgcagtgtacagaatcagtggcggagatcctactggacggttcgccatccagaccgacccaaacagcaacgacgggttagtcaccgtggtcaaaccaatcgactttgaaacaaataggatgtttgtccttactgttgctgcagaaaatcaagtgccattagccaagggaattcagcacccgcctcagtcaactgcaaccgtgtctgttacagttattgacgtaaatgaaaacccttattttgcccccaatcctaagatcattcgccaagaagaagggcttcatgccggtaccatgttgacaacattcactgctcaggacccagatcgatatatgcagcaaaatattagatacactaaattatctgatcctgccaattggctaaaaatagatcctgtgaatggacaaataactacaattgctgttttggaccgagaatcaccaaatgtgaaaaacaatatatataatgctactttccttgcttctgacaatggaattcctcctatgagtggaacaggaacgctgcagatctatttacttgatattaatgacaatgcccctcaagtgttacctcaagaggcagagacttgcgaaactccagaccccaattcaattaatattacagcacttgattatgacattgatccaaatgctggaccatttgcttttgatcttcctttatctccagtgactattaagagaaattggaccatcactcggcttaatggtgattttgctcagcttaatttaaagataaaatttcttgaagctggtatctatgaagttcccatcataatcacagattcgggtaatcctcccaaatcaaatatttccatcctgcgcgtgaaggtttgccagtgtgactccaacggggactgcacagatgtggacaggattgtgggtgcggggcttggcaccggtgccatcattgccatcctgctctgcatcatcatcctgcttatccttgtgctgatgtttgtggtatggatgaaacgccgggataaagaacgccaggccaaacaacttttaattgatccagaagatgatgtaagagataatattttaaaatatgatgaagaaggtggaggagaagaagaccaggactatgacttgagccagctgcagcagcctgacactgtggagcctgatgccatcaagcctgtgggaatccgacgaatggatgaaagacccatccacgccgagccccagtatccggtccgatctgcagccccacaccctggagacattggggacttcattaatgagggccttaaagcggctgacaatgaccccacagctccaccatatgactccctgttagtgtttgactatgaaggcagtggctccactgctgggtccttgagctcccttaattcctcaagtagtggtggtgagcaggactatgattacctgaacgactgggggccacggttcaagaaacttgctgacatgtatggtggaggtgatgactgaacttcagggtgaacttggtttttggacaagtacaaacaatttcaactgatattcccaaaaagcattcagaagctaggctttaactttgtagtctactagcacagtgcttgctggaggctttggcataggctgcaaaccaatttgggctcagagggaatatcagtgatccatactgtttggaaaaacactgagctcagttacacttgaattttacagtacagaagcactgggattttatgtgcctttttgtacctttttcagattggaattagttttctgtttaaggctttaatggtactgatttctgaaacgataagtaaaagacaaaatattttgtggtgggagcagtaagttaaaccatgatatgcttcaacacgcttttgttacattgcatttgcttttattaaaatacaaaattaaacaaacaaaaaaactcatggagcgattttattatcttgggggatgagaccatgagattggaaaatgtacattacttctagttttagactttagtttgtttttttttttttcactaaaatcttaaaacttactcagctggttgcaaataaagggagttttcatatcaccaatttgtagcaaaattgaattttttcataaactagaatgttagacacattttggtcttaatccatgtacacttttttatttctgtatttttccacttcactgtaaaaatagtatgtgtacataatgttttattggcatagtctatggagaagtgcagaaacttcagaacatgtgtatgtattatttggactatggattcaggttttttgcatgtttatatctttcgttatggataaagtatttacaaaacagtgacatttgattcaattgttgagctgtagttagaatactcaatttttaatttttttaatttttttattttttattttctttttggtttggggagggagaaaagttcttagcacaaatgttttacataatttgtaccaaaaaaaaaaaaaaaggaaaggaaagaaaggggtggcctgacactggtggcactactaagtgtgtgtttttttaaaaaaaaaatggaaaaaaaaaagcttttaaactggagagacttctgacaacagctttgcctctgtattgtgtaccagaatataaatgatacacctctgaccccagcgttctgaataaaatgctaattttggatctggaaaaaaaaaaaaa

SEQ ID NO: 82 - protein, 906 aa, Homo sapiens
MCRIAGALRTLLPLLAALLQASVEASGEIALCKTGFPEDVYSAVLSKDVHEGQPLLNVKFSNCNGKRKVQYESSEPADFKVDEDGMVYAVRSFPLSSEHAKFLIYAQDKETQEKWQVAVKLSLKPTLTEESVKESAEVEEIVFPRQFSKHSGHLQRQKRDWVIPPINLPENSRGPFPQELVRIRSDRDKNLSLRYSVTGPGADQPPTGIFIINPISGQLSVTKPLDREQIARFHLRAHAVDINGNQVENPIDIVINVIDMNDNRPEFLHQVWNGTVPEGSKPGTYVMTVTAIDADDPNALNGMLRYRIVSQAPSTPSPNMFTINNETGDIITVAAGLDREKVQQYTLIIQATDMEGNPTYGLSNTATAVITVTDVNDNPPEFTAMTFYGEVPENRVDIIVANLTVTDKDQPHTPAWNAVYRISGGDPTGRFAIQTDPNSNDGLVTVVKPIDFETNRMFVLTVAAENQVPLAKGIQHPPQSTATVSVTVIDVNENPYFAPNPKIIRQEEGLHAGTMLTTFTAQDPDRYMQQNIRYTKLSDPANWLKIDPVNGQITTIAVLDRESPNVKNNIYNATFLASDNGIPPMSGTGTLQIYLLDINDNAPQVLPQEAETCETPDPNSINITALDYDIDPNAGPFAFDLPLSPVTIKRNWTITRLNGDFAQLNLKIKFLEAGIYEVPIIITDSGNPPKSNISILRVKVCQCDSNGDCTDVDRIVGAGLGTGAIIAILLCIIILLILVLMFVVWMKRRDKERQAKQLLIDPEDDVRDNILKYDEEGGGEEDQDYDLSQLQQPDTVEPDAIKPVGIRRMDERPIHAEPQYPVRSAAPHPGDIGDFINEGLKAADNDPTAPPYDSLLVFDYEGSGSTAGSLSSLNSSSSGGEQDYDYLNDWGPRFKKLADMYGGGDD

SEQ ID NO: 83 - DNA, 3063 bp, Homo sapiens
cgccggcggggaagatgaccgcgggcgccggcgtgctccttctgctgctctcgctctccggcgcgctccgggcccataatgaggatcttacaactagagagacctgcaaggctgggttctctgaagatgattacacggcattaatctcccaaaatattctagaaggggaaaagctacttcaagtcaagttcagcagctgtgtggggaccaaggggacacaatatgagaccaacagcatggacttcaaagttggggcagatgggacagtcttcgccacccgggagctgcaggtcccctccgagcaggtggcgttcacggtgactgcatgggacagccagacagcagagaaatgggacgccgtggtgcggttgctggtggcccagacctcgtccccgcactctggacacaagccgcagaaaggaaagaaggtcgtggctctggacccctctccgcctccgaaggacaccctgctgccgtggccccagcaccagaacgccaacgggctgaggcggcgcaaacgggactgggtcatcccgcccatcaacgtgcccgagaactcgcgcgggcccttcccgcagcagctcgtgaggatccggtccgacaaagacaatgacatccccatccggtacagcatcacgggagtgggcgccgaccagccccccatggaggtcttcagcattgactccatgtccggccggatgtacgtcacaaggcccatggaccgggaggagcacgcctcttaccacctccgagcccacgctgtggacatgaatggcaacaaggtggagaaccccatcgacctgtacatctacgtcatcgacatgaatgacaaccgccctgagttcatcaaccaggtctacaacggctccgtggacgagggctccaagccaggcacctacgtgatgaccgtcacggccaacgatgctgacgacagcaccacggccaacgggatggtgcggtaccggatcgtgacccagaccccacagagcccgtcccagaatatgttcaccatcaacagcgagactggagatatcgtcacagtggcggctggcctggaccgagagaaagttcagcagtacacagtcatcgttcaggccacagatatggaaggaaatctcaactatggcctctcaaacacagccacagccatcatcacggtgacagatgtgaatgacaacccgccagaatttaccgccagcacgtttgcaggggaggtccccgaaaaccgcgtggagaccgtggtcgcaaacctcacggtgatggaccgagatcagccccactctccaaactggaatgccgtttaccgcatcatcagtggggatccatccgggcacttcagcgtccgcacagaccccgtaaccaacgagggcatggtcaccgtggtgaaggcagtcgactacgagctcaacagagctttcatgctgacagtgatggtgtccaaccaggcgcccctggccagcggaatccagatgtccttccagtccacggcaggggtgaccatctccatcatggacatcaacgaggctccctacttcccctcaaaccacaagctgatccgcctggaggagggcgtgccccccggcaccgtgctgaccacgttttcagctgtggaccctgaccggttcatgcagcaggctgtgagatactcaaagctgtcagacccagcgagctggctgcacatcaatgccaccaacggccagatcaccacggcggcagtgctggaccgtgagtccctctacaccaaaaacaacgtctacgaggccaccttcctggcagctgacaatgggatacccccggccagcggcaccgggaccctccagatctatctcattgacatcaacgacaacgcccctgagctgctgcccaaggaggcgcagatctgcgagaagcccaacctgaacgccatcaacatcacggcggccgacgctgacgtcgaccccaacatcggcccctacgtcttcgagctgccctttgtcccggcggccgtgcggaagaactggaccatcacccgcctgaacggtgactatgcccaactcagcttgcgcatcctgtacctggaggccgggatgtatgacgtccccatcatcgtcacagactctggaaaccctcccctgtccaacacgtccatcatcaaagtcaaggtgtgcccatgtgatgacaacggggactgcaccaccattggcgcagtggcagcggctggtctgggcaccggtgccatcgtggccatcctcatctgcatcctcatcctgctgaccatggtcctgctgtttgtcatgtggatgaagcggcgagagaaggagcgccacacgaagcagctgctcattgaccccgaggacgacgtccgcgacaacatcctcaagtatgacgaggaaggcggtggcgaggaggaccaggactacgacctcagccagctgcagcagccggaagccatggggcacgtgccaagcaaagcccctggcgtgcgtcgcgtggatgagcggccggtgggcgctgagccccagtacccgatcaggcccatggtgccgcacccaggcgacatcggtgacttcatcaatgagggactccgcgctgctgacaacgaccccacggcacccccctatgactccctgctggtcttcgactacgaggggagcggctccaccgcaggctccgtcagctccctgaactcatccagttccggggaccaagactacgattacctcaacgactgggggcccagattcaagaagctggcggacatgtatggaggtggtgaagaggattgactgacctcgcatcttcggaccgaagtgagagccgtgctcggacgccggaggagcaggactgagcagaggcggccggtcttcccgactccctgcggctgtgtccttagtgctgttaggaggccccccaatccccacgttgagctgtctagcatgagcacccacccccacagcgccctgcacccggccgctgcccagcaccgcgctggctggcactgaaggacagcaagaggcactctgtcttcacttgaatttcctagaacagaagcactgtttttaaaaaaaaaaaaaaaaaaagaag

SEQ ID NO: 84 - protein, 916 aa, Homo sapiens
MTAGAGVLLLLLSLSGALRAHNEDLTTRETCKAGFSEDDYTALISQNILEGEKLLQVKFSSCVGTKGTQYETNSMDFKVGADGTVFATRELQVPSEQVAFTVTAWDSQTAEKWDAVVRLLVAQTSSPHSGHKPQKGKKVVALDPSPPPKDTLLPWPQHQNANGLRRRKRDWVIPPINVPENSRGPFPQQLVRIRSDKDNDIPIRYSITGVGADQPPMEVFSIDSMSGRMYVTRPMDREEHASYHLRAHAVDMNGNKVENPIDLYIYVIDMNDNRPEFINQVYNGSVDEGSKPGTYVMTVTANDADDSTTANGMVRYRIVTQTPQSPSQNMFTINSETGDIVTVAAGLDREKVQQYTVIVQATDMEGNLNYGLSNTATAIITVTDVNDNPPEFTASTFAGEVPENRVETVVANLTVMDRDQPHSPNWNAVYRIISGDPSGHFSVRTDPVTNEGMVTVVKAVDYELNRAFMLTVMVSNQAPLASGIQMSFQSTAGVTISIMDINEAPYFPSNHKLIRLEEGVPPGTVLTTFSAVDPDRFMQQAVRYSKLSDPASWLHINATNGQITTAAVLDRESLYTKNNVYEATFLAADNGIPPASGTGTLQIYLIDINDNAPELLPKEAQICEKPNLNAINITAADADVDPNIGPYVFELPFVPAAVRKNWTITRLNGDYAQLSLRILYLEAGMYDVPIIVTDSGNPPLSNTSIIKVKVCPCDDNGDCTTIGAVAAAGLGTGAIVAILICILILLTMVLLFVMWMKRREKERHTKQLLIDPEDDVRDNILKYDEEGGGEEDQDYDLSQLQQPEAMGHVPSKAPGVRRVDERPVGAEPQYPIRPMVPHPGDIGDFINEGLRAADNDPTAPPYDSLLVFDYEGSGSTAGSVSSLNSSSSGDQDYDYLNDWGPRFKKLADMYGGGEED

SEQ ID NO: 85 - DNA, 2875 bp, Homo sapiens
acttgcgctgtcactcagcctggacgcgcttcttcgggtcgcgggtgcactccggcccggctcccgcctcggccccgatggacgccgcgttcctcctcgtcctcgggctgttggcccagagcctctgcctgtctttgggggttcctggatggaggaggcccaccaccctgtacccctggcgccgggcgcctgccctgagccgcgtgcggagggcctgggtcatccccccgatcagcgtatccgagaaccacaagcgtctcccctaccccctggttcagatcaagtcggacaagcagcagctgggcagcgtcatctacagcatccagggacccggcgtggatgaggagccccggggcgtcttctctatcgacaagttcacagggaaggtcttcctcaatgccatgctggaccgcgagaagactgatcgcttcaggctaagagcgtttgccctggacctgggaggatccaccctggaggaccccacggacctggagattgtagttgtggatcagaatgacaaccggccagccttcctgcaggaggcgttcactggccgcgtgctggagggtgcagtcccaggcacctatgtgaccagggcagaggccacagatgccgacgaccccgagacggacaacgcagcgctgcggttctccatcctgcagcagggcagccccgagctcttcagcatcgacgagctcacaggagagatccgcacagtgcaagtggggctggaccgcgaggtggtcgcggtgtacaatctgaccctgcaggtggcggacatgtctggagacggcctcacagccactgcctcagccatcatcacccttgatgacatcaatgacaatgcccccgagttcaccagggatgagttcttcatggaggccatagaggccgtcagcggagtggatgtgggacgcctggaagtggaggacagggacctgccaggctccccaaactgggtggccaggttcaccatcctggaaggcgaccccgatgggcagttcaccatccgcacggaccccaagaccaacgagggtgttctgtccattgtgaaggccctggactatgagagctgtgaacactacgaactcaaagtgtcggtgcagaatgaggccccgctgcaggcggctgcccttagggctgagcggggccaggccaaggtccgcgtgcatgtgcaggacaccaacgagccccccgtgttccaggagaacccacttcggaccagcctagcagagggggcacccccaggcactctggtggccaccttctctgcccgggaccctgacacagagcagctgcagaggctcagctactccaaggactacgacccggaagactggctgcaagtggacgcagccactggccggatccagacccagcacgtgctcagcccggcgtcccccttcctcaagggcggctggtacagagccatcgtcctggcccaggatgacgcctcccagccccgcaccgccaccggcaccctgtccatcgagatcctggaggtgaacgaccatgcacctgtgctggccccgccgccgccgggcagcctgtgcagcgagccacaccaaggcccaggcctcctcctgggcgccacggatgaggacctgcccccccacggggcccccttccacttccagctgagccccaggctcccagagctcggccggaactggagcctcagccaggtcaacgtgagccacgcgcgcctgcggccgcgacaccaggtccccgaaggcctgcaccgcctcagcctgctgctccgggactcggggcagccgccccagcagcgcgagcagcctctgaacgtgaccgtgtgccgctgcggcaaggacggcgtctgcctgccgggggccgcagcgctgctggcggggggcacaggcctcagcctgggcgcactggtcatcgtgctggccagcgccctcctgctgctggtgctggtcctgctcgtggcactccgggcgcggttctggaagcagtctcggggcaaggggctgctgcacggcccccaggacgaccttcgagacaatgtcctcaactacgatgagcaaggaggcggggaggaggaccaggacgcctacgacatcagccagctgcgtcacccgacagcgctgagcctgcctctgggaccgccgccacttcgcagagatgccccgcagggccgcctgcacccccagccaccccgagtgctgcccaccagccccctggacatcgccgacttcatcaatgatggcttggaggctgcagatagtgaccccagtgtgccgccttacgacacagccctcatctatgactacgagggtgacggctcggtggcggggacgctgagctccatcctgtccagccagggcgatgaggaccaggactacgactacctcagagactgggggccccgcttcgcccggctggcagacatgtatgggcacccgtgcgggttggagtacggggccagatgggaccaccaggccagggagggtctttctcctggggcactgctacccagacacagaggccggacagcctgaccctggggcgcaactggacatgccactccccggcctcgtggcagtgatggcccctgcagaggcagcctgaggtcaccgggcccgacccccctgggcctggggcagcctccttcctgtaggcgagggcccaagtctgggggcagaacctgagtgtggatggggcggccaggaagaggccccttcctgccggggtgggaagagtttctctccatcggccccatgcgggtcacctccctagtcccacctttgcctcctaccagtgaacctcatctttgtatgaaagacagcaacctcctgggtaaatctgaatgaaaaacgtgctagtctctttcatgcaaaaaaaaaaaaaaaaa

SEQ ID NO: 86 - protein, 814 aa, Homo sapiens
MDAAFLLVLGLLAQSLCLSLGVPGWRRPTTLYPWRRAPALSRVRRAWVIPPISVSENHKRLPYPLVQIKSDKQQLGSVIYSIQGPGVDEEPRGVFSIDKFTGKVFLNAMLDREKTDRFRLRAFALDLGGSTLEDPTDLEIVVVDQNDNRPAFLQEAFTGRVLEGAVPGTYVTRAEATDADDPETDNAALRFSILQQGSPELFSIDELTGEIRTVQVGLDREVVAVYNLTLQVADMSGDGLTATASAIITLDDINDNAPEFTRDEFFMEAIEAVSGVDVGRLEVEDRDLPGSPNWVARFTILEGDPDGQFTIRTDPKTNEGVLSIVKALDYESCEHYELKVSVQNEAPLQAAALRAERGQAKVRVHVQDTNEPPVFQENPLRTSLAEGAPPGTLVATFSARDPDTEQLQRLSYSKDYDPEDWLQVDAATGRIQTQHVLSPASPFLKGGWYRAIVLAQDDASQPRTATGTLSIEILEVNDHAPVLAPPPPGSLCSEPHQGPGLLLGATDEDLPPHGAPFHFQLSPRLPELGRNWSLSQVNVSHARLRPRHQVPEGLHRLSLLLRDSGQPPQQREQPLNVTVCRCGKDGVCLPGAAALLAGGTGLSLGALVIVLASALLLLVLVLLVALRARFWKQSRGKGLLHGPQDDLRDNVLNYDEQGGGEEDQDAYDISQLRHPTALSLPLGPPPLRRDAPQGRLHPQPPRVLPTSPLDIADFINDGLEAADSDPSVPPYDTALIYDYEGDGSVAGTLSSILSSQGDEDQDYDYLRDWGPRFARLADMYGHPCGLEYGARWDHQAREGLSPGALLPRHRGRTA

SEQ ID NO: 87 - DNA, 2947 bp, Homo sapiens
ctcctcccgggcgggataattgaacggcgcggccctggcccagcgttggctgccgaggctcggccggagcgtggagcccgcgccgctgccccaggaccgcgcccgcgcctttgtccgccgccgcccaccgcccgtcgcccgccgcccatggagcgcgccgcgccgtcgcgccgggtcccgcttccgctgctgctgctcggcggccttgcgctgctggcggccggagtggacgcggatgtcctcctggaggcctgctgtgcggacggacaccggatggccactcatcagaaggactgctcgctgccatatgctacggaatccaaagaatgcaggatggtgcaggagcagtgctgccacagccagctggaggagctgcactgtgccacgggcatcagcctggccaacgagcaggaccgctgtgccacgccccacggtgacaacgccagcctggaggccacatttgtgaagaggtgctgccattgctgtctgctggggagggcggcccaggcccagggccagagctgcgagtacagcctcatggttggctaccagtgtggacaggtcttccgggcatgctgtgtcaagagccaggagaccggagatttggatgtcgggggcctccaagaaacggataagatcattgaggttgaggaggaacaagaggacccatatctgaatgaccgctgccgaggaggcgggccctgcaagcagcagtgccgagacacgggtgacgaggtggtctgctcctgcttcgtgggctaccagctgctgtctgatggtgtctcctgtgaagatgtcaatgaatgcatcacgggcagccacagctgccggcttggagaatcctgcatcaacacagtgggctctttccgctgccagcgggacagcagctgcgggactggctatgagctcacagaggacaatagctgcaaagatattgacgagtgtgagagtggtattcataactgcctccccgattttatctgtcagaatactctgggatccttccgctgccgacccaagctacagtgcaagagtggctttatacaagatgctctaggcaactgtattgatatcaatgagtgtttgagtatcagtgccccgtgccctatcgggcatacatgcatcaacacagagggctcctacacgtgccagaagaacgtgcccaactgtggccgtggctaccatctcaacgaggagggaacgcgctgtgttgatgtggacgagtgcgcgccacctgctgagccctgtgggaagggacatcgctgcgtgaactctcccggcagtttccgctgcgaatgcaagacgggttactattttgacggcatcagcaggatgtgtgtcgatgtcaacgagtgccagcgctaccccgggcgcctgtgtggccacaagtgcgagaacacgctgggctcctacctctgcagctgttccgtgggcttccggctctctgtggatggcaggtcatgtgaagacatcaatgagtgcagcagcagcccctgtagccaggagtgtgccaacgtctacggctcctaccagtgttactgccggcgaggctaccagctcagcgatgtggatggagtcacctgtgaagacatcgacgagtgcgccctgcccaccgggggccacatctgctcctaccgctgcatcaacatccctggaagcttccagtgcagctgcccctcgtctggctacaggctggcccccaatggccgcaactgccaagacattgatgagtgtgtgactggcatccacaactgctccatcaacgagacctgcttcaacatccagggcggcttccgctgcctggccttcgagtgccctgagaactaccgccgctccgcagccacgctccagcaggagaagacagacacggtccgctgcatcaagtcctgccgccccaacgatgtcacatgcgtgttcgaccccgtgcacaccatctcccacaccgtcatctcgctgcctaccttccgcgagttcacccgccctgaagagatcatcttcctccgggccatcacgccaccgcatcctgccagccaggctaacatcatcttcgacatcacggaagggaacctgcgggactcttttgacatcatcaagcgttacatggacggcatgaccgtgggtgtcgtgcgccaggtgcggcccatcgtgggcccatttcatgccgtcctgaagctggagatgaactatgtggtcgggggcgtggtctcccaccgaaatgttgtcaacgtccacatcttcgtctctgagtactggttctgagggctggtctgccgcacagccgcaggtgcacctccaggccaaatcattgctgccagtgactgtggtctgtacttgtttataccctcagacttttttaatgttaggtatttgtagcattaggccaacatgtattaagctgagccagatgaataagtccatctgatgtattttcggtgtttaaaaaatgagcccagttgctcaactgtttggttgaaaaccttgctcattttttaatgcgaaggctaagtgtcaccccctttctctgcctctggctgggccttgctaagggccaaggaaagaaagacattttttagggggcagccagtccaaatgccaaaagaagaccagttcttgccctgattgtatgaaatttgacattttggcactttttttttttttttggccaatcagattttctatgttctaaggacatggctgctgtagaatagcacagacgtggatgataaattatccccagaagcagcatgacagaatgcctcggggagcacttggaagggaaattgcagttctgttgaaatagaggaaaatcccttggtaaagacacagcctgttaggctcgtgtgggcctccagtatgttcaccaggggaatggctgggatttctcggcactctgcatcatccatcttttcttataggtgggaaaataaacaactttgtgatcctcctg

SEQ ID NO: 88 - protein, 703 aa, Homo sapiens
MERAAPSRRVPLPLLLLGGLALLAAGVDADVLLEACCADGHRMATHQKDCSLPYATESKECRMVQEQCCHSQLEELHCATGISLANEQDRCATPHGDNASLEATFVKRCCHCCLLGRAAQAQGQSCEYSLMVGYQCGQVFRACCVKSQETGDLDVGGLQETDKIIEVEEEQEDPYLNDRCRGGGPCKQQCRDTGDEVVCSCFVGYQLLSDGVSCEDVNECITGSHSCRLGESCINTVGSFRCQRDSSCGTGYELTEDNSCKDIDECESGIHNCLPDFICQNTLGSFRCRPKLQCKSGFIQDALGNCIDINECLSISAPCPIGHTCINTEGSYTCQKNVPNCGRGYHLNEEGTRCVDVDECAPPAEPCGKGHRCVNSPGSFRCECKTGYYFDGISRMCVDVNECQRYPGRLCGHKCENTLGSYLCSCSVGFRLSVDGRSCEDINECSSSPCSQECANVYGSYQCYCRRGYQLSDVDGVTCEDIDECALPTGGHICSYRCINIPGSFQCSCPSSGYRLAPNGRNCQDIDECVTGIHNCSINETCFNIQGGFRCLAFECPENYRRSAATLQQEKTDTVRCIKSCRPNDVTCVFDPVHTISHTVISLPTFREFTRPEEIIFLRAITPPHPASQANIIFDITEGNLRDSFDIIKRYMDGMTVGVVRQVRPIVGPFHAVLKLEMNYVVGGVVSHRNVVNVHIFVSEYWF

SEQ ID NO: 89 - DNA, 1542 bp, Homo sapiens
ggtcgctttaagaaaggagtagctgtaatctgaagcctgctggacgctggattagaaggcagcaaaaaaagctctgtgctggctggagccccctcagtgtgcaggcttagagggactaggctgggtgtggagctgcagcgtatccacaggccccaggatgcaggccctggtgctactcctctgcattggagccctcctcgggcacagcagctgccagaaccctgccagccccccggaggagggctccccagaccccgacagcacaggggcgctggtggaggaggaggatcctttcttcaaagtccccgtgaacaagctggcagcggctgtctccaacttcggctatgacctgtaccgggtgcgatccagcacgagccccacgaccaacgtgctcctgtctcctctcagtgtggccacggccctctcggccctctcgctgggagcggagcagcgaacagaatccatcattcaccgggctctctactatgacttgatcagcagcccagacatccatggtacctataaggagctccttgacacggtcactgccccccagaagaacctcaagagtgcctcccggatcgtctttgagaagaagctgcgcataaaatccagctttgtggcacctctggaaaagtcatatgggaccaggcccagagtcctgacgggcaaccctcgcttggacctgcaagagatcaacaactgggtgcaggcgcagatgaaagggaagctcgccaggtccacaaaggaaattcccgatgagatcagcattctccttctcggtgtggcgcacttcaaggggcagtgggtaacaaagtttgactccagaaagacttccctcgaggatttctacttggatgaagagaggaccgtgagggtccccatgatgtcggaccctaaggctgttttacgctatggcttggattcagatctcagctgcaagattgcccagctgcccttgaccggaagcatgagtatcatcttcttcctgcccctgaaagtgacccagaatttgaccttgatagaggagagcctcacctccgagttcattcatgacatagaccgagaactgaagaccgtgcaggcggtcctcactgtccccaagctgaagctgagttatgaaggcgaagtcaccaagtccctgcaggagatgaagctgcaatccttgtttgattcaccagactttagcaagatcacaggcaaacccatcaagctgactcaggtggaacaccgggctggctttgagtggaacgaggatggggcgggaaccacccccagcccagggctgcagcctgcccacctcaccttcccgctggactatcaccttaaccagcctttcatcttcgtactgagggacacagacacaggggcccttctcttcattggcaagattctggaccccaggggcccctaatatcccagtttaatattccaataccctagaagaaaacccgagggacagcagattccacaggacacgaaggctgcccctgtaaggtttcaatgcatacaataaaagagctttatccctaacttctgtta

SEQ ID NO: 90 - protein, 418 aa, Homo sapiens
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 91 - DNA, 2490 bp, Homo sapiens
ggcgcagcggctccctataagcagaggagctgtccgtgtgctgaaacggcccgagaagctcgcccggagaacggggaggaatatgctgtggagctcctctgccatataaacaaaaagaggaaatctttcaaacatggctgaagcaaagacccactggcttggagcagccctgtctcttatccctttaattttcctcatctctggggctgaagcagcttcatttcagagaaaccagctgcttcagaaagaaccagacctcaggttggaaaatgtccaaaagtttcccagtcctgaaatgatcagggctttggagtacatagaaaacctccgacaacaagctcataaggaagaaagcagcccagattataatccctaccaaggtgtctctgtcccccttcagcaaaaagaaaatggcgatgaaagccacttgcccgagagggattcactgagtgaagaagactggatgagaataatactcgaagctttgagacaggctgaaaatgagcctcagtctgcaccaaaagaaaataagccctatgccttgaattcagaaaagaactttccaatggacatgagtgatgattatgagacacagcagtggccagaaagaaagcttaagcacatgcaattccctcctatgtatgaagagaattccagggataacccctttaaacgcacaaatgaaatagtggaggaacaatatactcctcaaagccttgctacattggaatctgtcttccaagagctggggaaactgacaggaccaaacaaccagaaacgtgagaggatggatgaggagcaaaaactttatacggatgatgaagatgatatctacaaggctaataacattgcctatgaagatgtggtcgggggagaagactggaacccagtagaggagaaaatagagagtcaaacccaggaagaggtgagagacagcaaagagaatatagaaaaaaatgaacaaatcaacgatgagatgaaacgctcagggcagcttggcatccaggaagaagatcttcggaaagagagtaaagaccaactctcagatgatgtctccaaagtaattgcctatttgaaaaggttagtaaatgctgcaggaagtgggaggttacagaatgggcaaaatggggaaagggccaccaggctttttgagaaacctcttgattctcagtctatttatcagctgattgaaatctcaaggaatttacagatacccccagaagacttaattgagatgctcaaaactggggagaagccgaatggatcagtggaaccggagcgggagcttgaccttcctgttgacctagatgacatctcagaggctgacttagaccatccagacctgttccaaaataggatgctctccaagagtggctaccctaaaacacctggtcgtgctgggactgaggccctaccagacgggctcagtgttgaggatattttaaatcttttagggatggagagtgcagcaaatcagaaaacgtcgtattttcccaatccatataaccaggagaaagttctgccaaggctcccttatggtgctggaagatctagatcgaaccagcttcccaaagctgcctggattccacatgttgaaaacagacagatggcatatgaaaacctgaacgacaaggatcaagaattaggtgagtacttggccaggatgctagttaaataccctgagatcattaattcaaaccaagtgaagcgagttcctggtcaaggctcatctgaagatgacctgcaggaagaggaacaaattgagcaggccatcaaagagcatttgaatcaaggcagctctcaggagactgacaagctggccccggtgagcaaaaggttccctgtggggcccccgaagaatgatgataccccaaataggcagtactgggatgaagatctgttaatgaaagtgctggaatacctcaaccaagaaaaggcagaaaagggaagggagcatattgctaagagagcaatggaaaatatgtaagctgctttcattaattaccctactttcattcctcccaccccaagcaaatcccaacatttctcttcagtgtgttgacttctatcctgttaacactgtaatatctttaaatgatgtacaggcagatgaaaccaggtcactggggagtctgcttcatttcctctgagctgttatcttgtgtatggatatgtgtaaatgttatgactccttgataaaaaatttattatgtccattattcaagaaagatatctatgactgtgtttaatagtatatctaatggctgtggcattgttgatgctcacatatgataaaaaagtgtcctataattctattgaaagtttttaatatttattgaattattttgttactgtctgtagtgttttgtggagtactggaccaaaaaaataaagcattataaatatatagttttatttataaggccttttctattgtgtgttttactgttgattaataaatgttatttctggacaaaaaaaaaaaaaaaa

SEQ ID NO: 92 - protein, 617 aa, Homo sapiens
MAEAKTHWLGAALSLIPLIFLISGAEAASFQRNQLLQKEPDLRLENVQKFPSPEMIRALEYIENLRQQAHKEESSPDYNPYQGVSVPLQQKENGDESHLPERDSLSEEDWMRIILEALRQAENEPQSAPKENKPYALNSEKNFPMDMSDDYETQQWPERKLKHMQFPPMYEENSRDNPFKRTNEIVEEQYTPQSLATLESVFQELGKLTGPNNQKRERMDEEQKLYTDDEDDIYKANNIAYEDVVGGEDWNPVEEKIESQTQEEVRDSKENIEKNEQINDEMKRSGQLGIQEEDLRKESKDQLSDDVSKVIAYLKRLVNAAGSGRLQNGQNGERATRLFEKPLDSQSIYQLIEISRNLQIPPEDLIEMLKTGEKPNGSVEPERELDLPVDLDDISEADLDHPDLFQNRMLSKSGYPKTPGRAGTEALPDGLSVEDILNLLGMESAANQKTSYFPNPYNQEKVLPRLPYGAGRSRSNQLPKAAWIPHVENRQMAYENLNDKDQELGEYLARMLVKYPEIINSNQVKRVPGQGSSEDDLQEEEQIEQAIKEHLNQGSSQETDKLAPVSKRFPVGPPKNDDTPNRQYWDEDLLMKVLEYLNQEKAEKGREHIAKRAMENM

SEQ ID NO: 93 - DNA, 716 bp, Homo sapiens
aggctcagtataaatagcagccaccgctccctggcaggcagggacccgcagctcagctacagcacagatcaggtgaggagcacaccaaggagtgatttttaaaacttactctgttttctctttcccaacaagattatcatttcctttaaaaaaaatagttatcctggggcatacagccataccattctgaaggtgtcttatctcctctgatctagagagcaccatgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcagcgatgccagagagaatatccagagattctttggccatggtgcggaggactcgctggctgatcaggctgccaatgaatggggcaggagtggcaaagaccccaatcacttccgacctgctggcctgcctgagaaatactgagcttcctcttcactctgctctcaggagatctggctgtgaggccctcagggcagggatacaaagcggggagagggtacacaatgggtatctaataaatacttaagaggtggaatttgtggaaact

SEQ ID NO: 94 - protein, 122 aa, Homo sapiens
MKLLTGLVFCSLVLGVSSRSFFSFLGEAFDGARDMWRAYSDMREANYIGSDKYFHARGNYDAAKRGPGGAWAAEVISDARENIQRFFGHGAEDSLADQAANEWGRSGKDPNHFRPAGLPEKY

SEQ ID NO: 95 - DNA, 1651 bp, Homo sapiens
gacctagagaggtcccaggacacgccactgtcccgccttccccattgcccgccccactggccagtccccacgcccacacacccaaggctgccccatctggcgctgattatcctgctgctgccgccaccgctgctgctgctctgcaaaattcagctgctgcctctgtcttgaggaccccagcgcctttcccccggggccatgctgcctgcagccacagcctccctcctggggcccctcctcactgcctgcgccctgctgccttttgcccagggccagacccccaactacaccagacccgtgttcctgtgcggaggggatgtgaagggggaatcaggttacgtggcaagtgaggggttccccaacctctacccccctaataaggagtgcatctggaccataacggtccccgagggccagactgtgtccctctcattccgagtcttcgacctggagctgcaccccgcctgccgctacgatgctctggaggtcttcgctgggtctgggacttccggccagcggctcggacgcttttgtgggaccttccggcctgcgcccctagtcgcccccggcaaccaggtgaccctgaggatgacgacggatgagggcacaggaggacgaggcttcctgctctggtacagcgggcgggccacctcgggcactgagcaccaattttgcggggggcggctggagaaggcccagggaaccctgaccacgcccaactggcccgagtccgattaccccccgggcatcagctgttcctggcacatcatcgcgcccccggaccaggtcatcgcgctgaccttcgagaagtttgacctggagccggacacctactgccgctatgactcggtcagcgtgttcaacggagccgtgagcgacgactcccggaggctggggaagttctgcggcgacgcagtcccgggctccatctcctccgaagggaatgaactcctcgtccagttcgtctcagatctcagtgtcaccgctgatggcttctcagcctcctacaagaccctgccgcggggcactgccaaagaagggcaagggcccggccccaaacggggaactgagcctaaagtcaagctgccccccaagtcccaacctccggagaaaacagaggaatctccttcagcccctgatgcacccacctgcccaaagcagtgccgccggacaggcaccttgcagagcaacttctgtgccagcagccttgtggtgactgcgacagtgaagtccatggttcgggagccaggggagggccttgccgtgactgtcagtcttattggtgcttataaaactggaggactggacctgccttctccacccactggtgcctccctgaagttttacgtgccttgcaagcagtgcccccccatgaagaaaggagtcagttatctgctgatgggccaggtagaagagaacagaggccccgtccttcctccagagagctttgtggttctccaccggcccaaccaggaccagatcctcaccaacctaagcaagaggaagtgcccctctcaacctgtgcgggctgctgcgtcccaggactgagacgcaggccagccccggcccctagccctcaggccttctttcttatccaaataaatgtttcttaatgaggaaaaaaaaaaaaaaaaaaaaaaaaaaaaaaaaa

SEQ ID NO: 96 - protein, 449 aa, Homo sapiens
MLPAATASLLGPLLTACALLPFAQGQTPNYTRPVFLCGGDVKGESGYVASEGFPNLYPPNKECIWTITVPEGQTVSLSFRVFDLELHPACRYDALEVFAGSGTSGQRLGRFCGTFRPAPLVAPGNQVTLRMTTDEGTGGRGFLLWYSGRATSGTEHQFCGGRLEKAQGTLTTPNWPESDYPPGISCSWHIIAPPDQVIALTFEKFDLEPDTYCRYDSVSVFNGAVSDDSRRLGKFCGDAVPGSISSEGNELLVQFVSDLSVTADGFSASYKTLPRGTAKEGQGPGPKRGTEPKVKLPPKSQPPEKTEESPSAPDAPTCPKQCRRTGTLQSNFCASSLVVTATVKSMVREPGEGLAVTVSLIGAYKTGGLDLPSPPTGASLKFYVPCKQCPPMKKGVSYLLMGQVEENRGPVLPPESFVVLHRPNQDQILTNLSKRKCPSQPVRAAASQD

SEQ ID NO: 97 - DNA, 32 bp, Artificial Sequence
  primer
aagcttccaccatgcacagctttcctccactg

SEQ ID NO: 98 - DNA, 28 bp, Artificial Sequence
  primer2
ggccggcctcaatttttcctgcagttga